(19) United States
(12) Patent Application Publication
(10) Pub. No.: US 2026/0085107 A1
(43) Pub. Date: Mar. 26, 2026

(54) PRODUCTION OF PROTEINS, INCLUDING SECRETED PROTEINS

(71) Applicant: Ginkgo Bioworks, Inc., Boston, MA (US)

(72) Inventors: Sudeep Agarwala, Cambridge, MA (US); Ruchita Anil Chawla, Munich (DE); Aaron Zev Berman, Boston, MA (US); Ishaan Joshan Dev, Arcadia, CA (US); Brett Philip Irwin, Wakefield, MA (US); Roy Marella, Boston, MA (US); Elzabeth Lauren Meier, Somerville, MA (US); Cameron Nowack, Cambridge, MA (US); Nathan W. Schmidt, Milton, MA (US); Sneha Srikrishnan, Walpole, MA (US)

(73) Assignee: Ginkgo Bioworks, Inc., Boston, MA (US)

(21) Appl. No.: 19/110,994

(22) PCT Filed: Sep. 13, 2023
(86) PCT No.: PCT/US2023/074066

§ 371 Date: Mar. 12, 2025

Related U.S. Application Data
(60) Provisional application No. 63/405,933, filed on Sep. 13, 2022.

(51) Int. Cl. C07K 14/79; C07K 14/47; C12N 9/90; C12N 15/62; C12N 15/81; C12R 1/685; C12R 1/84

(52) U.S. Cl. CPC C07K 14/79; C07K 14/4702; C07K 14/4728; C12N 9/90; C12N 15/625; C12N 15/815; C07K 2319/036; C12R 2001/685; C12R 2001/84; C12Y 503/04001

(57) ABSTRACT

This disclosure provides expression systems comprising secretion signals that promote production and/or secretion of proteins of interest, as well as one or more polynucleotides encoding chaperone proteins (e.g., CRT and/or PDIA3) which, as demonstrate herein, enhance production and/or secretion of proteins. Moreover, genetically modified host cells comprising these expression systems are capable of producing high levels of protein of interest, such as bovine lactoferrin (bLF), bovine lactoglobulin (bLG), or ovalbumin (Ova).

### CROSS-REFERENCE TO RELATED APPLICATIONS

This application claims the benefit under 35 U.S.C. § 119(e) of U.S. Provisional Application No. 63/405,933, filed Sep. 13, 2022, the entire contents of which is hereby incorporated by reference in its entirety.

### REFERENCE TO AN ELECTRONIC SEQUENCE LISTING

The contents of the electronic sequence listing (G091970103WO00-SEQ-ROS.xml; Size: 335,839 bytes; and Date of Creation: Sep. 13, 2023) is herein incorporated by reference in its entirety.

### FIELD OF INVENTION

The present disclosure relates to genetically modified host cells, expression systems, and uses thereof for the high production of proteins, including globular and secreted proteins.

### BACKGROUND

Certain host cells have been used in the production of bioproducts (e.g., proteins, nucleic acids, small molecules, etc.) due, in part, to the strong and regulatable characteristics of their native promoters and expression systems. Using host cells to produce a bioproduct (as opposed to isolating a bioproduct from a natural source) can be commercially advantageous. It would be advantageous to provide improved host cells capable of high expression of various bioproducts.

### SUMMARY

This disclosure provides expression systems comprising novel secretion signals and/or one or more polynucleotides encoding a CRT protein and/or a PDIA3 protein which, as demonstrated herein, enhance production and/or secretion of proteins. Moreover, this disclosure demonstrates that genetically modified host cells comprising these expression systems are capable of producing high levels of a protein of interest, such as bovine lactoferrin (bLF), bovine lactoglobulin (bLG), or ovalbumin (Ova).

In some aspects, the disclosure relates to polypeptides comprising a secretion signal.

In some embodiments, a secretion signal comprises, from N-terminus to C-terminus: (a) a pre-sequence comprising the structure of M-A′-Q-B′-L-C′-L-D′-LL-E′ (SEQ ID NO: 173), wherein M is methionine, Q is glutamine, and L is leucine, and A′ is 0-4 amino acids in length, B′ is 0-2 amino acids in length, C′ is 1-6 amino acids in length, D′ is 4 amino acids in length, and E′ is 5 amino acids in length, wherein each amino acid of A′, B′, C′, D′ and E′ is chosen from any amino acid; and (b) a pro-sequence comprising an amino acid sequence having at least 80% identity to the amino acid sequence: APANTTTEDETAQIPAEAVIDYSDLEGDFDAAALPLSNSTNNGLSSTNTTIASIAAKEE GVSLDKR (SEQ ID NO: 61). In some embodiments, A′ is 0 or 4 amino acids in length. In some embodiments, C′ is 1, 2 or 6 amino acids in length.

In some embodiments, a secretion signal comprises, from N-terminus to C-terminus: (a) a pre-sequence comprising the amino acid sequence LXXXXLL (SEQ ID NO: 15), wherein X is chosen from any amino acid; and (b) a pro-sequence comprising an amino acid sequence having at least 80% identity to the amino acid sequence: APANTTTEDETAQIPAEAVIDYSDLEGDFDAAALPLSNSTNNGLSSTNTTIASIAAKEE GVSLDKR (SEQ ID NO: 61). In some embodiments, no more than 10 amino acids separate the amino acid sequence LXXXXLL (SEQ ID NO: 15) of the pre-sequence from the amino acid sequence having at least 80% identity to the amino acid sequence APANTTTEDETAQIPAEAVIDYSDLEGDFDAAALPLSNSTNNGLSSTNTTIASIAAKEE GVSLDKR (SEQ ID NO: 61) of the pro-sequence.

In some embodiments, a secretion signal comprises, from N-terminus to C-terminus: (a) a pre-sequence comprising the amino acid sequence LXXXXLL (SEQ ID NO: 15), wherein X is chosen from any amino acid; and (b) a pro-sequence comprising the amino acid sequence: APX³NX⁵TX⁷EX⁹EX¹¹X¹²QX¹⁴PAEAX¹⁹X²⁰X²¹YX²³X²⁴X²⁵EGDX²⁹DX³¹AX³³LPX³⁶X³⁷X³⁸STNX⁴²GX⁴⁴X⁴⁵X⁴⁶X⁴⁷NTTX⁵¹ASIAAKEEGVSLDKR (SEQ ID NO: 82), wherein: X³ is valine (V) or alanine (A); X⁵ is threonine (T) or alanine (A); X⁷ is threonine (T) or alanine (A); X⁹ is aspartic acid (D) or glycine (G); X¹¹ is threonine (T) or alanine (A); X¹² is threonine (T) or alanine (A); X¹⁴ is isoleucine (I) or threonine (T); X¹⁹ is valine (V) or alanine (A); X²⁰ is isoleucine (I) or alanine (A); X²¹ is glycine (G), aspartic acid (D), or threonine (T); X²³ is leucine (L), serine (S), or arginine (R); X²⁴ is aspartic acid (D) or glycine (G); X²⁵ is leucine (L) or serine (S); X²⁹ is phenylalanine (F), serine (S), or valine (V); X³¹ is valine (V) or alanine (A); X³³ is valine (V) or alanine (A); X³⁶ is phenylalanine (F) or leucine (L); X³⁷ serine (S) or proline (P); X³⁸ is asparagine (N), serine (S), or aspartic acid (D); X⁴² is asparagine (N) or aspartic acid (D); X⁴ is leucine (L) or serine (S); X⁴⁵ is leucine (L) or serine (S); X⁴⁶ is phenylalanine (F) or serine (S); X⁴⁷ is isoleucine (I) or threonine (T); and X⁵¹ is isoleucine (I) or threonine (T). In some embodiments, no more than 10 amino acids separate the amino acid sequence LXXXXLL (SEQ ID NO: 15) of the pre-sequence from the amino acid sequence APX³NX⁵TX⁷EX⁹EX¹¹X¹²QX¹⁴PAEAX¹⁹X²⁰X²¹YX²³X²⁴X²⁵EGDX²⁹DX³¹AX³³LPX³⁶X³⁷X³⁸STNX⁴²GX⁴⁴X⁴⁵X⁴⁶X⁴⁷NTTX⁵¹ASIAAKEEGVSLDKR (SEQ ID NO: 82) of the pro-sequence.

In some embodiments, the pre-sequence comprises a portion that is ten or more amino acids in length and that comprises the amino acid sequence LXXXXLL (SEQ ID NO: 15), and wherein at least five of the amino acids of the portion are selected from the group consisting of leucine (L) and isoleucine (I).

In some embodiments, the pre-sequence comprises the amino acid sequence LX²X³X⁴X⁵LLX⁸X⁹X¹⁰X¹¹X¹² (SEQ ID NO: 17), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); and X¹² is alanine (A) or cytosine (C).

In some embodiments, the pre-sequence comprises an amino acid sequence having at least 80% identity to one or more of: the amino acid sequence MTKPTQVLVRSVSILFFITLLHLVVA (SEQ ID NO: 1); the amino acid sequence MQLYLTLLFLLSFVEC (SEQ ID NO: 9); and the amino acid sequence

(SEQ ID NO: 2)
MQHFLSLLLAVSLLTTTYA.

In some embodiments, a secretion signal comprises, from N-terminus to C-terminus: (a) a pre-sequence comprising an amino acid sequence having at least 80% identity to one or more of: the amino acid sequence MTKPTQVLVRSVSILFFITLLHLVVA (SEQ ID NO: 1); the amino acid sequence MQLYLTLLFLLSFVEC (SEQ ID NO: 9); and the amino acid sequence MQHFLSLLLAVSLLTTTYA (SEQ ID NO: 2); and (b) a pro-sequence comprising an amino acid sequence having at least 80% identity to the amino acid sequence: APANTTTEDETAQIPAEAVIDYSDLEGDFDAAALPLSNSTNNGLSSTNTTIASIAAKEE GVSLDKR (SEQ ID NO: 61). In some embodiments, no more than 10 amino acids separate the amino acid sequence having at least 80% identity to one or more of the amino acid sequence MTKPTQVLVRSVSILFFITLLHLVVA (SEQ ID NO: 1), the amino acid sequence MQLYLTLLFLLSFVEC (SEQ ID NO: 9), and the amino acid sequence MQHFLSLLLAVSLLTTTYA (SEQ ID NO: 2) of the pre-sequence from the amino acid sequence having at least 80% identity to the amino acid sequence APANTTTEDETAQIPAEAVIDYSDLEGDFDAAALPLSNSTNNGLSSTNTTIASIAAKEE GVSLDKR (SEQ ID NO: 61) of the pro-sequence.

In some embodiments, the pre-sequence comprises the amino acid sequence of:

(SEQ ID NO: 1)
MTKPTQVLVRSVSILFFITLLHLVVA;
(SEQ ID NO: 9)
MQLYLTLLFLLSFVEC;
or
(SEQ ID NO: 2)
MQHFLSLLLAVSLLTTTYA.

In some embodiments, the pro-sequence does not comprise the amino acid sequence of

(SEQ ID NO: 59)
APVNTTTEDETAQIPAEAVIGYLDLEGDFDVAVLPFSNSTNNGLLFINTT
IASIAAKEEGVSLDKR.

In some embodiments, the pro-sequence comprises the amino acid sequence:

(SEQ ID NO: 61)
APANTTTEDETAQIPAEAVIDYSDLEGDFDAAALPLSNSTNNGLSSTNTT
IASIAAKEEGVSLDKR.

In some embodiments, a secretion signal comprises the amino acid sequence LX²X³X⁴X⁵LLX⁸X⁹X¹⁰X¹¹X¹²APX¹⁵NX¹⁷TX¹⁹EX²¹EX²³X²⁴QX²⁶PAEAX³¹X³²X³³YX³⁵X³⁶X³⁷EGDX⁴¹DX⁴³AX⁴⁵LPX⁴⁸X⁴⁹X⁵⁰STNX⁵⁴GX⁵⁶X⁵⁷X⁵⁸X⁵⁹NTTX⁶³ASIAAKEEGVSLDKR (SEQ ID NO: 153), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); X¹² is alanine (A) or cytosine (C); X¹⁵ is valine (V) or alanine (A); X¹⁷ is threonine (T) or alanine (A); X¹⁹ is threonine (T) or alanine (A); X²¹ is aspartic acid (D) or glycine (G); X²³ is threonine (T) or alanine (A); X²⁴ is threonine (T) or alanine (A); X²⁶ is isoleucine (I) or threonine (T); X³¹ is valine (V) or alanine (A); X³² is isoleucine (I) or alanine (A); X³³ is glycine (G), aspartic acid (D), or threonine (T); X³⁵ is leucine (L), serine (S), or arginine (R); X³⁶ is aspartic acid (D) or glycine (G); X³⁷ is leucine (L) or serine (S); X⁴¹ is phenylalanine (F), serine (S), or valine (V); X⁴³ is valine (V) or alanine (A); X⁴⁵ is valine (V) or alanine (A); X⁴⁸ is phenylalanine (F) or leucine (L); X⁴⁹ serine (S) or proline (P); X⁵⁰ is asparagine (N), serine (S), or aspartic acid (D); X⁵⁴ is asparagine (N) or aspartic acid (D); X⁵⁶ is leucine (L) or serine (S); X⁵⁷ is leucine (L) or serine (S); X⁵⁸ is phenylalanine (F) or serine (S); X⁵⁹ is isoleucine (I) or threonine (T); and X⁶³ is isoleucine (I) or threonine (T). In some embodiments, the secretion signal does not comprise the amino acid sequence of APVNTTTEDETAQIPAEAVIGYLDLEGDFDVAVLPFSNSTNNGLLFINTTIASIAAKEE GVSLDKR (SEQ ID NO: 59). In some embodiments, the secretion signal comprises the amino acid sequence LX²X³X⁴X⁵LLX⁸X⁹X¹⁰X¹¹X¹²APANTTTEDETAQIPAEAVIDYSDLEGDFDAAALPLSNS TNNGLSSTNTTIASIAAKEEGVSLDKR (SEQ ID NO: 157), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); and X¹² is alanine (A) or cytosine (C). In some embodiments, the secretion signal further comprises the amino acid sequence: MTKPTQVLVRSVSI (SEQ ID NO: 154); MQLY (SEQ ID NO: 155); or MQHFLSL (SEQ ID NO: 156).

In some embodiments, a secretion signal comprises an amino acid sequence having at least 80% identity to the one or more of:

(SEQ ID NO: 107)
MTKPTQVLVRSVSILFFITLLHLVVAAPANTTTEDETAQIPAEAVIDYS
DLEGDFDAAALPLSNSTNNGLSSTNTTIASIAAKEEGVSLDKR;
(SEQ ID NO: 108)
MQLYLTLLFLLSFVECAPANTTTEDETAQIPAEAVIDYSDLEGDFDAAA
LPLSNSTNNGLSSTNTTIASIAAKEEGVSLDKR;
and
(SEQ ID NO: 115)
MQHFLSLLLAVSLLTTTYAAPANTTTEDETAQIPAEAVIDYSDLEGDFD
AAALPLSNSTNNGLSSTNTTIASIAAKEEGVSLDKR.

In some embodiments, a secretion signal comprises the amino acid sequence of:

(SEQ ID NO: 107)
MTKPTQVLVRSVSILFFITLLHLVVAAPANTTTEDETAQIPAEAVIDYS
DLEGDFDAAALPLSNSTNNGLSSTNTTIASIAAKEEGVSLDKR;
(SEQ ID NO: 108)
MQLYLTLLFLLSFVECAPANTTTEDETAQIPAEAVIDYSDLEGDFDAAA
LPLSNSTNNGLSSTNTTIASIAAKEEGVSLDKR;
or
(SEQ ID NO: 115)
MQHFLSLLLAVSLLTTTYAAPANTTTEDETAQIPAEAVIDYSDLEGDFD
AAALPLSNSTNNGLSSTNTTIASIAAKEEGVSLDKR.

In some embodiments, a secretion signal comprises, from N-terminus to C-terminus, a pre-sequence from a first species (or derived from a pre-sequence from a first species) and a pro-sequence from a second species (or derived from a pro-sequence from a second species), wherein: (a) the pre-sequence comprises the amino acid sequence of: WFSWIVG (SEQ ID NO: 18); MRFPSIFTAVLF (SEQ ID NO: 19); SSALA (SEQ ID NO: 20); IVGLF (SEQ ID NO: 21); MTKPTQVLV (SEQ ID NO: 22); MKLATAFTILTA (SEQ ID NO: 23); ETPRASLSLGRW (SEQ ID NO: 24); WHAVMVFVLCG (SEQ ID NO: 25); MRFPSIFT (SEQ ID NO: 222); MKX³X⁴X⁵X⁶AX⁸LSX¹¹X¹²X¹³LX¹⁴L (SEQ ID NO: 26), wherein X³ is phenylalanine (F) or leucine (L), X⁴ is serine (S) or phenylalanine (F), X⁵ is alanine (A) or valine (V), X⁶ is glycine (G) or proline (P), X⁸ is valine (V) or leucine (L), X¹¹ is tryptophan (W) or leucine (L), X¹² is serine (S) or glycine (G), X¹³ is serine (S) or alanine (A), and X¹⁴ is leucine (L) or glycine (G); or MX²X³X⁴X⁵ (SEQ ID NO: 223), wherein X² is arginine (R) or glutamine (Q), X³ is histidine (H) or glutamine (Q), X⁴ is valine (V) or phenylalanine (F), and X⁵ is leucine (L) or tryptophan (W); (b) the pro-sequence comprises the amino acid sequence of: RYVVGDDEQ (SEQ ID NO: 64); IVAKSGI (SEQ ID NO: 65); IPDEAIAN (SEQ ID NO: 66); QTSISDDEEPIVVEINGQKV (SEQ ID NO: 67); INTTLTEEALEKSGISIDDL (SEQ ID NO: 68); PVFAEIDNK (SEQ ID NO: 69); DDLKESYAN (SEQ ID NO: 70); PVENVDD (SEQ ID NO: 71); IDQEQLTNG (SEQ ID NO: 72); PVDSGAKGKYSR (SEQ ID NO: 73); NDGVGVGMSTIKEEDFGKHF (SEQ ID NO: 74); TTIASIA (SEQ ID NO: 224); YVVGDDEQ (SEQ ID NO: 225); PVFAEIDNKPVVYIVNTTKA (SEQ ID NO: 226); ESIVAKSGITLDDLKESYAN (SEQ ID NO: 227); NTTIX⁵X⁶X⁷A (SEQ ID NO: 63), wherein X⁵ is alanine (A), leucine (L), or tyrosine (Y), X⁶ is alanine (A), serine (S), asparagine (N), or glutamic acid (E), and X⁷ is alanine (A), isoleucine (I), serine (S), glutamic acid (E), or glutamine (Q); AAX³EEGX⁷SLDKR (SEQ ID NO: 221), wherein X³ is lysine (K) or alanine (A), and X⁷ is valine (V) or serine (S); X¹NTTIAX⁷X⁸AX¹⁰X¹¹EEGVX¹⁶ (SEQ ID NO: 75), wherein X¹ is valine (V) or isoleucine (I), X⁷ is aspartic acid (D), serine (S), or glutamic acid (E), X⁸ is isoleucine (I) or glutamine (Q), X¹⁰ is alanine (A) or leucine (L), X¹¹ is alanine (A) or lysine (K), and X¹⁶ is serine (S) or leucine (L); X¹X²X³X⁴X⁵DDEX⁹ (SEQ ID NO: 76), wherein X¹ is arginine (R) or glutamine (Q), X² is tyrosine (Y) or threonine (T), X³ is valine (V) or serine (S), X⁴ is valine (V) or isoleucine (I), X⁵ is glycine (G) or serine (S), and X⁹ is glutamine (Q) or glutamic acid (E); AX²LPFSNX⁸TNX¹¹GX¹³X¹⁴FX¹⁶NTTI (SEQ ID NO: 77), wherein X² is valine (V) or leucine (L), X⁸ is serine (S) or glycine (G), X¹¹ is asparagine (N) or threonine (T), X¹³ is isoleucine (I) or leucine (L), X¹⁴ is serine (S), leucine (L), or methionine (M), and X¹⁶ is valine (V) or isoleucine (I); X¹AQX⁴PAEAX⁹IGX¹²LDLX¹⁶X¹⁷X¹⁸X¹⁹D (SEQ ID NO: 78), wherein X¹ is threonine (T) or serine (S), X⁴ is isoleucine (I) or valine (V), X⁹ is valine (V) or isoleucine (I), X¹² is tyrosine (Y) or phenylalanine (F), X¹⁶ is glutamic acid (E) or threonine (T), X¹⁷ is aspartic acid (D) or glycine (G), X¹⁸ is aspartic acid (D), serine (S), or alanine (A), and X¹⁹ is glutamic acid (E) or phenylalanine (F); X¹GX³X⁴X⁵X⁶X⁷DX⁹IX¹¹P (SEQ ID NO: 79), wherein X¹ is lysine (K) or serine (S), X³ is lysine (K) or arginine (R), X⁴ is tyrosine (Y) or phenylalanine (F), X⁵ is serine (S) or leucine (L), X⁶ is arginine (R) or glutamic acid (E), X⁷ is glutamine (Q) or threonine (T), X⁹ is leucine (L) or isoleucine (I), and X¹¹ is isoleucine (I) or phenylalanine (F); X¹X²NX⁴TX⁶E (SEQ ID NO: 80), wherein X¹ is asparagine (N) or proline (P), X² is glycine (G) or alanine (A), X⁴ is glycine (G) or threonine (T), and X⁶ is serine (S) or threonine (T); PAEAVIX⁷Y (SEQ ID NO: 228), wherein X⁷ is aspartic acid (D) or glycine (G); KEEX⁴X⁵X⁶X⁷X⁸KR (SEQ ID NO: 229), wherein X⁴ is glycine (G) or glutamic acid (E), X⁵ is valine (V) or alanine (A), X⁶ is serine (S) or lysine (K), X⁷ is leucine (L) or asparagine (N), and X⁸ is aspartic acid (D) or glycine (G); GDFDX⁵AX⁷LP (SEQ ID NO: 230), wherein X⁵ is valine (V) or alanine (A), and X⁷ is valine (V) or alanine (A); X¹SNST (SEQ ID NO: 231), wherein X¹ is leucine (L) or phenylalanine (F); GLSX⁴TN (SEQ ID NO: 232), wherein X⁴ is serine (S) or phenylalanine (F); PX²SNSTNNGLSX¹²TNTTIASI (SEQ ID NO: 233), wherein X² is leucine (L) or phenylalanine (F), and X¹² is serine (S) or phenylalanine (F); or X¹X²X³IPX⁶EAX⁹X¹⁰X¹¹X¹²X¹³X¹⁴X¹⁵X¹⁶X¹⁷DX¹⁹X²⁰ (SEQ ID NO: 234), wherein X¹ is threonine (T) or aspartic acid (D), X² is alanine (A) or leucine (L), X³ is glutamine (Q) or isoleucine (I), X⁶ is alanine (A) or aspartic acid (D), X⁹ is valine (V) or isoleucine (I), X¹⁰ is isoleucine (I) or alanine (A), X¹¹ is aspartic acid (D), glycine (G), or asparagine (N), X¹² is tyrosine (Y) or arginine (R), X¹³ serine (S) or tyrosine (Y), X¹⁴ is aspartic acid (D) or valine (V); X¹⁵ is leucine (L) or valine (V), X¹⁶ is glutamic acid (E) or glycine (G), X¹⁷ is glycine (G) or aspartic acid (D), X¹⁹ is phenylalanine (F) or glutamic acid (E), and X²⁰ is aspartic acid (D) or glutamine (Q); or (c) a combination thereof.

In some embodiments, a secretion signal comprises, from N-terminus to C-terminus, a pre-sequence from a first species (or derived from a pre-sequence from a first species) and a pro-sequence from a second species (or derived from a pro-sequence from a second species), wherein: the secretion signal comprises the amino acid sequence: CX²X³X⁴X⁵X⁶X⁷X⁸APX¹¹NTTT (SEQ ID NO: 146), wherein X² is leucine (L), phenylalanine (F), or glycine (G), X³ is leucine (L), phenylalanine (F), or valine (V), X⁴ is asparagine (N) or valine (V), X⁵ is valine (V) or leucine (L), X⁶ is serine (S), alanine (A), or valine (V), X⁷ is serine (S) or alanine (A), X⁸ is alanine (A) or glycine (G), and X¹¹ is valine (V) or alanine (A); X¹AAPX⁵X⁶TTTEDE (SEQ ID NO: 147), wherein X¹ is leucine (L), serine (S), or alanine (A), X⁵ is alanine (A) or valine (V), and X⁶ is asparagine (N) or serine (S); AAPIX⁵X⁶X⁷X⁸S (SEQ ID NO: 148), wherein X⁵ is asparagine (N) or lysine (K), X⁶ is isoleucine (I) or phenylalanine (F), X⁷ is threonine (T) or asparagine (N), and X⁸ is serine (S) or aspartic acid (D); X¹X²X³X⁴X⁵X⁶X⁷X⁸X⁹ (SEQ ID NO: 149), wherein X¹ is glutamine (Q) or asparagine (N), X² is valine (V) or histidine (H), X³ is tryptophan (W) or phenylalanine (F), X⁴ is phenylalanine (F), leucine (L), or histidine (H), X⁵ is serine (S) or alanine (A), X⁶ is tryptophan (W), leucine (L), or valine (V), X⁷ is isoleucine (I), leucine (L), or methionine (M), X⁸ is valine (V) or leucine (L), and X⁹ is glycine (G), alanine (A), or phenylalanine (F); X¹X²X³X⁴X⁵X⁶AX⁸X⁹ (SEQ ID NO: 150), wherein X¹ lysine (K) or asparagine (N), X² is glycine (G), asparagine (N), or aspartic acid (D), X³ is asparagine (N), glycine (G), or lysine (K), X⁴ is leucine (L), tyrosine (Y), or glycine (G), X⁵ is serine (S) or asparagine (N), X⁶ is serine (S), arginine (R), or glycine (G), X⁸ is asparagine (N), aspartic acid (D), or serine (S), and X⁹ is threonine (T), leucine (L), or glutamic acid (E); X¹RX³X⁴X⁵X⁶X⁷X⁸X⁹X¹⁰X¹¹X¹²X¹³X¹⁴X¹⁵X¹⁶ (SEQ ID NO: 151), wherein X¹ is methionine (M), valine (V), or glutamine (Q), X³ is phenylalanine (F) or glutamine (Q), X⁴ is leucine (L) or valine (V), X⁵ is serine (S) or tryptophan (W), X⁶ is phenylalanine (F) or leucine (L), X⁷ is leucine (L) or serine (S), X⁸ is threonine (T), leucine (L), phenylalanine (F), or tryptophan (W), X⁹ is alanine (A), leucine (L), or isoleucine (I), X¹⁰ is valine (V) or leucine (L), X¹¹ is leucine (L), glycine (G), or serine (S), X¹² is leucine (L) or phenylalanine (F), X¹³ is valine (V), leucine (L), or phenylalanine (F), X¹⁴ is valine (V) or leucine (L), X¹⁵ is serine (S) or cytosine (C), and X¹⁶ is alanine (A) or phenylalanine (F); X¹X²X³X⁴X⁵X⁶X⁷X⁸X⁹X¹⁰X¹¹X¹²X¹³X¹⁴X¹⁵X¹⁶X¹⁷IX¹⁹X²⁰ (SEQ ID NO: 152), wherein X¹ is aspartic acid (D), valine (V), or glutamic acid (E), X² is valine (V), tyrosine (Y), or proline (P), X³ is proline (P), isoleucine (I), or serine (S), X⁴ is glycine (G) or valine (V), X⁵ is threonine (T), asparagine (N), or arginine (R), X⁶ is serine (S), threonine (T), or phenylalanine (F), X⁷ is glutamine (Q), threonine (T), or leucine (L), X⁸ is glycine (G), lysine (K), or glutamic acid (E), X⁹ is valine (V), alanine (A), or glutamine (Q), X¹⁰ is glutamic acid (E) or aspartic acid (D), X¹¹ is phenylalanine (F), serine (S), or isoleucine (I), X¹² is isoleucine (I) or proline (P), X¹³ is phenylalanine (F) or valine (V), X¹⁴ is alanine (A) or proline (P), X¹⁵ is lysine (K) or glutamine (Q), X¹⁶ is glutamic acid (E), serine (S), or glutamine (Q), X¹⁷ is alanine (A) or glycine (G), X¹⁹ is isoleucine (I), threonine (T), or asparagine (N), and X²⁰ is glutamic acid (E), leucine (L), or alanine (A); AAPX⁴X⁵X⁶X⁷X⁸X⁹X¹⁰X¹¹X¹² (SEQ ID NO: 235), wherein X⁴ is alanine (A) or valine (V), X⁵ is asparagine (N) or aspartic acid (D), X⁶ is serine (S) or threonine (T), X⁷ is threonine (T) or glycine (G), X⁸ is threonine (T) or alanine (A), X⁹ is glutamic acid (E) or lysine (K), X¹⁰ is glycine (G) or aspartic acid (D), X¹¹ is glutamic acid (E) or lysine (K), and X¹² is threonine (T) or tyrosine (Y); AX²KEEX⁶X⁷X⁸X⁹X¹⁰KREAEA (SEQ ID NO: 236), wherein X² is alanine (A) or threonine (T), X⁶ is glycine (G) or glutamic acid (E), X⁷ is valine (V) or alanine (A); X⁸ is serine (S) or lysine (K), X⁹ is leucine (L) or asparagine (N), and X¹⁰ is aspartic acid (D) or glycine (G); SLLX⁴X⁵SX⁷X⁸LAAPX¹³NTTTEDE (SEQ ID NO: 237), wherein X⁴ is alanine (A), phenylalanine (F), leucine (L), or serine (S), X⁵ is leucine (L) or alanine (A), X⁷ is leucine (L) or serine (S), X⁸ is leucine (L) or valine (V), and X¹³ is alanine (A) or valine (V); or MX²X³X⁴X⁵X⁶X⁷X⁸X⁹ (SEQ ID NO: 238), wherein X² is alanine (A), lysine (K), or arginine (R), X³ is leucine (L), glutamine (Q), or arginine (R), X⁴ is phenylalanine (F) or valine (V), X⁵ is valine (V) or tryptophan (W), X⁶ is alanine (A), phenylalanine (F), or proline (P), X⁷ is leucine (L), alanine (A), or serine (S), X⁸ is leucine (L), valine (V), or tryptophan (W), and X⁹ is leucine (L) or isoleucine (I).

In some embodiments, a secretion signal further comprises a C-terminal cleavage sequence. In some embodiments, the C-terminal cleavage sequence comprises the amino acid sequence of EAEA (SEQ ID NO: 104), KR, or a combination thereof.

In some embodiments, a polypeptide comprising the section signal further comprises the amino acid sequence of a protein of interest, wherein the amino acid sequence of the protein of interest is positioned C-terminal to the amino acid sequence of the secretion signal. In some embodiments, the protein of interest is lactoferrin (LF), lactoglobulin (LG), or ovalbumin (Ova). In some embodiments, the protein of interest is bovine lactoferrin (bLF), bovine lactoglobulin (bLG), or ovalbumin (OVA).

In some aspects, the disclosure relates to nucleic acids encoding a polypeptide comprising a secretion signal provided herein.

In some embodiments, a nucleic acid encoding the polypeptide further comprises a nucleic acid sequence of a promoter, wherein the nucleic acid sequence of the promoter is operably linked to the nucleic acid sequence encoding the polypeptide. In some embodiments, the promoter is a constitutive promoter. In some embodiments, the promoter is an inducible promoter. In some embodiments, the inducible promoter is on that is activated during glucose or thiamine limitation.

In some embodiments, a nucleic acid encoding the polypeptide further comprises a nucleic acid sequence of a terminator, wherein the nucleic acid sequence of the terminator is operably linked to the nucleic acid sequence encoding the polypeptide. In some embodiments, the terminator is a tFDH1 terminator, a tTEF1 terminator, or a tAOX1 terminator.

In some embodiments, an expression vector comprises the nucleic acid sequence.

In some aspects, the disclosure relates to host cells for expressing a protein of interest.

In some embodiments, a host cell comprises a nucleic acid sequence as provided herein.

In some embodiments, a host cell comprises an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal as provided herein.

In some embodiments, a host cell comprises one or more genetic modifications that result in the overexpression of a gene encoding a calreticulin (CRT) protein (or a homolog thereof) and/or a protein disulfide isomerase family A member 3 (PDIA3) protein (or a homolog thereof), and optionally further comprising one or more genetic modifications that result in the overexpression of a HAC1 protein (or a homolog thereof).

In some embodiments, a host cell comprises: (a) one or more genetic modifications that result in the overexpression of a gene encoding a calreticulin (CRT) protein (or a homolog thereof) and/or a protein disulfide isomerase family A member 3 (PDIA3) protein (or a homolog thereof); and (b) a nucleic acid sequence encoding a polypeptide comprising a secretion signal and the amino acid sequence of a protein of interest; optionally wherein the host cell further comprises one or more genetic modifications that result in the overexpression of a HAC1 protein (or a homolog thereof). In some embodiments, a host cell comprises: (a) one or more genetic modifications that result in the overexpression of a gene encoding a calreticulin (CRT) protein (or a homolog thereof) and/or a protein disulfide isomerase family A member 3 (PDIA3) protein (or a homolog thereof); and (b) one or more genetic modifications that result in the overexpression of a HAC1 protein (or a homolog thereof).

In some embodiments, the host cell comprises one or more genetic modifications that result in the overexpression of an endogenous CRT protein (or a homolog thereof). In some embodiments, a host cell comprises one or more genetic modifications that result in the overexpression of an exogenous CRT protein (or a homolog thereof). In some embodiments, a CRT protein (or homolog thereof) is selected from the group consisting of Anopheles christyi CRT, Arabidopsis thaliana CRT1, Chlorocebus aethiops CALR, Gigaspora rosea Calreticulin family-domain-containing protein, Mucor ambiguus CRT, and Mus musculus CALR. In some embodiments, at least one of the one or more genetic modifications is a genomic integration of an expression cassette encoding for a CRT protein (or a homolog thereof). In some embodiments, at least one of the one or more genetic modifications is a transformation of a vector comprising an expression cassette encoding for a CRT protein (or a homolog thereof).

In some embodiments, a host cell comprises one or more genetic modifications that result in the overexpression of an endogenous PDIA3 protein (or a homolog thereof). In some embodiments, a host cell comprises one or more genetic modifications that result in the overexpression of an exogenous PDIA3 protein (or a homolog thereof). In some embodiments, the PDIA3 protein (or homolog thereof) is selected from the group consisting of Anopheles christyi Protein disulfide-isomerase, Arabidopsis thaliana Protein disulfide isomerase-like 1-1, Arabidopsis thaliana Protein disulfide isomerase-like 1-2, Arabidopsis thaliana Protein disulfide isomerase-like 1-6, Chlorocebus aethiops PDIA3, Dictyostelium discoideum Protein disulfide-isomerase 2, Mucor ambiguus Protein disulfide isomerase-like 2-1-like, and Mus musculus PDIA3. In some embodiments, at least one of the one or more genetic modifications is a genomic integration of an expression cassette encoding for a PDIA3 protein (or a homolog thereof). In some embodiments, at least one of the one or more genetic modifications is a transformation of a vector comprising an expression cassette encoding for a PDIA3 protein (or a homolog thereof).

In some embodiments, a host cell comprises one or more genetic modifications that result in the overexpression of an endogenous HAC1 protein (or a homolog thereof). In some embodiments, a host cell comprises one or more genetic modifications that result in the overexpression of an exogenous HAC1 protein (or a homolog thereof). In some embodiments, the HAC1 protein (or homolog thereof) is selected from the group consisting of Saccharomyces cerevisiae HAC1 and Homo sapiens XBP1. In some embodiments, at least one of the one or more genetic modifications is a genomic integration of an expression cassette encoding for a HAC1 protein (or a homolog thereof). In some embodiments, at least one of the one or more genetic modifications is a transformation of a vector comprising an expression cassette encoding for a HAC1 protein (or a homolog thereof).

In some embodiments, a host cell comprises a nucleic acid sequence encoding a polypeptide, wherein the polypeptide comprises a secretion signal and the amino acid sequence of a protein of interest.

In some embodiments, the host cell is a eukaryotic cell. In some embodiments, the host cell is a yeast cell. In some embodiments, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica. In some embodiments, the yeast cell is Pichia pastoris. In some embodiments, the host cell is a mold cell. In some embodiments, the mold cell is Aspergillus, Trichoderma, Humicola, Neurospora, Penicillium, Cephalosporium, Myceliophthora, Thermomyces, or Chrysosporium. In some embodiments, the mold cell is Aspergillus niger, Aspergillus nidulans, Aspergillus awamori, Aspergillus sojae, Aspergillus oryzae, Trichoderma reesei, Trichoderma viride, Chrysosporium lucknowense, Fusarium gramineum, Fusarium venenatum, or Neurospora crassa. In some embodiments, the mold cell is Aspergillus niger.

In some aspects, the disclosure relates to methods of manufacturing a polypeptide. In some embodiments, a method of manufacturing a polypeptide comprises culturing a host cell described herein. In some embodiments, the level of secretion of the polypeptide is increased relative to the respective level for a host cell that is the same except that it lacks the one or more genetic modifications of the host cell. In some embodiments, the method further comprises obtaining the polypeptide from a culture, culture medium, cell-free spent culture medium, and/or cell-containing culture medium, and/or biomass used in, during or produced by culturing the host cell.

### BRIEF DESCRIPTION OF DRAWINGS

The following drawings form part of the present specification and are included to further demonstrate certain aspects of the present disclosure, which may be better understood by reference to one or more of these drawings in combination with the detailed description of specific embodiments presented in this disclosure. The drawings are illustrative only and are not required for enablement of the disclosure.

In the drawings:

FIG. 1 provides a schematic of an exemplary secretion signal, including its pre-region (or pre-sequence) and its pro-region (or pro-sequence).

FIGS. 2A-2B provide an overview of a secretion signal combinatorial library. FIG. 2A. Schematic of secretion signal combinatorial library. Pre-regions (or pre-sequences) and pro-regions (or pro-sequences) of secretion signals were identified using S. cerevisiae and Pichia αMFs as search queries, and these pre-regions (or pre-sequences) and pro-regions (or pro-sequences) were used to generate a combinatorial secretion signal library. FIG. 2B. Machine learning was used to select diverse secretion peptides for library design.

FIG. 3 shows results from a secondary secretion library screen. Total bLF (mg/L) levels are shown on the y-axis, and secretion signals that were tested in the secondary screen are provided on the x-axis.

FIGS. 4A-4D provide exemplary schematics for expression system components. FIG. 4A depicts a transcriptional unit for expression of a secreted protein (e.g., bLF, bLG, or OVA). FIG. 4B depicts a transcriptional unit for expression of a calreticulin (CRT) protein (e.g., GrCalreticulin), and a transcriptional unit for the expression of a PDIA3 protein (e.g., ArPDIA3). FIG. 4C depicts a transcriptional unit for expression of a HAC1 protein (e.g., ScHAC1). FIG. 4D depicts a transcriptional unit for expression of a PDI1 protein and a transcriptional unit for expression of a GPX1 protein.

FIG. 5 shows results from experiments testing the impact of overexpression of a calreticulin protein and/or PDIA3 protein on bLF titer levels.

FIG. 6 depicts an exemplary transcriptional unit for expression and secretion of ovalbumin in Pichia.

FIG. 7 depicts an exemplary transcriptional unit for expression and secretion of bLF in Aspergillus niger.

FIG. 8 depicts an exemplary transcriptional unit for expression and secretion of ovalbumin in Aspergillus niger.

### DETAILED DESCRIPTION

Aspects of the disclosure relate to expression systems comprising a secretion signal and/or one or more polynucleotides encoding a CRT protein and/or a PDIA3 protein which, as demonstrated herein, were found to enhance production and/or secretion of proteins to a surprising degree, both individually and collectively. In some embodiments, the expression systems described herein further comprise one or more polynucleotides encoding: a PDI1 protein; a GPX1 protein; a HAC protein; a synthetic transcription factor; or any combination thereof. Additional aspects of the disclosure relate to expression systems for production (and secretion) of a protein of interest [e.g., bovine lactoferrin (bLF), bovine lactoglobulin (bLG), or ovalbumin (Ova)].

In addition, aspects of the disclosure relate to genetically modified host cells comprising an expression system. In some embodiments, a genetically modified host cell further comprises one or more genetic modifications, wherein the genetic modification(s) result in: (a) the expression of a gene encoding a mutant HSF1 protein (or a homolog thereof); and/or (b) the downregulation of one or more of the genes SSY1, HSL1, PAS_chr2-1_0053, PAS_chr2-1_0404, PAS_chr4_0550, PAS_chr1-3_0135, and/or PAS_chr1-3_0285 (or a homolog of any of these genes).

As demonstrated herein, these genetically modified host cells are capable of enhanced production of a protein of interest. Without wishing to be bound by any particular theory, the present disclosure suggests that this high level of production may be achieved via one or more of the following mechanisms (which may act individually or concertedly (e.g., synergistically)): promoting protein maturation; reducing the level of protein lost to aggregation or misfolding; reducing the amount of protein which is misfolded and then degraded by the cell; binding to misfolded proteins and preventing them from being exported from the endoplasmic reticulum (ER) to the Golgi apparatus; binding to oligosaccharides which are bound to proteins but comprise a terminal glucose residue, and targeting them for degradation; modulating folding of newly synthesized glycoproteins; complexing with lectins to mediate protein folding by promoting formation of disulfide bonds in their glycoprotein substrates; helping the cell accumulate sufficient resources (e.g., amino acids) to make high levels of protein; increasing the cytosolic volume available for producing a protein of interest; and/or increasing the secretion of the protein of interest.

### Secretion Signals and Polypeptides Comprising the Same

Aspects of the disclosure relate to secretion signals.

In some embodiments, a secretion signal is at the N-terminus of a polypeptide of interest (e.g., a fusion protein comprises the polypeptide of interest and a secretion signal at the N-terminus of the polypeptide of interest). In some embodiments, in a fusion protein comprising a polypeptide of interest fused to a secretion signal, the initial M (Met) codon at the N-terminus of the polypeptide is deleted. In some embodiments, a sequence of a polypeptide of interest (or a sequence of any of various polypeptides noted herein) terminates at the C-terminus with an asterisk (*) indicating a stop codon. In some embodiments, the secretion signal acts to achieve localization to a secretory pathway compartment, e.g., the endoplasmic reticulum (ER), Golgi apparatus, a vacuole, plasma membrane, periplasmic space, or extracellular space.

In some aspects, the disclosure relates to polypeptides comprising a secretion signal. In some embodiments, a polypeptide comprises: a secretion signal described herein; and an amino acid sequence of a protein of interest. In some embodiments, the amino acid sequence of the protein of interest is positioned C-terminal to the amino acid sequence of the secretion signal.

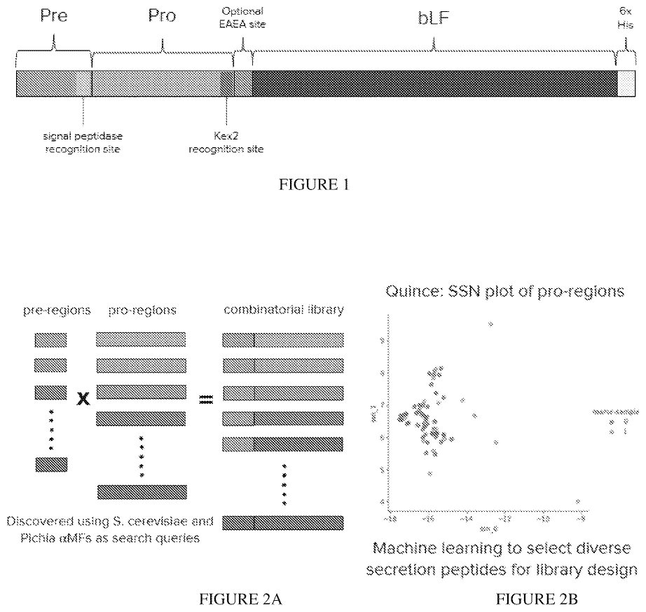

In some embodiments, a secretion signal comprises, from N-terminus to C-terminus: a pre-sequence (or pre-region); and a pro-sequence (or pro-region) (see e.g., FIG. 1). Without wishing to be bound by any particular theory, the pre-sequence (or pre-region) is thought to serve the purpose of directing a polypeptide to the endoplasmic reticulum, and the pro-sequence (or pro-region) is thought to have multiple roles in secretion, such as facilitating: glycosylation (ER to Golgi transport); proper protein folding; and aspects of late secretory processing, such as COPII vesicle sorting and vacuolar targeting.

In some embodiments, in addition to producing secreted proteins, the utility of a pre-sequence is to achieve localization to one or more compartments in the secretory pathway (e.g., ER, Golgi apparatus, Vacuole) the plasma membrane, the periplasmic space, and/or the extracellular space.

In some embodiments, a secretion signal further comprises a cleavage sequence. In some embodiments, a cleavage sequence is a sequence recognized and cleaved by a protease in a host cell. In some embodiments, a secretion signal further comprises two cleavage sequences, such that a fusion protein comprising a signal peptide and a polypeptide of interest comprises, in order from N-terminus to C-terminus: a pre-sequence, a first cleavage site, a pro-sequence, a second cleavage site, and the polypeptide of interest. In some embodiments, wherein the fusion protein comprises two cleavage sites: after the pre-sequence mediates its function (e.g., directing the polypeptide to the endoplasmic reticulum or other compartment), the fusion protein is cleaved at the first cleavage site to remove the pre-sequence; then the pro-sequence mediates its function, after which it is also optionally cleaved.

In some embodiments, a secretion signal comprises only a pre-sequence and, optionally, a cleavage site (e.g., the secretion signal does not comprise a pro-sequence). Indeed, the inventors have found that several pre-sequences [e.g., Pre1 (3075301), Pre2 (3075303), Pre3 (3075305) and Pre4 (3075307)] were able to mediate secretion and/or expression of a protein of interest (e.g., bLF) in the absence of a pro-sequence. In some embodiments, a secretion signal comprises only a pro-sequence and, optionally, a cleavage site (e.g., the secretion signal does not comprise a pre-sequence).

### Pre-Sequence

The secretion signals described herein may comprise a pre-sequence.

In some embodiments, a pre-sequence is 15-30 amino acids in length, 15-25 amino acids in length, 16-24 amino acids in length, 17-23 amino acids in length, 18-22 amino acids in length, 15-22 amino acids in length, 16-23 amino acids in length, 17-24 amino acids in length, 18-25 amino acids in length, 15-23 amino acids in length, 16-24 amino acids in length, 17-25 amino acids in length, 20-25 amino acids in length, 15-20 amino acids in length, 16-21 amino acids in length, 17-22 amino acids in length, 18-23 amino acids in length, or 19-24 amino acids in length.

In some embodiments, a pre-sequence comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an amino acid sequence listed in TABLE 1. In some embodiments, a pre-sequence comprises an amino acid sequence having no more than 5, no more than 4, no more than 3, no more than 2, or no more than 1 amino acid difference(s) (substitution, deletion, or addition) with an amino acid sequence listed in TABLE 1. In some embodiments, a pre-sequence comprises or consists of an amino acid sequence listed in TABLE 1.

In some embodiments, a pre-sequence comprises a high leucine (L) content. For example, in some embodiments, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% of the amino acids of a pre-sequence are leucine (L). In some embodiments, 15%-40%, 20%-40%, 21%-40%, 22%-40%, 23%-40%, 24%-40%, 25%-40%, 26%-40%, 27%-40%, 28%-40%, 29%-40%, or 30%-40% of the amino acids of a pre-sequence are leucine (L).

In some embodiments, a pre-sequence comprises the amino acid sequence LXXXXLL (SEQ ID NO: 15), wherein X is independently chosen from any amino acid. In some embodiments, a pre-sequence comprises the amino acid sequence LXXXXLL (SEQ ID NO: 15), wherein X is independently chosen from any naturally-occurring amino acid. Exemplary pre-sequences of TABLE 1 that comprise the amino acid sequence LXXXXLL (SEQ ID NO: 15) include Pre-Seq IDs: 3075317, 3075319, 3075325, 3075313, 3075301, 3075303, and 3075321.

In some embodiments, a pre-sequence comprises a portion that is 10 or more amino acids in length, 11 or more amino acids in length, or 12 or more amino acids in length and that comprises the amino acid sequence LXXXXLL (SEQ ID NO: 15), wherein at least 4, at least 5, at least 6, or at least 7 of the amino acids of the portion are selected from leucine (L) and/or isoleucine (I).

In some embodiments, a pre-sequence comprises the amino acid sequence LX²X³X⁴X⁵LL (SEQ ID NO: 16), wherein: X² is phenylalanine (F), proline (P), threonine (T), serine (S), valine (V), or leucine (L); X³ is phenylalanine (F), tryptophan (W), proline (P), alanine (A), valine (V), or leucine (L); X⁴ is proline (P), serine (S), alanine (A), valine (V), isoleucine (I), or leucine (L); and X⁵ is phenylalanine (F), glycine (G), alanine (A), serine (S), threonine (T), or leucine (L).

In some embodiments, a pre-sequence comprises the amino acid sequence LX²X³X⁴X⁵LLX⁸X⁹X¹⁰X¹¹X¹² (SEQ ID NO: 17), wherein: X² is phenylalanine (F), proline (P), threonine (T), serine (S), valine (V), or leucine (L); X³ is phenylalanine (F), tryptophan (W), proline (P), alanine (A), valine (V), or leucine (L); X⁴ is proline (P), serine (S), alanine (A), valine (V), isoleucine (I), or leucine (L); X⁵ is phenylalanine (F), glycine (G), alanine (A), serine (S), threonine (T), or leucine (L); X⁸ is histidine (H), serine (S), threonine (T), valine (V), or leucine (L); X⁹ is phenylalanine (F), serine (S), threonine (T), alanine (A), valine (V), or leucine (L); X¹⁰ is serine (S), threonine (T), glycine (G), alanine (A), or valine (V); X¹¹ is glycine (G), alanine (A), valine (V), asparagine (N), glutamic acid (E), serine (S), or tyrosine (Y); and X¹² is alanine (A), valine (V), asparagine (N), glutamic acid (E), cytosine (C), or leucine (L).

In some embodiments, no more than 10 amino acids, no more than 9 amino acids, no more than 8 amino acids, no more than 7 amino acids, no more than 6 amino acids, or no more than 5 amino acids separate the amino acid sequence LXXXXLL (SEQ ID NO: 15) of the pre-sequence from the amino acid sequence of the pro-sequence. In some embodiments, 5-10 amino acids, 5-9 amino acids, 5-8, amino acids 5-7 amino acids, 5-6 amino acids, 6-10 amino acids 6-9 amino acids, 6-8 amino acids, 7-10 amino acids, 7-9 amino acids, 7-8 amino acids, 8-10 amino acids, 8-9 amino acids, or 9-10 amino acids separate the amino acid sequence LXXXXLL (SEQ ID NO: 15) of the pre-sequence from the amino acid sequence of the pre-sequence.

In some embodiments, a pre-sequence comprises the amino acid sequence LLL. Exemplary pre-sequences of TABLE 1 that comprise the amino acid sequence LLL include Pre-Seq IDs: 3075303, 3075313, 3075319, 3075323, and 3075327.

In some embodiments, a pre-sequence comprises the amino acid sequence LC. Exemplary pre-sequences of TABLE 1 that comprise the amino acid sequence LC include Pre-Seq IDs: 3075305, 3075307, 3075315, and 3075325.

In some embodiments, a pre-sequence comprises the amino acid sequence TAV. Exemplary pre-sequences of TABLE 1 that comprise the amino acid sequence TAV include Pre-Seq IDs: 3075309, 3075311, and 3075323.

In some embodiments, a pre-sequence comprises the amino acid sequence WFSWIVG (SEQ ID NO: 18). In some embodiments, a pre-sequence comprises the amino acid sequence MRFPSIFTAVLF (SEQ ID NO: 19). In some embodiments, a pre-sequence comprises the amino acid sequence SSALA (SEQ ID NO: 20). In some embodiments, a pre-sequence comprises the amino acid sequence IVGLF (SEQ ID NO: 21). In some embodiments, a pre-sequence comprises the amino acid sequence MTKPTQVLV (SEQ ID NO: 22). In some embodiments, a pre-sequence comprises the amino acid sequence MKLATAFTILTA (SEQ ID NO: 23). In some embodiments, a pre-sequence comprises the amino acid sequence ETPRASLSLGRW (SEQ ID NO: 24). In some embodiments, a pre-sequence comprises the amino acid sequence WHAVMVFVLCG (SEQ ID NO: 25). In some embodiments, a pre-sequence comprises the amino acid sequence MRFPSIFT (SEQ ID NO: 222).

In some embodiments, a pre-sequence comprises the amino acid sequence MKX³X⁴X⁵X⁶AX⁸LSX¹¹X¹²X¹³LX¹⁴L (SEQ ID NO: 26), wherein: X³ is phenylalanine (F) or leucine (L); X⁴ is serine (S) or phenylalanine (F); X⁵ is alanine (A) or valine (V); X⁶ is glycine (G) or proline (P); X⁸ is valine (V) or leucine (L); X¹¹ is tryptophan (W) or leucine (L); X¹² is serine (S) or glycine (G); X¹³ is serine (S) or alanine (A); and X¹⁴ is leucine (L) or glycine (G).

In some embodiments, a pre-sequence comprises the amino acid sequence MX²X³X⁴X⁵ (SEQ ID NO: 223), wherein: X² is arginine (R) or glutamine (Q); X³ is histidine (H) or glutamine (Q); X⁴ is valine (V) or phenylalanine (F); and X⁵ is leucine (L) or tryptophan (W).

In some embodiments, a pre-sequence comprises a structure of M-A′-Q-B′-L-C′-L-D′-LL-E′ (SEQ ID NO: 173), where M is methionine, Q is glutamine, and L is leucine, and A′ is 0-5 amino acids in length, B′ is 0-3 amino acids in length, C′ is 0-7 amino acids in length, D′ is 0-5 amino acids in length, and E′ is 0-6 amino acids in length, wherein any amino acid of A′, B′, C′, D′ and E′ is any amino acid.

In some embodiments, A′ is 0 amino acids in length, 1 amino acid in length, 2 amino acids in length, 3 amino acids in length, 4 amino acids in length, or 5 amino acids in length. In some embodiments, A′ is 0-1 amino acids in length, 0-2 amino acids in length, 0-3 amino acids in length, 0-4 amino acids in length, 1-2 amino acids in length, 1-3 amino acids in length, 1-4 amino acids in length, 1-5 amino acids in length, 2-3 amino acids in length, 2-4 amino acids in length, 2-5 amino acids in length, 3-4 amino acids in length, 3-5 amino acids in length, or 4-5 amino acids in length. In some embodiments, any amino acid of A′ is independently selected from any naturally occurring amino acid. In some embodiments, any amino acid of A′ is independently selected from the group consisting of threonine (T), lysine (K), and proline (P).

In some embodiments, B′ is 0 amino acids in length, 1 amino acid in length, 2 amino acids in length, or 3 amino acids in length. In some embodiments, B′ is 0-1 amino acids in length, 0-2 amino acids in length, 1-2 amino acids in length, 1-3 amino acids in length, or 2-3 amino acids in length. In some embodiments, any amino acid of B′ is independently selected from any naturally occurring amino acid. In some embodiments, any amino acid of B′ is independently selected from the group consisting of valine (V), histidine (H), and phenylalanine (F).

In some embodiments, C′ is 0 amino acids in length, 1 amino acid in length, 2 amino acids in length, 3 amino acids in length, 4 amino acids in length, 5 amino acids in length, 6 amino acids in length, or 7 amino acids in length. In some embodiments, C′ is 0-1 amino acids in length, 0-2 amino acids in length, 0-3 amino acids in length, 0-4 amino acids in length, 0-5 amino acids in length, 0-6 amino acids in length, 1-2 amino acids in length, 1-3 amino acids in length, 1-4 amino acids in length, 1-5 amino acids in length, 1-6 amino acids in length, 1-7 amino acids in length, 2-3 amino acids in length, 2-4 amino acids in length, 2-5 amino acids in length, 2-6 amino acids in length, 2-7 amino acids in length, 3-4 amino acids in length, 3-5 amino acids in length, 3-6 amino acids in length, 3-7 amino acids in length, 4-5 amino acids in length, 4-6 amino acids in length, 4-7 amino acids in length, 5-6 amino acids in length, 5-7 amino acids in length, or 6-7 amino acids in length. In some embodiments, any amino acid of C′ is independently selected from any naturally occurring amino acid. In some embodiments, any amino acid of C′ is independently selected from the group consisting of valine (V), arginine (R), serine (S), isoleucine (I), and phenylalanine (F).

In some embodiments, D′ is 0 amino acids in length, 1 amino acid in length, 2 amino acids in length, 3 amino acids in length, 4 amino acids in length, or 5 amino acids in length. In some embodiments, D′ is 0-1 amino acids in length, 0-2 amino acids in length, 0-3 amino acids in length, 0-4 amino acids in length, 1-2 amino acids in length, 1-3 amino acids in length, 1-4 amino acids in length, 1-5 amino acids in length, 2-3 amino acids in length, 2-4 amino acids in length, 2-5 amino acids in length, 3-4 amino acids in length, 3-5 amino acids in length, or 4-5 amino acids in length. In some embodiments, any amino acid of D′ is independently selected from any naturally occurring amino acid. In some embodiments, any amino acid of D′ is independently selected from the group consisting of alanine (A), valine (V), threonine (T), serine (S), isoleucine (I), leucine (L), and phenylalanine (F).

In some embodiments, E′ is 0 amino acids in length, 1 amino acid in length, 2 amino acids in length, 3 amino acids in length, 4 amino acids in length, 5 amino acids in length, or 6 amino acids in length. In some embodiments, E′ is 0-1 amino acids in length, 0-2 amino acids in length, 0-3 amino acids in length, 0-4 amino acids in length, 0-5 amino acids in length, 1-2 amino acids in length, 1-3 amino acids in length, 1-4 amino acids in length, 1-5 amino acids in length, 1-6 amino acids in length, 2-3 amino acids in length, 2-4 amino acids in length, 2-5 amino acids in length, 2-6 amino acids in length, 3-4 amino acids in length, 3-5 amino acids in length, 3-6 amino acids in length, 4-5 amino acids in length, 4-6 amino acids in length, or 5-6 amino acids in length. In some embodiments, any amino acid of E′ is independently selected from any naturally occurring amino acid. In some embodiments, any amino acid of E′ is independently selected from the group consisting of alanine (A), valine (V), leucine (L), threonine (T), serine (S), tyrosine (Y), histidine (H), cytosine (C), glutamic acid (E), and phenylalanine (F).

In some embodiments, a pre-sequence comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence MTKPTQVLVRSVSILFFITLLHLVVA (SEQ ID NO: 1). In some embodiments, a pre-sequence comprises an amino acid sequence having no more than 3, no more than 2, or no more than 1 amino acid differences (substitutions, deletions, or additions) with the amino acid sequence MTKPTQVLVRSVSILFFITLLHLVVA (SEQ ID NO: 1). In some embodiments, a pre-sequence comprises or consists of the amino acid sequence

(SEQ ID NO: 1)
MTKPTQVLVRSVSILFFITLLHLVVA.

In some embodiments, a pre-sequence comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence MQLYLTLLFLLSFVEC (SEQ ID NO: 9). In some embodiments, a pre-sequence comprises an amino acid sequence having no more than 3, no more than 2, or no more than 1 amino acid differences (substitutions, deletions, or additions) with the amino acid sequence MQLYLTLLFLLSFVEC (SEQ ID NO: 9). In some embodiments, a pre-sequence comprises or consists of the amino acid sequence MQLYLTLLFLLSFVEC (SEQ ID NO: 9).

In some embodiments, a pre-sequence comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence MQHFLSLLLAVSLLTTTYA (SEQ ID NO: 2). In some embodiments, a pre-sequence comprises an amino acid sequence having no more than 3, no more than 2, or no more than 1 amino acid differences (substitutions, deletions, or additions) with the amino acid sequence MQHFLSLLLAVSLLTTTYA (SEQ ID NO: 2). In some embodiments, a pre-sequence comprises or consists of the amino acid sequence MQHFLSLLLAVSLLTTTYA (SEQ ID NO: 2).

TABLE 1
Sequences of Exemplary Pre-Sequences

| | SEQ | Amino Acid | SEQ | | |
|---|---|---|---|---|---|
| Pre-Seq | ID | Sequence | ID | | |
| ID: | NO: | | NO: | Nucleic Acid Sequence | Origon |
| 3075301 | 1 | MTKPTQVLVR | 27 | Atgacgaagccgacacaagttcttgt | M1 killer toxin |
| (Pre1) | | SVSILFFITLLH | | gaggtcagtctctatattattctttatcac | [Saccharomyces |
| | | LVVA | | tctgctccatctagttgtagct | paradoxus]\|AII19506.1 |
| 3075303 | 2 | MQHFLSLLLA | 28 | atgcagcatttcctttccctactgttagc | alpha-amylase [Trametes |
| (Pre2) | | VSLLTTTYA | | cgttagtttgctcactacaacgtacgct | versicolor FP-101664 |
| | | | | | SS1]\|XP_008036125.1 |
| 3075305 | 3 | MNVWHAVMV | 29 | Atgaacgtttggcacgcagtaatggtg | glycoside hydrolase |
| (Pre3) | | FVLCGVVVAA | | tttgtcctatgtggggttgtagtggctgc | [Gloeophyllum trabeum |
| | | G | | cggc | ATCC 11539]\| |
| | | | | | XP_007868578.1 |
| 3075307 | 4 | MRQVWFSWIV | 30 | Atgaggcaagtctggttttcatggata | Full = Dolichyl- |
| (Pre4) | | GLFLCFFNVSS | | gttggccttttcttgtgttttttcaatgtaa | diphosphooligosaccharide- |
| | | A | | gcagtgca | protein |
| | | | | | glycosyltransferase |
| | | | | | subunit 1\|P41543.1\|\| |
| | | | | | Saccharomyces cerevisiae |
| 3075309 | 5 | MRFPSIFTAVL | 31 | atgcgattcccgtcaatctttactgccgt | Mf(alpha)1p |
| (Pre5) | | FAASSALA | | cttgtttgctgcgtctagcgcactagct | [Saccharomyces cerevisiae |
| | | | | | YJM1383]\|AJW01277.1 |
| 3075311 | 6 | MKLATAFTILT | 32 | Atgaagctagccaccgcattcacaat | Alkaline extracellular |
| (Pre6) | | AVLAAPLAAP | | acttacggctgtgctcgcggctccttta | protease\|P09230.1\| |
| | | | | gctgcaccc | Yarrowia lipolytica |
| 3075313 | 7 | MKFSAGAVLS | 33 | Atgaaattttcagcaggggctgttttaa | Protein disulfide- |
| (Pre7) | | WSSLLLASSVF | | gttggtcttccctcctgttggcctogtct | isomerase\|P17967.2\| |
| | | A | | gtgttcgcg | Saccharomyces cerevisiae |
| | | | | | S288C |
| 3075315 | 8 | MKAFTSLLCGL | 34 | atgaaggcgttcacatcgttgctttgcg | \|Carboxypeptidase Y\| |
| (Pre8) | | GLSTTLA | | gattaggcctgagcaccactctagca | P00729.1\|Saccharomyces |
| | | | | | cerevisiae |
| 3075317 | 9 | MQLYLTLLFLL | 35 | Atgcaactatacttgacacttttattcct | Endo-1,3(4)-beta- |
| (Pre9) | | SFVEC | | gctctcctttgtagagtgt | glucanase 1\|P53753.1 |
| | | | | | Saccharomyces cerevisiae |
| 3075319 | 10 | MFSLKALLPLA | 36 | Atgttcagtcttaaagcactattgccgtt | Saccharopepsin\|P07267.1 |
| (Pre 10) | | LLLVSANQVA | | agccctgttgctcgtcagcgcgaacca | Saccharomyces cerevisiae |
| | | A | | ggtagctgct | |
| 3075321 | 11 | MARFVALVLL | 37 | Atggcccgttttgtggcgttagtactac | Beta-2-microglobulin\| |
| (Pre11) | | GLLSLSGLDA | | tcggcctgctttcattgtccggtttggat | P01888.2\|P. pastoris |
| | | | | gca | recode 1 |
| 3075323 | 12 | MSLFTSLPFLL | 38 | atgtcactcttcacttccttgccctttctg | Mannose-binding protein |
| (Pre 12) | | LTAVTASC | | ctattaaccgccgtcacagcatcttgc | C\|O02659.1\|Bos taurus |
| 3075325 | 13 | MKLFVPALLSL | 39 | atgaaactgttcgtgcccgcgcttttatc | Lactotransferrin\| |
| (Pre 13) | | GALGLCLA | | actaggcgccttggggttgtgtcttgca | P24627.2\|Bos taurus |
| 3075327 | 14 | METPRASLSLG | 40 | Atggaaactcctcgagcctcattgagt | Cathelicidin-1\|P22226.2\| |
| (Pre14) | | RWSLWLLLLG | | cttggaagatggagcttatggctactgc | Bos taurus |
| | | LALPSASA | | tgttggggctcgctttgccgtccgcatc | |
| | | | | tgct | |

### Pro-Sequence

The secretion signals described herein may comprise a pro-sequence.

In some embodiments, a pro-sequence is 5-105 amino acids in length, 10-105 amino acids in length, 20-105 amino acids in length, 30-105 amino acids in length, 40-105 amino acids in length, 50-105 amino acids in length, 5-90 amino acids in length, 10-90 amino acids in length, 20-90 amino acids in length, 30-90 amino acids in length, 40-90 amino acids in length, 50-90 amino acids in length, 60-70 amino acids in length, 60-69 amino acids in length, 60-68 amino acids in length, 60-67 amino acids in length, 60-66 amino acids in length, 61-70 amino acids in length, 61-69 amino acids in length, 61-68 amino acids in length, 61-67 amino acids in length, 61-66 amino acids in length, 62-70 amino acids in length, 62-69 amino acids in length, 62-68 amino acids in length, 62-67 amino acids in length, 62-66 amino acids in length, 63-70 amino acids in length, 63-69 amino acids in length, 63-68 amino acids in length, 63-67 amino acids in length, 63-66 amino acids in length, 64-70 amino acids in length, 64-69 amino acids in length, 64-68 amino acids in length, 64-67 amino acids in length, 64-66 amino acids in length, 65-70 amino acids in length, 65-69 amino acids in length, 65-68 amino acids in length, 65-67 amino acids in length, or 65-66 amino acids in length. In some embodiments, a pro-sequence is 64 amino acids in length, 65 amino acids in length, 66 amino acids in length, 67 amino acids in length, or 68 amino acids in length.

In some embodiments, a pro-sequence comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an amino acid sequence listed in TABLE 2. In some embodiments, a pro-sequence comprises an amino acid sequence having no more than 8, no more than 7, no more than 6, no more than 5, no more than 4, no more than 3, no more than 2, or no more than 1 amino acid difference(s) (substitution, deletion, or addition) with an amino acid sequence listed in TABLE 2. In some embodiments, a pro-sequence comprises or consists of an amino acid sequence listed in TABLE 2.

In some embodiments, a pro-sequence comprises the amino acid sequence NTTIXXXA (SEQ ID NO: 62), wherein X is independently chosen from any amino acid. In some embodiments, a pre-sequence comprises the amino acid sequence NTTIXXXA (SEQ ID NO: 62), wherein X is independently chosen from any naturally-occurring amino acid. Exemplary pre-sequences of TABLE 1 that comprise the amino acid sequence NTTIXXXA (SEQ ID NO: 62) include Pro-Seq IDs: 3075261, 3075271, 3075273, 3075289, 3075291, 3075295, 3075297, and 3075299.

In some embodiments, a pro-sequence comprises the amino acid sequence RYVVGDDEQ (SEQ ID NO: 64). In some embodiments, a pro-sequence comprises the amino acid sequence IVAKSGI (SEQ ID NO: 65). In some embodiments, a pro-sequence comprises the amino acid sequence IPDEAIAN (SEQ ID NO: 66). In some embodiments, a pro-sequence comprises the amino acid sequence QTSISDDEEPIVVEINGQKV (SEQ ID NO: 67). In some embodiments, a pro-sequence comprises the amino acid sequence INTTLTEEALEKSGISIDDL (SEQ ID NO: 68). In some embodiments, a pro-sequence comprises the amino acid sequence PVFAEIDNK (SEQ ID NO: 69). In some embodiments, a pro-sequence comprises the amino acid sequence DDLKESYAN (SEQ ID NO: 70). In some embodiments, a pro-sequence comprises the amino acid sequence PVENVDD (SEQ ID NO: 71). In some embodiments, a pro-sequence comprises the amino acid sequence IDQEQLTNG (SEQ ID NO: 72). In some embodiments, a pro-sequence comprises the amino acid sequence PVDSGAKGKYSR (SEQ ID NO: 73). In some embodiments, a pro-sequence comprises the amino acid sequence NDGVGVGMSTIKEEDFGKHF (SEQ ID NO: 74). In some embodiments, a pro-sequence comprises the amino acid sequence TTIASIA (SEQ ID NO: 224). In some embodiments, a pro-sequence comprises the amino acid sequence YVVGDDEQ (SEQ ID NO: 225). In some embodiments, a pro-sequence comprises the amino acid sequence PVFAEIDNKPVVYIVNTTKA (SEQ ID NO: 226). In some embodiments, a pro-sequence comprises the amino acid sequence

(SEQ ID NO: 227)
ESIVAKSGITLDDLKESYAN.

In some embodiments, a pro-sequence comprises the amino acid sequence NTTIX⁵X⁶X⁷A (SEQ ID NO: 63), wherein: X⁵ is alanine (A), leucine (L), or tyrosine (Y); X⁶ is alanine (A), serine (S), asparagine (N), or glutamic acid (E); and X⁷ is alanine (A), isoleucine (I), serine (S), glutamic acid (E), or glutamine (Q).

In some embodiments, a pro-sequence comprises the amino acid sequence AAX³EEGX⁷SLDKR (SEQ ID NO: 221), wherein: X³ is lysine (K) or alanine (A); and X⁷ is valine (V) or serine (S).

In some embodiments, a pro-sequence comprises the amino acid sequence X¹NTTIAX⁷X⁸AX¹⁰X¹¹EEGVX¹⁶ (SEQ ID NO: 75), wherein: X¹ is valine (V) or isoleucine (I); X⁷ is aspartic acid (D), serine (S), or glutamic acid (E); X⁸ is isoleucine (I) or glutamine (Q); X¹⁰ is alanine (A) or leucine (L); X¹¹ is alanine (A) or lysine (K); and X¹⁶ is serine (S) or leucine (L).

In some embodiments, a pro-sequence comprises the amino acid sequence X¹X²X³X⁴X⁵DDEX⁹ (SEQ ID NO: 76), wherein: X¹ is arginine (R) or glutamine (Q); X² is tyrosine (Y) or threonine (T); X³ is valine (V) or serine (S); X⁴ is valine (V) or isoleucine (I); X⁵ is glycine (G) or serine (S); and X⁹ is glutamine (Q) or glutamic acid (E).

In some embodiments, a pro-sequence comprises the amino acid sequence AX²LPFSNX⁸TNX¹¹GX¹³X¹⁴FX¹⁶NTTI (SEQ ID NO: 77), wherein: X² is valine (V) or leucine (L); X⁸ is serine (S) or glycine (G); X¹¹ is asparagine (N) or threonine (T); X¹³ is isoleucine (I) or leucine (L); X¹⁴ is serine (S), leucine (L), or methionine (M); and X¹⁶ is valine (V) or isoleucine (I).

In some embodiments, a pro-sequence comprises the amino acid sequence X¹AQX⁴PAEAX⁹IGX¹²LDLX¹⁶X¹⁷X¹⁸X¹⁹D (SEQ ID NO: 78), wherein: X¹ is threonine (T) or serine (S); X⁴ is isoleucine (I) or valine (V); X⁹ is valine (V) or isoleucine (I); X¹² is tyrosine (Y) or phenylalanine (F); X¹⁶ is glutamic acid (E) or threonine (T); X¹⁷ is aspartic acid (D) or glycine (G); X¹⁸ is aspartic acid (D), serine (S), or alanine (A); and X¹⁹ is glutamic acid (E) or phenylalanine (F).

In some embodiments, a pro-sequence comprises the amino acid sequence X¹GX³X⁴X⁵X⁶X⁷DX⁹IX¹¹P (SEQ ID NO: 79), wherein: X¹ is lysine (K) or serine (S); X³ is lysine (K) or arginine (R); X⁴ is tyrosine (Y) or phenylalanine (F); X⁵ is serine (S) or leucine (L); X⁶ is arginine (R) or glutamic acid (E); X⁷ is glutamine (Q) or threonine (T); X⁹ is leucine (L) or isoleucine (I); and X¹¹ is isoleucine (I) or phenylalanine (F).

In some embodiments, a pro-sequence comprises the amino acid sequence X¹X²NX⁴TX⁶E (SEQ ID NO: 80), wherein: X¹ is asparagine (N) or proline (P); X² is glycine (G) or alanine (A); X⁴ is glycine (G) or threonine (T); and X⁶ is serine (S) or threonine (T).

In some embodiments, a pro-sequence comprises the amino acid sequence PAEAVIX⁷Y (SEQ ID NO: 228), wherein X⁷ is aspartic acid (D) or glycine (G).

In some embodiments, a pro-sequence comprises the amino acid sequence KEEX⁴X⁵X⁶X⁷X⁸KR (SEQ ID NO: 229), wherein: X⁴ is glycine (G) or glutamic acid (E); X⁵ is valine (V) or alanine (A); X⁶ is serine (S) or lysine (K); X⁷ is leucine (L) or asparagine (N); and X⁸ is aspartic acid (D) or glycine (G).

In some embodiments, a pro-sequence comprises the amino acid sequence GDFDX⁵AX⁷LP (SEQ ID NO: 230), wherein: X⁵ is valine (V) or alanine (A); and X⁷ is valine (V) or alanine (A).

In some embodiments, a pro-sequence comprises the amino acid sequence X¹SNST (SEQ ID NO: 231), wherein X¹ is leucine (L) or phenylalanine (F).

In some embodiments, a pro-sequence comprises the amino acid sequence GLSX⁴TN (SEQ ID NO: 232), wherein X⁴ is serine (S) or phenylalanine (F).

In some embodiments, a pro-sequence comprises the amino acid sequence PX²SNSTNNGLSX¹²TNTTIASI (SEQ ID NO: 233), wherein: X² is leucine (L) or phenylalanine (F); and X¹² is serine (S) or phenylalanine (F).

In some embodiments, a pro-sequence comprises the amino acid sequence X¹X²X³IPX⁶EAX⁹X¹⁰X¹¹X¹²X¹³X¹⁴X¹⁵X¹⁶X¹⁷DX¹⁹X²⁰ (SEQ ID NO: 234), wherein: X¹ is threonine (T) or aspartic acid (D); X² is alanine (A) or leucine (L); X³ is glutamine (Q) or isoleucine (I); X⁶ is alanine (A) or aspartic acid (D); X⁹ is valine (V) or isoleucine (I); X¹⁰ is isoleucine (I) or alanine (A); X¹¹ is aspartic acid (D), glycine (G), or asparagine (N); X¹² is tyrosine (Y) or arginine (R); X¹³ serine (S) or tyrosine (Y); X¹⁴ is aspartic acid (D) or valine (V); X¹⁵ is leucine (L) or valine (V); X¹⁶ is glutamic acid (E) or glycine (G); X¹⁷ is glycine (G) or aspartic acid (D); X¹⁹ is phenylalanine (F) or glutamic acid (E); and X²⁰ is aspartic acid (D) or glutamine (Q).

In some embodiments, a pro-sequence comprises the amino acid sequence APX³NX⁵TX⁷EX⁹EX¹¹X¹²QX¹⁴PAEAX¹⁹X²⁰X²¹YX²³X²⁴X²⁵EGDX²⁹DX³¹AX³³LPX³⁶X³⁷X³⁸STNX⁴²GX⁴⁴X⁴⁵X⁴⁶X⁴⁷NTTX⁵¹AX⁵³IAAKEEGVX⁶²LX⁶⁴KR (SEQ ID NO: 81), wherein: X³ is valine (V) or alanine (A); X⁵ is threonine (T) or alanine (A); X⁷ is threonine (T) or alanine (A); X⁹ is aspartic acid (D) or glycine (G); X¹¹ is threonine (T) or alanine (A); X¹² is threonine (T) or alanine (A); X¹⁴ is isoleucine (I) or threonine (T); X¹⁹ is valine (V), isoleucine (I), or alanine (A); X²⁰ is isoleucine (I) or alanine (A); X²¹ is glycine (G), aspartic acid (D), or threonine (T); X²³ is leucine (L), serine (S), or arginine (R); X²⁴ is aspartic acid (D) or glycine (G); X²⁵ is leucine (L) or serine (S); X²⁹ is phenylalanine (F), serine (S), or valine (V); X³¹ is valine (V) or alanine (A); X³³ is valine (V) or alanine (A); X³⁶ is phenylalanine (F) or leucine (L); X³⁷ serine (S) or proline (P); X³⁸ is asparagine (N), serine (S), or aspartic acid (D); X⁴² is asparagine (N) or aspartic acid (D); X⁴⁴ is leucine (L) or serine (S); X⁴⁵ is leucine (L) or serine (S); X⁴⁶ is phenylalanine (F) or serine (S); X⁴⁷ is isoleucine (I) or threonine (T); X⁵¹ is isoleucine (I) or threonine (T); X⁵³ is serine (S) or asparagine (N); X⁶² is serine (S) or threonine (T); and X⁶ is asparagine (N) or aspartic acid (D).

In some embodiments, a pro-sequence comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence APANTTTEDETAQIPAEAVIDYSDLEGDFDAAALPLSNSTNNGLSSTNTTIASIAAKEE GVSLDKR (SEQ ID NO: 61). In some embodiments, the pre-sequence comprises an amino acid sequence having no more than 6, no more than 5, no more than 4, no more than 3, no more than 2, or no more than 1 amino acid differences (substitutions, deletions, or additions) with the amino acid sequence APANTTTEDETAQIPAEAVIDYSDLEGDFDAAALPLSNSTNNGLSSTNTTIASIAAKEE GVSLDKR (SEQ ID NO: 61). In some embodiments, the pro-sequence comprises or consists of the amino acid sequence:

(SEQ ID NO: 61)
APANTTTEDETAQIPAEAVIDYSDLEGDFDAAALPLSNSTNNGLSSTN
TTIASIAAKEEGVSLDKR.

TABLE 2
Sequences of Exemplary Pro-Sequences

| | SEQ | | SEQ | | |
|---|---|---|---|---|---|
| Pro-Seq | ID | Amino Acid | ID | | |
| ID: | NO: | Sequence | NO: | Nucleic Acid Sequence | Origin |
| 3075259 | 41 | APVAAPEAEA | 83 | gcgcctgttgcagctcccgaggccgaagct | Uncharacterized |
| (Pro1) | | GGRGNFGNSG | | gggggtagaggaaacttcggcaatagcgg | protein\|A0A0J0XX36\| |
| | | PPIWKR | | accaccgatatggaagagg | Cutaneotrichosporon |
| | | | | | oleaginosum |
| 3075261 | 42 | APINITSSDPAI | 84 | gcgccaataaacattacaagcagtgaccct | LAQU0S08e04698g1_1\| |
| (Pro2) | | PSESISGFLDLT | | gccatcccctccgagtcaatttctgggtttctt | A0A0P1KVC2\| |
| | | DAEDLALLPVS | | gatctgaccgatgcagaagatttagctttgct | Lachancea quebecensis |
| | | NGTHSGVLIVN | | accagtttctaatggaactcattctggcgtctt | |
| | | TTILAQAFGSD | | gatcgtgaatacgactattctcgctcaggcat | |
| | | DVLTKR | | tcggatcggacgatgtacttacaaagagg | |
| 3075263 | 43 | APIEAPTSDETP | 85 | gcccctatcgaagcaccaacttcagatgag | Uncharacterized |
| (Pro3) | | APTEPSGRFLE | | acacccgcgccaaccgaaccgagtggcag | protein\|A0A1E4SLJ7\| |
| | | QDIIFPQQAINA | | gtttctggagcaagacattatattccctcagc | Suhomyces tanzawaensis |
| | | QTSISDDEEPIV | | aagctattaatgctcagacgtccatctcggat | NRRL Y-17324 |
| | | VEINGQKVILLI | | gacgaagaacccattgtggtagagataaac | |
| | | NTTLTEEALEK | | ggacaaaaagttattctcctaatcaacactac | |
| | | SGISIDDLNAL | | attgaccgaagaggcattggaaaagagcg | |
| | | AGNSTVSKR | | ggatatctattgatgacttaaatgctttggccg | |
| | | | | gtaattctactgtctccaaacga | |
| 3075265 | 44 | APVSISGQSLK | 86 | gcaccagtgtccattagcggccaaagtttga | Uncharacterized |
| (Pro4) | | R | | agcga | protein\|A0A1E4TVC8\| |
| | | | | | Pachysolen tannophilus |
| | | | | | NRRL Y-2460 |
| 3075267 | 45 | APVSGASNSTS | 87 | gccccggtgtccggggcatctaactcaacc | Mating factor alpha\| |
| (Pro5) | | MEVPGEAVKY | | agtatggaagtaccaggcgaggctgtcaaa | A0A1E5RDH2\| |
| | | FLDLSDSPDMA | | tattttttagacctatcggatagccctgatatg | Hanseniaspora osmophila |
| | | LVPINNGNTTG | | gctcttgttcccataaataatggaaacacaac | |
| | | IMFVNTTVIDQ | | gggtattatgttcgttaacactactgtcatcga | |
| | | AYAETTSLSRK | | ccaagcgtacgcagaaactacctctctgagt | |
| | | R | | agaaagcgt | |
| 3075269 | 46 | AAIDQEQLING | 88 | gcagccatagatcaggaacaattaaccaat | Mating factor alpha-1\| |
| (Pro6) | | TYIDIPQESILSF | | ggcacgtatatcgacattccacaagagtcaa | A0A1E5RQF6\| |
| | | LDLTDSPEVSV | | ttctttcgttcctggatttgactgatagtccgga | Hanseniaspora uvarum |
| | | YPIKEGSKTGLI | | agtgtctgtttaccctatcaaagaggggtcca | |
| | | FVNSTIVDQAY | | | |
| | | SETTALTRKR | | | |
| 3075271 | 47 | APIKFNDSSPA | 89 | gccccgataaagttcaacgattcgtccccag | LANO_0F00936g1_1\| |
| (Pro7) | | LPLESISGYLDL | | cactcccccttgagagcattagtgggtattta | A0A1G4K5X1\| |
| | | TGAEDLALLPV | | gacctgacaggtgcggaagatttggctctac | Lachancea nothofagi |
| | | SNATHTGILVV | | tgcctgtgtctaatgctacccacacgggaat | CBS 11611\| |
| | | NTTILASALAS | | cttggtcgtaaataccactattttagcctctgct | |
| | | ESNYNKR | | ttggcatcagaatcaaactacaacaaaaga | |
| 3075273 | 48 | APVNTTTEDET | 90 | gcgcctgtaaatacgaccactgaggatgaa | Mating factor alpha |
| (Pro8) | | AQIPAEAIIGYL | | acagcacaaataccagccgaagctatcattg | A0A291L9R2\| |
| | | DLEGDFDIAVL | | gttacttggaccttgagggagattttgatattg | Saccharomyces paradoxus |
| | | PFSNSTNNGLL | | ccgtgttacccttcagtaactcaacaaataac | |
| | | FINTTIANIAAE | | ggcctactctttatcaatactaccattgcaaac | |
| | | EEGVTLNKR | | atagctgctgaagaagagggggttactttga | |
| | | | | acaaacga | |
| 3075275 | 49 | KPQHYKR | 91 | aagcctcagcattataaacga | Plectin-like isoform x3 |
| (Pro9) | | | | | \|A0A2H5S501\| |
| | | | | | Rhizophagus irregularis |
| 3075277 | 50 | APVAKDATNT | 92 | gccccagtcgcgaaagacgcaacgaacac | Mating factor alpha\| |
| (Pro10) | | TDASSVQIPAE | | tacagatgctagctccgtgcaaatacctgct | A7TE98\| |
| | | AVIGYLDLEQS | | gaagctgtaattggttacctagatttagagca | Vanderwaltozyma |
| | | NDVAMLQFSN | | gtcaaatgacgttgccatgcttcaattttctaa | polyspora |
| | | STNNGILFVNS | | cagtactaataatgggatcttgttcgttaacag | |
| | | TILKAAYAEAN | | taccatcctcaaggcagcttatgcagaagct | |
| | | ANSNSNTKR | | aatgccaactctaattcaaacacaaagcga | |
| 3075279 | 51 | APVESIFANQP | 93 | gcccccgtagaatcaatatttgctaaccaac | Uncharacterized |
| (Pro11) | | DSSLTDTNDG | | ctgacagctccttgacggataccaatgatgg | protein\|C4R1N1\| |
| | | VGVGMSTIKEE | | ggtgggagtcggcatgtcgactattaagga | Komagataella phaffii |
| | | DFGKHFVENQI | | ggaagacttcggtaaacattttgttgagaatc | |
| | | LDEAVIMSLKL | | agatcctggatgaagcagttattatgtctctta | |
| | | RKGVNLFFLD | | aattacgaaagggtgttaacctattctttctcg | |
| | | DIGLATELIGN | | acgatatcggattggctacagaacttataggt | |
| | | KIAQIEAIDLSE | | aataagattgcgcaaatagaggcaattgact | |
| | | RLAQSWTNIR | | taagtgaacgtttggctcagtcttggactaac | |
| | | KNRLFGKR | | atccgcaaaaatcggctttttggaaagaga | |
| 3075281 | 52 | APVSVNDAKEI | 94 | gctcctgtatcggtcaatgatgcgaaggaaa | Mating factor alpha\| |
| (Pro12) | | AATFPQEALLG | | ttgccgcaactttcccgcaggaggctttacta | G8JP74\| |
| | | FLDLTDAENIV | | ggttttttggacctgaccgatgcagaaaacat | Eremothecium cymbalariae |
| | | ILSLVDEEKSGI | | agtgatcctctcacttgttgacgaggaaaaat | |
| | | ALVNKTIWAT | | ccggcattgctttggttaataaacgatatgg | |
| | | ARSEQAAGISK | | gccacagccaggagcgagcaagctgctgg | |
| | | R | | aattagtaagcga | |
| 3075283 | 53 | APVDSGAKGK | 95 | gctccagtagattcgggcgcaaaaggtaag | Piso0_003304 protein\| |
| (Pro13) | | YSRTDLIIPDEA | | tactctcgaaccgaccttataatccccgatga | G8YIQ7\|Pichia |
| | | IANRYVVGDD | | ggcgattgccaacaggtatgttgtgggagat | sorbitophila |
| | | EQPVFAEIDNK | | gacgaacaacctgtctttgctgaaatcgataa | |
| | | PVVYIVNTTKA | | taaaccggttgtgtatattgttaatacgactaa | |
| | | ESIVAKSGITLD | | ggcagagtcaattgtcgctaagagcgggat | |
| | | DLKESYANAT | | aacattggacgatctgaaagaaagttacgcc | |
| | | KEEEAKNGKR | | aacgctactaaagaggaagaggcaaagaa | |
| | | | | cggaaaacgg | |
| 3075285 | 54 | APVENINIKDN | 96 | gcccccgttgagaacataaatatcaaggac | Mating factor alpha\| |
| (Pro14) | | GNGTSEADVP | | aatgggaacggtacctcagaagctgatgtc | K0KH35\| |
| | | GTSQGVEFPFA | | cctggaacatcgcagggcgtggaatttccat | Wickerhamomyces ciferrii |
| | | KEAIIEAVSLG | | tcgcaaaagaggctattattgaagcggtatct | |
| | | NDIAPIVLNDA | | ctgggtaatgatattgcaccgatcgtactaaa | |
| | | VYFVNTTTVD | | cgatgctgtctactttgttaatacgactactgtt | |
| | | KELESKLGKR | | gacaaggagttggaaagtaaattaggaaaa | |
| | | | | cgg | |
| 3075287 | 55 | APVNITESANG | 97 | gcgccagttaatattacggagtccgccaacg | Putative secreted |
| (Pro15) | | EAEADVPGTS | | gggaagctgaagctgacgtcccgggcaca | protein\|K0KT23 |
| | | QGVEFPFSKEA | | tcacaaggagtggaatttcccttcagcaagg | Wickerhamomyces ciferrii |
| | | IIEAVSLGNDIA | | aggccataatcgaagcagtatctctaggtaa | |
| | | PIVLDDAVYFI | | cgatattgcacctatagttctggatgatgcgg | |
| | | NTTIVDQELGS | | tatattttattaatactaccatcgtcgaccagg | |
| | | KLGKR | | agcttggtagtaaactcggaaaacgt | |
| 3075289 | 56 | APVSTETDIDD | 98 | gctccagtgtccacagaaacggacatcgat | Mating factor alpha\| |
| (Pro16) | | LPISVPEEALIG | | gatttgcccataagtgttccggaggaagccc | Q6CMM5\| |
| | | FIDLTGDEVSL | | ttattggctttattgatctgaccggagatgagg | Kluyveromyces lactis |
| | | LPVNNGTHTGI | | tcagcttattgcctgtaaacaatggtactcata | |
| | | LFLNTTIAEAA | | ctgggatactattcctcaatacaactattgcg | |
| | | FADKDDLKKR | | gaagctgcatttgcagacaaggatgacttaa | |
| | | | | aaaagcgt | |
| 3075291 | 57 | APVENVDDSA | 99 | gcaccagtagaaaacgtggatgactccgcc | Mating factor alpha\| |
| (Pro17) | | QVPEEAIIGYID | | caggtccccgaggaagcgattatcggatac | Q874L5\| |
| | | FEGASDVAILP | | atagatttcgagggcgcttcggatgttgctatt | Kluyveromyces delphensis |
| | | FSNSTDSGLMF | | cttccttttagtaatagcacagactcagggtta | |
| | | VNTTIYNEATT | | atgtttgttaatacgaccatctataacgaagct | |
| | | AVEGESVEKR | | actactgcggtcgagggtgaatctgttgaaa | |
| | | | | agcga | |
| 3075293 | 58 | APIDVSLAKR | 100 | gctcctatcgatgtgtctcttgccaagcgt | Mating factor alpha |
| (Pro18) | | | | | W6MQJ6\| |
| | | | | | Kuraishia capsulata |
| 3075295 | 59 | APVNTTTEDET | 101 | gcaccggtcaatacaaccacggaagatga | Mf(alpha)1p |
| (Pro19) | | AQIPAEAVIGY | | gactgcccaaatacccgctgaagcggtgat | AJW01277.1\| |
| | | LDLEGDFDVA | | cggctacctggaccttgagggagatttcgac | Saccharomyces cerevisiae |
| | | VLPFSNSTNNG | | gttgctgttctaccatttagtaacagcactaat | |
| | | LLFINTTIASIA | | aacgggttattgtttattaatacaacgattgca | |
| | | AKEEGVSLDK | | tctatcgccgccaaagaagagggtgtatcac | |
| | | R | | tggataagcga | |
| 3075297 | 60 | APANTTTEDET | 102 | gcaccggccaatacaaccacggaagatga | Mf(alpha)1p |
| (Pro20) | | AQIPAEAVIGY | | gactgctcaaatacccgctgaagcggtgatc | YJM1383]\| |
| | | LDLEGDFDVA | | ggctacctggaccttgagggagatttcgacg | Saccharomyces cerevisiae |
| | | VLPFSNSTNNG | | tcgcagttctaccatttagtaacagcactaata | |
| | | LSFINTTIASIA | | acgggttatcctttattaatacaacgattgcttc | |
| | | AKEEGVSLDKR | | tatcgccgcgaaagaagagggtgtatcact | |
| | | | | cgataagcga | |
| 3075299 | 61 | APANTTTEDET | 103 | gcaccggccaatacaaccacggaagatga | Mf(alpha)1p |
| (Pro21) | | AQIPAEAVIDY | | gactgctcaaatacccgctgaagcggtgatc | [Saccharomyces |
| | | SDLEGDFDAA | | gactacagcgatcttgagggcgacttcgatg | cerevisiae YJM1383]\| |
| | | ALPLSNSTNNG | | cagccgctctgccattatcgaacagtactaat | AJW01277.1\| |
| | | LSSTNTTIASIA | | aacgggttgtcttcaaccaatacaacgattgc | Saccharomyces cerevisiae |
| | | AKEEGVSLDK | | ctctattgcagcaaaagaagagggagtatcc | |
| | | R | | ctagataagcga | |

### Cleavage Sequence

A secretion signal may further comprise a cleavage sequence. As cleavage of a cleavage sequence will depend on the presence of a protease corresponding to a cleavage sequence, the choice of which cleavage sequence to use may depend on the organism in which one seeks to express the secreted protein (i.e., one may wish to utilize a cleavage sequence corresponding to a protease that a host cell expresses).

In some embodiments, a secretion signal comprises, from N-terminus to C-terminus: a pre-sequence (or pre-region); a pro-sequence (or pro-region); and a cleavage sequence. In some embodiments, a fusion polypeptide comprising a secretion signal and a polypeptide of interest comprises one or more linkers (e.g., between the pre-sequence and the pro-sequence of the secretion signal, and/or between the pro-sequence and the sequence of the polypeptide of interest).

In some embodiments, a C-terminal cleavage sequence comprises an amino acid sequence listed in TABLE 3.

TABLE 3
Amino Acid Sequences of Exemplary Cleavage Sequences

| SEQ | Cleav- | Preferred | | |
|---|---|---|---|---|
| ID NO: | Seq ID: | Organism(s) | Sequence | Protease |
| 104 | EAEA | Pichia pastoris | EAEA | Ste13 |
| n/a | KR | Pichia pastoris, other | KR | Kex2 |
| | | yeasts, and fungi | | |

In some embodiments, a cleavage site is: a ADAM metallopeptidase with thrombospondin type 1 motif-13 (ADAMTS13) cleavage site, a byovirus Ma protease cleavage site, a byovirus RNA-2-encoded protease cleavage site, a Caspase-3 cleavage site, a Cathepsin L cleavage site, a chemotrypsin a Coagulation Factor IXa cleavage site, a Coagulation Factor VIIa cleavage site, a Coagulation Factor Xa cleavage site, a Coagulation Factor XIIa cleavage site, a Coagulation Factor XIa cleavage site, a comovirus 24K protease cleavage site, a DPPIV cleavage site, a Factor Xa protease cleavage site, a Furin cleavage site, a genenase I cleavage site, a Granzyme B cleavage site, a heparin cleavage site, a Kell blood group cleavage site, a Kex2 cleavage site, a Matrix Metalloproteinase-2 (MMP-2) cleavage site, a Matrix Metalloproteinase-9 (MMP-9) cleavage site, a MMP cleavage site, a Mouse mast cell protease-7 (mMCP-7) cleavage site, a nepovirus 24K protease cleavage site, a PAR 2 cleavage site, a PAR3 cleavage site, a PAR4 cleavage site, a parsnip yellow fleck virus (PYVF) 3C-like protease cleavage site, a picorna virus 3C protease cleavage site, a plasma kallikrein cleavage site, a Plasmin cleavage site, a potyvirus HC protease cleavage site, a potyvirus Ma protease cleavage site, a potyvirus P1 (P35) protease cleavage site, a PreScission (Human Rhinovirus 3C Protease) cleavage site, a protease-activated G protein-coupled receptor-1 (PARI) cleavage site, a rhinovirus 2A protease cleavage site, a rice tungro spherical virus (RTSV) 3C-like protease cleavage site, a Ste13 cleavage site, a subtilisin (e.g., PC2, PC1/PC3, PACE4, PC4, PC5/PC6, LPC/PC7IPC8/SPC7 and SKI-1) cleavage site, a Thrombin cleavage site, a tissue-type Plasminogen activator (tPA) cleavage site, a Tobacco etch virus (TEV) protease cleavage site, a tobacco vein mottling virus (TVMV) protease cleavage site, a Tryptase-F cleavage site, or a urokinase-type Plasminogen activator (uPA) cleavage site a VP4 of IPNV cleavage site, an aphthovirus L protease cleavage site, an endothelin-converting enzyme-1 (ECE-1) cleavage site, an enterokinase cleavage site, an enterokinase cleavage site, an enterovirus 2A protease cleavage site, an Epx1 cleavage site from Pichia pastoris (VSAAP), or an Igase cleavage site; the sequences of these cleavage sites have been described in the art.

In some embodiments, if the host cell is Aspergillus niger, the cleavage site is that recognized by a protease which is: prolyl-alanyl-specific endoprotease (EndoPro) (van Schaick et al. 2021 J. Proteome 20: 4875); KexB (Jalving et al. 2000 App. Env. Microb. 66:363); PepAa, PepAb, PepAc, or PepAd (Wang et al. 2008 Fung. Genet. Biol. 45: 17); PepC (Frederick et al. 1993 Gene 125:57); PepD (Jarai et al. 1994 Gene 139: 51); PepE (Jarai et al. 1994 Gene 145: 171); or PepF (van den Hombergh et al. 1994 Gene 151: 73); or any other Aspergillus niger protease that has been described in the art.

In various embodiments, the different components of a secretion signal (e.g., pre sequence, pro sequence, cleavage sites, etc.) can be derived from one, two, or more than two different genes from two or more different organisms. For example, a cleavage site can be derived from the same or different gene than the pre sequence and/or the pro sequence. In some embodiments, wherein the cleavage site is recognized by a protease which is not native (endogenous) to the host cell, a gene encoding the protease can be introduced into the host cell.

### Exemplary Secretion Signals

One having ordinary skill in the art will appreciate that the secretion signals described herein may comprise any combination of a pre-sequence provided herein (see e.g., TABLE 1), a pro-sequence provided herein (see e.g., TABLE 2), and optionally a cleavage sequence provided herein (see e.g., TABLE 3).

In some embodiments, a secretion signal comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an amino acid sequence listed in TABLE 4. In some embodiments, a secretion signal comprises an amino acid sequence having no more than 10, no more than 9, no more than 8, no more than 7, no more than 6, no more than 5, no more than 4, no more than 3, no more than 2, or no more than 1 amino acid difference (substitution, deletion, or addition) with an amino acid sequence listed in TABLE 4. In some embodiments, a secretion signal comprises or consists of an amino acid sequence listed in TABLE 4.

In some embodiments, a secretion signal comprises the amino acid sequence CX²X³X⁴X⁵X⁶X⁷X⁸APX¹¹NTTT (SEQ ID NO: 146), wherein: X² is leucine (L), phenylalanine (F), or glycine (G); X³ is leucine (L), phenylalanine (F), or valine (V); X⁴ is asparagine (N) or valine (V); X⁵ is valine (V) or leucine (L); X⁶ is serine (S), alanine (A), or valine (V); X⁷ is serine (S) or alanine (A); X⁸ is alanine (A) or glycine (G); and X¹¹ is valine (V) or alanine (A).

In some embodiments, a secretion signal comprises the amino acid sequence X¹AAPX⁵X⁶TTTEDE (SEQ ID NO: 147), wherein: X¹ is leucine (L), serine (S), or alanine (A); X⁵ is alanine (A) or valine (V); and X⁶ is asparagine (N) or serine (S).

In some embodiments, a secretion signal comprises the amino acid sequence AAPIX⁵X⁶X⁷X⁸S (SEQ ID NO: 148), wherein: X⁵ is asparagine (N) or lysine (K); X⁶ is isoleucine (I) or phenylalanine (F); X⁷ is threonine (T) or asparagine (N); and X⁸ is serine (S) or aspartic acid (D).

In some embodiments, a secretion signal comprises the amino acid sequence X¹X²X³X⁴X⁵X⁶X⁷X⁸X⁹ (SEQ ID NO: 149), wherein: X¹ is glutamine (Q) or asparagine (N); X² is valine (V) or histidine (H); X³ is tryptophan (W) or phenylalanine (F); X⁴ is phenylalanine (F), leucine (L), or histidine (H); X⁵ is serine (S) or alanine (A); X⁶ is tryptophan (W), leucine (L), or valine (V); X⁷ is isoleucine (I), leucine (L), or methionine (M); X⁸ is valine (V) or leucine (L); and X⁹ is glycine (G), alanine (A), or phenylalanine (F).

In some embodiments, a secretion signal comprises the amino acid sequence X¹X²X³X⁴X⁵X⁶AX⁸X⁹ (SEQ ID NO: 150), wherein: X¹ lysine (K) or asparagine (N); X² is glycine (G), asparagine (N), or aspartic acid (D); X³ is asparagine (N), glycine (G), or lysine (K); X⁴ is leucine (L), tyrosine (Y), or glycine (G); X⁵ is serine (S) or asparagine (N); X⁶ is serine (S), arginine (R), or glycine (G); X⁸ is asparagine (N), aspartic acid (D), or serine (S); and X⁹ is threonine (T), leucine (L), or glutamic acid (E).

In some embodiments, a secretion signal comprises the amino acid sequence X¹RX³X⁴X⁵X⁶X⁷X⁸X⁹X¹⁰X¹¹X¹²X¹³X¹⁴X¹⁵X¹⁶ (SEQ ID NO: 151), wherein: X¹ is methionine (M), valine (V), or glutamine (Q); X³ is phenylalanine (F) or glutamine (Q); X⁴ is leucine (L) or valine (V); X⁵ is serine (S) or tryptophan (W); X⁶ is phenylalanine (F) or leucine (L); X⁷ is leucine (L) or serine (S); X⁸ is threonine (T), leucine (L), phenylalanine (F), or tryptophan (W); X⁹ is alanine (A), leucine (L), or isoleucine (I); X¹⁰ is valine (V) or leucine (L); X¹¹ is leucine (L), glycine (G), or serine (S); X¹² is leucine (L) or phenylalanine (F); X¹³ is valine (V), leucine (L), or phenylalanine (F); X¹⁴ is valine (V) or leucine (L); X¹⁵ is serine (S) or cytosine (C); and X¹⁶ is alanine (A) or phenylalanine (F).

In some embodiments, a secretion signal comprises the amino acid sequence X¹X²X³X⁴X⁵X⁶X⁷X⁸X⁹X¹⁰X¹¹X¹²X¹³X¹⁴X¹⁵X¹⁶X¹⁷IX¹⁹X²⁰ (SEQ ID NO: 152), wherein: X¹ is aspartic acid (D), valine (V), or glutamic acid (E); X² is valine (V), tyrosine (Y), or proline (P); X³ is proline (P), isoleucine (I), or serine (S); X⁴ is glycine (G) or valine (V); X⁵ is threonine (T), asparagine (N), or arginine (R); X⁶ is serine (S), threonine (T), or phenylalanine (F); X⁷ is glutamine (Q), threonine (T), or leucine (L); X⁸ is glycine (G), lysine (K), or glutamic acid (E); X⁹ is valine (V), alanine (A), or glutamine (Q); X¹⁰ is glutamic acid (E) or aspartic acid (D); X¹¹ is phenylalanine (F), serine (S), or isoleucine (I); X¹² is isoleucine (I) or proline (P); X¹³ is phenylalanine (F) or valine (V); X¹⁴ is alanine (A) or proline (P); X¹⁵ is lysine (K) or glutamine (Q); X¹⁶ is glutamic acid (E), serine (S), or glutamine (Q); X¹⁷ is alanine (A) or glycine (G); X¹⁹ is isoleucine (I), threonine (T), or asparagine (N); and X²⁰ is glutamic acid (E), leucine (L), or alanine (A).

In some embodiments, a secretion signal comprises the amino acid sequence AAPX⁴X⁵X⁶X⁷X⁸X⁹X¹⁰X¹¹X¹² (SEQ ID NO: 235), wherein: X⁴ is alanine (A) or valine (V); X⁵ is asparagine (N) or aspartic acid (D); X⁶ is serine (S) or threonine (T); X⁷ is threonine (T) or glycine (G); X⁸ is threonine (T) or alanine (A); X⁹ is glutamic acid (E) or lysine (K); X¹⁰ is glycine (G) or aspartic acid (D); X¹¹ is glutamic acid (E) or lysine (K); and X¹² is threonine (T) or tyrosine (Y).

In some embodiments, a secretion signal comprises the amino acid sequence AX²KEEX⁶X⁷X⁸X⁹X¹⁰KREAEA (SEQ ID NO: 236), wherein: X² is alanine (A) or threonine (T); X⁶ is glycine (G) or glutamic acid (E); X⁷ is valine (V) or alanine (A); X⁸ is serine (S) or lysine (K); X⁹ is leucine (L) or asparagine (N); and X¹⁰ is aspartic acid (D) or glycine (G).

In some embodiments, a secretion signal comprises the amino acid sequence SLLX⁴X⁵SX⁷X⁸LAAPX¹³NTTTEDE (SEQ ID NO: 237), wherein: X⁴ is alanine (A), phenylalanine (F), leucine (L), or serine (S); X⁵ is leucine (L) or alanine (A); X⁷ is leucine (L) or serine (S); X⁸ is leucine (L) or valine (V); and X¹³ is alanine (A) or valine (V).

In some embodiments, a secretion signal comprises the amino acid sequence MX²X³X⁴X⁵X⁶X⁷X⁸X⁹ (SEQ ID NO: 238), wherein: X² is alanine (A), lysine (K), or arginine (R); X³ is leucine (L), glutamine (Q), or arginine (R); X⁴ is phenylalanine (F) or valine (V); X⁵ is valine (V) or tryptophan (W); X⁶ is alanine (A), phenylalanine (F), or proline (P); X⁷ is leucine (L), alanine (A), or serine (S); X⁸ is leucine (L), valine (V), or tryptophan (W); and X⁹ is leucine (L) or isoleucine (I).

In some embodiments, a secretion signal comprises the amino acid sequence LX²X³X⁴X⁵LLX⁸X⁹X¹⁰X¹¹X¹²APX¹⁵NX¹⁷TX¹⁹EX²¹EX²³X²⁴QX²⁶PAEAX³¹X³²X³³YX³⁵X³⁶X³⁷EGDX⁴¹DX⁴³AX⁴⁵LPX⁴⁸X⁴⁹X⁵⁰STNX⁵⁴GX⁵⁶X⁵⁷X⁵⁸X⁵⁹NTTX⁶³ASIAAKEEGVSLDKR (SEQ ID NO: 153), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); X¹² is alanine (A) or cytosine (C); X¹⁵ is valine (V) or alanine (A); X¹⁷ is threonine (T) or alanine (A); X¹⁹ is threonine (T) or alanine (A); X²¹ is aspartic acid (D) or glycine (G); X²³ is threonine (T) or alanine (A); X²⁴ is threonine (T) or alanine (A); X²⁶ is isoleucine (I) or threonine (T); X³¹ is valine (V) or alanine (A); X³² is isoleucine (I) or alanine (A); X³³ is glycine (G), aspartic acid (D), or threonine (T); X³⁵ is leucine (L), serine (S), or arginine (R); X³⁶ is aspartic acid (D) or glycine (G); X³⁷ is leucine (L) or serine (S); X⁴¹ is phenylalanine (F), serine (S), or valine (V); X⁴³ is valine (V) or alanine (A); X⁴⁵ is valine (V) or alanine (A); X⁴⁸ is phenylalanine (F) or leucine (L); X⁴⁹ serine (S) or proline (P); X⁵⁰ is asparagine (N), serine (S), or aspartic acid (D); X⁵⁴ is asparagine (N) or aspartic acid (D); X⁵⁶ is leucine (L) or serine (S); X⁵⁷ is leucine (L) or serine (S); X⁵⁸ is phenylalanine (F) or serine (S); X⁵⁹ is isoleucine (I) or threonine (T); and X⁶³ is isoleucine (I) or threonine (T);
optionally wherein the secretion signal further comprises an N-terminal amino acid sequence of: MTKPTQVLVRSVSI (SEQ ID NO: 154); MQLY (SEQ ID NO: 155); or MQHFLSL (SEQ ID NO: 156).

In some embodiments, a secretion signal comprises the amino acid sequence LX²X³X⁴X⁵LLX⁸X⁹X¹⁰X¹¹X¹²APANTTTEDETAQIPAEAVIDYSDLEGDFDAAALPLSNS TNNGLSSTNTTIASIAAKEEGVSLDKR (SEQ ID NO: 157), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); and X¹² is alanine (A) or cytosine (C); optionally wherein the secretion signal further comprises an N-terminal amino acid sequence of: MTKPTQVLVRSVSI (SEQ ID NO: 154); MQLY (SEQ ID NO: 155); or MQHFLSL (SEQ ID NO: 156).

In some embodiments, a secretion signal further comprises an N-terminal amino acid sequence MTKPTQVLVRSVSI (SEQ ID NO: 154). In some embodiments, a secretion signal further comprises an N-terminal amino acid sequence MQLY (SEQ ID NO: 155). In some embodiments, a secretion signal further comprises an N-terminal amino acid sequence MQHFLSL (SEQ ID NO: 156).

In some embodiments, a secretion signal comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence MTKPTQVLVRSVSILFFITLLHLVVAAPANTTTEDETAQIPAEAVIDYSDLEGDFDAA ALPLSNSTNNGLSSTNTTIASIAAKEEGVSLDKR (SEQ ID NO: 107). In some embodiments, a pre-sequence comprises an amino acid sequence having no more than 7, no more than 6, no more than 5, no more than 4, no more than 3, no more than 2, or no more than 1 amino acid differences (substitutions, deletions, or additions) with the amino acid sequence MTKPTQVLVRSVSILFFITLLHLVVAAPANTTTEDETAQIPAEAVIDYSDLEGDFDAA ALPLSNSTNNGLSSTNTTIASIAAKEEGVSLDKR (SEQ ID NO: 107). In some embodiments, a secretion signal comprises or consists of the amino acid sequence of

(SEQ ID NO: 107)
MTKPTQVLVRSVSILFFITLLHLVVAAPANTTTEDETAQIPAEAVIDYS
DLEGDFDAAALPLSNSTNNGLSSTNTTIASIAAKEEGVSLDKR.

In some embodiments, a secretion signal comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence MQLYLTLLFLLSFVECAPANTTTEDETAQIPAEAVIDYSDLEGDFDAAALPLSNSTNN GLSSTNTTIASIAAKEEGVSLDKR (SEQ ID NO: 108). In some embodiments, the pre-sequence comprises an amino acid sequence having no more than 7, no more than 6, no more than 5, no more than 4, no more than 3, no more than 2, or no more than 1 amino acid differences (substitutions, deletions, or additions) with the amino acid sequence MQLYLTLLFLLSFVECAPANTTTEDETAQIPAEAVIDYSDLEGDFDAAALPLSNSTNN GLSSTNTTIASIAAKEEGVSLDKR (SEQ ID NO: 108). In some embodiments, a secretion signal comprises or consists of the amino acid sequence of

(SEQ ID NO: 108)
MQLYLTLLFLLSFVECAPANTTTEDETAQIPAEAVIDYSDLEGDFDAAA
LPLSNSTNNGLSSTNTTIASIAAKEEGVSLDKR.

In some embodiments, a secretion signal comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence MQHFLSLLLAVSLLTTTYAAPANTTTEDETAQIPAEAVIDYSDLEGDFDAAALPLSNS TNNGLSSTNTTIASIAAKEEGVSLDKR (SEQ ID NO: 115). In some embodiments, the pre-sequence comprises an amino acid sequence having no more than 7, no more than 6, no more than 5, no more than 4, no more than 3, no more than 2, or no more than 1 amino acid differences (substitutions, deletions, or additions) with the amino acid sequence MQHFLSLLLAVSLLTTTYAAPANTTTEDETAQIPAEAVIDYSDLEGDFDAAALPLSNS TNNGLSSTNTTIASIAAKEEGVSLDKR (SEQ ID NO: 115). In some embodiments, a secretion signal comprises or consists of the amino acid sequence of

(SEQ ID NO: 115)
MQHFLSLLLAVSLLTTTYAAPANTTTEDETAQIPAEAVIDYSDLEGDFD
AAALPLSNSTNNGLSSTNTTIASIAAKEEGVSLDKR.

In some embodiments, a secretion signal does not comprise the amino acid sequence of

(SEQ ID NO: 158)
APVNTTTEDETAQIPAEAVIGYLDLEGDFDVAVLPFSNSTNNGLLFINT
TIASIAAKEEGVQLDKR.

In some embodiments, a secretion signal comprises a pre-sequence and pro-sequence from the same protein (i.e., a pre-sequence and a pro-sequence of a secretion signal of a naturally occurring protein). For example, in some embodiments, a secretion signal comprises the amino acid sequence

(SEQ ID NO: 119)
MKFTLATLLVLATAAIAAPVAAPEAEAGGRGNFGNSGPPIWKR

(or sectag228). In some embodiments, a secretion signal comprises the amino acid sequence MKLKYFLLIFVFTTVLAKPQHYKR (SEQ ID NO: 139) (or sectag998). In some embodiments, the present disclosure pertains to a fusion protein comprising, from N- to C-terminus: a secretion signal comprising sectag228 or sectag994, and a polypeptide of interest, or a polynucleotide encoding such a fusion protein.

TABLE 4
Amino Acid Sequences of Exemplary Secretion Signals

| SEQ | | Description | | | |
|---|---|---|---|---|---|
| ID NO: | SecTag ID: | Pre-Seq ID: | Pro-Seq ID: | Cleav-Seq ID: | Sequence |
| 106 | sectag687 | 3075321 | 3075299 | KR | MARFVALVLLGLLSLSGLDAAPANTTTEDETA |
| | | | | | DAAALPLSNSTNNGLSSTNTTIASIAAKEEGVS |
| 107 | sectag909 | 3075301 | 3075299 | KR | MTKPTQVLVRSVSILFFITLLHLVVAAPANTTI |
| | | | | | LEGDFDAAALPLSNSTNNGLSSTNTTIASIAAK |
| 108 | sectag923 | 3075317 | 3075299 | KR | MQLYLTLLFLLSFVECAPANTTTEDETAQIPAE |
| | | | | | LPLSNSTNNGLSSTNTTIASIAAKEEGVSLDKR |
| 109 | sectag153 | 3075309 | 3075299 | KR | MRFPSIFTAVLFAASSALAAPANTTTEDETAQI |
| | | | | | AALPLSNSTNNGLSSTNTTIASIAAKEEGVSLD |
| 110 | sectag938 | 3075327 | 3075299 | KR | METPRASLSLGRWSLWLLLLGLALPSASAAPA |
| | | | | | DYSDLEGDFDAAALPLSNSTNNGLSSTNTTIAS |
| 111 | sectag225 | 3075313 | 3075299 | KR | MKFSAGAVLSWSSLLLASSVFAAPANTTTEDE |
| | | | | | DFDAAALPLSNSTNNGLSSTNTTIASIAAKEEG |
| 112 | sectag101 | 3075307 | 3075283 | KR | MRQVWFSWIVGLFLCFFNVSSAAPVDSGAKG |
| | | | | | VVGDDEQPVFAEIDNKPVVYIVNTTKAESIVA |
| | | | | | KEEEAKNGKR |
| 113 | sectag108 | 3075307 | 3075297 | KR | MRQVWFSWIVGLFLCFFNVSSAAPANTTTEDE |
| | | | | | DFDVAVLPFSNSTNNGLSFINTTIASIAAKEEGY |
| 114 | sectag974 | 3075319 | 3075299 | KR | MFSLKALLPLALLLVSANQVAAAPANTTTEDE |
| | | | | | DFDAAALPLSNSTNNGLSSTNTTIASIAAKEEG |
| 115 | sectag983 | 3075303 | 3075299 | KR | MQHFLSLLLAVSLLTTTYAAPANTTTEDETAC |
| | | | | | AAALPLSNSTNNGLSSTNTTIASIAAKEEGVSL |
| 116 | sectag159 | 3075325 | 3075299 | KR | MKLFVPALLSLGALGLCLAAPANTTTEDETA( |
| | | | | | AAALPLSNSTNNGLSSTNTTIASIAAKEEGVSL |
| 117 | sectag691 | 3075307 | 3075259 | KR | MRQVWFSWIVGLFLCFFNVSSAAPVAAPEAE/ |
| 118 | sectag960 | 3075303 | 3075283 | KR | MQHFLSLLLAVSLLTTTYAAPVDSGAKGKYSI |
| | | | | | DDEQPVFAEIDNKPVVYIVNTTKAESIVAKSGI |
| | | | | | AKNGKR |
| 115 | sectag168 | 3075303 | 3075299 | KR | MQHFLSLLLAVSLLTTTYAAPANTTTEDETAç |
| | | | | | AAALPLSNSTNNGLSSTNTTIASIAAKEEGVSL |
| 120 | sectag003 | 3075321 | 3075283 | KR | MARFVALVLLGLLSLSGLDAAPVDSGAKGKY |
| | | | | | GDDEQPVFAEIDNKPVVYIVNTTKAESIVAKS( |
| | | | | | EAKNGKR |
| 121 | sectag987 | 3075305 | 3075265 | KR | MNVWHAVMVFVLCGVVVAAGAPVSISGQSL |
| 122 | sectag759 | 3075309 | 3075291 | KR | MRFPSIFTAVLFAASSALAAPVENVDDSAQVP. |
| | | | | | PFSNSTDSGLMFVNTTIYNEATTAVEGESVEKI |
| 123 | sectag135 | 3075325 | 3075283 | KR | MKLFVPALLSLGALGLCLAAPVDSGAKGKYS |
| | | | | | DDEQPVFAEIDNKPVVYIVNTTKAESIVAKSGI |
| | | | | | AKNGKR |
| 124 | sectag157 | 3075325 | 3075297 | KR | MKLFVPALLSLGALGLCLAAPANTTTEDETA( |
| | | | | | VAVLPFSNSTNNGLSFINTTIASIAAKEEGVSLI |
| 125 | sectag961 | 3075303 | 3075285 | KR | MQHFLSLLLAVSLLTTTYAAPVENINIKDNGN |
| | | | | | FAKEAIIEAVSLGNDIAPIVLNDAVYFVNTTTV |
| 126 | sectag687 | 3075321 | 3075299 | KREAEA | MARFVALVLLGLLSLSGLDAAPANTTTEDETA |
| | | | | | DAAALPLSNSTNNGLSSTNTTIASIAAKEEGVS |
| 127 | sectag909 | 3075301 | 3075299 | KREAEA | MTKPTQVLVRSVSILFFITLLHLVVAAPANTT] |
| | | | | | LEGDFDAAALPLSNSTNNGLSSTNTTIASIAAK |
| 128 | sectag923 | 3075317 | 3075299 | KREAEA | MQLYLTLLFLLSFVECAPANTTTEDETAQIPAI |
| | | | | | LPLSNSTNNGLSSTNTTIASIAAKEEGVSLDKR |
| 129 | sectag153 | 3075309 | 3075299 | KREAEA | MRFPSIFTAVLFAASSALAAPANTTTEDETAQ. |
| | | | | | AALPLSNSTNNGLSSTNTTIASIAAKEEGVSLD |
| 130 | sectag938 | 3075327 | 3075299 | KREAEA | METPRASLSLGRWSLWLLLLGLALPSASAAP/ |
| | | | | | DYSDLEGDFDAAALPLSNSTNNGLSSTNTTIA |
| 131 | sectag225 | 3075313 | 3075299 | KREAEA | MKFSAGAVLSWSSLLLASSVFAAPANTTTEDI |
| | | | | | DFDAAALPLSNSTNNGLSSTNTTIASIAAKEEC |
| 132 | sectag101 | 3075307 | 3075283 | KREAEA | MRQVWFSWIVGLFLCFFNVSSAAPVDSGAKG |
| | | | | | VVGDDEQPVFAEIDNKPVVYIVNTTKAESIVA |
| | | | | | KEEEAKNGKREAEA |
| 133 | sectag108 | 3075307 | 3075297 | KREAEA | MRQVWFSWIVGLFLCFFNVSSAAPANTTTED] |
| | | | | | DFDVAVLPFSNSTNNGLSFINTTIASIAAKEEG |
| 134 | sectag974 | 3075319 | 3075299 | KREAEA | MFSLKALLPLALLLVSANQVAAAPANTTTED] |
| | | | | | DFDAAALPLSNSTNNGLSSTNTTIASIAAKEEC |
| 135 | sectag983 | 3075303 | 3075299 | KREAEA | MQHFLSLLLAVSLLTTTYAAPANTTTEDETA( |
| | | | | | AAALPLSNSTNNGLSSTNTTIASIAAKEEGVSL |
| 136 | sectag159 | 3075325 | 3075299 | KREAEA | MKLFVPALLSLGALGLCLAAPANTTTEDETA( |
| | | | | | AAALPLSNSTNNGLSSTNTTIASIAAKEEGVSL |
| 137 | sectag691 | 3075307 | 3075259 | KREAEA | MRQVWFSWIVGLFLCFFNVSSAAPVAAPEAE |
| | | | | | EAEA |
| 138 | sectag960 | 3075303 | 3075283 | KREAEA | MQHFLSLLLAVSLLTTTYAAPVDSGAKGKYS. |
| | | | | | DDEQPVFAEIDNKPVVYIVNTTKAESIVAKSG. |
| | | | | | AKNGKREAEA |
| 135 | sectag168 | 3075303 | 3075299 | KREAEA | MQHFLSLLLAVSLLTTTYAAPANTTTEDETA( |
| | | | | | AAALPLSNSTNNGLSSTNTTIASIAAKEEGVSL |
| 140 | sectag003 | 3075321 | 3075283 | KREAEA | MARFVALVLLGLLSLSGLDAAPVDSGAKGKY |
| | | | | | GDDEQPVFAEIDNKPVVYIVNTTKAESIVAKS |
| | | | | | EAKNGKREAEA |
| 141 | sectag987 | 3075305 | 3075265 | KREAEA | MNVWHAVMVFVLCGVVVAAGAPVSISGQSL |
| 171 | sectag759 | 3075309 | 3075291 | KREAEA | MRFPSIFTAVLFAASSALAAPVENVDDSAQVP |
| | | | | | PFSNSTDSGLMFVNTTIYNEATTAVEGESVEK |
| 142 | sectag135 | 3075325 | 3075283 | KREAEA | MKLFVPALLSLGALGLCLAAPVDSGAKGKYS |
| | | | | | DDEQPVFAEIDNKPVVYIVNTTKAESIVAKSG. |
| | | | | | AKNGKREAEA |
| 143 | sectag157 | 3075325 | 3075297 | KREAEA | MKLFVPALLSLGALGLCLAAPANTTTEDETA( |
| | | | | | VAVLPFSNSTNNGLSFINTTIASIAAKEEGVSLI |
| 144 | sectag961 | 3075303 | 3075285 | KREAEA | MQHFLSLLLAVSLLTTTYAAPVENINIKDNGN |
| | | | | | FAKEAIIEAVSLGNDIAPIVLNDAVYFVNTTTV |

| | | | |
|---|---|---|---|
| 145 | alphaMF | with EAEA cleavage sequence | MRFPSIFTAVLFAASSALAAPVNTTTEDETAQ |
| | native | | AVLPFSNSTNNGLLFINTTIASIAAKEEGVSLD |
| 119 | sectag228 | from Uncharacterized protein, | MKFTLATLLVLATAAIAAPVAAPEAEAGGRG |
| | | Cutaneotrichosporon oleaginosum, | |
| | | A0A0J0XX36 | |
| 139 | sectag994 | from Plectin-like isoform x3, | MKLKYFLLIFVFTTVLAKPQHYKR |
| | | Rhizophagus irregularis (strain | |
| | | DAOM 181602/DAOM 197198/MUCL 43194 | |

### Proteins of Interest

The secretion signals described herein may be affixed to a protein of interest (or heterologous protein) that one has interest in expressing in a cell.

Exemplary proteins of interest and their corresponding amino acids of interest are provided in TABLE 5. Note that in TABLE 5 and elsewhere herein, the initial codon M (Met) has been deleted from the amino acid sequences as not absolutely necessary; the corresponding start codon (AUG or ATG) has similarly been deleted from various nucleotide sequences.

In some embodiments, a protein of interest is a lactoferrin. In some embodiments, the lactoferrin protein is bovine lactoferrin (bLF). In some embodiments, the bLF protein comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an amino acid sequence of SEQ ID NO: 159. In some embodiments, the bLF protein comprises or consists of an amino acid sequence of SEQ ID NO: 159.

In some embodiments, a protein of interest is a lactoglobulin. In some embodiments, the lactoglobulin protein is bovine lactoglobulin (bLG). In some embodiments, the bLG protein comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an amino acid sequence of any one of SEQ ID NOs: 160-165. In some embodiments, the bLG protein comprises or consists of an amino acid sequence of any one of SEQ ID NOs: 160-165.

In some embodiments, a protein of interest is an ovalbumin (OVA) protein. In some embodiments, the OVA protein comprises an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an amino acid sequence of any one of SEQ ID NOs: 166-170. In some embodiments, the OVA protein comprises or consists of an amino acid sequence of any one of SEQ ID NOs: 166-170.

TABLE 5
Amino Acid Sequences of Exemplary Proteins of Interest

| SEQ | | |
|---|---|---|
| ID NO: | Descr. | Sequence |
| 159 | bLF (Bos | APRKNVRWCTISQPEWFKCRRWQWRMKKLGAPSITCVRRAFALECIRAIAEKK |
| | taurus; | ADAVTLDGGMVFEAGRDPYKLRPVAAEIYGTKESPQTHYYAVAVVKKGSNFQ |
| | UniProt | LDQLQGRKSCHTGLGRSAGWIIPMGILRPYLSWTESLEPLQGAVAKFFSASCVP |
| | P24627) | CIDRQAYPNLCQLCKGEGENQCACSSREPYFGYSGAFKCLQDGAGDVAFVKET |
| | | TVFENLPEKADRDQYELLCLNNSRAPVDAFKECHLAQVPSHAVVARSVDGKED |
| | | LIWKLLSKAQEKFGKNKSRSFQLFGSPPGQRDLLFKDSALGFLRIPSKVDSALYL |
| | | GSRYLTTLKNLRETAEEVKARYTRVVWCAVGPEEQKKCQQWSQQSGQNVTCA |
| | | TASTTDDCIVLVLKGEADALNLDGGYIYTAGKCGLVPVLAENRKSSKHSSLDC |
| | | VLRPTEGYLAVAVVKKANEGLTWNSLKDKKSCHTAVDRTAGWNIPMGLIVNQ |
| | | TGSCAFDEFFSQSCAPGADPKSRLCALCAGDDQGLDKCVPNSKEKYYGYTGAF |
| | | RCLAEDVGDVAFVKNDTVWENTNGESTADWAKNLNREDFRLLCLDGTRKPVT |
| | | EAQSCHLAVAPNHAVVSRSDRAAHVKQVLLHQQALFGKNGKNCPDKFCLFKS |
| | | ETKNLLFNDNTECLAKLGGRPTYEEYLGTEYVTAIANLKKCSTSPLLEACAFLTR |
| 160 | bLG (Equus | TNIPQTMQDLDLQEVAGKWHSVAMAASDISLLDSESAPLRVYIEKLRPTPEDNL |
| | caballus; | EIILREGENKGCAEKKIFAEKTESPAEFKINYLDEDTVFALDTDYKNYLFLCMKN |
| | UniProt | AATPGQSLVCQYLARTQMVDEEIMEKFRRALQPLPGRVQIVPDLTRMAERCRI |
| | P02758) | |
| 161 | bLG | IIVTQTMKDLDVQKVAGTWYSLAMAASDISLLDAQSAPLRVYVEELKPTPGGD |
| | (Rangifer | LEILLQKWENGKCAQKKIIAEKTEIPAVFKIDALNENKVLVLDTDYKKYLLFCM |
| | tarandus; | ENSAEPEQSLACQCLVRTPEVDDEAMEKFDKALKALPMHIRLSFNPTQLEEQCR |
| | UniProt | V |
| | Q00P86) | |
| 162 | bLG (Bos | LIVTQTMKGLDIQKVAGTWYSLAMAASDISLLDAQSAPLRVYVEELKPTPEGDL |
| | taurus; | EILLQKWENDECAQKKIIAEKTKIPAVFKIDALNENKVLVLDTDYKKYLLFCME |
| | UniProt: | NSAEPEQSLVCQCLVRTPEVDDEALEKFDKALKALPMHIRLSFNPTQLEEQCHI |
| | B5B0D4) | |
| 163 | bLG (Ovis | IIVTQTMKGLDIQKVAGTWHSLAMAASDISLLDAQSAPLRVYVEELKPTPEGNL |
| | aries; | EILLQKWENGECAQKKIIAEKTKIPAVFKIDALNENKVLVLDTDYKKYLLFCME |
| | UniProt | NSAEPEQSLACQCLVRTPEVDNEALEKFDKALKALPMHIRLAFNPTQLEGQCHV |
| | P67976) | |
| 164 | bLG (Capra | IIVTQTMKGLDIQKVAGTWYSLAMAASDISLLDAQSAPLRVYVEELKPTPEGNL |
| | hircus; | EILLQKWENGECAQKKIIAEKTKIPAVFKIDALNENKVLVLDTDYKKYLLFCME |
| | UniProt | NSAEPEQSLACQCLVRTPEVDKEALEKFDKALKALPMHIRLAFNPTQLEGQCHV |
| | P02756) | |
| 165 | bLG | IVVPQTVENLDLQKVAGTWHSLAIAASDISLLDAETAPLRVYVQELRPTPEGNL |
| | (Arctocephalus | EIVLRKWEDGRCPEQKVVAEKTKVPAEFKINYLEENKIFVLDTDYKNYLFFCME |
| | tropicalis; | NTDAPEQRLMCQYLARTLKVDNEVMGKFNRALETLPVHMQIIPDLTQGKEQCH |
| | UniProt | V |
| | W5QN41) | |
| 166 | OVA | GSIGAASMEFCFDVFKELKVHHANDNMLYSPFAILSTLAMVFLGAKDSTRTQIN |
| | (Coturnix; | KVVHFDKLPGFGDSIEAQCGTSANVHSSLRDILNQITKQNDAYSFSLASRLYAQ |
| | UniProt: | ETYTVVPEYLQCVKELYRGGLESVNFQTAADQARGLINAWVESQINGIIRNILQ |
| | Q6V115) | PSSVDSQTAMVLVNAIAFKGLWEKAFKAEDTQTIPFRVTEQESKPVQMMHQIG |
| | | SFKVASMASEKMKILELPFASGTMSMLVLLPDDVSGLEQLESTISFEKLTEWTSS |
| | | SIMEERKVKVYLPRMKMEEKYNLTSLLMAMGITDLFSSSANLSGISSVGSLKIPQ |
| | | AVHAAYAEINEAGRDVVGSAEAGVDATEEFRADHPFLFCVKHIETNAILLFGRC |
| | | VSP |
| 167 | OVA | GSIGAASTEFCFDVFKELKVQHVNENIFYSPLSIISALSMVYLGARENTRAQIDK |
| | (Fulmarus | VVHFDKITGFGETIESQCGTSVSVHTSLKDMFTQITKPSDNYSLSFASRLYAEET |
| | glacialis; | YPILPEYLQCVKELYKGGLETTSFQTAADQARELINSWVESQTNGMIKNILQPGS |
| | NCBI XP_009580141.1) | VDPQTEMVLVNAIYFKGMWEKAFKDEDTQAVPFRMTEQESKTVQMMYQIGSF |
| | | KVAVMASEKMKILELPYASGELSMLVMLPDDVSGLEQELTAITFEKLMEWTSS |
| | | NMMEERKMKVYLPRMKMEEKYNLTSVLMALGVTDLFSSSANLSGISSAESLK |
| | | MSEAVHEAFVEIYEAGSEVVGSTGAGMEVTSVSEEFRADHPFLFLIKHNPTNSIL |
| | | FFGRCFSP |
| 168 | OVA (Phalacrocorax | GSIGAASSEFCFDIFKELKVQHVNENIFYSPLSHSALSMVYLGARENTRAQIDKV |
| | carbo; | VPFDKITASGESIESQVQKIQCSTSVSVHTSLKDIFTQITKSSDNHSLSFASRLYAE |
| | NCBI XP_009507609.1) | ETYPILPEYLQCVKELYEGGLETISFQTAADQARELINSWIESQTNGRIKNILQPG |
| | | SVDPQTEMVLVNAIYFKGMWEKAFKDEDTQAVPFRMTEQESKPVQVMHQIGS |
| | | FKVAVLASEKIKILELPYASGELSMLVLLPDDVSGLEQLETAITFEKLMEWTSPN |
| | | IMEERKIKVFLPRMKIEEKYNLTSVLMALGITDLFSPLANLSGISSAESLKMSEAI |
| | | HEAFVEISEAGSEVIGSTEAEVEVINDPEEFRADHPFLFLIKHNPTNSILFFGRCFS |
| | | P |
| 169 | OVA | GSIGAASTEFCFDMFKELKVHHVNENIIYSPLSIISILSMVFLGARENTKTQMEKV |
| | (Dromaius novaehollandiae; | IHFDKITGFGESLESQCGTSVSVHASLKDILSEITKPSDNYSLSLASKLYAEETYP |
| | UniProt | VLPEYLQCIKELYKGSLETVSFQTAADQARELINSWVETQTNGVIKNFLQPGSV |
| | E2RVI8) | DPQTEMVLVDAIYFKGTWEKAFKDEDTQEVPFRITEQESKPVQMMYQAGSFKV |
| | | ATVAAEKMKILELPYASGELSMFVLLPDDISGLEQLETTISIEKLSEWTSSNMME |
| | | DRKMKVYLPHMKIEEKYNLTSVLVALGMTDLFSPSANLSGISTAQTLKMSEAIH |
| | | GAYVEIYEAGSEMATSTGVLVEAASVSEEFRVDHPFLFLIKHNPSNSILFFGRCIF |
| | | P |
| 170 | OVA | GSIGTASAEFCFDVFKELKVHHVNENIFYSPLSIISALSMVYLGARENTKTQMEK |
| | (Struthio | VIHFDKITGLGESMESQCGTGVSIHTALKDMLSEITKPSDNYSLSLASRLYAEQT |
| | camelus | YAILPEYLQCIKELYKESLETVSFQTAADQARELINSWIESQTNGVIKNFLQPGSV |
| | australis; | DSQTELVLVNAIYFKGMWEKAFKDEDTQEVPFRITEQESRPVQMMYQAGSFKV |
| | NCBI XP_009676351.1) | ATVAAEKIKILELPYASGELSMLVLLPDDISGLEQLETTISFEKLTEWTSSNMME |
| | | DRNMKVYLPRMKIEEKYNLTSVLIALGMTDLFSPAANLSGISAAESLKMSEAIH |
| | | AAYVEIYEADSEIVSSAGVQVEVTSDSEEFRVDHPFLFLIKHNPTNSVLFFGRCIS |
| | | P |

### Transcriptional Units

Aspects of the disclosure relate to transcriptional units encoding a gene (e.g., a protein of interest, Calreticulin, PDIA3, HAC1, PDI1, GPX1, HSF1, etc.). In some embodiments, a sequence of nucleotides that codes for at least one RNA molecule is a gene of interest. In some embodiments, a transcriptional unit comprises additional sequences for expression, transcription, and/or translation of a bioprotein (e.g., RNA or protein) encoded thereby, e.g., a 5′-UTR (5′-untranslated region), a leader sequence, and/or a 3′-UTR (3′-untranslated region), and/or one or more introns. In some embodiments, a transcriptional unit comprises one or more transcription terminators. In some embodiments, a transcriptional unit comprises one or more transcription terminators downstream of other components of the transcriptional unit.

In some embodiments, a promoter of the transcriptional unit is operably linked to a coding sequence (e.g., a gene). A coding sequence and a regulatory sequence (e.g., a promoter sequence) are said to be “operably joined” or “operably linked” when the coding sequence and the regulatory sequence are covalently linked and/or the expression or transcription of the coding sequence is under the influence or control of the regulatory sequence.

In some embodiments, a promoter comprises a TATA box, or similar sequence, which is capable of directing RNA polymerase II to initiate RNA synthesis at the appropriate transcription initiation site for a particular polynucleotide sequence. In some embodiments, a promoter may additionally comprise other sequences, generally but not always positioned upstream of the TATA box, referred to as upstream promoter elements, which influence the transcription initiation rate.

In certain organisms (e.g., yeasts), a promoter, including upstream promoter elements, may be understood to encompass a sequence spanning from up to 1500 base pairs (bp) upstream of the start codon of the gene to the base abutting (e.g., immediately upstream of) the first base of the start codon of the gene. In some embodiments, the 5′-UTR region is the region of an mRNA that begins at the transcription start site and ends directly upstream from the start codon. In some embodiments, a promoter comprises a 5′-UTR, which comprises the region from the +1 position of the transcriptional start to the base abutting (immediately upstream of) the start codon (e.g., ATG) of the gene. In some embodiments, a promoter comprises the core promoter and the 5′ untranslated region (5′-UTR). For any particular promoter, the exact 5′ and 3′ ends of the promoter sequence may be defined differently by different sources, scientific references, etc.

Various promoters that are useful for expressing a heterologous gene include, but are not limited to: a strong promoter, constitutive promoter, an inducible promoter, a heterologous promoter (e.g., a promoter not situated as it would be in a wild-type context), an endogenous promoter, a synthetic promoter, a promoter whose expression is driven by a transcription factor binding to an operator wherein the transcription factor itself is expressed using a heterologous promoter, a synthetic promoter, a full-length or truncated promoter, a weak promoter, or a promoter which is a component in an expression system. Various promoters are disclosed in the art, for example: Jensen et al. 1998 Appl. Environ. Microbiol. 64:82-7; Kosuri et al. 2013 Proc. Natl. Acad. Sci. U.S.A., 110:14024-9; Deuschle et al. 1986 EMBO J., 5:2987-94; Danino et al. 2015 Biochim. Biophys. Acta Gene Regulatory Mechanisms 1849: 1116; Henke et al. 2021 Microorganisms, 9, 204; Giebelmann et al. 2018 Biotechnol. J., 14, 1800417. Additional promoters and expression systems are disclosed in: international patent publication WO2022051696A1 and international patent publication WO2022108839A1 [e.g., any of the promoters of SEQ ID NOs: 29 to 61 or variants thereof], which are also incorporated by reference in this application. In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. Non-limiting examples of inducible promoters include chemically-regulated promoters and physically-regulated promoters. For chemically-regulated promoters, the transcriptional activity can be regulated by one or more compounds, such as alcohol, an antibiotic such as tetracycline, a carbon source such as galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. Non-limiting examples of a constitutive promoter include GAP, GCW14, TDH3, PGK1, PKC1, PDC1, TEF2, RPL18B, SSA1, TDH2, PYK1, TPI1, HXT3, HXT7, ACT1, ADH1, ADH2, ENO2, and SOD1, and variants of any of them.

In some embodiments, various promoters useful for work in hosts such as Pichia include but are not limited to: 0319, p0374, p0472, p0547, pAOX1, pAOX176, pAOX2, pAOX2-mutant, pAOX737-ΔD+3D, pCAT1, pCAT1-mutant, pDAS1, pDAS2, pFDH1, pFLD1, pGAP, and pPMP20. These are described in the literature, including but not limited to: Curran et al. Metab Eng, 19 (2013), pp. 88-97; Dai et al. Yi Chuan Xue Bao, 27 (2000), pp. 641-646; Gao et al. 2021 Synth. Syst. Biotech. 6: 110; Hartner et al. Nucleic Acids Res, 36 (2008), p. e76; Ito et al. Nucleic Acids Res, 48 (2020), pp. 13000-13012; Karaoglan et al. Protein Expr Purif, 121 (2016), pp. 112-117; Massahi et al. Biochem Eng J, 138 (2018), pp. 111-120; Shen et al. Microb Cell Factories, 15 (2016), p. 178; Sheng et al. Synthetic Biology Journal, 1 (2020), pp. 709-721; Vogl et al. ACS Synth Biol, 5 (2016), pp. 172-186; Xuan et al. FEMS Yeast Res, 9 (2009), pp. 1271-1282. In some embodiments, promoters useful for work in hosts such as Aspergillus are described in the literature, including but not limited to: Meyer et al. 2011 Biotech. Lett. 33: 469; Fleibner et al. 2010 App. Microb. Biotech. 87: 1255; and Lubertozzi et al. 2009 Biotech. Adv. 27. Other inducible promoters or constitutive promoters known to one of ordinary skill in the art are also contemplated.

In some embodiments, a promoter is a synthetic promoter as described in WO 2022/108839 A1 or WO 2022/051696 A1, the entire contents of each of which are incorporated herein by reference. A synthetic promoter may be a promoter provided in Table 6 of WO 2022/108839 A1 or a promoter having a sequence that is at least 90%, at least 91%, at least 92%, at least 93%, at least 94%, at least 95%, at least 96%, at least 97%, at least 98%, or at least 99% identical to a nucleic acid sequence in Table 6 of WO 2022/108839 A1, or a functional fragment thereof. A synthetic promoter may be a promoter provided in any of Tables 15-18 of WO 2022/051696 A1 or a promoter having a sequence that is at least 90%, at least 91%, at least 92%, at least 93%, at least 94%, at least 95%, at least 96%, at least 97%, at least 98%, or at least 99% identical to a nucleic acid sequence in any of Tables 15-18 of WO 2022/051696 A1, or a functional fragment thereof.

In some embodiments, a promoter of a transcriptional unit described herein comprises a sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a sequence of TABLE 6. In some embodiments, a promoter of a transcriptional unit described herein comprises or consists of a sequence listed in TABLE 6.

In some embodiments, transcriptional units optionally comprise a transcriptional terminator. In some embodiments, a transcriptional terminator is capable of terminating transcription (e.g., transcription of a transcription factor, a transcriptional activator, or a bioproduct). In some embodiments, the transcriptional terminator is a forward terminator. When located downstream of a polynucleotide sequence primed for transcription, a forward transcriptional terminator will cause transcription to terminate following transcription of the polynucleotide.

In some embodiments, terminators useful for work in hosts such as Pichia include but are not limited to: AOX1, AOX1 with an inserted NotI restriction site, and various additional terminators described herein or described in the literature, including but not limited to: Curran et al. Metab Eng, 19 (2013), pp. 88-97; Dai et al. Yi Chuan Xue Bao, 27 (2000), pp. 641-646; Gao et al. 2021 Synth. Syst. Biotech. 6: 110; Hartner et al. Nucleic Acids Res, 36 (2008), p. e76; Ito et al. Nucleic Acids Res, 48 (2020), pp. 13000-13012; Karaoglan et al. Protein Expr Purif, 121 (2016), pp. 112-117; Massahi et al. Biochem Eng J, 138 (2018), pp. 111-120; Shen et al. Microb Cell Factories, 15 (2016), p. 178; Sheng et al. Synthetic Biology Journal, 1 (2020), pp. 709-721; Vogl et al. ACS Synth Biol, 5 (2016), pp. 172-186; Xuan et al. FEMS Yeast Res, 9 (2009), pp. 1271-1282. In some embodiments, terminators useful for work in hosts such as Aspergillus are described in the literature, including but not limited to: Meyer et al. 2011 Biotech. Lett. 33: 469; Fleibner et al. 2010 App. Microb. Biotech. 87: 1255; and Lubertozzi et al. 2009 Biotech. Adv. 27. Other terminators known to one of ordinary skill in the art are also contemplated.

In some embodiments, a transcriptional terminator of a transcriptional unit described herein comprises a sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a sequence of TABLE 7. In some embodiments, a transcriptional terminator of a transcriptional unit described herein comprises or consists of a sequence listed in TABLE 7.

TABLE 6
Nucleic Acid Sequences of Exemplary Promoters

| SEQ | | |
|---|---|---|
| ID NO: | Descr. | Sequence |
| 171 | pGCW14 | CAGGTGAACCCACCTAACTATTTTTAACTGGGATCCAGTGAGCTCGCTGGGTG |
| | | AAAGCCAACCATCTTTTGTTTCGGGGAACCGTGCTCGCCCCGTAAAGTTAATT |
| | | TTTTTTTCCCGCGCAGCTTTAATCTTTCGGCAGAGAAGGCGTTTTCATCGTAG |
| | | CGTGGGAACAGAATAATCAGTTCATGTGCTATACAGGCACATGGCAGCAGTCA |
| | | CTATTTTGCTTTTTAACCTTAAAGTCGTTCATCAATCATTAACTGACCAATCA |
| | | GATTTTTTGCATTTGCCACTTATCTAAAAATACTTTTGTATCTCGCAGATACG |
| | | TTCAGTGGTTTCCAGGACAACACCCAAAAAAAGGTATCAATGCCACTAGGCAG |
| | | TCGGTTTTATTTTTGGTCACCCACGCAAAGAAGCACCCACCTCTTTTAGGTTT |
| | | TAAGTTGTGGGAACAGTAACACCGCCTAGAGCTTCAGGAAAAACCAGTACCTG |
| | | TGACCGCAATTCACCATGATGCAGAATGTTAATTTAAACGAGTGCCAAATCAA |
| | | GATTTCAACAGACAAATCAATCGATCCATAGTTACCCATTCCAGCCTTTTCGT |
| | | CGTCGAGCCTGCTTCATTCCTGCCTCAGGTGCATAACTTTGCATGAAAAGTCC |
| | | AGATTAGGGCAGATTTTGAGTTTAAAATAGGAAATATAAACAAATATACCGCG |
| | | AAAAAGGTTTGTTTATAGCTTTTCGCCTGGTGCCGTACGGTATAAATACATAC |
| | | TCTCCTCCCCCCCCTGGTTCTCTTTTTCTTTTGTTACTTACATTTTACCGTTC |
| | | CGTCACTCGCTTCACTCAACAACAAAA |
| 174 | PglaA | TGAGTTCATCCTGCAGAATACCGCGGCGTTCCACATCTGATGCCATTGGCGGA |
| | | GGGGTCCGGACGGTCAGGAACTTAGCCTTATGAGATGAATGATGGACGTGTCT |
| | | GGCCTCGGAAAAGGATATATGGGGATCATAATAGTACTAGCCATATTAATGAA |
| | | GGGCATATACCACGCGTTGGACCTGCGTTATAGCTTCCCGTTAGTTATAGTAC |
| | | CATCGTTATACCAGCCAATCAAGTCACCACGCACGACCGGGGACGGCGAATCC |
| | | CCGGGAATTGAAAGAAATTGCATCCCAGGCCAGTGAGGCCAGCGATTGGCCAC |
| | | CTCTCCAAGGCACAGGGCCATTCTGCAGCGCTGGTGGATTCATCGCAATTTCC |
| | | CCCGGCCCGGCCCGACACCGCTATAGGCTGGTTCTCCCACACCATCGGAGATT |
| | | CGTCGCCTAATGTCTCGTCCGTTCACAAGCTGAAGAGCTTGAAGTGGCGAGAT |
| | | GTCTCTGCAGGAATTCAAGCTAGATGCTAAGCGATATTGCATGGCAATATGTG |
| | | TTGATGCATGTGCTTCTTCCTTCAGCTTCCCCTCGTGCAGATGAGGTTTGGCT |
| | | ATAAATTGAAGTGGTTGGTCGGGGTTCCGTGAGGGGCTGAAGTGCTTCCTCCC |
| | | TTTTAGACGCAACTGAGAGCCTGAGCTTCATCCCCAGCATCATTACACCTCAG |
| | | CC |
| 175 | pGAP_ver2 | CGACTATTATCGATCAATGAAATCCATCAAGATTGAAATCTTAAAATTGCCCC |
| | | TTTCACTTGACAGGATCCTTTTTTGTAGAAATGTCTTGGTGTCCTCGTCCAAT |
| | | CAGGTAGCCATCTCTGAAATATCTGGCTCCGTTGCAACTCCGAACGACCTGCT |
| | | GGCAACGTAAAATTCTCCGGGGTAAAACTTAAATGTGGAGTAATGGAACCAGA |
| | | AACGTCTCTTCCCTTCTCTCTCCTTCCACCGCCCGTTACCGTCCCTAGGAAAT |
| | | TTTACTCTGCTGGAGAGCTTCTTCTACGGCCCCCTTGCAGCAATGCTCTTCCC |
| | | AGCATTACGTTGCGGGTAAAACGGAGGTCGTGTACCCGACCTAGCAGCCCAGG |
| | | GATGGAAAAGTCCCGGCCGTCGCTGGCAATAATAGCGGGCGGACGCATGTCAT |
| | | GAGATTATTGGAAACCACCAGAATCGAATATAAAAGGCGAACACCTTTCCCAA |
| | | TTTTGGTTTCTCCTGACCCAAAGACTTTAAATTTAATTTATTTGTCCCTATTT |
| | | CAATCAATTGAACAACTATCAAAACACG |
| 176 | pGAP-G1 + | GATCCTTTTTTGTAGAAATGTCTTGGTGTCCTCGACCAATCAGGTAGCCATCC |
| | Kozak | CTGAAATACCTGGCTCCGTGGCAACACCGAACGACCTGCTGGCAACGTTAAAT |
| | | TCTCCGGGGTAAAACTTAAATGTGGAGTAATAGAACCAGAAACGTCTCTTCCC |
| | | TTCTCTCTCCTTCCACCGCCCGTTACCGTCCCTAGGAAATTTTACTCTGCTGG |
| | | AGAGCTTCTTCTACGGCCCCCTTGCAGCAATGCTCTTCCCAGCATTACGTTGC |
| | | GGGTAAAACGGAGGTCGTGTACCCGACCTAGCAGCCCAGGGATGGAAAGTCCC |
| | | GGCCGTCGCTGGCAATAACTGCGGGCGGACGCATGTCTTGAGATTATTGGAAA |
| | | CCACCAGAATCGAATATAAAAGGCGAACACCTTTCCCAATTTTGGTTTCTCCT |
| | | GACCCAAAGACTTTAAATTTAATTTATTTGTCCCTATTTCAATCAATTGAACA |
| | | ACTATCAAAACACA |
| 177 | P(G6_492) | GATCCTTTTTTGTAGAAATGTCTTGGTGTCCTCGTCCAATCAGGTAGCCATCT |
| | a.k.a | CTGAAATATCTGGCTCCGTTGCAACTCCGAACGACCTGCTGGCAACGTAAGAT |
| | pGAP | ACTCCGAGGTAAAACTTAAATGTGGAGTAATGGAACCAGAAACGTCTCTTCCT |
| | (G6_492) | TTCTCTCTCCTTCCACCGCCCGTTACCGTCCCTAGGAAGTTTTACTCTGCTGG |
| | | AGAGCTTCTTCTACGGCCCCCTTGCAGCAATGCTCTTCCCAGCATTACGTTGC |
| | | GGGTAAAACGGAGGTCTTGTACCCGACCTAGCAGCCCAGGGATGGAAAAGTCC |
| | | CGGCCGTTGCTGGCAATAATAGCGGGCGGACGCATGTCATGAGATTACTGGAA |
| | | ACCACCAGAATCGAACATAAAAGGCGAACACCTGTCCTAAATTAGGTTTCTCC |
| | | TGACCCAAAGACTTTAAATTTAATTTATTTGTCCCTATTTCAATCAATTGAAC |
| | | AACTATCAAAACACA |
| 178 | P(THI4) | CCGTGATTCACTCTGTCAATGATTACCCCTCTCCTACCCGATTTGGGACTTTT |
| | | TCTTCAGTCTTGGGGACTTTTTTTCATATGACTTGACCTTGCTTTCCCAATAG |
| | | GGAAGGACTCACCCATGGATGATTAAGTTTGGATTACTCGTTTAGGAAATAGT |
| | | AGCCATGAATCAATTTGAATCATACCATCATGAAATAGGGTTAGGCTGTAAAT |
| | | GCCTCAAAAATGGCTCTTGAGGCTGGATTTTTGGGTATTGGAATGTTGGTAGC |
| | | AATTGGTATAAAAGGCCATTTGTATTTCACTTTTTTGTCCTTCATACTTTACT |
| | | CTTCTCAACTTTGGAAACTTCAATAAATCATC |
| 179 | 4xtetO- | GAGCTCCCTAGGTGTCCCTATCAGTGATAGAGACTTGCCCCATTCGCTAAGCC |
| | P(AOX1) | CACTCCCTATCAGTGATAGAGAAGCTAGACCTTACGGATTGGTGCTCCCTATC |
| | | AGTGATAGAGAGGTCGAACATCTGCTATAAGCGCTCCCTATCAGTGATAGAGA |
| | | AAAGTGAAAGTCGAGCTCGGTACCCAACCCCTACTTGACAGCAATATATAAAC |
| | | AGAAGGAAGCTGCCCTGTCTTAAACCTTTTTTTTTATCATCATTATTAGCTTA |
| | | CTTTCATAATTGCGACTGGTTCCAATTGACAAGCTTTTGATTTTAACGACTTT |
| | | TAACGACAACTTGAGAAGATCAAAAAACAACTAATTATTCGAAACG |
| 180 | 4xvanO- | GAGCTCCCTAGGTGATTGGATCCAATCTTGCCCCATTCGCTAAGCCCACATTG |
| | P(AOX1) | GATCCAATAGCTAGACCTTACGGATTGGTGCATTGGATCCAATGGTCGAACAT |
| | | CTGCTATAAGCGCATTGGATCCAATAAAGTGAAAGTCGAGCTCGGTACCCAAC |
| | | CCCTACTTGACAGCAATATATAAACAGAAGGAAGCTGCCCTGTCTTAAACCTT |
| | | TTTTTTTATCATCATTATTAGCTTACTTTCATAATTGCGACTGGTTCCAATTG |
| | | ACAAGCTTTTGATTTTAACGACTTTTAACGACAACTTGAGAAGATCAAAAAAC |
| | | AACTAATTATTCGAAACG |
| 181 | 2xvanO- | GAGCTCCCTAGGTGATTGGATCCAATCTTGCCCCATTCGCTAAGCCCACATTG |
| | P(AOX1) | GATCCAATAAAGTGAAAGTCGAGCTCGGTACCCAACCCCTACTTGACAGCAAT |
| | | ATATAAACAGAAGGAAGCTGCCCTGTCTTAAACCTTTTTTTTTATCATCATTA |
| | | TTAGCTTACTTTCATAATTGCGACTGGTTCCAATTGACAAGCTTTTGATTTTA |
| | | ACGACTTTTAACGACAACTTGAGAAGATCAAAAAACAACTAATTATTCGAAAC |
| | | G |
| 182 | 1xtetO- | GAGCTCCCTAGGTGTCCCTATCAGTGATAGAGAAAAGTGAAAGTCGAGCTCGG |
| | P(AOX1) | TACCCAACCCCTACTTGACAGCAATATATAAACAGAAGGAAGCTGCCCTGTCT |
| | | TAAACCTTTTTTTTTATCATCATTATTAGCTTACTTTCATAATTGCGACTGGT |
| | | TCCAATTGACAAGCTTTTGATTTTAACGACTTTTAACGACAACTTGAGAAGAT |
| | | CAAAAAACAACTAATTATTCGAAACG |
| 183 | 8xtetO- | GAGCTCCCTAGGTGTCCCTATCAGTGATAGAGACTTGCCCCATTCGCTAAGCC |
| | P(AOX1) | CACTCCCTATCAGTGATAGAGAAGCTAGACCTTACGGATTGGTGCTCCCTATC |
| | | AGTGATAGAGAGGTCGAACATCTGCTATAAGCGCTCCCTATCAGTGATAGAGA |
| | | TCGTCGACCTAGCTCTGTCTTAGTCCCTATCAGTGATAGAGATAACATGCCTC |
| | | TCACTAACATGGTCCCTATCAGTGATAGAGACTACTGGGGCCACGATTCGTGT |
| | | GTCCCTATCAGTGATAGAGATCTGCGTAATACTACTCGCGTGTTCCCTATCAG |
| | | TGATAGAGAAAAGTGAAAGTCGAGCTCGGTACCCAACCCCTACTTGACAGCAA |
| | | TATATAAACAGAAGGAAGCTGCCCTGTCTTAAACCTTTTTTTTTATCATCATT |
| | | ATTAGCTTACTTTCATAATTGCGACTGGTTCCAATTGACAAGCTTTTGATTTT |
| | | AACGACTTTTAACGACAACTTGAGAAGATCAAAAAACAACTAATTATTCGAAA |
| | | CG |
| 184 | 4xbmO- | GAGCTCCCTAGGTGCGGAATGAACTTTCATTCCGCTTGCCCCATTCGCTAAGC |
| | P(AOX1) | CCACCGGAATGAACTTTCATTCCGAGCTAGACCTTACGGATTGGTGCCGGAAT |
| | | GAACTTTCATTCCGGGTCGAACATCTGCTATAAGCGCCGGAATGAACTTTCAT |
| | | TCCGAAAGTGAAAGTCGAGCTCGGTACCCAACCCCTACTTGACAGCAATATAT |
| | | AAACAGAAGGAAGCTGCCCTGTCTTAAACCTTTTTTTTTATCATCATTATTAG |
| | | CTTACTTTCATAATTGCGACTGGTTCCAATTGACAAGCTTTTGATTTTAACGA |
| | | CTTTTAACGACAACTTGAGAAGATCAAAAAACAACTAATTATTCGAAACG |

TABLE 7
Nucleic Acid Sequences of Exemplary Terminators

| SEQ | | |
|---|---|---|
| ID NO: | Descr. | Sequence |
| 185 | tAOX1_ | TCAAGAGGATGTCAGAATGCCATTTGCCTGAGAGATGCAGGCTTCATTTTTGAT |
| | 650bp | ACTTTTTTATTTGTAACCTATATAGTATAGGATTTTTTTTGTCATTTTGTTTCT |
| | | TCTCGTACGAGCTTGCTCCTGATCAGCCTATCTCGCAGCTGATGAATATCTTGT |
| | | GGTAGGGGTTTGGGAAAATCATTCGAGTTTGATGTTTTTCTTGGTATTTCCCAC |
| | | TCCTCTTCAGAGTACAGAAGATTAAGTGAGACGTTCGTTTGTGCAAGCTTCAAC |
| | | GATGCCAAAAGGGTATAATAAGCGTCATTTGCAGCATTGTGAAGAAAACTATGT |
| | | GGCAAGCCAAGCCTGCGAAGAATGTATTTTAAGTTTGACTTTGATGTATTCACT |
| | | TGATTAAGCCATAATTCTCGAGTATCTATGATTGGAAGTATGGGAATGGTGATA |
| | | CCCGCATTCTTCAGTGTCTTGAGGTCTCCTATCAGATTATGCCCAACTAAAGCA |
| | | ACCGGAGGAGGAGATTTCATGGTAAATTTCTCTGACTTTTGGTCATCAGTAGAC |
| | | TCGAACTGTGAGACTATCTCGGTTATGACAGCAGAAATGTCCTTCTTGGAGACA |
| | | GTAAATGAAGTCCCACCAATAAAGAAATCCTTGTTATCAGGAACAAACTTCTTG |
| | | TT |
| 186 | tFDH1 | TTGAAATGTATTTAATTTGATATTAAGTAAATGAATGATTATGACTTTATGAAT |
| | | TCGCAATGTTTTCTCCTTGATTATTTCTGTATTGTATTGGAATGATTATAGAAT |
| | | ACTCATATATTGATTATAGTATTAGCACATAAAACGTTTGTTGTTAAACTCACT |
| | | TCCGTACGCAACCATTTCTATTTCTAGCTATCTTGATAAGGT |
| 187 | tTEF | TACTGACAATAAAAAGATTCTTGTTTTCAAGAACTTGTCATTTGTATAGTTTTT |
| | | TTATATTGTAGTTGTTCTATTTTAATCAAATGTTAGCGTGATTTATATTTTTTT |
| | | TCGCCTCGACATCATCTGCCCAGATGCGAAGTTAAGTGCGCAGAAAGTAATATC |
| | | ATGCGTCAATCGTATGTGAATGCTGGTCGCTATACTG |
| 188 | Tsynth1 | TATATATATATAACTGTCTAGAAATAAAGAGTATCATCTTTCAAA |
| 189 | RPS2tt - | TAGTCAAATATTAATCTATTTCACCTGTTCAAACTTTACTTAATGTACAAATGT |
| | no T2S | GGTAGTTATTAGTTTTGCAACGGAACTTGTTCCATAATCTGGTCCTCTGGGACA |
| | | GCAAACTGTCTTTCACTAGTAGCGCCAGTTTCGGGAGTCCACACAGCATTAGTC |
| | | ACCGGTGCACCAGCACTAATCTCACGACCTTCTGGGTGTTTAAATGGGCAGTTA |
| | | GGGTTGCGGCATCCAGCTGCAAACTTACAATCCTCATCAATTGGATGAGTGAAA |
| | | AAACAGTTTGGTCTGGTACAACTGTTGCCTTCACGACACAGTACAGGAGTAGTT |
| | | GCGTGACGTCTTGGGCACTTGTAATTACGGCATGATTTACCAAATCGACATTGT |
| | | TCCAAAGCCCTCTGTTGTTTTTGTTGTTTCTCTTCTTCGGTGATCTTGTGTTCA |
| | | GGTGATCGATGAGCCTTTGGACAGTCCGGATTAGAGCGCT |
| 190 | tDHAS | ACGGGAAGTCTTTACAGTTTTAGTTAGGAGCCCTTATATATGACAGTAATGCTA |
| | | GTACGTTTTGTTTTGTTTAATTAATAACTTAGTTTATGTTAGCCTAGTATAGAC |
| | | TCCATCAATTTTTTTTGTTATTACGTAAGCCGCGATGATAATATCTGATGAAAA |
| | | ATTCCTATCAGAAAATAATTTATCAAAAGTTTCATGCGATATGAGACTAAGTAG |
| | | AATAGGGACTCCCAAAGTGTCAGTCACAAGGGTCATTCCCGTTCGTAATGTGGT |
| | | GATAGCGAGGAGAAAACCTGTCAGAGCAAGTAACACCGACGCAAAGACATGGCT |
| | | AATGAAAGAAGAGCAGAGAAGAATAAGACAGAAGGAGCAGGAGATGAAACAAAG |
| | | GCTAGAGGAACTAGAAAGGTTCAAAACAAAAGTACAGAAATCATATATAAGGAA |
| | | AGAGGATAGGCATTTGGCACAAGAGATAGAAAAGGATCTTGACATAATCACTGA |
| | | TGATTACAATTTGG |
| 191 | TgpdA | AGATCTAATCAGGACGGCAAACTCAATTCAGAAGTGTGCTGTGAGTGAGACTGA |
| | | TTGCCGAGCGCAGACGACTCTCGTGGAACCCGGCTTGTGGAGAAGCTTGAGAAG |
| | | GTCTTAACTCCTAGCGTAAAAGCTCATGATGACGTACAATTTAATGAAATGATA |
| | | CAATGTTCATATTTCCCGTTCAAATTTCCGGCCTTGGTCAGTGCGTAAGATGTC |
| | | CACGATTGAATACTAACTCAGTATGGGTTTGGTAGCATTGGCAATGTAGTTATA |
| | | AGCATGCACCGGTTGAAGACGTCGGCCCCAGATGCAATGCTGCGGTGGTGACTA |
| | | AGCTCTGCAGTGAATGGAATGCGTTTCTTTGATCGACTTCGGCGTGCCGCGGGA |
| | | TTTTCTCGGCGCTTCTACTGGTGCAGAAAGGACGATACCACTGGCTTTCGGTCC |
| | | ATGCCACATCCCAGTCTCCCGGGAAATTCATTGCATACTTTAAGAAACAAACTG |
| | | ATCTCCATAATTTCCGTCTTTAGAGTTCACTTGGTACTTTTGGGTGGATCGAGG |
| | | GGTGTCCGCGGCCATCCAAGTCACGTGGAGGGCAGCTAGACCACGGATTTTAGA |
| | | GCTACATTGATCCAAGACTCCTGGACCGGCCTCATGGGCC |

### Nucleic Acids Encoding a Polypeptide Comprising a Secretion Signal

Aspects of the disclosure relate to nucleic acids (e.g., transcriptional units) encoding a secreted protein.

In some embodiments, a nucleic acid encoding a secreted protein is operably linked to a promoter. In some embodiments, a nucleic acid encoding a secreted protein is operably linked to a constitutive promoter. In some embodiments, a nucleic acid encoding a secreted protein is operably linked to an inducible promoter (e.g., activated during glucose or thiamine limitation or induced by methanol). In some embodiments, a nucleic acid encoding a secreted protein is operably linked to a synthetic promoter (as used herein), such as a synthetic promoter provided in Table 6 of WO 2022/108839 A1 or a promoter having a sequence that is at least 90%, at least 91%, at least 92%, at least 93%, at least 94%, at least 95%, at least 96%, at least 97%, at least 98%, or at least 99% identical to a nucleic acid sequence in Table 6 of WO 2022/108839 A1, or a functional fragment thereof. In some embodiments, a nucleic acid encoding a secreted protein is operably linked to a synthetic promoter (as used herein), such as a synthetic promoter provided in Tables 15-18 of WO 2022/051696 A1 or a promoter having a sequence that is at least 90%, at least 91%, at least 92%, at least 93%, at least 94%, at least 95%, at least 96%, at least 97%, at least 98%, or at least 99% identical to a nucleic acid sequence in Tables 15-18 of WO 2022/051696 A1, or a functional fragment thereof.

In some embodiments, a nucleic acid encoding a secreted protein is operably linked to a promoter having a sequence that is at least 90%, at least 91%, at least 92%, at least 93%, at least 94%, at least 95%, at least 96%, at least 97%, at least 98%, or at least 99% identical to a nucleic acid sequence as shown in TABLE 6, or a functional fragment thereof. In some embodiments, the synthetic promoter comprises a polynucleotide having not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 50 nucleotide substitutions, insertions, additions, or deletions relative to a nucleic acid sequence as shown in TABLE 6, or a functional fragment thereof. In some embodiments, the synthetic promoter has a nucleic acid sequence as shown in TABLE 6, or a functional fragment thereof.

In some embodiments, a nucleic acid encoding a secreted protein is operably linked to a terminator. In some embodiments, the terminator comprises a sequence that is at least 90%, at least 91%, at least 92%, at least 93%, at least 94%, at least 95%, at least 96%, at least 97%, at least 98%, or at least 99% identical to a nucleic acid sequence as shown in TABLE 7. In some embodiments, the terminator comprises a polynucleotide having not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 50 nucleotide substitutions, insertions, additions, or deletions relative to a nucleic acid sequence as shown in TABLE 7. In some embodiments, the terminator has a nucleic acid sequence as shown in TABLE 7.

In some embodiments, a nucleic acid encoding a polypeptide comprising a secretion signal having a structure as described in TABLE 15.

### Expression Systems

Aspects of the disclosure relate to expression systems for production of a protein and to host cells comprising the same. The expression systems described herein may comprise one or more polynucleotides that (individually or collectively) encode a protein of interest and: (1) a calreticulin (CRT) protein; (2) a protein disulfide isomerase family A member 3 (PDIA3) protein; (3) a protein disulfide isomerase 1 (PDI1) protein; (4) a glutathione peroxidase 1 (GPX1) protein; (5) a HAC1 protein; or any combination thereof. PDI1, GPX1 and HAC1 have been reported to improve expression of heterologous genes in hosts, including Pichia (see e.g., Delic et al. 2012 Free Rad. Biol. Med. 52: 2000; Prattipati et al. Enz. Microb. Tech. 140: 109633; Navone et al. 2021 Microb. Cell Fact. 20: article 8; Guerfal et al. 2010 Microb. Cell Fact. 9: article 49; and Ben Azoun et al. 2016 Microb. Biotech. 9: 355). In some embodiments, an expression system comprises one or more polynucleotides that (collectively) encode: a secreted protein; a calreticulin (CRT) protein; a protein disulfide isomerase family A member 3 (PDIA3) protein; a protein disulfide isomerase 1 (PDI1) protein; a glutathione peroxidase 1 (GPX1) protein; and/or a HAC1 protein; or a homolog of any of these proteins. Any of these proteins, e.g., the protein of interest or any of the others such as CRT or PDIA3, may include a secretion signal, including but not limited to any secretion signal as disclosed above or known in the art.

A expression system may further comprise one or more polynucleotides encoding a transcription factor that regulates (increase or decrease) expression of: a protein of the expression system; a calreticulin (CRT) protein of the expression system; a protein disulfide isomerase family A member 3 (PDIA3) protein of the expression system; a protein disulfide isomerase 1 (PDI1) protein of the expression system; a glutathione peroxidase 1 (GPX1) protein of the expression system; and/or a HAC1 protein of the expression system.

### Calreticulin (CRT) Proteins

In some embodiments, an expression system comprises a polynucleotide having a transcriptional unit encoding a calreticulin (CRT) protein (or a homolog thereof).

Without wishing to be bound by any particular theory, the present disclosure notes that CRT proteins have been reported as having various functions, including the ability to act as chaperone for protein folding. In addition to Ca2+, calreticulin binds and regulates proteins and mRNAs and affects their intracellular processing. As reported herein, the inventors have found that overexpression of a CRT protein can increase secretion of a protein of interest. Indeed, the inventors unexpectedly found that overexpression of a mammalian CRT protein in yeast can increase secretion of a protein of interest.

Exemplary CRT proteins are provided in TABLE 8. Additional CRT proteins (and homologs thereof) are known to those having ordinary skill in the art. In some embodiments, a CRT homolog includes but is not limited to any of: a calreticulin (or a precursor or isoform thereof), a calreticulin-like predicted protein, or a calreticulin-domain-containing protein of the following species and accession number: Homo sapiens, NP_004334.1; Colobus angolensis palliatus, XP_011810081.1; Piliocolobus tephrosceles, XP_023044476.1; Nomascus leucogenys, XP_003275645.1; Gorilla gorilla, XP_004060167.1; Saimiri boliviensis, XP_010327764.1; Callithrix jacchus, XP_002761834.1; Rhinopithecus roxellana, XP_030792600.1; Mustela putorius furo, XP_004748382.1; Rhinopithecus roxellana, XP_010359924.2; Canis lupus dingo, XP_025313509.1; Felis catus, XP_003981971.2; Carlito syrichta, XP_008046220.1; Hyaena, XP_039108198.1; Zalophus californianus, XP_027443401.1; Panthera tigris, XP_007098250.2; Suricata suricatta, XP_029774364.1; Equus quagga, XP_046494324.1; Ailuropoda melanoleuca, XP_002921056.1; Lemur catta, XP_045406339.1; Equus caballus, XP_001504932.1; Myotis davidii, XP_006768269.1; Sus scrofa, NP_001167604.1; Camelusferus, XP_006175440.3; Mus caroli, XP_021025190.1; Meriones unguiculatus, XP_021487879.1; Phacochoerus africanus, XP_047632859.1; Neotoma lepida, OBS76566.1; Tursiops truncatus, XP_004311443.2; Sousa chinensis, TEA31070.1; Rattus norvegicus, NP_071794.1; Fukomys damarensis, XP_010611683.1; Equus przewalskii, XP_008507800.1; Sturnira hondurensis, XP_036897756.1; Peromyscus maniculatus bairdii, XP_006996280.1; Oryctolagus cuniculus, NP_001075704.1; Cricetulus griseus, NP_001231051.1; Phodopus roborovskii, CAH6813733.1; Heterocephalus glaber, XP_004872463.1; Eptesicus fuscus, XP_008156151.1; Gigaspora margarita, CAG8779253.1; Cetraspora pellucida, CAG8605900.1; Dentiscutata erythropus, CAG8603985.1; Cetraspora pellucida, CAG8729077.1; Acaulospora morrowiae, CAG8488323.1; Scutellospora calospora, CAG8434477.1; Glomus cerebriforme, RIA96337.1; Funneliformis mosseae, CAG8440116.1; Rhizophagus clarus, GES93729.1; Ambispora gerdemannii, CAG8536145.1; Paraglomus occultum, CAG8606921.1; Claroideoglomus candidum, CAG8564351.1; Trichuris suis, KFD48565.1; Conidiobolus coronatus NRRL 28638, KXN69228.1; Trichinella pseudospiralis, KRY85451.1; Rhizoclosmatium globosum, ORY45358.1; Entomophthora muscae, KAF7757414.1; Absidia repens, ORZ12634.1; Batrachochytrium dendrobatidis JEL423, OAJ37882.1; Sinocyclocheilus anshuiensis, XP_016362922.1; Blyttiomyces helicus, RK087563.1; Puntigrus tetrazona, XP_043078636.1; Trichinella patagoniensis, KRY21440.1; Protopterus annectens, XP_043940593.1; Carassius auratus, XP_026052918.1; Cyprinus carpio, XP_042567583.1; Sinocyclocheilus rhinocerous, XP_016411312.1; Mauremys mutica, XP_044850839.1; Plakobranchus ocellatus, GFN79069.1; Chytriomyces confervae, TPX69219.1; Terrapene carolina triunguis, XP_024073034.1; Dendronephthya gigantea, XP_028404701.1; Endogone sp. FLAS-F59071, RUS20887.1; Biomphalaria glabrata, XP_013075154.1; Caretta caretta, XP_048687472.1; Chelmon rostratus, XP_041804531.1; Pimephales promelas, XP_039538315.1; Danio rerio, NP_571122.2; Mauremys reevesii, XP_039370197.1; Gopherus evgoodei, XP_030401854.1; Boleophthalmus pectinirostris, XP_020784762.1; Volvox carterif nagariensis, XP_002953987.1; Trichoplax adhaerens, XP_002115090.1; Haemonchus contortus, CDJ90114.1; Trachemys scripta elegans, XP_034648490.1; Chelonoidis abingdonii, XP_032646361.1; Anisakis simplex, AXS78236.1; Syngnathus scovelli, XP_049602453.1; Dermochelys coriacea, XP_038235165.1; Chelydra serpentina, KAG6930294.1; Oreochromis niloticus, XP_003448535.1; Piromyces finnis, ORX56483.1; Neocallimastix californiae, ORY26197.1; Anaeromyces robustus, ORX81537.1; Blyttiomyces helicus, RK087563.1; Basidiobolus meristosporus CBS 931.73, ORX91885.1; Rhizoclosmatium globosum, ORY45358.1; Brugia malayi, XP_001896170.1; Angiostrongylus cantonensis, KAE9413981.1; Dirofilaria immitis, AAD03405.2; Auanema sp. JU1783, CAH6623671.1; Wuchereria bancrofti, EJW84212.1; Acanthocheilonema viteae, VBB30442.1; Necator americanus, XP_013301410.1; Anisakis simplex, AXS78236.1; Loa loa, XP_003142911.1; Dictyocaulus viviparus, KJH45499.1; Tropilaelaps mercedesae, OQR80264.1; Litomosoides sigmodontis, VDK79370.1; Brugia pahangi, VDN93748.1; Onchocerca volvulus, P11012.2; Glomus cerebriforme, RIA96337.1; Halicephalobus sp. NKZ332, KAE9556681.1; Strongylus vulgaris, VDM68834.1; Rhizophagus irregularis, PKK67974.1; Caenorhabditis auriculariae, CAD6186773.1; Nippostrongylus brasiliensis, VDL85107.1; Haemonchus placei, VD046167.1; Ancylostoma duodenale, KIH65872.1; Umbelopsis vinacea, KAG2187185.1; Dendronephthya gigantea, XP_028404701.1; Rhizophagus clarus, GBC10675.1; Chytriomyces confervae, TPX69219.1; Acaulospora morrowiae, CAG8488323.1; Rhizophagus clarus, GES93729.1; Soboliphyme baturini, VDP03056.1; Oesophagostomum dentatum, KHJ88254.1; Caenorhabditis elegans, NP_504575.1; Lytechinus variegatus, XP_041474977.1; Brienomyrus brachyistius, XP_048845014.1; Cetraspora pellucida, CAG8605900.1; Varroa destructor, XP_022654216.1; Oncorhynchus mykiss, XP_036841641.1; Dreissena polymorpha, KAH3867407.1; Euglena gracilis, Q9ZNY3.1; Batrachochytrium salamandrivorans, KAH6589913.1; Coregonus clupeaformis, XP_041751835.2; Plakobranchus ocellatus, GFN79069.1; Galendromus occidentalis, XP_003742982.1; Ambispora gerdemannii, CAG8536145.1; Hydra vulgaris, XP_047123121.1; Funneliformis mosseae, CAG8440116.1; Meloidogyne enterolobii, CAD2183095.1; Capitella teleta, ELU06446.1; Salmo salar, XP_014045728.1; or Adineta steineri, CAF1289689.1; or any protein comprising at least 50% sequence overall identity to any of these proteins.

In some embodiments, a homolog of a CRT protein has a sequence which is at least about 60%, 70%, 80%, 90%, or 95% identical to an amino acid sequence listed in TABLE 8 or elsewhere herein, or any amount in between any of these percentages.

In some embodiments, an expression system comprises a polynucleotide having a transcriptional unit encoding a CRT protein (or a homolog thereof), wherein the CRT protein has at least about 60%, 70%, 80%, 90%, or 95% identity to an amino acid sequence listed in TABLE 8 or elsewhere herein, or any amount in between any of these percentages.

In some embodiments, a nucleic acid encoding for a CRT protein is operably linked to a promoter, such as a constitutive promoter (as described herein), an inducible promoter (as described herein), or a synthetic promoter (as described herein).

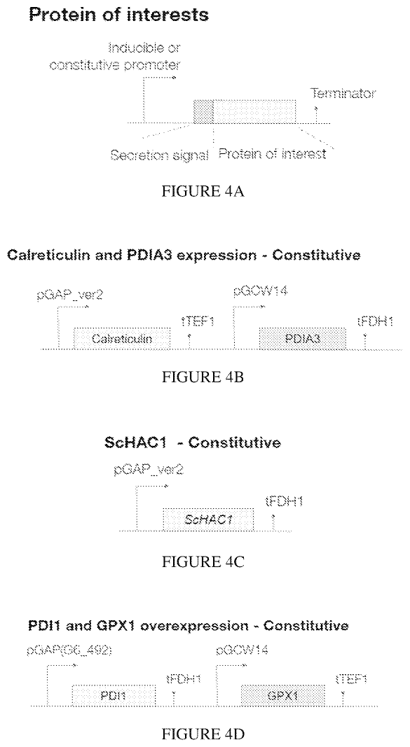

In some embodiments, a transcriptional unit encoding a CRT protein (or a homolog thereof) has a structure as depicted in FIG. 4B.

TABLE 8
Amino Acid Sequences of Exemplary CRT Proteins

| SEQ | | |
|---|---|---|
| ID NO: | Descr. | Sequence |
| 192 | Gigaspora | MRRSTIFFVLGLLTLTSLTSADIYLKETFSDDDWEKRWVHSKHKEDLGKFKVT |
| | rosea CRT | AGEFYAHEIESRGLQTTEDARFYAISTKFDKIIDNTDKDLVVQYSVKHEQNID |
| | (UniProt | CGGGYVKLLPSEFDALSFKGESLYNIMFGPDICGMNRKVHFIVHHKGENKELK |
| | A0A397UMK4) | KSIKAPSDQVTHLYTLILKPDHTYKILIDNEEEASGTLEEDFDLLPPEEITDP |
| | | TAKKPEDWEDLAEIPDPDDHKPEDWVDHPATIPDPDAKKPDDWDDEMDGDWEP |
| | | PQISNPDYKGEWKPKKIPNPKYKGEWKAPMIPNPDYVPEPNLHAFKTEFIGFD |
| | | LWQVRSGTIFDNILITDDVETAEKFANETFVKFRDAEKEAKKKLEELEKEQDE |
| | | ADDKDKKDGDKKDDDDDIIDLDVKLGDDGEVKVTKPEKDEKEKEKVKDEKEKE |
| | | KVKDEKDKEKVKDEKEKESKDKVKTEEVKKEEKAKETKDEKDEMDKLLDDLEE |
| | | ELGLPPKPKKEDHKLPIKDEL |
| 193 | Chlorocebus | MLLSVPLLLGLLGLAAAEPAVYFKEQFLDGDGWTSRWIESKHKSDFGKFVLSS |
| | aethiops | GKFYGDEEKDKGLQTSQDARFYALSASFEPFSNKGQTLVVQFTVKHEQNIDCG |
| | CRT | GGYVKLFPNSLDQTDMHGDSEYNIMFGPDICGPGTKKVHVIFNYKGKNVLINK |
| | (UniProt | DIRCKDDEFTHLYTLIVRPDNTYEVKIDNSQVESGSLEDDWDFLPPKKIKDPD |
| | Q4VIT5) | ASKPEDWDERAKIDDPTDSKPEDWDKPEHIPDPDAKKPEDWDEEMDGEWEPPV |
| | | IQNPEYKGEWKPRQIDNPDYKGTWIHPEIDNPEYSPDPSIYAYDNFGVLGLDL |
| | | WQVKSGTIFDNFLITNDEAYAEEFGNETWGVTKAAEKQMKDKQDEEQRLKEEE |
| | | EDKKRKEEEEAEDKEDDEDKDEDEEDEEDKEEDEEEDVPGQAKDEL |
| 194 | Mus | MLLSVPLLLGLLGLAAADPAIYFKEQFLDGDAWTNRWVESKHKSDFGKFVLSS |
| | musculus | GKFYGDLEKDKGLQTSQDARFYALSAKFEPFSNKGQTLVVQFTVKHEQNIDCG |
| | CRT | GGYVKLFPSGLDQKDMHGDSEYNIMFGPDICGPGTKKVHVIFNYKGKNVLINK |
| | (UniProt | DIRCKDDEFTHLYTLIVRPDNTYEVKIDNSQVESGSLEDDWDFLPPKKIKDPD |
| | P14211) | AAKPEDWDERAKIDDPTDSKPEDWDKPEHIPDPDAKKPEDWDEEMDGEWEPPV |
| | | IQNPEYKGEWKPRQIDNPDYKGTWIHPEIDNPEYSPDANIYAYDSFAVLGLDL |
| | | WQVKSGTIFDNFLITNDEAYAEEFGNETWGVTKAAEKQMKDKQDEEQRLKEEE |
| | | EDKKRKEEEEAEDKEDDDDRDEDEDEEDEKEEDEEESPGQAKDEL |
| 195 | Piromyces | MRFGTTLAILSFCAAAFGKVYFHETFDDDSWEKHWVQSTYKDDYGKFKISNGK |
| | finnis CRT | EFRADPVKSRGLQTSQNAKFYSISAPFDEAFNNKEKDLIVQFSVRHEQNIDCG |
| | (UniProt | GGYIKVLPPNIDPKEFNGETPYNIMFGSDICGANKKTHLILSYKGKNHLIKKE |
| | A0A1Y1VHI6) | IPTEDDTYTHLYTLVIKPDQTYSVSIDNVEKASGSFEDWDFLEPKTIPDPEKT |
| | | KPADWVDDEYIDDPNDKKPDDWDEDEPQYIDDPEAEKPEDWDDDMDGEWEAPK |
| | | SENPKYKGKWTPKKIKNPEYKGKWIQPEIPNPDYFDDKEIYVYDSGFVGFDLW |
| | | QVKAGSIFDDIVVTDDAEEAKTFATEVMDKIKAEKEDEEKVAAERKKIMDEKN |
| | | KRAAKKAEEMNEKRRKLERENHPEAYADEEESNDDSEEEMEETKEEEKEEVKE |
| | | ETKEETKEEAKEEEKKENVKDEL |
| 240 | Anopheles | MRTLAVLFAAFLAVNAKVYFEEGFKDDSWQKTWVQSEHKGVEYGKFVHTAGKF |
| | christyi | YNDAETDKGLQTSQDARFYALSNKFTPFSNKDDTLVIQFSVKHEQNIDCGGGY |
| | CRT | LKVFDCSKDLHGETPYLVMFGPDICGPGTKKVHVIFSYKGKNHLINKDIRCKD |
| | (UniProt | DVFTHFYTLVVRADNTYEVLIDNEKVESGSLEDDWDFLPPKKVKDPEAKKPED |
| | A0A182K1M4) | WDDRATIPDPDDTKPEDWDKPEHIPDPDATKPDDWDDEMDGEWEPPMIDNPEY |
| | | KGEWKPKQIDNPAYKGVWVHPEIDNPEYVEDNSLYLREEVCAVGIDVWQVKSG |
| | | TIFDNFMVTNDLEEAKKVAASVKETQEGEKKVKDAQEAEERKKAEEEAAAEEA |
| | | AKDDEDEDDDDDADNALPGEATELEDEGHDEL |
| 241 | Arabidopsis | MAKLNPKFISLILFALVVIVSAEVIFEEKFEDGWEKRWVKSDWKKDDNTAGEW |
| | thaliana | KHTAGNWSGDANDKGIQTSEDYRFYAISAEFPEFSNKDKTLVFQFSVKHEQKL |
| | CRT | DCGGGYMKLLSDDVDQTKFGGDTPYSIMFGPDICGYSTKKVHAILTYNGTNHL |
| | (UniProt | IKKEVPCETDQLTHVYTFVLRPDATYSILIDNVEKQTGSLYSDWDLLPAKKIK |
| | Calreti- | DPSAKKPEDWDDKEYIPDPEDTKPAGYDDIPKEIPDTDAKKPEDWDDEEDGEW |
| | culin-1) | TAPTIPNPEYNGEWKPKKIKNPAYKGKWKAPMIDNPEFKDDPELYVFPKLKYV |
| | | GVELWQVKSGSLFDNVLVSDDPEYAKKLAEETWGKHKDAEKAAFDEAEKKREE |
| | | EESKDAPAESDAEEEAEDDDNEGDDSDNESKSEETKEAEETKEAEETDAAHDE |
| | | L |
| 242 | Mucor | MKIPTIAAVLGLAAVVSAEVFLHETFSDGEGWKDRWTASEHREDLGKLEVSPG |
| | ambiguus | KWFADEAYNAGLRTTEDYRFYATSTKIPKPFNNKGKDLVIQFDVKNEQDIDCG |
| | CRT | GSYLKIFGDLDPKTFNGDSEYNIMFGPDICGPKAMVHAIFNYNGTNHDLKKSI |
| | (UniProt | SAPKDTLTHTYTLVVKPDQTYEILIDGKSKADGSLLEDWDFLPPKTIKDPNAS |
| | A0A0C9MAY0) | KPEDWVEEAMIVDETDVKPANYDDIPEFIPDPEAKKPEDWDDDMDGEWEAPSI |
| | | ANPEYQGEWSPKKIPNPLYKGEWVHPEIDNPEYKVDNEIYAYDFANVGIDVWQ |
| | | VKSGTVFDNILITDDIEEAKKVLDETKALHSSEEAAQAAFNEKIQAEAKAKAE |
| | | AEGAATPDEGAEKIDLEQFEPPVKFDEVPPAAAEALEKAKEEEILEAIQEEAE |
| | | KKVEEEDAKKPVKDEL |

### Protein Disulfide Isomerase Family a Member 3 (PDIA3) Proteins

In some embodiments, an expression system comprises a polynucleotide having a transcriptional unit encoding a protein disulfide isomerase family A member 3 (PDIA3) protein (or a homolog thereof).

Without wishing to be bound by any particular theory, the present disclosure notes that PDIA3 proteins have been reported as having various functions, including the ability to form a complex with calreticulin (CRT) and to act as a further chaperone for protein folding. PDIA3 is thought to enhance protein folding by promoting formation of disulfide bonds. As reported herein, the inventors have unexpectedly found that overexpression of a PDIA3 protein can increase secretion of a protein of interest. Indeed, the inventors unexpectedly found that overexpression of a mammalian PDIA3 protein in yeast can increase secretion of a protein of interest.

Exemplary PDIA3 proteins are provided in TABLE 9. Additional PDIA3 proteins (and homologs thereof) are known to those having ordinary skill in the art. In some embodiments, a PDIA3 homolog includes but is not limited to: Protein disulfide-isomerase, Anabas testudineus, A0A3Q1J9A0; Protein disulfide-isomerase, Anaeromyces robustus, A0A1Y1VSB5; Thioredoxin domain-containing protein, Anaeromyces robustus, A0A1Y1VTS1; Protein disulfide-isomerase, Anopheles christyi, A0A182JR00; DnaJ protein ERDJ3A, Arabidopsis thaliana, Q9SR96; NADPH-dependent thioredoxin reductase 3, Arabidopsis thaliana, 022229; Protein disulfide isomerase-like 1-1, Arabidopsis thaliana, Q9XI01; Protein disulfide isomerase-like 1-2, Arabidopsis thaliana, Q9SRG3; Protein disulfide isomerase-like 1-6, Arabidopsis thaliana, Q66GQ3; Protein disulfide-isomerase A3, Bos taurus, P38657; Protein disulfide-isomerase A5, Bos taurus, Q2KIL5; Thioredoxin reductase, Brassica napus, A0A078HAC7; Probable protein disulfide-isomerase A4, Caenorhabditis elegans, P34329; NADPH-dependent thioredoxin reductase 3, Camelina sativa, XP_010505953.1; Protein disulfide-isomerase, Carassius auratus, A0A6P6JDW2; Protein disulfide isomerase like, testis expressed, Cebus imitator, A0A2K5RAN3; Protein disulfide-isomerase A3, Chlorocebus aethiops, Q4VIT4; Thioredoxin domain-containing protein, Chytriomyces confervae, A0A507FS64; Protein disulfide-isomerase, Coregonus sp. ‘balchen’, A0A6F9B7I1; DnaJ homolog subfamily C member 10, Corvus moneduloides, A0A8C3D7I4; Protein disulfide-isomerase, Cricetulus griseus, Q91Z81; Protein disulfide-isomerase, Cyberlindnera jadinii, A0A0H5C3Z6; protein disulfide-isomerase A3-like, Dermacentor silvarum, XP_037563384; Protein disulfide-isomerase 2, Dictyostelium discoideum, Q54EN4; Thioredoxin domain-containing protein, Gigaspora rosea, A0A397VAR5; Thioredoxin domain-containing protein, Glomus cerebriforme, A0A397SNC8; J domain-containing protein, Gossypium barbadense, A0A5J5W6J3; Thioredoxin reductase, Gossypium mustelinum, A0A5D2VKA6; J domain-containing protein, Gossypium raimondii, A0A0D2VYE3; Protein disulfide-isomerase A3, Homo sapiens, P30101; Protein disulfide-isomerase A5, Homo sapiens, Q14554; Protein disulfide-isomerase-like protein of the testis, Homo sapiens, Q8N807; Thioredoxin domain-containing protein, Jimgerdemannia flammicorona, A0A433D7Z6; Protein disulfide isomerase, Lichtheimia corymbifera JMRC:FSU:9682, A0A068SF51; Uncharacterized protein, Lichtheimia ramosa, A0A077WWW3; Protein disulfide-isomerase-like protein of the testis, Macaca fascicularis, Q95LMO; Protein disulfide isomerase-like 2-1-like, Mucor ambiguus, A0A0C9MV02; Uncharacterized protein, Mucor lusitanicus, A0A168N752; Protein disulfide-isomerase A3, Mus musculus, P27773; Protein disulfide-isomerase, Mustela putorius furo, M3YG84; Protein disulfide-isomerase, Myotis lucifugus, G1PDK8; Thioredoxin domain-containing protein, Neocallimastix californiae, A0A1Y2A098; Thioredoxin-like protein, Neocallimastix californiae, A0A1Y2A7H2; Protein disulfide-isomerase, Neocallimastix californiae, A0A1Y2AUI3; Uncharacterized protein, Neocallimastix californiae, A0A1Y2EV84; Protein disulfide-isomerase, Onychostoma macrolepis, A0A7J6BJY2; Thioredoxin reductase NTRC, Oryza sativa subsp. japonica, Q70G58; Uncharacterized protein, Parasitella parasitica, A0A0B7NP58; Uncharacterized protein, Phycomyces blakesleeanus NRRL 1555(−), A0A162WWE6; Thioredoxin domain-containing protein, Piromyces finnis, A0A1Y1UWN5; Thioredoxin-like protein, Piromyces finnis, A0A1Y1V0A8; Protein disulfide-isomerase, Piromyces sp. E2, A0A1Y3N9L4; Thioredoxin reductase, Raphanus sativus, A0A6J0MKF9; Protein disulfide-isomerase A3, Rattus norvegicus, P11598; Protein disulfide-isomerase-like protein of the testis, Rattus norvegicus, Q5XI02; Thioredoxin-domain-containing protein, Rhizoclosmatium globosum, A0A1Y2CUK4; Thioredoxin-domain-containing protein, Rhizophagus irregularis, A0A2N0RXQ5; Protein disulfide-isomerase, Rhizopus stolonifer, A0A367JQ22; Protein disulfide-isomerase, Sinocyclocheilus rhinocerous, A0A673GJ53; DnaJ homolog subfamily C member 10, Terrapene carolina triunguis, A0A674K1L9; and Protein disulfide-isomerase, Xiphophorus maculatus, M4AF75.

In some embodiments, a homolog of PDIA3 has a sequence which is at least about 60%, 70%, 80%, 90%, or 95% identical to an amino acid sequence listed in TABLE 9, or any amount in between any of these percentages.

In some embodiments, an expression system comprises a polynucleotide having a transcriptional unit encoding a PDIA3 protein (or a homolog thereof), wherein the PDIA3 protein has at least about 60%, 70%, 80%, 90%, or 95% identity to an amino acid sequence listed in TABLE 9, or any amount in between any of these percentages.

In some embodiments, the nucleic acid encoding the PDIA3 protein is operably linked to a promoter, such as a constitutive promoter (as described herein), an inducible promoter (as described herein), or a synthetic promoter (as described herein).

In some embodiments, a transcriptional unit encoding a protein disulfide isomerase family A member 3 (PDIA3) protein (or a homolog thereof) has a structure as depicted in FIG. 4B.

TABLE 9
Amino Acid Sequences of Exemplary PDIA3 Proteins

| SEQ ID | | |
|---|---|---|
| NO: | Descr. | Sequence |
| 196 | Anaeromyces | MRFSKLFKIVASVALAAKVAAEGNVVSLTKDDYEVTLEEAPLALVKYFAP |
| | robustus | WCGHCKALAPEFVKAADALKEQNILLAEVDCTVESDICNEVGVRGYPTLK |
| | PDIA3 | VYRNGKASDYKGQRTAESIISYMKKQSLPDLTAIKAEDFETFSTSDKVVV |
| | (Uniprot | VGFVKEGSDEYKALEANAKELREQFVFGYIDDAELAKKAGAAVPGIVVYK |
| | A0A1Y1VSB5) | QFDEGKAVLEGEITEESIKNFVNIESVPLMDELGPENYSKYMESGLPLVY |
| | | LFTSDAEDKKTVGAWCEAIAKKVQGKLNFVYIDAVKFSGHGKFLGLKETW |
| | | PAVIIQDVKHNTKYPFPQDKKIEKEELEKFIDDFQAGKLEPFLKSQDVPE |
| | | KQEDGIYNVVAKTFDEVVLDKSKDVLLLFYAPWCGHCKKLAPTYKEIAEE |
| | | INENNDKVLIARMDATENDIPSTSPWNNLEGFPTVILFKADDKNTAVVYE |
| | | GDRSKESILEFIKKNAVNEFKVPEKKEEEKKEEEEKKEEKKEEEKKEEKD |
| | | DEKVKDEL |
| 197 | Dictyostelium | MKLINICIFIFAIICIESTFGFYTDNSNVINLTKKNFQQQVLNSQQNWMV |
| | discoideum | EFYAPWCGHCKSLKPEYEKVSNNLKGLVKIGAINCDEEKELCGQYQIQGF |
| | PDIA3 | PTLKFFSTNPKTGKKGQPEDYQGARSASEIAKFSLAKLPSNHIQKVSQDN |
| | (Uniprot | INKFLTGTSDAKALLFTDKPKTTDLYKALSVDFFKTLTLGEARNLNKETL |
| | Q869Z0) | EKFNIDKFPTLLVFTNDDGETFTKFDGKLTHSTIYKFLEPFSKKSNNDNN |
| | | NNNNNNNNEESTKTTTTEKDPASEKFIEIKDEKSFEKSCSTGLCIVALFD |
| | | QSSIDDKELNEKYLELLNTVSQNFIGRMKFVWVDVSVHDKIVPQFDLSGT |
| | | PNIFVINNSKKRYTPFMGSFSDESLNSFFKSVLSGLKKAIPFTDSPKFNS |
| | | QQKKQKDEL |
| 198 | Lichtheimia | MAFFRNLFLLLLVVNAAFLGYDHFKGGDIAVSLVDNAKRLDVPTVQNHLQ |
| | ramose PDIA3 | QTWTSIKSTNPEKLAGHVNAAFDQLKGFNSFSDVVDHVRAKVSGAVGGSA |
| | (Uniprot | DMGRIILEDNVFVLNDKNFDKVIDGSRPALVEFYAPWCGHCKKLAPTYAE |
| | A0A077X008) | LGEAFSTVQDRVVIAKVNADEQRDLGARFGIQGFPTLKWFPKGVTTPDGI |
| | | EDYRGGRDLDSLSKFVHEKSGVRPRVKSTKSDVVVLDSQNFNSIVKDPKT |
| | | NVLVEFYAPWCGHCKNLAPTYEKVATAFANEPNCKVAKIDADSERAIGTE |
| | | YEISGFPTIKFFAAGEDKEPVAYEGPRTEAGFIEFLNKQCGTHRLVGGSL |
| | | DATAGRIADLDQLAIKFASTSDKVAREAIQKEATVVAGELGTRNAKFYGI |
| | | VMKKVLEKGDGFIKTENARLDKIIKSNTVTASKVDDFTVRKNILAAFDKK |
| | | AKPVTKDEL |
| 243 | Anopheles | MSCRLVLVCLCALVAVAFAGEADVLDLTDSDFSTRVAETETTLVMFYAPW |
| | christyi | CGHCKKLKPEYAKAAELLRGEDPPIALAKVDCTEGGKDTCNKFSVSGYPT |
| | PDIA3 | LKIFKNGEVSQEYNGPREATGIAKYMKSIVGPASKDLLTLEAFEAFLKVQ |
| | (UniProt | ETSVVGFFQKESDLKGVFLKYADSQRERLRFGHSSAPAVLEKQGDTDAVY |
| | A0A182JR00) | LFRARQLANKFEPDFVKFEGTSKQELADFVKANFHGLAGVRSRDTTSDFK |
| | | NPLVVVYYAVDYVKNPKGTNYWRNRVLKVAKEFVGRVNFAVSAKDDFQHE |
| | | LNEYGYDYTGDKPLVLARDAKNQKFIMKDEFSVENLQAFATELEEGSLEP |
| | | YVKSEPVPESNDGPVKVAVAKNFDDVVVNNGVDTLVEFYAPWCGHCKKLT |
| | | PTLEELGTKLKDEAVSIVKMDATANDVPSHFEVRGFPTLYWLPKDAKSSP |
| | | TRYEGGREVDDFVKYIAKHATSELKGFDRSGSAKKTEL |
| 244 | Mucor | MSFFKKLFFLLVAVNAAFLGYDYYQGGNIAFNLVENAKQLNGDKLHGYLA |
| | ambiguus | EIKSTTPEKLAGHVNNAFAQLKNINSASDILTIIREKTAPVTGAGAGSIE |
| | PDIA3 | WDGNVVVLTDANFKNVIDGSKPALVEFYAPWCGHCKNLAPVYAQLGDAFS |
| | (UniProt | SSKDKVLVAKIDADQHRDTGALFGVQGFPTLKWFPKGVHSPEGVEDYKGG |
| | A0A0C9MV02 | RDLNSLAAFIKEKSGVAPRIKSQKSDVVTLTTKNFHEVALNPKKNVLVEF |
| | | YASWCGHCKNLAPIWEKIGSTFANEENCVIAKIDADEERDIGSEFDISGF |
| | | PTIKFFPAGESEPVAYEGGRTEAAFVEFLNKHCGTQRKVGGGLEAAAGRI |
| | | AKLDELAIRFIKNAGEREKIHAEAVEAAKEIGTRYGTYYAKIMEKMLANG |
| | | EKFLETERARLAKIAGSDDVSSAKLDDFGIRQNILGAFDKKASPVKN |
| 245 | Arabidopsis | MAFKGFACFSILLLLSLFVSSIRSEETKEFVLTLDHSNFTETISKHDFIV |
| | thaliana | VEFYAPWCGHCQKLAPEYEKAASELSSHNPPLALAKIDASEEANKEFANE |
| | Protein | YKIQGFPTLKILRNGGKSVQDYNGPREAEGIVTYLKKQSGPASVEIKSAD |
| | disulfide | SATEVVGEKNVVAVGVFPKLSGDEFDSFMALAEKLRADYDFAHTLDAKFL |
| | isomerase- | PRGESVEGPAVRLFKPFDELFVDSKDFNGEALEKFVKESSIPLVTVFDSD |
| | like 1-2 | PNNHPYVAKFFESPATKAMMFVNFTGATAEALKSKYREVATSNKDQSLAF |
| | (UniProt | LVGDAESSQGAFQYFGLEESQVPLIIIQTPDNKKYLKVNVEVDQIESWFK |
| | Q9SRG3) | DFQDGKVAVHKKSQPIPAENNEPVKVVVAESLDDIVFKSGKNVLIEFYAP |
| | | WCGHCQKLAPILDEVALSFQNDPSVIIAKLDATANDIPSDTFDVKGFPTI |
| | | YFRSASGNVVVYEGDRTKEDFINFVEKNSEKKPTSHGEESTKSEEPKKTE |
| | | ETAAKDEL |

### Protein Disulfide Isomerase 1 (PDI1) Proteins

In some embodiments, an expression system comprises a polynucleotide having a transcriptional unit encoding a protein disulfide isomerase 1 (PDI1) protein (or a homolog thereof).

Without wishing to be bound by any particular theory, the present disclosure notes that PDI1 proteins have been reported as having various functions, including the ability to promote protein folding by enhancing disulfide bond formation.

Exemplary PDI1 proteins are provided in TABLE 10. Additional PDI1 proteins (and homologs thereof) are known to those having ordinary skill in the art. In some embodiments, a homolog of PDI1 has a sequence which is at least about 60%, 70%, 80%, 90%, or 95% identical to an amino acid sequence listed in TABLE 10, or any amount in between any of these percentages. In some embodiments, a PDI1 homolog includes but is not limited to: AaceriAFR718Wp, Ashbya aceris (nom. inval.), AGO10797.1; hypothetical protein CANARDRAFT_200175, Candida arabinofermentans NRRL YB-2248, ODV84852.1; hypothetical protein B5S28_g3516, Candida boidinii, OWB57562.1; protein disulfide-isomerase precursor, Candida californica, KAG0684010.1; hypothetical protein CANINC_004119, Candida inconspicua, TID16667.1; unnamed protein product, Arabidopsis arenosa, CAE5964463.1; unnamed protein product, Arabidopsis lyrata, CAH8258467.1; protein disulfide isomerase-like 1-2, Arabidopsis lyrata subsp. lyrata, XP_020891315.1; protein disulfide isomerase-like 1-1, Arabidopsis lyrata subsp. lyrata, XP_002890462.1; Thioredoxin domain, Arabidopsis suecica, KAG7590471.1; Thioredoxin domain, Arabidopsis suecica, KAG7597864.1; PDI-like 1-1, Arabidopsis thaliana, NP_173594.1; PDI-like 1-1, Arabidopsis thaliana, NP_849696.1; PDI-like 1-2, Arabidopsis thaliana, NP_177875.1; PDIL1-1, Arabidopsis thaliana, OAP14434.1; unnamed protein product, Arabidopsis thaliana, CAA0339760.1; unnamed protein product, Arabidopsis thaliana, CAD5313395.1; unnamed protein product, Arabidopsis thaliana, CAA0230058.1; Thioredoxin domain, Arabidopsis thaliana x Arabidopsis arenosa, KAG7587379.1; Thioredoxin-like superfamily, Arabidopsis thaliana x Arabidopsis arenosa, KAG7592544.1; hypothetical protein AALP_AA2G217000, Arabis alpina, KFK42144.1; protein disulfide-isomerase al, Arabis alpina, KFK44284.1; unnamed protein product, Arabis nemorensis, VVA95632.1; unnamed protein product, Arabis nemorensis, VVA92097.1; protein disulfide isomerase, Ascoidea rubescens DSM 1968, XP_020045277.1; uncharacterized protein BABINDRAFT_15951, Babjeviella inositovora NRRL Y-12698, XP_018986277.1; hypothetical protein Bca52824_011879, Brassica carinata, KAG2318666.1; hypothetical protein Bca52824_014254, Brassica carinata, KAG2321041.1; hypothetical protein Bca52824_015944, Brassica carinata, KAG2322731.1; hypothetical protein Bca52824_025054, Brassica carinata, KAG2313497.1; hypothetical protein Bca52824_032173, Brassica carinata, KAG2303522.1; hypothetical protein Bca52824_053211, Brassica carinata, KAG2281991.1; hypothetical protein Bca52824_063565, Brassica carinata, KAG2269010.1; hypothetical protein Bca52824_072371, Brassica carinata, KAG2265292.1; hypothetical protein Bca52824_085073, Brassica carinata, KAG2254937.1; protein disulfide isomerase, Brassica carinata, ABB17025.1; hypothetical protein DY000_02055297, Brassica cretica, KAF3493604.1; hypothetical protein DY000_02064173, Brassica cretica, KAF3520020.1; hypothetical protein F2Q68_00046619, Brassica cretica, KAF2608350.1; hypothetical protein F2Q69_00051794, Brassica cretica, KAF3523460.1; hypothetical protein F2Q69_00054751, Brassica cretica, KAF3490462.1; hypothetical protein F2Q70_00045601, Brassica cretica, KAF2594443.1; hypothetical protein HID58_081513, Brassica napus, KAH0864302.1; protein disulfide isomerase-like 1-1, Brassica napus, XP_013643816.1; protein disulfide isomerase-like 1-1, Brassica napus, XP_013728290.1; protein disulfide isomerase-like 1-1, Brassica napus, XP_048592731.1; protein disulfide isomerase-like 1-1, Brassica napus, XP_013722128.2; protein disulfide isomerase-like 1-1, Brassica napus, XP_013695693.1; protein disulfide isomerase-like 1-2, Brassica napus, XP_013650033.2; protein disulfide isomerase-like 1-2, Brassica napus, XP_013696245.2; protein disulfide isomerase-like 1-2, Brassica napus, XP_013685367.2; unnamed protein product, Brassica napus, CAF2204970.1; unnamed protein product, Brassica napus, CAF1913314.1; unnamed protein product, Brassica napus, CAF2162359.1; unnamed protein product, Brassica napus, CAF2256190.1; unnamed protein product, Brassica napus, CAF2084821.1; unnamed protein product, Brassica napus, CAF1927578.1; unnamed protein product, Brassica oleracea, VDD56518.1; PREDICTED: protein disulfide isomerase-like 1-2, Brassica oleracea var. oleracea, XP_013592529.1; PREDICTED: protein disulfide isomerase-like 1-2, Brassica oleracea var. oleracea, XP_013619039.1; PREDICTED: protein disulfide isomerase-like 1-1, Brassica oleracea var. oleracea, XP_013599001.1; PREDICTED: protein disulfide isomerase-like 1-1, Brassica oleracea var. oleracea, XP_013605424.1; PREDICTED: protein disulfide isomerase-like 1-1, Brassica oleracea var. oleracea, XP_013584715.1; disulfide isomerase-like protein 1-2, Brassica oleracea var. viridis, QAX33932.1; hypothetical protein BRARA_F01587, Brassica rapa, RID58281.1; hypothetical protein BRARA_G01064, Brassica rapa, RID53687.1; hypothetical protein BRARA_G03521, Brassica rapa, RID56315.1; hypothetical protein BRARA_H02269, Brassica rapa, RID51618.1; protein disulfide isomerase-like 1-1, Brassica rapa, XP_009103284.1; protein disulfide isomerase-like 1-1, Brassica rapa, XP_009110169.1; protein disulfide isomerase-like 1-1, Brassica rapa, XP_009149696.1; protein disulfide isomerase-like 1-2, Brassica rapa, XP_009106410.1; protein disulfide isomerase-like 1-2, Brassica rapa, XP_009128231.1; unnamed protein product, Brassica rapa, CAG7893751.1; unnamed protein product, Brassica rapa, CAG7902029.1; unnamed protein product, Brassica rapa, CAG7899472.1; unnamed protein product, Brassica rapa, CAG7869634.1; hypothetical protein IGI04_006552, Brassica rapa subsp. trilocularis, KAG5410235.1; hypothetical protein IGI04_022850, Brassica rapa subsp. trilocularis, KAG5392887.1; hypothetical protein IGI04_032176, Brassica rapa subsp. trilocularis, KAG5390635.1; hypothetical protein HII13_005140, Brettanomyces bruxellensis, KAF6006463.1; PDI1, Brettanomyces bruxellensis, VUG20389.1; uncharacterized protein BRETT_001532, Brettanomyces bruxellensis, XP_041134583.1; protein disulfide isomerase, Brettanomyces bruxellensis AWRI1499, EIF47220.1; DEKNAAC103978, Brettanomyces naardenensis, VEU22897.1; uncharacterized protein FOA43_002660, Brettanomyces nanus, XP_038778873.1; PREDICTED: protein disulfide isomerase-like 1-1, Camelina sativa, XP_010498632.1; PREDICTED: protein disulfide isomerase-like 1-1, Camelina sativa, XP_010477422.1; PREDICTED: protein disulfide isomerase-like 1-1, Camelina sativa, XP_010459897.1; PREDICTED: protein disulfide isomerase-like 1-2, Camelina sativa, XP_010428829.1; PREDICTED: protein disulfide isomerase-like 1-2, Camelina sativa, XP_010471945.1; PREDICTED: protein disulfide isomerase-like 1-2, Camelina sativa, XP_010416695.1; hypothetical protein CARUB_v10020120 mg, Capsella rubella, EOA35017.1; protein disulfide isomerase-like 1-1, Capsella rubella, XP_006307302.1; protein disulfide isomerase-like 1-2, Capsella rubella, XP_006302119.2; protein disulfide-isomerase precursor, Clavispora lusitaniae, KAF5210732.1; putative disulfide-isomerase, Clavispora lusitaniae, QFZ27122.1; hypothetical protein CLUG_04029, Clavispora lusitaniae ATCC 42720, XP_002616788.1; CYFA0S05e00144g1_1, Cyberlindnera fabianii, CDR40383.1; Protein disulfide-isomerase, Cyberlindnera fabianii, ONH68046.1; protein disulfide isomerase, Cyberlindnera jadinii NRRL Y-1542, XP_020072714.1; uncharacterized protein AC631_04205, Debaryomyces fabryi, XP_015466153.1; DEHA2E23628p, Debaryomyces hansenii CBS767, XP_460327.1; AFR718Wp, Eremothecium gossypii ATCC 10895, NP_986266.2; HCL656Cp, Eremothecium sinecaudum, XP_017986491.1; protein disulfide isomerase-like 1-1, Eutrema salsugineum, XP_006416267.1; protein disulfide isomerase-like 1-2, Eutrema salsugineum, XP_006390081.1; related to Protein disulfide-isomerase, Hanseniaspora guilliermondii, SGZ39575.1; Protein disulfide-isomerase, Hanseniaspora opuntiae, OEJ91430.1; Protein disulfide-isomerase, Hanseniaspora osmophila, OEJ86534.1; hypothetical protein FOG48_02163, Hanseniaspora uvarum, KAF0268696.1; hypothetical protein FOG51_02482, Hanseniaspora uvarum, KAF0272758.1; Protein disulfide-isomerase, Hanseniaspora uvarum, OEJ92874.1; protein disulfide isomerase, Hanseniaspora valbyensis NRRL Y-1626, OBA26417.1; hypothetical protein KAFR_0D00300, Kazachstania africana CBS 2517, XP_003956812.1; uncharacterized protein KABA2_02S17138, Kazachstania barnettii, XP_041405376.1; protein disulfide-isomerase precursor, Kazachstania exigua, KAG0668310.1; hypothetical protein KNAG_0C00380, Kazachstania naganishii CBS 8797, XP_022463398.1; similar to Saccharomyces cerevisiae YDR518W EUG1 Protein disulfide isomerase of the endoplasmic reticulum lumen, function overlaps with that of Pdi1p, Kazachstania saulgeensis, SMN20110.1; protein disulfide-isomerase precursor, Kazachstania unispora, KAG0661603.1; unnamed protein product, Kluyveromyces dobzhanskii CBS 2104, CD092705.1; Pdi1/Eug1, Kluyveromyces lactis, QEU60284.1; uncharacterized protein KLLA0_C01111g, Kluyveromyces lactis, XP_452244.1; protein disulfide isomerase precursor, Kluyveromyces marxianus, AAD42032.1; protein disulfide-isomerase precursor, Kluyveromyces marxianus, KAG0675425.1; protein disulfide-isomerase, Kluyveromyces marxianus DMKU3-1042, XP_022673588.1; BA75_04463T0, Komagataella pastoris, ANZ77325.1; protein disulphide isomerase, Komagataella pastoris, ACF17572.1; protein disulphide isomerase, Komagataella pastoris, CAC33587.1; Protein disulfide isomerase, multifunctional protein resident in the endoplasmic reticulum lumen, Komagataella phaffii GS115, XP_002494292.1; uncharacterized protein KUCA_T00005923001, Kuraishia capsulata CBS 1993, XP_022461910.1; LADA_0H00958g1_1, Lachancea dasiensis, SCU96441.1; LAFE_0G01002g1_1, Lachancea fermentati, SCW03012.1; uncharacterized protein LALA0_SI1e00782g, Lachancea lanzarotensis, XP_022630498.1; LAME_0G00936g1_1, Lachancea meyersii CBS 8951, SCU98886.1; LAMI_0F00958g1_1, Lachancea mirantina, SCU95097.1; LANO_0G00958g1_1, Lachancea nothofagi CBS 11611, SCV02905.1; LAQUOS15e00782g1_1, Lachancea quebecensis, CUS24271.1; LAFA_0G00936g1_1, Lachancea sp. CBS 6924, SCU95550.1; KLTHOF01100p, Lachancea thermotolerans CBS 6340, XP_002554257.1; unnamed protein product, Microthlaspi erraticum, CAA7013418.1; unnamed protein product, Microthlaspi erraticum, CAA7047850.1; hypothetical protein NCAS_0B08940, Naumovozyma castelihi CBS 4309, XP_003675348.1; hypothetical protein NDAI_0A00310, Naumovozyma dairenensis CBS 421, XP_003667434.1; hypothetical protein KL909_003691, Ogataea angusta, KAG7823088.1; hypothetical protein KL921_005210, Ogataea angusta, KAG7805897.1; hypothetical protein KL939_003800, Ogataea angusta, KAG7857015.1; hypothetical protein KL941_004507, Ogataea angusta, KAG7844025.1; hypothetical protein KL943_004327, Ogataea angusta, KAG7832879.1; uncharacterized protein KL928_004711, Ogataea angusta, XP_043058203.1; hypothetical protein KL915_003613, Ogataea haglerorum, KAG7694646.1; hypothetical protein KL929_003555, Ogataea haglerorum, KAG7796364.1; hypothetical protein KL944_003792 Ogataea haglerorum, KAG7800219.1; hypothetical protein KL950_003711, Ogataea haglerorum, KAG7705275.1; hypothetical protein KL951_003595, Ogataea haglerorum, KAG7695153.1; uncharacterized protein KL911_004105, Ogataea haglerorum, XP_043053374.1; hypothetical protein KL938_004952, Ogataea parapolymorpha, KAG7876018.1; Protein disulfide-isomerase, Ogataea parapolymorpha DL-1, XP_013934024.1; uncharacterized protein OGAPHI_005380, Ogataea philodendri, XP_046059813.1; hypothetical protein KL907_003645, Ogataea polymorpha, KAG7903618.1; hypothetical protein KL937_003657, Ogataea polymorpha, KAG7878415.1; uncharacterized protein OGAPODRAFT_12847, Ogataea polymorpha, XP_018211818.1; hypothetical protein PACTADRAFT_47665, Pachysolen tannophilus NRRL Y-2460, ODV97814.1; hypothetical protein JL09_g1660, Pichia kudriavzevii, KGK39153.1; Protein disulfide-isomerase, Pichia kudriavzevii, ONH76889.1; uncharacterized protein C5L36_0A02930, Pichia kudriavzevii, XP_029319168.1; hypothetical protein PMKS-003341, Pichia membranifaciens, GAV29836.1; hypothetical protein PICMEDRAFT_70120, Pichia membranifaciens NRRL Y-2026, XP_019019601.1; PREDICTED: protein disulfide isomerase-like 1-1, Raphanus sativus, XP_018461926.1; PREDICTED: protein disulfide isomerase-like 1-1, Raphanus sativus, XP_018484508.1; PREDICTED: protein disulfide isomerase-like 1-2, Raphanus sativus, XP_018455739.1; PREDICTED: protein disulfide isomerase-like 1-2, Raphanus sativus, XP_018455748.1; PREDICTED: protein disulfide isomerase-like 1-2, Raphanus sativus, XP_018468717.1; hypothetical protein SCDLUD_003835, Saccharomycodes ludwigii, XP_045933490.1; hypothetical protein N665_0089s0008, Sinapis alba, KAF8109887.1; hypothetical protein N665_0188s0507, Sinapis alba, KAF8103579.1; hypothetical protein N665_0407s0012, Sinapis alba, KAF8092632.1; hypothetical protein N665_0541s0021, Sinapis alba, KAF8088456.1; hypothetical protein N665_6720s0001, Sinapis alba, KAF8044801.1; PREDICTED: protein disulfide isomerase-like 1-1, Tarenaya hassleriana, XP_010521263.1; unnamed protein product, Thlaspi arvense, CAH2067738.1; unnamed protein product, Thlaspi arvense, CAH2033558.1; hypothetical protein TDEL_0C06720, Torulaspora delbrueckii, XP_003680772.1; uncharacterized protein HG536_0B00480, Torulaspora globosa, XP_037137862.1; hypothetical protein HG537_0B00490, Torulaspora sp. CBS 2947, QLQ78700.1; hypothetical protein Kpol_2000p104, Vanderwaltozyma polyspora DSM 70294, XP_001646994.1; hypothetical protein WICANDRAFT_93648, Wickerhamomyces anomalus NRRL Y-366-8, XP_019038138.1; putative secreted protein, Wickerhamomyces ciferrii, XP_011271682.1; hypothetical protein WICMUC_003977, Wickerhamomyces mucosus, KAH3672923.1; hypothetical protein WICPIJ_002013, Wickerhamomyces pijperi, KAH3686988.1; related to Protein disulfide-isomerase, Zygosaccharomyces baiii ISA1307, CDH13292.1; protein disulfide-isomerase precursor, Zygosaccharomyces mellis, GCF00127.1; PDI1 (YCL043C) and EUG1 (YDR518W), Zygosaccharomyces parabailii, AQZ18915.1; PDI1 (YCL043C) and EUG1 (YDR518W), Zygosaccharomyces parabailii, AQZ14037.1; hypothetical protein ZYGR_0K00760, Zygosaccharomyces rouxii, GAV48571.1; and uncharacterized protein HG535_0A00480, Zygotorulaspora mrakii, XP_037141837.1.

In some embodiments, an expression system comprises a polynucleotide having a transcriptional unit encoding a PDI1 protein (or a homolog thereof), wherein the PDI1 protein has at least about 60%, 70%, 80%, 90%, or 95% identity to an amino acid sequence listed in TABLE 10, or any amount in between any of these percentages.

In some embodiments, the nucleic acid encoding the PDI1 protein is operably linked to a promoter, such as a constitutive promoter (as described herein), an inducible promoter (as described herein), or a synthetic promoter (as described herein).

In some embodiments, a transcriptional unit encoding a protein disulfide isomerase 1 (PDI1) protein (or a homolog thereof) has a structure as depicted in FIG. 4D.

TABLE 10
Amino Acid Sequences of Exemplary PDI1 Proteins

| SEQ | | |
|---|---|---|
| ID NO: | Descr. | Sequence |
| 199 | Pichia pastoris | MQFNWNIKTVASILSALTLAQASDQEAIAPEDSHVVKLTEATFESFITSNPHV |
| | PDI1 (NCBIGQ68_ | LAEFFAPWCGHCKKLGPELVSAAEILKDNEQVKIAQIDCTEEKELCQGYEIKG |
| | 05219T0) | YPTLKVFHGEVEVPSDYQGQRQSQSIVSYMLKQSLPPVSEINATKDLDDTIAE |
| | | AKEPVIVQVLPEDASNLESNTTFYGVAGTLREKFTFVSTKSTDYAKKYTSDST |
| | | PAYLLVRPGEEPSVYSGEELDETHLVHWIDIESKPLFGDIDGSTFKSYAEANI |
| | | PLAYYFYENEEQRAAAADIIKPFAKEQRGKINFVGLDAVKFGKHAKNLNMDEE |
| | | KLPLFVIHDLVSNKKFGVPQDQELTNKDVTELIEKFIAGEAEPIVKSEPIPEI |
| | | QEEKVFKLVGKAHDEVVFDESKDVLVKYYAPWCGHCKRMAPAYEELATLYAND |
| | | EDASSKVVIAKLDHTLNDVDNVDIQGYPTLILYPAGDKSNPQLYDGSRDLESL |
| | | AEFVKERGTHKVDALALRPVEEEKEAEEEAESEADAHDEL |

### Glutathione Peroxidase 1 (GPX1) Proteins

In some embodiments, an expression system comprises a polynucleotide having a transcriptional unit encoding a glutathione peroxidase (GPX1) protein (or a homolog thereof).

Without wishing to be bound by any particular theory, the present disclosure notes that GPX1 proteins have been reported as having various functions, including the ability to protect cells against oxidative damage by catalyzing the reduction of organic hydroperoxides and hydrogen peroxide (H2O2) using glutathione. Toledano et al. 2013 Antioxid. Redox Signal 18: 1699.

Exemplary GPX1 proteins are provided in TABLE 11. Additional GPX1 proteins (and homologs thereof) are known to those having ordinary skill in the art. In some embodiments, a homolog of GPX1 includes but is not limited to: AaceriAFL039Cp, Ashbya aceris AGO13118.1; peroxiredoxin HYR1, Candida auris, XP_028890744.1; hypothetical protein CA7LBN_000571, Candida auris, QWW21825.1; hypothetical protein B5S28_g2387, Candida boidinii, OWB56484.1; hypothetical protein BVG19_g2086, Candida boidinii, OUM52855.1; hypothetical protein B5S32_g1485, Candida boidinii, OWB77323.1; peroxiredoxin HYR1, Candida haemuloni, XP_025340580.1; CIC11C00000005432, Candida intermedia, SGZ57738.1; glutathione peroxidase-like protein, Candida oleophila, AEY94433.1; peroxiredoxin HYR1, Candida pseudohaemulonii, XP_024715211.1; glutathione peroxidase-like peroxiredoxin Hyr1p, Candida railenensis, CAH2352981.1; GPX2, Candida subhashii, XP_049265885.1; glutathione peroxidase, Ascoidea rubescens DSM 1968, XP_020049141.1; uncharacterized protein BABINDRAFT_177818, Babjeviella inositovora NRRL Y-12698, XP_018983180.1; GPX3, Candida africana, KAG8202395.1; peroxiredoxin HYR1, Candida albicans P34048, KGU22529.1; peroxiredoxin, Candida albicans SC5314, XP_714295.1; hydrogen peroxide resistance protein, putative, Candida dubliniensis CD36, XP_002420878.1; GPX2, Candida oxycetoniae, XP_049180930.1; peroxiredoxin HYR1, Candida tropicalis MYA-3404, XP_002548683.1; hypothetical protein CLUG_05152, Clavispora lusitaniae ATCC 42720, XP_002615137.1; Peroxiredoxin HYR1, Cyberlindnera fabianii, ONH67665.1; glutathione peroxidase, Cyberlindnera jadinii NRRL Y-1542, XP_020069848.1; Peroxiredoxin HYR1, Debaryomyces fabryi, XP_015465158.1; DEHA2F09526p, Debaryomyces hansenii CBS767, XP_460775.2; Hypothetical protein Ecym_2257, Eremothecium cymbalariae DBVPG #7215, XP_003644820.1; AFL039Cp, Eremothecium gossypii ATCC 10895, NP_985509.1; HER033Wp, Eremothecium sinecaudum, XP_017988308.1; GSHPx-domain-containing protein, Hyphopichia burtonii NRRL Y-1933, XP_020078783.1; unnamed protein product, Kluyveromyces dobzhanskii CBS 2104, CD095641.1; uncharacterized protein KLLA0_F06732g, Kluyveromyces lactis, XP_455385.1; Gpx2, Kluyveromyces lactis, QEU59086.1; peroxiredoxin HYR1, Kluyveromyces marxianus DMKU3-1042, XP_022674733.1; peroxiredoxin hyr1, Kluyveromyces marxianus, KAG0677360.1; peroxiredoxin HYR1, Kluyveromyces marxianus, QGN14601.1; BA75_02278T0, Komagataella pastoris, ANZ75783.1; Thiol peroxidase that functions as a hydroperoxide receptor, Komagataella phaffii GS115, XP_002491803.1; uncharacterized protein KUCA_T00000275001, Kuraishia capsulata CBS 1993, XP_022456332.1; LADA_0A02674g1_1, Lachancea dasiensis, SCU77880.1; LAFE_0C06018g1_1, Lachancea fermentati, SCW00521.1; uncharacterized protein LALA0_S13e03114g, Lachancea lanzarotensis, XP_022630995.1; LAME_0C08152g1_1, Lachancea meyersii CBS 8951, SCU84084.1; LAMI_0F07932g1_1, Lachancea mirantina, SCU96841.1; LANO_0F01618g1_1, Lachancea nothofagi CBS 11611, SCU99369.1; LAQU0S02e10902g1_, Lachancea quebecensis, CUS21315.1; LAFA_0C03268g1_1, Lachancea sp. CBS 6924, SCU81199.1; KLTH0H05588p, Lachancea thermotolerans CBS 6340, XP_002556122.1; hypothetical protein PGUG_01527, Meyerozyma guilliermondii ATCC 6260, EDK37429.2; hypothetical protein PGUG_01527, Meyerozyma guilliermondii ATCC 6260, XP_001485856.1; Peroxiredoxin HYR1, Meyerozyma sp. JA9, RLV87932.1; Piso0_004054, Millerozyma farinosa CBS 7064, CCE83478.1; hypothetical protein NCAS_0F00650, Naumovozyma castelihi CBS 4309, XP_003676905.1; hypothetical protein NDAI_0D02480, Naumovozyma dairenensis CBS 421, XP_003669805.1; hyr1p, Saccharomyces arboricola H-6, EJS43249.1; gpx2p, Saccharomyces arboricola H-6, EJS44691.1; Hyr1p, Saccharomyces cerevisiae, EGA61848.1; peroxiredoxin HYR1, Saccharomyces cerevisiae S288C, NP_012303.1; glutathione peroxidase GPX2, Saccharomyces cerevisiae S288C, NP_009803.3; Gpx2p, Saccharomyces cerevisiae YJM1387, AJQ14782.1; Gpx2p, Saccharomyces cerevisiae YJM1415, AJP82604.1; Gpx2p, Saccharomyces cerevisiae YJM1418, AJP83383.1; Gpx2p, Saccharomyces cerevisiae YJM1439, AJP84917.1; Gpx2p, Saccharomyces cerevisiae YJM1479, AJP88322.1; Crystal structure of glutathione-dependent phospholipid peroxidase Hyr1 from the yeast Saccharomyces cerevisiae, Saccharomyces cerevisiae, 3CMI_A; EM14S01-3B_G0053580.mRNA.1.CDS.1, Saccharomyces cerevisiae, CAD6620203.1; peroxiredoxin HYR1, Saccharomyces cerevisiae, PTN13363.1; hypothetical protein SCEPF1_0044000400, Saccharomyces cerevisiae, GES70023.1; glutathione peroxidase-like peroxiredoxin 2, Saccharomyces cerevisiae, GFP67031.1; SX2_G0016480.mRNA.1.CDS.1, Saccharomyces cerevisiae, CAD6600729.1; XXYS1_4_G0018630.mRNA.1.CDS.1, Saccharomyces cerevisiae, CAD6600612.1; HYR1-like protein, Saccharomyces eubayanus, XP_018221642.1; Hyr1, Saccharomyces paradoxus, XP_033767077.1; Gpx2, Saccharomyces paradoxus, XP_033764833.1; hypothetical protein SCDLUD_000615, Saccharomycodes ludwigii, XP_045936939.1; uncharacterized protein SAPINGB_P005029, Saprochaete ingens, XP_031855635.1; hypothetical protein TPHA_0J02050, Tetrapisispora phaffii CBS 4417, XP_003687459.1; hypothetical protein TDEL_0A04230, Torulaspora delbrueckii, XP_003678966.1; hypothetical protein TDEL_0B03940, Torulaspora delbrueckii, XP_003679734.1; uncharacterized protein HG536_0A05790, Torulaspora globosa, XP_037137439.1; hypothetical protein HG537_0A05740, Torulaspora sp. CBS 2947, QLQ78327.1; hypothetical protein Kpol_1002p86, Vanderwaltozyma polyspora DSM 70294, XP_001647296.1; hypothetical protein WICANDRAFT_33882, Wickerhamomyces anomalus NRRL Y-366-8, XP_019037569.1; Phospholipid hydroperoxide glutathione peroxidase, mitochondrial, Wickerhamomyces ciferrii, XP_011271094.1; hypothetical protein WICMUC_004474, Wickerhamomyces mucosus, KAH3672047.1; YALI0E02310p, Yarrowia lipolytica CLIB122, XP_503454.1; hypothetical protein YALI1_E02906g, Yarrowia lipolytica, AOW04848.1; Peroxiredoxin HYR1, Yarrowia sp. B02, KAG5366561.1; Peroxiredoxin HYR1, Yarrowia sp. E02, KAG5356989.1; probable Peroxiredoxin HYR1, Zygosaccharomyces bailii ISA1307, CDH12493.1; probable Peroxiredoxin HYR1, Zygosaccharomyces bailii, SJM83001.1; peroxiredoxin hyr1, Zygosaccharomyces mellis, GCF00733.1; GPX2 (YBR244W), Zygosaccharomyces parabailii, AQZ16207.1; GPX2 (YBR244W), Zygosaccharomyces parabailii, AQZ09810.1; (ZYROOD13288g), Zygosaccharomyces parabailii, AQZ16764.1; (ZYROOD13288g), Zygosaccharomyces parabailii, AQZ12536.1; thioredoxin-like protein, Zygosaccharomyces rouxii, KAH9202981.1; hypothetical protein ZYGR_0BB01580, Zygosaccharomyces rouxii, GAV56381.1; uncharacterized protein ZYRO0G21758g, Zygosaccharomyces rouxii, XP_002498924.1; hypothetical protein ZYGR_0AK05710, Zygosaccharomyces rouxii, GAV54069.1; and uncharacterized protein HG535_0B01660, Zygotorulaspora mrakii, XP_037142856.1. In some embodiments, a homolog of GPX1 has a sequence which is at least about 60%, 70%, 80%, 90%, or 95% identical to an amino acid sequence listed in TABLE 11, or any amount in between any of these percentages.

In some embodiments, an expression system comprises a polynucleotide having a transcriptional unit encoding a GPX1 protein (or a homolog thereof), wherein the GPX1 protein has at least about 60%, 70%, 80%, 90%, or 95% identity to an amino acid sequence listed in TABLE 11, or any amount in between any of these percentages.

In some embodiments, the nucleic acid encoding the GPX1 protein is operably linked to a promoter, such as a constitutive promoter (as described herein), an inducible promoter (as described herein), or a synthetic promoter (as described herein).

In some embodiments, a transcriptional unit encoding a glutathione peroxidase (GPX1) protein (or a homolog thereof) has a structure as depicted in FIG. 4D.

TABLE 11
Amino Acid Sequences of Exemplary GPX1 Proteins

| SEQ | | |
|---|---|---|
| ID NO: | Descr. | Sequence |
| 201 | Pichia pastoris | MSSFYDLAPLDKKGEPFPFEQLKGKVVLIVNVASKCGFTPQYTELEKLYK |
| | GPX1 (NCBIGQ68_ | DHKDEGLTIVGFPCNQFGHQEPGNDEEIGQFCQLNFGVTFPILKKIDVNG |
| | 00445T0) | SEADPVYEFLKSKKSGLLGFKGIKWNFEKFLIDKQGNVIERYSSLTKPSS |
| | | IESKIEELLKK |
| 202 | Saccharomyces | MSEFYKLAPVDKKGQPFPFDQLKGKVVLIVNVASKCGFTPQYKELEALYK |
| | cerevisiae Hyr1p | RYKDEGFTIIGFPCNQFGHQEPGSDEEIAQFCQLNYGVTFPIMKKIDVNG |
| | (EGA61848.1) | GNEDPVYKFLKSQKSGMLGLRGIKWNFEKFLVDKKGKVYERYSSLTKPSS |
| | | LSETIEELLKEVE |

### HAC1 Proteins

In some embodiments, an expression system comprises a polynucleotide having a transcriptional unit encoding a HAC1 protein (or a homolog thereof).

Without wishing to be bound by any particular theory, the present disclosure notes that HAC1 proteins have been reported as having various functions, including the ability to enhance protein secretion.

Exemplary HAC1 proteins are provided in TABLE 12. Additional HAC1 proteins (and homologs thereof) are known to those having ordinary skill in the art. In some embodiments, a homolog of HAC1 has a sequence which is at least about 60%, 70%, 80%, 90%, or 95% identical to an amino acid sequence listed in TABLE 12, or any amount in between any of these percentages. In some embodiments, a homolog of HAC1 includes but is not limited to: hypothetical protein, Asgard group archaeon, MCP8718148.1; AaceriACR216Cp, Ashbya aceris (nom. inval.), AGO11461.1; uncharacterized protein BABINDRAFT_169569, Babjeviella inositovora NRRL Y-12698, XP_018982282.1; hypothetical protein GWM34_00345, Candida africana, KAG8204544.1; hypothetical protein MG1_00598, Candida albicans GC75, KGR03812.1; hypothetical protein MGI_00593, Candida albicans P75016, KHC76018.1; hypothetical protein MGM_00592, Candida albicans P75063, KGU36189.1; transcriptional activator HAC1, Candida albicans P76055, KHC40640.1; transcriptional activator HAC1, Candida albicans P76067, KHC43434.1; hypothetical protein MGS_00596, Candida albicans P78042, KHC85199.1; hypothetical protein MEQ_00591, Candida albicans P87, KGU14942.1; hypothetical protein MEO_00596, Candida albicans P94015, KGQ91868.1; transcription factor, Candida albicans SC5314, XP_019330657.1; conserved hypothetical protein, Candida albicans WO-1, EEQ42577.1; hypothetical protein FOB64_006133, Candida albicans, KAF6063127.1; hypothetical protein CJJ07_002320, Candida auris, PSK77776.1; transcriptional activator hael, Candida auris, QRG35958.1; transcriptional activator hael, Candida auris, XP_028889751.1; nuclear protein HAC1, putative, Candida dubliniensis CD36, XP_002417244.1; hypothetical protein G210_1715, Candida maltosa Xu316, EMG47836.1; predicted protein, Candida tropicalis MYA-3404, XP_002549979.1; hypothetical protein Cantr_08192, Candida viswanathii, RCK60783.1; hypothetical protein Cantr_05453, Candida viswanathii, RCK56067.1; hypothetical protein CLUG_03727, Clavispora lusitaniae ATCC 42720, XP_002616486.1; Transcriptional activator HAC1, Cyberlindnera fabianii, ONH68652.1; HAC1, Cyberlindnera jadinii, CEP24118.1; hypothetical protein Ecym_1506, Eremothecium cymbalariae DBVPG #7215, XP_003644547.1; ACR216Cp, Eremothecium gossypii ATCC 10895, NP_983618.2; HDL529Wp, Eremothecium sinecaudum, XP_017987211.1; XBP1, Homo sapiens; Transcriptional activator HAC1, Hanseniaspora osmophila, OEJ85032.1; hypothetical protein HYPBUDRAFT_158623, Hyphopichia burtonii NRRL Y-1933, XP_020074116.1; hypothetical protein KAFR_0C03510, Kazachstania africana CBS 2517, XP_003956478.1; transcription factor HAC1, Kazachstania barnettii, XP_041404513.1; hypothetical protein C6P45_000270, Kazachstania exigua, KAG0666309.1; similar to Saccharomyces cerevisiae YFLO31W HAC1 Basic leucine zipper (bZIP) transcription factor (ATF/CREB1 homolog) that regulates the unfolded protein response, Kazachstania saulgeensis, SMN18560.1; unnamed protein product, Kluyveromyces dobzhanskii CBS 2104, CD095739.1; Hac1, Kluyveromyces lactis, QEU59188.1; uncharacterized protein KLLA0_F08976g, Kluyveromyces lactis, XP_455488.1; basic-leucine zipper (bZIP) transcription factor, Kluyveromyces marxianus DMKU3-1042, XP_022674633.1; hypothetical protein C6P43_002536, Kluyveromyces marxianus, KAG0671368.1; basic-leucine zipper (bZIP) transcription factor, Kluyveromyces marxianus, QGN14499.1; LADA_0C11408g1_1, Lachancea dasiensis, SCU83431.1; LAFE_0B08966g1_1, Lachancea fermentati, SCW00078.1; uncharacterized protein LALA0_S15e00716g, Lachancea lanzarotensis, XP_022631132.1; LAME_0A07206g1_1, Lachancea meyersii CBS 8951, SCU79099.1; LAMI_0G16864g1_1, Lachancea mirantina, SCV02208.1; LANO_0D10198g1_1, Lachancea nothofagi CBS 11611, SCU90893.1; LAQUOS06e04962g1_1, Lachancea quebecensis, CUS22740.1; LAFA_0A01112g1_1, Lachancea sp. CBS 6924, SCU77326.1; KLTH0G18568p, Lachancea thermotolerans CBS 6340, XP_002555833.1; Piso0_004325, Millerozyma farinosa CBS 7064, CCE83739.1; hypothetical protein NCAS_0C03780, Naumovozyma castelihi CBS 4309, XP_003675733.1; hypothetical protein NDAI_0G03120, Naumovozyma dairenensis CBS 421, XP_003671332.2; HAC1p Basic leucine zipper (bZIP) transcription factor, Saccharomyces boulardii (nom. inval.), KOH50922.1; YFLO31Wp-like protein, Saccharomyces cerevisiae AWRI1631, EDZ72446.1; Hac1p, Saccharomyces cerevisiae AWRI796, EGA75098.1; Hac1p, Saccharomyces cerevisiae CEN.PK113-7D, EIW10734.1; Hac1p, Saccharomyces cerevisiae EC1118, CAY79417.1; Hac1p, Saccharomyces cerevisiae FostersB, EGA58892.1; Hac1p, Saccharomyces cerevisiae FostersO, EGA62515.1; Hac1p, Saccharomyces cerevisiae JAY291, EEU04232.1; bZIP protein, Saccharomyces cerevisiae RM11-1a, EDV09811.1; transcription factor HAC1, Saccharomyces cerevisiae S288C, NP_116622.1; Hac1p, Saccharomyces cerevisiae VL3, EGA87058.1; Hac1p, Saccharomyces cerevisiae x Saccharomyces kudriavzevii VIN7, EHN07414.1; Hac1p, Saccharomyces cerevisiae x Saccharomyces kudriavzevii VIN7, EHN02603.1; conserved protein, Saccharomyces cerevisiae YJM789, EDN59119.1; transcription factor HAC1, Saccharomyces cerevisiae, PTN13857.1; Y55_G0049890.mRNA.1.CDS.1, Saccharomyces cerevisiae, CAD6469134.1; EM14S01-3B_G0049200.mRNA.1.CDS.1, Saccharomyces cerevisiae, CAD6622865.1; SX2_G0049830.mRNA.1.CDS.1, Saccharomyces cerevisiae, CAD6626058.1; transcription factor HAC1, Saccharomyces cerevisiae, PTN39216.1; HN1_G0049840.mRNA.1.CDS.1, Saccharomyces cerevisiae, CAD6623120.1; XXYS1_4_G0051920.mRNA.1.CDS.1, Saccharomyces cerevisiae, CAD6623523.1; Transcriptional activator HAC1, Saccharomyces cerevisiae, KAF4001172.1; Alanine—glyoxylate aminotransferase 1, Saccharomyces cerevisiae, ONH78992.1; Hacd, Saccharomyces cerevisiae, BAA05513.1; HAC1-like protein, Saccharomyces eubayanus, XP_018222508.1; Hac1, Saccharomyces paradoxus, XP_033766036.1; transcription factor that binds to CRE motif, Saccharomyces pastorianus, QID84752.1; hypothetical protein SCDLUD_000612, Saccharomycodes ludwigii, XP_045936936.1; hypothetical protein G9P44_002014, Scheffersomyces stipitis, KAG2735800.1; hypothetical protein TBLA_0G03420, Tetrapisispora blattae CBS 6284, XP_004181798.1; hypothetical protein TPHA_0D00380, Tetrapisispora phaffii CBS 4417, XP_003685116.1; hypothetical protein TDEL_0C00610, Torulaspora delbrueckii, XP_003680161.1; uncharacterized protein HG536_0B06660, Torulaspora globosa, XP_037138473.1; hypothetical protein HG537_0B06600, Torulaspora sp. CBS 2947, QLQ79312.1; hypothetical protein Kpol_1035p38, Vanderwaltozyma polyspora DSM 70294, XP_001645083.1; Transcriptional activator HAC1, Wickerhamomyces ciferrii, XP_011274629.1; hypothetical protein WICMUC_002835, Wickerhamomyces mucosus, KAH3675179.1; hypothetical protein WICPIJ_001578, Wickerhamomyces pijperi, KAH3687468.1; uncharacterized protein ZBAI_01420, Zygosaccharomyces bailii ISA1307, CDH09636.1; uncharacterized protein ZBAI_05687, Zygosaccharomyces bailii ISA1307, CDH13901.1; uncharacterized protein ZBIST_4677, Zygosaccharomyces bailii, SJM88488.1; hypothetical protein ZYGM_000655, Zygosaccharomyces mellis, GCF01550.1; HAC1 (YFL031W), Zygosaccharomyces parabailii, AQZ19104.1; HAC1 (YFL031W), Zygosaccharomyces parabailii, AQZ15209.1; hypothetical protein ZYGR_0AS01310, Zygosaccharomyces rouxii, GAV54808.1; uncharacterized protein ZYRO0F03102g, Zygosaccharomyces rouxii, XP_002497332.1; and uncharacterized protein HG535_0D05810, Zygotorulaspora mrakii, XP_037144599.1.

In some embodiments, an expression system comprises a polynucleotide having a transcriptional unit encoding a HAC1 protein (or a homolog thereof), wherein the HAC1 protein has at least about 60%, 70%, 80%, 90%, or 95% identity to an amino acid sequence listed in TABLE 12, or any amount in between any of these percentages.

In some embodiments, the nucleic acid encoding the HAC1 protein is operably linked to a promoter, such as a constitutive promoter (as described herein), an inducible promoter (as described herein), or a synthetic promoter (as described herein).

In some embodiments, a transcriptional unit encoding a HAC1 protein (or a homolog thereof) has a structure as depicted in FIG. 4C.

TABLE 12
Amino Acid Sequences of Exemplary HAC1 Proteins

| SEQ | | |
|---|---|---|
| ID NO: | Descr. | Sequence |
| 203 | Saccharomyces | MEMTDFELTSNSQSNLAIPTNFKSTLPPRKRAKTKEEKEQRRIERILRNR |
| | cerevisiae HAC1 | RAAHQSREKKRLHLQYLERKCSLLENLLNSVNLEKLADHEDALTCSHDAF |
| | (UniProt P41546-1) | VASLDEYRDFQSTRGASLDTRASSHSSSDTFTPSPLNCTMEPATLSPKSM |
| | | RDSASDQETSWELQMFKTENVPESTTLPAVDNNNLFDAVASPLADPLCDD |
| | | IAGNSLPFDNSIDLDNWRNPAVITMTRKLQ* |

### Transcription Factors

In some embodiments, an expression system further comprises one or more polynucleotides encoding a transcription factor that regulates (increase or decrease) expression of: a secreted protein of the expression system; a calreticulin (CRT) protein of the expression system; a protein disulfide isomerase family A member 3 (PDIA3) protein of the expression system; a protein disulfide isomerase 1 (PDI1) protein of the expression system; a glutathione peroxidase 1 (GPX1) protein of the expression system; and/or a HAC1 protein of the expression system.

In some embodiments, a transcription factor is a synthetic transcription factor.

In some embodiments, a transcription factor (e.g., a synthetic transcription factor) increases the rate of transcription of a gene of interest by binding to a synthetic output promoter operably linked to the gene of interest. A transcription factor can work alone, or can work with other proteins in a complex by recruiting components of and/or stabilizing a complex comprising an RNA polymerase at the synthetic output promoter. In some embodiments, a transcription factor (e.g., a synthetic transcription factor) comprises at least one of: (1) a DNA-binding domain, which binds to a specific DNA sequence, and/or (2) a transcriptional activation domain (e.g., a trans-acting domain; TAD), which can interact with another protein such as a RNA polymerase, another protein, or another component in a complex comprising the RNA polymerase.

Without wishing to be bound by any particular theory, it is noted that a transcription factor (e.g., a synthetic transcription factor) can increase expression from a synthetic output promoter by various mechanisms, including but not limited to: stabilizing the binding of RNA polymerase to the promoter; catalyzing the acetylation of histone proteins via histone acetyltransferase (HAT) activity; weakening the association of DNA with histones and making the DNA more accessible to transcription; and/or recruiting coactivator or corepressor proteins to the transcription complex. In some embodiments, a transcription factor comprises a signal-sensing domain (SSD) (e.g., a ligand binding domain), which senses external signals and, in response, transmits these signals to the rest of the transcription complex, resulting in up-regulation of expression of the gene of interest.

Various transcription factors, and their structures and functions, are described in the literature, including: Latchman 1997 Int. J. Biochem. Cell Biology. 29 (12): 1305-12; Karin 1990 The New Biologist. 2 (2): 126-31; Babu et al. 2004 Current Opinion in Structural Biology. 14 (3): 283-91; Roeder 1996 Trends in Biochemical Sciences. 21 (9): 327-35; Nikolov et al. 1997 Proc. Nat. Acad. Sci. United States of America. 94 (1): 15-22; Lee et al. 2000 Annual Review of Genetics. 34: 77-137; Mitchell et al. 1989 Science. 245 (4916): 371-8; Ptashne et al. 1997 Nature. 386 (6625): 569-77; Jin et al. 2014 Nucleic Acids Research. 42 (Database issue): D1182-7; and Matys et al. 2006 Nucleic Acids Research. 34 (Database issue): D108-10.

In some embodiments, a transcription factor (e.g., a synthetic transcription factor) is as described in WO 2022/051696 A1, the entire contents of which are incorporated herein by reference. For example, a synthetic transcription factor may be a transcription factor provided in any of Tables 7-14 of WO 2022/051696 A1.

The amino acid sequences of exemplary synthetic transcription factors are provided in TABLE 13. In some embodiments, an expression system comprises a polynucleotide encoding a synthetic transcription factor having at least about 60%, 70%, 80%, 90%, or 95% identity to an amino acid listed in TABLE 13, or any amount in between any of these percentages.

In some embodiments, the nucleic acid encoding the synthetic transcription factor is operably linked to a promoter, such as a constitutive promoter (as described herein), an inducible promoter (as described herein), or a synthetic promoter (as described herein).

TABLE 13
Amino Acid Sequences of Exemplary Synthetic Transcription Factors

| SEQ | | |
|---|---|---|
| ID NO: | Description | Sequence |
| 204 | TetR-SV40_NLS- | MSRLDKSKVINSALELLNEVGIEGLTTRKLAQKLGVEQPTLYWHVKNKR |
| | Linker1_long- | ALLDALAIEMLDRHHTHFCPLEGESWQDFLRNNAKSFRCALLSHRDGAK |
| | VPH TAD | VHLGTRPTEKQYETLENQLAFLCQQGFSLENALYALSAVGHFTLGCVLE |
| | | DQEHQVAKEERETPTTDSMPPLLRQAIELFDHQGAEPAFLFGLELIICG |
| | | LEKQLKCESGSEFPPKKKRKVGSTSGSGKPGSGEGSTKGDALDDFDLDM |
| | | LGSDALDDFDLDMLGSDALDDFDLDMLGSDALDDFDLDMLINSRSSGSP |
| | | KKKRKVGSGGGSGGSGSPSGQISNQALALAPSSAPVLAQTMVPSSAMVP |
| | | LAQPPAPAPVLTPGPPQSLSAPVPKSTQAGEGTLSEALLHLQFDADEDL |
| | | GALLGNSTDPGVFTDLASVDNSEFQQLLNQGVSMSHSTAEPMLMEYPEA |
| | | ITRLVTGSQRPPDPAPTPLGTSGLPNGLSGDEDFSSIADMDFSALLSQI |
| | | SSSGQGGGGSGFSVDTSALLDLFSPSVTVPDMSLPDLDSSLASIQELLS |
| | | PQEPPRPPEAENSSPDSGKQLVHYTAQPLFLLDPGSVDTGSNDLPVLFE |
| | | LGEGSYFSEGDGFAEDPTISLLTGSEPPKAKDPTVS |
| 205 | VanR_AM- | MDMPRIKPGQRVMMALRKMIASGEIKSGERIAEIPTAAALGVSRMPVRI |
| | SV40_NLS- | ALRSLEQEGLVVRLGARGYAARGVSSDQIRDAIEVRGVLEGFAARRLAE |
| | Linker1_long- | RGMTAETHARFVVLIAEGEALFAAGRLNGEDLDRYAAYNQAFHDTLVSA |
| | B112_TAD \| | AGNGAVESALARNGFEPFAAAGALALDLMDLSAEYEHLLAAHRQHQAVL |
| | bsaI, bmbI, | DAVSCGDAEGAERIMRDHALAAIRNAKVFEAAASAGAPLGAAWSIRADE |
| | aarI removed | FPPKKKRKVGSTSGSGKPGSGEGSTKGEFPGITLRIQETDMLYKGDTLY |
| | | LDWLEDGIAELVFDAPGSVNKLDTAVASLGEAIGVLEQQSDLIWETLTV |
| | | KDAKVNFDSGLEKFEEAIPSADDFDPVAERRSSGEFRAERHSGGTDLCF |

### Genetically Modified Host Cells

Aspects of the disclosure relate to genetically modified host cells comprising an expression system as described herein.

In some embodiments, a genetically modified host cell comprises a genetic modification that results in overexpression of a heterologous gene (e.g., a gene encoding a secreted protein, a gene encoding a calreticulin (CRT) protein, a gene encoding a protein disulfide isomerase family A member 3 (PDIA3) protein, a gene encoding a protein disulfide isomerase 1 (PDI1) protein, a gene encoding a glutathione peroxidase 1 (GPX1) protein, and/or a gene encoding a HAC1 protein of an expression system described herein), as compared to a reference or control host cell which does not comprise the corresponding genetic modifications.

In some embodiments, a genetically modified host cell comprises a genetic modification that results in overexpression, as compared to a reference or control host cell which does not comprise the corresponding genetic modifications, of a gene encoding a calreticulin (CRT) protein. In some embodiments, a genetically modified host cell comprises a polynucleotide having a transcriptional unit encoding for a CRT protein, wherein the genetically modified host cell is capable of overexpressing (constitutively or inducibly) the CRT protein.

In some embodiments, a genetically modified host cell comprises a genetic modification that results in overexpression, as compared to a reference or control host cell which does not comprise the corresponding genetic modifications, of a gene encoding a protein disulfide isomerase family A member 3 (PDIA3) protein. In some embodiments, a genetically modified host cell comprises a polynucleotide having a transcriptional unit encoding for a PDIA3 protein, wherein the genetically modified host cell is capable of overexpressing (constitutively or inducibly) the PDIA3 protein.

In some embodiments, a genetically modified host cell comprises a genetic modification that results in overexpression, as compared to a reference or control host cell which does not comprise the corresponding genetic modifications, of a gene encoding a protein disulfide isomerase 1 (PDI1) protein. In some embodiments, a genetically modified host cell comprises a polynucleotide having a transcriptional unit encoding for a PDI1 protein, wherein the genetically modified host cell is capable of overexpressing (constitutively or inducibly) the PDI1 protein.

In some embodiments, a genetically modified host cell comprises a genetic modification that results in overexpression, as compared to a reference or control host cell which does not comprise the corresponding genetic modifications, of a gene encoding a glutathione peroxidase 1 (GPX1) protein. In some embodiments, a genetically modified host cell comprises a polynucleotide having a transcriptional unit encoding for a GPX1 protein, wherein the genetically modified host cell is capable of overexpressing (constitutively or inducibly) the GPX1 protein.

In some embodiments, a genetically modified host cell comprises a genetic modification that results in overexpression, as compared to a reference or control host cell which does not comprise the corresponding genetic modifications, of a gene encoding a HAC1 protein. In some embodiments, a genetically modified host cell comprises a polynucleotide having a transcriptional unit encoding for a HAC1 protein, wherein the genetically modified host cell is capable of overexpressing (constitutively or inducibly) the HAC1 protein.

In some embodiments, a genetically modified host cell further comprises one or more genetic modifications that introduce a mutant heat shock transcription factor 1 (HSF1) gene (e.g., by mutating a native HSF1 gene, or adding one or more copies of a native or heterologous HSF1 mutant (including homologs of a native HSF1)) and/or downregulate (e.g., reduce the expression, abundance and/or activity of) one or more endogenous genes, such as SSY1, HSL1, PAS_chr2-1_0053, PAS_chr2-1_0404, PAS_chr4_0550, PAS_chr1-3_0135, and/or PAS_chr1-3_0285 (or a homolog of any of them).

In some embodiments, a host cell (or genetically modified host cell) is a eukaryotic cell. Suitable eukaryotic host cells include, but are not limited to, fungal cells (e.g., yeast cells), algal cells, plant cells, insect cells, and animal cells, including mammalian cells. In some embodiments, the host cell is a mammalian cell, an insect cell, an arthropod cell, a fish cell, an amphibian cell, a reptilian cell, a bird cell, a plant cell, etc., or a fungal cell other than a yeast.

In some embodiments, the host cell (or genetically modified host cell) is a yeast. Suitable yeast host cells include, but are not limited to: Candida, Hansenula, Saccharomyces (e.g., S. cerevisiae), Schizosaccharomyces, Pichia, Kluyveromyces, and Yarrowia (e.g., Y. lipolytica). In some embodiments, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica.

In some embodiments, the host cell (or genetically modified host cell) is a methylotrophic yeast, such as Pichia pastoris. In some embodiments, the host cell (or genetically modified host cell) is a non-yeast. In some embodiments, the host cell is not Saccharomyces.

In some embodiments, methylotrophic yeast cells in a plurality of methylotrophic yeast cells may individually or as a collective group comprise genetic modifications according to any embodiment of the present disclosure.

In some embodiments, a yeast host cell (or genetically modified yeast host cell) includes any of: a member of the genera Pichia, Komagataella, Candida, Dipodascus, Galactomyces, Hansenula, Kluyveromyces (e.g., K. lactis), Magnusiomyces, Ogatae, Phaffomyces, Saccharomyces (e.g., S. cerevisiae), Schizosaccharomyces, Starmera, Starmerella, Sugiyamaella, Trichomonascus, Wickerhamomyces, Wickerhamiella, Williopsis, Yarrowia, or Zygoascus; or a member of Komagataella Clade, Phaffomyces Clade, Dipodascaceae, Phaffomycetaceae, or Trichomonascaceae. In some embodiments, the methylotrophic yeast host cell is a member of the genera Pichia or Komagataella. In some embodiments, the yeast host cell is any of: Pichia pastoris, Pichia pseudopastoris, Pichia stipitis, Pichia membranifaciens, Pichia methanolica, Pichia finlandica, Pichia trehalophila, Pichia kodamae, Pichia opuntiae, Pichia thermotolerans, Pichia salictaria, Pichia quercuum, Pichia pijperi, Pichia angusta, Komagataella phaffii, Komagataella pastoris, Komagataella pseudopastoris, Komagataella kurtzmanii, Komagataella mondaviorum, Wickerhamomyces anomalus, Candida albicans, Candida lusitaniae, Ogataea glucozyma, Candida blankii, Candida boidinii, Candida orba, Candida petrohuensis, Candida santjacobensis, Candida sorboxylosa, Candida sp., Dipodascus albidus, Galactomyces geotrichum, Hansenula polymorpha, Kluyveromyces lactis, Magnusiomyces magnusii, Phaffomyces antillensis, Phaffomyces opuntiae, Phaffomyces thermotolerans, Saccharomyces carlsbergensis, Saccharomyces diastaticus, Saccharomyces norbensis, Saccharomyces kluyveri, Schizosaccharomyces pombe, Starmerella bombicola, Sugiyamaella smithiae, Trichomonascus petasosporus, Wickerhamiella domercqiae, Yarrowia lipolytica, or Zygoascus hellenicus.

Aspects of the disclosure relate to genetically modified Pichia pastoris host cells. Without wishing to be bound by any particular theory, the present disclosure notes that some reports in the scientific literature reassigned P. pastoris to the genus Komagataella, and various strains of P. pastoris were separated into K. phaffii, K. pastoris, and K. pseudopastoris. In some embodiments, Pichia pastoris is identical to Komagataella phaffii, and Komagataella phaffii is sometimes referred to by its former species name Pichia pastoris. As used in this disclosure, Pichia pseudopastoris is interchangeable with Komagataella pseudopastoris. These various genera and species, and the relationships between them, are described in the scientific literature, for example: Feng et al. 2020 Yeast, 37(2):237-245; De Schutter et al. 2009 Nat. Biotechnol., 27 (6): 561-566; Heistinger et al. 2018 Mol. Cell. Biol., 38 Issue 2 e00398-17; Kurtzman, 2005 Int. J. Syst. Evol. Microbiol. 55: 973-976; Kurtzman 2011 Antonie van Leeuwenhoek, 99:13-23; Kurtzman 2013 Antonie van Leeuwenhoek, 104:339-347; Kurtzman, 2012 Antonie van Leeuwenhoek, 101: 859-868; Naumov, 2018 Antonie van Leeuwenhoek, 111:1197-1207; and Yamada et al. 1995 Biosci. Biotech. Biochem., 59: 439-444. In some embodiments, a methylotrophic yeast host cell is an undescribed species of Pichia or Komagataella. In some embodiments, a host cell is a Pichia sp. or Komagataella sp.

In certain embodiments, the host cell (or genetically modified host cell) is a mold cell such as, Aspergillus (e.g., Aspergillus niger).

In certain embodiments, the host cell (or genetically modified host cell) is an algal cell such as, Chlamydomonas (e.g., C. Reinhardtii) and Phormidium (P. sp. ATCC 29409).

The present disclosure is also suitable for use with a variety of animal cell types, including mammalian cells, for example, human (including 293, HeLa, W138, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

### HSF1 Mutants

In some embodiments, a genetically modified host cell comprises one or more genetic modifications that introduce a mutant HSF1 gene (e.g., by mutating a native HSF1 gene, or adding one or more copies of a native or heterologous HSF1 mutant (including homologs of a native HSF1)). The host cells of this disclosure may be modified to comprise a mutant HSF1 using any method known in the art, including, by way of non-limiting example, by mutating the host's native HSF1 in situ, adding one or more copies of a native HSF1 harboring the relevant mutations, and/or adding one or more copies of a heterologous HSF1 homolog harboring the relevant mutations.

HSF1 is reportedly a major transcription factor that coordinates the expression of many heat shock proteins in the heat shock response (HSR). The HSR ensures proper protein folding within a cell during a variety of stressful conditions, including temperature stress, but also hypoxia, oxidative stress, and exposure to contaminants, UV light or methanol. In addition to ensuring proper protein folding, various heat shock proteins stabilize newly synthesized proteins and prevent unwanted protein aggregation.

The wild-type Pichia pastoris HSF1 amino acid sequence is set forth as follows:

(SEQ ID NO: 206)
MSDFPKVEQASPSEGMPQVQTNNAPEIEEIEDIIRDTPNVMDVLGNGSQS
WDGTNSNGAAMGSLDTVPANKLVAYNKYDNNIYPDPLQPLSDTALPNSMG
YYSMIKAGDKGLKPQSFRKTKKKPTPSGPKTRPAFVMKLWNMVHDPSNQA
FIRWLPDGKSFQVTNREDFLKHVLPKYFKHNNFASFVRQLNMYGWHKVQD
VGNGSLTANEELWQFENPNFIRDREDLLDQIVRNKSKPGEDDENIDFGLV
LNELETIKMNQMAISEDLRRIRQDNETLWQEHYLARERHKTQAETLEKMM
RFLASVYGNNSKLLSEPTNDEFQKSSGAPQRHDTSNISKPTNAASKKLLM
LTDHAHKPSTNGSSSTSGAATGDVTPTVLPSHNSSVASQHPFIQEIVNRS
NQNLAPINSMPSPGTFFPELNEQLNESASQKVKNHSSMMQNVEDNINQQG
ESIKQIHEWINKLAPTSSTTNSKKTDSDAIADDDFDVNDFFLPHTPVDEP
GATSIPIIEELTPTDSLKRENGAGEGDNSASKRAKK.

The Pichia pastoris HSF1 amino acid sequence comprising an R166S substitution (in bold and underlined type and marked with an asterisk) is set forth as follows:

(SEQ ID NO: 207)
MSDFPKVEQASPSEGMPQVQTNNAPEIEEIEDIIRDTPNVMDVLGNGSQS
WDGTNSNGAAMGSLDTVPANKLVAYNKYDNNIYPDPLQPLSDTALPNSMG
YYSMIKAGDKGLKPQSFRKTKKKPTPSGPKTRPAFVMKLWNMVHDPSNQA
FIRWLPDGKSFQVTNS*EDFLKHVLPKYFKHNNFASFVRQLNMYGWHKVQ
DVGNGSLTANEELWQFENPNFIRDREDLLDQIVRNKSKPGEDDENIDFGL
VLNELETIKMNQMAISEDLRRIRQDNETLWQEHYLARERHKTQAETLEKM
MRFLASVYGNNSKLLSEPTNDEFQKSSGAPQRHDTSNISKPTNAASKKLL
MLTDHAHKPSTNGSSSTSGAATGDVTPTVLPSHNSSVASQHPFIQEIVNR
SNQNLAPINSMPSPGTFFPELNEQLNESASQKVKNHSSMMQNVEDNINQQ
GESIKQIHEWINKLAPTSSTTNSKKTDSDAIADDDFDVNDFFLPHTPVDE
PGATSIPIIEELTPTDSLKRENGAGEGDNSASKRAKK.

In some embodiments, a genetically modified host cell for increased expression of a heterologous gene comprises a homolog of a Pichia pastoris HSF1 gene, wherein an amino acid corresponding to R166 is mutated, for example, to serine or a conservative substitution for serine. HSF1 homologs from various species are known to those having ordinary skill in the art. In some embodiments, a homolog of HSF1 has a sequence which is at least about 60%, 70%, 80%, 90%, or 95% identical to SEQ ID NO: 207, or any amount in between any of these percentages.

In some embodiments, the host cell and the homolog of HSF1 are from the same species or genus (e.g., a Yarrowia cell comprising a mutant Yarrowia HSF1; or a Ogataea cell comprising a mutant Ogataea HSF1; etc.). In some embodiments, the host cell and the homolog of HSF1 are from different species or genera. In some embodiments, if the host cell is Saccharomyces (e.g., Saccharomyces cerevisiae), then the mutant HSF1 protein is not a mutant of the HSF1 homolog from Saccharomyces cerevisiae.

In some embodiments in which a host cell comprises a mutant HSF1 protein (or homolog thereof), the expression level of the mutant HSF1 protein in the host cell is at least 10% (e.g., 10% more), 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 250%, 300%, 400%, 500%, 1,000%, or more than 1,000% greater than the level of a wild-type HSF1 in a reference or control host cell which does not comprise the genetic modifications. In some experiments related to the Examples described herein, some Pichia pastoris host cells produced a mutant HSF1 at a level up to 35 times higher than endogenous (wild-type) HSF1.

A sequence encoding a mutant HSF1 protein in a host cell may be operably linked to a promoter or other regulator sequence as described herein.

### Downregulation of Genes to Improve Protein Production

In some embodiments, a genetically modified host cell comprises one or more genetic modifications that downregulate (e.g., reduce the expression, abundance and/or activity of) one or more endogenous genes, such as SSY1, HSL1, PAS_chr2-1_0053, PAS_chr2-1_0404, PAS_chr4_0550, PAS_chr1-3_0135, and/or PAS_chr1-3_0285 (or a homolog of any of them). Such downregulation may be achieved using any method known in the art, including, by way of non-limiting example, by reducing promoter strength, knocking out, replacing a native gene with a less active or abundant homolog from another species, and/or mutating a native or heterologous gene to attenuate expression or activity.

In some embodiments, downregulation of a gene can be accomplished using any technique known in the art. In some embodiments, downregulation of a gene includes: deleting the gene or a part thereof so that the gene no longer expresses a functional protein; making a frameshift mutation in the coding segment; deleting the promoter for the gene; replacing the native promoter with a weak or weaker promoter, or a regulatable promoter which is then regulated to be inactive; deleting the start codon; deleting or altering the native ribosome binding site of the gene or the region between the native ribosome binding site and the translational start of the gene; introducing a premature stop codon in the gene; introducing a heterologous nucleic acid and/or making an alteration to the sequence of the gene such that the protein product thereof (or the mRNA encoding it) is unstable, inactive, less active, or no longer transported to a cellular compartment wherein it would normally function; introducing a promoter which is downstream (3′) of the gene, is oriented in the opposite direction, and is stronger than the native promoter; altering the codon usage such that a decreased amount of the mRNA of the gene is translated; altering one or more of the intercistronic regions; introducing an agent such as a siRNA or antibody which interferes with and/or causes the destruction of the mRNA and/or protein corresponding to the gene; or using any other method known in the art now or in the future. As a non-limiting example, polymerase chain reaction (PCR)-based methods may be used (see, e.g., Gardner et al. 2014 Methods Mol Biol., 1205:45-78) or gene-editing techniques may be used to genetically modify the host cells of the disclosure. For example, genes may be deleted through gene replacement (e.g., with a marker, including a selection marker). A gene may also be truncated through the use of a transposon system (see, e.g., Poussu et al. 2005 Nucleic Acids Res., 33(12): e104).

### Downregulation of SSY1

In some embodiments, one or more genetic modifications to a host cell described in this disclosure result in the downregulation of (e.g., reduce the expression, abundance and/or activity of) SSY1 (or a homolog thereof).

Without wishing to be bound by any particular theory, the present disclosure notes that SSY1, in Saccharomyces, is reported to be part of a tripartite nutrient sensor of extracellular amino acids in the plasma membrane that is known to modulate expression levels of amino acid transporter genes. Klasson et al. 1999 Mol. Cell. Biol. 19: 5405-16; and Iraqui et al. 1999 Mol. Cell. Biol. 19: 989-1001. Other members of this nutrient sensor complex are reported to be PTR3, and SSY5. Mutation of the SSY1 gene is reported to result in diminished amino acid uptake from the media but also lead to increased intracellular levels of many amino acids. In some embodiments, SSY1 is also known as PAS_chr4_0019.

The amino acid sequence of the Pichia pastoris SSY1 is:

(SEQ ID NO: 208)
MDQDDLFPNRGSSSSSTLPDSTSLRSVDTELVKEVLEATDPVDLNHVDKL
HFTNLFGMHLKQDQREFASDFEKFNKETLMEERLRSKVSKVLSSQQKSNH
RLRVVGSSDTWDQKSDSNSFKTIPLNETEWNENVPIDLEKNFETIQDDVS
SVEKGFIPVSRRNFVSEWYHKPKRYDIQRKLKTRNLLNIALGGTIGVGIL
LSSGKGFSIAGPLGCLIGFMITGMVVLATMLSFCEMVTLLPLCGGVSGVA
SRFVDDAFGFALGIGYWFSYTIGLPTEIIAATIMLSYYEHLHVPGPSTSA
WVVFFIVVIVSINLCDVRVYGEVEYFSTIIKVLALLVLIIFMVVLNAGGV
APSHEYIGFRYWDSSKTNRTEFISNGPFRPTFDLADKGLGSFNGIGGNLG
RFCSVLVACVLAAYSYVGTEIVLIAGGESQNPRKAIPAATKVIYWRIIFF
YMLAIFVIGLNINSGDPRLLRFYTDGGAPADSQEQQDIQSVMDRNNGNNC
HFTLLKWGGFSNGNQSPWIIALQSAGLCSFAAVLNAFLIYFALTAGSSQL
YASSRTLYYLSIQGKVPKVFGICSKRGVPYISVLFTGSFSTLAFFAVEQN
TVVVFSRYLSICASAGLIVWTGMCLSFIRFYYGLQLRPDIITRNDDNYPY
RSPFQPYLAYFGFCMGSILVLSSGFVVFLNGHWSTTFFFTSYGSLILLFV
CYFGYKILRRTSIQRLDQLDLDSGRREIDRIIWEEEKDYTVTVKGWIRFI
IKKEVY.

SSY1 homologs from various species are known to those having ordinary skill in the art. In some embodiments, a homolog of SSY1 has a sequence which is at least about 60%, 70%, 80%, 90%, or 95% identical to SEQ ID NO: 208, or any amount in between any of these percentages.

### Downregulation of HSL1

In some embodiments, one or more genetic modifications to a host cell described in this disclosure result in the downregulation of (e.g., reduce the expression, abundance and/or activity of) the native HSL1 gene (or a homolog thereof).

HSL1, in Saccharomyces, is reportedly a protein kinase localized to the bud neck septin ring of dividing yeast cells that is involved in regulating the G2 to M transition in the mitotic cycle. Martinez et al. 2006 Mol. Cell. Biol. 26 (24): 9162-9176. Mutations in HSL1 reportedly cause defects in cell division that lead to elongated cell shape/pseudohyphal growth. Without wishing to be bound by any particular theory, the present disclosure suggests that downregulating this gene might lead to an increase in the volume available within the cell to accumulate protein expressed from a heterologous gene.

The amino acid sequence of the Pichia pastoris HSL1 is:

(SEQ ID NO: 209)
MTVDSSFALRAAQSASRNQHDYQDQPIDKVVSSVMSANKRLSQASTNTNS
SKRKSKNHVGPWKLGRTLGRGSTGRVRLAKHSTTGQLAAVKIVPKSSFLE
QKAKDKGIAATSHRIDSNGLPYGIEREIIIMKLINHPNIMGLYDVWENKG
ELYLVLEYIEGGELFDYLIKNGRQPESEAVRYFKQIIDGVSYCHQFSICH
RDLKPENLLLDKNSNIKIADFGMAALETKERLLETSCGSPHYASPEIIAG
KDYHGSPSDVWSCGIILFALLTGRLPFDDPNIRNLLIKVQSGVFTMPEYL
SKEAKDLITRMLHVDPTRRIKILDVYNHPLIQKYTDTLNFDHSYNQSTVN
VVNSESPIDTVDEDILQNLQTLWKGVDRRDIISKLKNSNISSEKVFYRLL
LKYRDDHSEYVLPSRRNSKKRLSNSLPRSTSIVTTTIKDDNGNTLESKTE
IIRAAPTSLSSKSLAKDPIKFQRNNIKASTSRKHVSLKSSSSRKSLMKKN
VSMNSVKSSAAPPRLPFANINENKSELKDFSFLCDHIFNSNRGFDEEPLL
DPSSDFLFCEDETIVSNDVPLYNTPKKVNSNVLKDSTNVVTSAKRKEAKN
LPKLPKDESYLSLAVTGDRTSIPDNSRNFSLDPRAASANKRNVSDTASSV
LTKLGVRLSTIDIYKEYNGSSSSSTKSLNSNLNSNSSTTLTSASKPQAVT
KPVGSKNAMSYQRESFLPLPTGLTFSGKLHYSSSSSTRDLASLLRPEAPK
LTLKEYNSKVKKLDSVKEIAVSRKNSYRQSKAIVDEPAKKYEETILNNDT
PACIDEFSFDYHDLTDSSVHVAEPVTFAKTKDVTYYSPIEEKFADVESSN
DAQNTDHAISEKSIKHHRKVASDGTHDSNLNIIVQPFNDIRNSLFVDNTV
NEIIEEDSRERVSEKSSNPRLSRFSQYSLSMGEPSFNQKRFTKVSIYGDP
DETSNLNLEKLVNSTKDRGSLPTQYSTIFDVVDDEGNPIESKQQYESQPE
KVQRGDQVKRASQFSNTLSDNDIDLGRAQSTRRRIMNQFHSIVPKRDAPR
PPSHARVSPMDPIFKNSTEASTKETLQVNEKSLSKTGNEDSLKKPTWFSK
LFNSLTKPKTTTKESKPNHTEVIQSSISSQRLWEIFKTTINNKQKEKTVS
KVTYDHNERLISGVIPARLTGRALHFNVQIKDGEQSLVVITHHKGSKKAF
RNLIIFVDQTIQVN.

HSL1 homologs from various species are known to those having ordinary skill in the art. In some embodiments, a homolog of HSL1 has a sequence which is at least about 60%, 70%, 80%, 90%, or 95% identical to SEQ ID NO: 209, or any amount in between any of these percentages.

### Downregulation of PAS_chr2-1_0053

In some embodiments, one or more genetic modifications to a host cell described in this disclosure result in the downregulation of (e.g., reduce the expression, abundance and/or activity of) PAS_chr2-1_0053 (or a homolog thereof), where PAS_chr2-1_0053 is a hypothetical protein with no known function.

The amino acid sequence of the Pichia pastoris PAS_chr2-1_0053 is:

(SEQ ID NO: 210)
MAGIKVGSGTSRNGKYQKQSKIQCIESEDIVLYQPFQTTLYAISKNGWDE
ILHRHDHWMQDHEVNFVRGNWFRFWEKV.

PAS_chr2-1_0053 homologs have not been identified.

### Downregulation of PAS_chr2-1_0404

In some embodiments, one or more genetic modifications to a host cell described in this disclosure result in the downregulation of (e.g., reduce the expression, abundance, level and/or activity of) PAS_chr2-1_0404 (or a homolog thereof).

Without wishing to be bound by any particular theory, the present disclosure notes that PAS_chr2-1_0404 is reported (Uniprot ID C4R0K1_KOMPG) as a transcriptional activator and/or repressor, belonging to the HSF family. Uniprot ID C4R0K1_KOMPG notes a predicted HSF Domain at aa 60 to 84.

The amino acid sequence of the Pichia pastoris PAS_chr2-1_0404 is:

(SEQ ID NO: 211)
MPESRTSKGSIKSVPKKSAFVHKLYTMLSNEELNDLIWWTGPEESGTFAL
LPGAEFSKVLSTYFKHANVSSFVRQLHMYGFHKVSEQPLVQGDTIPKVTW
EFRHSNGKFRKGNEDSLPLIKRRSTSSSSKSITTDYVKFRVHDQYSYFPQ
DLQYPQNPQQSNQEGETEHTQQPVLYHPQPTVYYHPNVGVPPAPPAPALA
PPPFSHLGQPMQPMSHQQPQQQIQQQHHHQPQSQHGQPQQPQQQYRQYTS
HPPPALLPFTPMYNMRPMTASPTNMEPMVLPTYHSMRLIETELEVKQSHL
QSKSDALIKQVHDYNKHIPSLVQLIPPHFEPKVDRNIQHSRLSGIEASVR
NRISKLSQPNPQPIHSAHSSFVSSKRNSSLVDPLQDLPLTAPVPNTGSGF
LSAHRGFYVPKTNSVSSSSSLPVTHDLPARRTPTPFKDRNPGDSSVESSH
SQLRPSIFRVHTKEEPVKAEKSSIFSNKDDSIFSNVHASSIFSQKTSIAS
QRSSLSMILNKPTVDSRDSITGSPLRKVSVHTIDEESTSSSNKRSHDDSN
GFEDKRGKLMKLDS.

PAS_chr2-1_0404 homologs from various species are known to those having ordinary skill in the art. In some embodiments, a homolog of PAS_chr2-1_0404 has a sequence which is at least about 60%, 70%, 80%, 90%, or 95% identical to SEQ ID NO: 211, or any amount in between any of these percentages.

### Downregulation of PAS_chr4_0550

In some embodiments, one or more genetic modifications to a host cell described in this disclosure result in the downregulation of (e.g., reduce the expression, abundance, level and/or activity of) PAS_chr4_0550 (or a homolog thereof).

Without wishing to be bound by any particular theory, the present disclosure notes that PAS_chr4_0550 is poorly characterized in the scientific literature, but is putatively annotated as a pyridoxine 4-dehydrogenase, involved in Vitamin B6 metabolism. In the record for Uniprot C4R885, PAS_chr4_0550 is reported to contain a predicted NADP-dependent oxidoreductase domain. Without wishing to be bound by any particular theory, the present disclosure notes that there is some literature surrounding the link between Vitamin B6 metabolism and Iron homeostasis/Heme production in the mitochondria. Parra et al. 2018 Cells. 7(7):84. doi: 10.3390/cells7070084.

The amino acid sequence of the Pichia pastoris PAS_chr2-1_0550 is:

(SEQ ID NO: 212)
MVVAIEGGTGLGLMNLTWKPTPTPIDDAIETIRYAVEEAGVRYLNGGEFY
NFPLDSNLNLQYIQEFAKRYPELYKKVSLSVKGAVSLVDVSPDSSPENLE
KSISNITKHLPNNFLPIFEPARIDKRYSIEETIKNLSKFVEDGRIGGISL
SEVGADTIRRAAKVAPIACVEVEFSLLTRDILHNGVLAACEDLNIPIIAY
SPLGRGFLTGTINSKADIPEGDIRLSLERFNDDEVIEHNLKLVHGLKKIA
DKKGVTLAQLSLAWLRKFGDKHVKVLPIPSCSSPRRVAENTKEISLTDSE
FQEITDFAESVPIKGGRYNKASEAVLNG.

PAS_chr4_0550 homologs from various species are known to those having ordinary skill in the art. In some embodiments, a homolog of PAS_chr4_0550 has a sequence which is at least about 60%, 70%, 80%, 90%, or 95% identical to SEQ ID NO: 212, or any amount in between any of these percentages.

### Downregulation of PAS_chr1-3_0135

In some embodiments, one or more genetic modifications to a host cell described in this disclosure result in the downregulation of (e.g., reduce the expression, abundance and/or activity of) PAS_chr1-3_0135 (or a homolog thereof).

PAS_chr1-3_0135 is poorly characterized in the scientific literature, but reported as a Sit4p protein phosphatase-associating protein that may regulate Sit4p phosphatase activity. The Sit4 phosphatase is reportedly involved in controlling cell division progression in complex with its Sit4-associated proteins. In Saccharomyces cerevisiae, deletion of Sit4-associated proteins reportedly leads to defects in cell division and an increase in cell size. Luke et al. 1996 Mol. Cell. Biol. 16(6): 2744-2755. Uniprot ID C4R885_KOMPG and NCBI: A0A65339.1 describe the gene PAS_chr1-3_0135 as an aldo-ketose reductase domain-containing protein, and InterPro predicts a NADP_OxRdtase_dom. Without wishing to be bound by any particular theory, the present disclosure suggests that downregulating the gene PAS_chr1-3_0135 might lead to an increase in the volume available within the cell to accumulate protein expressed from a heterologous gene.

The amino acid sequence of the Pichia pastoris PAS_chr2-1_0135 is:

(SEQ ID NO: 213)
MHHSSTEVSAIWPFFNNSYSNVAINKILQEIENEEHDNDQDAGLTAVQLL
QTIRENDDIQKANDTQEPTTPSRSKESKSNKSSKTTSSGHLNKRLLNNLL
IQPNLINELNAGKNSKLIAYITQEKVLSTLIDYCLESLDLKTDYVEDIDD
DNEDDFNEDGSPVGHDEEEITFQYDEDNNTGKDNEKAAPADQSDSSSKRL
LKRATIAMHILSSDQTPVVYNQFLSSHQYGLISKIWEGVFNRDIAEYFID
KRHNIVMMNGFIAIIENLAEMNVNGLMNFIRFQQTKDSDSLSKHFVNFIP
YFPQFSDLLLKLISMDKPYNPIGLIELLVDQDLIGQILEKLRVYYDDCII
QDNLLIFLNGLVNISSNVGYWDDQQNNMENEMNDGNNGSNEANISAVNGN
NTANIGPNDLTRDMVSTSKVNTMINIILNYGDYGLVTCISLFIEIIRKNN
SDYDEFDWIMAANDLTSTPNSRDPIYLGVMLKLFIINLPAIVNKYLTDEY
YERKQELNTFSSEGQRIDFDGKIRRKMIVSSIGKEIEPLGYERFKIMELI
AELLHCSNMMLLNQSSKLDYLLFKRDELRRANQTEKLVHDALTDSILKPV
EDAIEDLKIADDTSLHSFETLKKIDSKYIECTYDLSVGNSFKFSLLVSQA
LPRIILKMDKFPWNNFMHNVIFDLVQQIFNGKLIDDTEGTNDDENQDGDS
EAEKSTEYEQNTHGFEDPLCFNKLLIVSLFGEYDAFDQDLPPDGRFARPK
EIPGSFNLPAYILYACEKSKLSEEQANVKLGYMGHLVLVAQEVVKFQSII
ENFGIHKEQLKTRKLEGEEIADETDDDEGETEDTEEEEQNTENGNTYDTV
ITNSEQFSSIKGDLPDIYKISSTKIYTRLYKQLCTSFGEGRFQKWTDFIN
NELSVVREQYNQVLGGVNEGEVEIDEVPRNPNAIVLDNGDSEEFRKPFDE
QESETETEDEESEEEENNDNDRIREDEEAISDDNSSYDSDE.

PAS_chr1-3_0135 homologs from various species are known to those having ordinary skill in the art. In some embodiments, a homolog of PAS_chr1-3_0135 has a sequence which is at least about 60%, 70%, 80%, 90%, or 95% identical to SEQ ID NO: 213, or any amount in between any of these percentages.

### Downregulation of PAS_chr1-3_0285

In some embodiments, one or more genetic modifications to a host cell described in this disclosure result in the downregulation of (e.g., reduce the expression, abundance and/or activity of) PAS_chr1-3_0285 (or a homolog thereof).

Without wishing to be bound by any particular theory, the present disclosure notes that PAS_chr1-3_0285 is a hypothetical protein with an unknown function. A BLAST search for the protein sequence did not identify any characterization information for the gene encoding this sequence.

The amino acid sequence of the Pichia pastoris PAS_chr2-1_0285 is:

(SEQ ID NO: 214)
MKIRVPFLILSCLSTAICIVNSSLAIYLCKKVSFEPTLVALAVGELIPLA
GIVVVGTSQGGVLFMGCLLVSCVASLASGITWLARIVKNVERMAKYLESY
YNLTICLISLFILSQVFKIVLTHFIPHSTPKIEFPKDEESFALETILNYK
ASAQTLVQETNIPTHLPNEGQATWCNDNNDSVKIHEPAQYSQDIKSSMQL
GSERSSQQCFDPEEYKVHNHKESLKSKRSLEFEKNIIRNISNSLLPPVLQ
QGKSDISYLRNEMMGTPEKSHQDNENHDSEGKLFIDDLSDIPESYPQKDL
WNSNRTGNISHVSLRNWNENYTDWNQRQERKGVTTELYQLNPEFRNTEQL
FHNNDASSVEHMEQETSFGAPSLYSFSNNKLRQAESQDSSTVEANVGLTL
MTNVGSKQTLIQEEEGLDIQSKGKQRRSSIATFKNTSPIKKLRELKNEIR
SKNSVHHKSNSSLTSSIHVGMTFASAPTSPVKRKTHSLSKSMSCFHVSSQ
SIHRGDRSLRTVHGSPEKPNLSTLRHYPPLSDNPSSRESSQGSSCPSMFV
GQYDREKWTKLKSKEEVIV.

PAS_chr1-3_0285 homologs from various species are known to those having ordinary skill in the art. In some embodiments, a homolog of PAS_chr1-3_0285 has a sequence which is at least about 60%, 70%, 80%, 90%, or 95% identical to SEQ ID NO: 214, or any amount in between any of these percentages.

### Variants of Genes and Proteins Disclosed Herein

In some embodiments, the disclosure provides variants (which can be artificial or natural) of genes or proteins disclosed herein (e.g., presented in whole or in part, and/or referenced, for example, by an accession number, in this disclosure). In some embodiments, a sequence disclosed herein is designated a “reference” sequence.

A variant can comprise one or more mutations (e.g., nucleotide substitutions, insertions, additions, or deletions) and/or share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity” refers to the relatedness of the sequences of two polypeptides or polynucleotides when the sequences are aligned, and the term “percent identity” refers to the percentage of residues (amino acids or nucleotides) that are identical when two or more polypeptide or polynucleotide sequences are aligned. In some embodiments, sequence identity and/or percent identity is determined across the entire length of a sequence, while in other embodiments, sequence identity and/or percent identity is determined over a region of a sequence.

Percent identity of polypeptide or polynucleotide sequences can be calculated by any of the methods known to one of ordinary skill in the art. For example, percent identity can be determined using the algorithm of Karlin and Altschul 1990 Proc. Natl. Acad. Sci. U.S.A. 87:2264-68, modified as in Karlin and Altschul 1993 Proc. Natl. Acad. Sci. U.S.A. 90:5873-77. Such an algorithm is incorporated into the NBLAST® and XBLAST® programs (version 2.0) of Altschul et al. 1990 J. Mol. Biol. 215:403-10. BLAST® protein searches can be performed, for example, with the XBLAST program, score=50, wordlength=3. Where gaps exist between two sequences, Gapped BLAST® can be utilized, for example, as described in Altschul et al. 1997 Nucleic Acids Res. 25(17):3389-3402. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

A second example of a local alignment technique is based on the Smith-Waterman algorithm (Smith, T. F. & Waterman, M. S. 1981 J. Mol. Biol. 147:195-197). An example of a global alignment technique is the Needleman-Wunsch algorithm (Needleman, S. B. & Wunsch, C. D. 1970 J. Mol. Biol. 48:443-453), which is based on dynamic programming. A further example of a global alignment technique is the Fast Optimal Global Sequence Alignment Algorithm (FOGSAA).

In some embodiments, the percent identity of two polypeptide sequences is determined by aligning the two amino acid sequences of the polypeptides, calculating the number of identical amino acids, and dividing by the length of one of the polypeptide sequences. In some embodiments, the percent identity of two polynucleotide sequences is determined by aligning the two nucleotide sequences of the polynucleotides, calculating the number of identical nucleotides and dividing by the length of one of the polynucleotide sequences.

For multiple sequence alignments, computer programs including Clustal Omega (Sievers et al. 2011 Mol Syst Biol. 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul 1990 Proc. Natl. Acad. Sci. U.S.A. 87:2264-68, modified as in Karlin and Altschul 1993 Proc. Natl. Acad. Sci. U.S.A. 90:5873-77 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. 1981 J. Mol. Biol. 147:195-197) or the Needleman-Wunsch algorithm (Needleman, S. B. & Wunsch, C. D. 1970 J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al. 2011 Mol Syst Biol. 7:539).

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a variant of any sequence disclosed herein is a homologous sequence, a paralogous sequence (e.g., a sequence arising from the duplication of a gene) or an orthologous sequence (e.g., a sequence which diverges after a speciation event) thereof.

In some embodiments, a variant of a gene disclosed herein is codon-optimized for expression in a particular host, but the variant gene still encodes the same amino acid sequence as the gene disclosed herein.

In some embodiments, a variant of a gene disclosed herein comprises a conservative substitution. As used in this application, a “conservative substitution” refers to an amino acid substitution that does not alter the relative charge or size characteristics or functional activity of the protein in which the amino acid substitution is made.

In some instances, an amino acid is characterized by its R group (see e.g., TABLE 14). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group include lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Functionally equivalent variants of polypeptides may include conservative amino acid substitutions. Non-limiting examples of conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in TABLE 14.

TABLE 14
Non-limiting Examples of Conserved Amino Acid Substitutions

| Original | | Conservative Amino |
|---|---|---|
| Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R group | Ala, Ser |
| Gln (Q) | polar uncharged R group | Asn, Asp, Glu |
| Glu (E) | negatively charged R group | Asn, Asp, Gln |
| Gly (G) | nonpolar aliphatic R group | Ala, Ser |
| His (H) | positively charged R group | Arg, Tyr, Trp |
| Ile (I) | nonpolar aliphatic R group | Leu, Met, Val |
| Leu (L) | nonpolar aliphatic R group | Ile, Met, Val |
| Lys (K) | positively charged R group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

In some embodiments, a variant comprising a mutation can be designed in silico, or can be made by any method known to one of ordinary skill in the art, including but not limited to those described in: Kunkel 1985 Proc. Nat. Acad. Sci. U.S.A. 82: 488-492; Molecular Cloning: A Laboratory Manual, Sambrook et al. eds., Fourth Edition, Cold Spring Harbor Laboratory Press, Cold Spring Harbor, New York, 2012; and/or Current Protocols in Molecular Biology, Ausubel et al., eds., John Wiley & Sons, Inc., New York, 2010. In some embodiments, methods for producing variants include circular permutation. See, for example: Yu and Lutz, 2011 Trends Biotechnol. 29(1):18-25; and Weiner et al. 2005 Bioinformatics, 1; 21(7):932-7.

### Culturing Host Cells

A host cell (or genetically modified host cell) described herein can be grown using any culture medium, equipment and/or method described in the art (e.g., as appropriate for a particular organism), which can be selected and/or modified for producing a maximum amount of active bioproduct (e.g., protein of interest) from the host cell. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the host cells (and genetically modified host cells) described in this application can be performed in culture vessels and deepwell plates known and used in the art. In some embodiments, an aerated reaction vessel (e.g., a stirred tank reactor) is used to culture the cells. In some embodiments, a bioreactor or fermenter is used to culture the host cells. Thus, in some embodiments, the host cells are used in fermentation. Any type of bioreactor or fermenter known in the art may be compatible with aspects of the disclosure.

The present invention is further illustrated by the following Examples, which should not be construed as limiting. If a reference incorporated in this application contains a term whose definition is incongruous or incompatible with the definition of the same term as defined in the present disclosure, the meaning ascribed to the term in this disclosure shall govern. Mention of any reference, article, publication, patent, patent publication, and patent application cited in this application is not, and should not be taken as, an acknowledgment or suggestion that they constitute valid prior art or form part of the common general knowledge of a skilled artisan.

### Definitions

A “protein of interest,” as used herein, refers to a protein that one has interest in expressing in a host cell.

A “secreted protein”, as used herein, refers to a protein that is capable of being secreted outside of a cell (e.g., host cell) after synthesis in the cell. A secreted protein comprises a secretion signal (e.g., a secretion signal disclosed herein) and a protein of interest (e.g., as described herein).

As used herein, a “cleavage sequence” is an optional component of a secretion signal and refers to an amino acid sequence that is bound and cleaved by a protease (e.g., Kex2), thereby removing a secretion signal from a protein (e.g., protein of interest) to which it is affixed.

As used herein, a “transcriptional unit” refers to a sequence of nucleotides that codes for at least one RNA molecule, along with the sequences necessary for its instantiation, such as a promoter.

As used herein, a “promoter” refers to a regulatory region of DNA which directs the transcription of a sequence of DNA into RNA. An “inducible promoter,” as used herein, is a promoter controlled by the presence or absence of a molecule. A “constitutive promoter,” as used herein, refers to an unregulated promoter that allows continuous transcription of a gene.

A “fragment” of a promoter refers to a portion less than the full-length promoter sequence. A “functional fragment” of a promoter refers to a biologically active portion of a promoter sequence. A “biologically active portion” of a promoter has the same or a similar type of activity as the full-length promoter, although the level of activity of the biologically active portion of the promoter may vary compared to the level of activity of the full-length promoter.

“Synthetic” refers to a sequence (e.g., a nucleic acid sequence or an amino acid sequence) that is not naturally occurring, or to a component which includes one or more sequences that are not naturally occurring. A synthetic sequence may comprise two or more naturally occurring sequences that are combined to form a new sequence that is not naturally occurring.

“Naturally occurring” refers to something (e.g., a nucleic acid or polypeptide) that can be found in nature. For example, a naturally occurring nucleic acid or polypeptide sequence is one that can be isolated from a source in nature and has not otherwise been modified by a human in a laboratory. A naturally occurring sequence may be synthesized in any way and need not be extracted from a natural source.

As used herein, a “expression system” refers to a non-naturally occurring system that enables expression of genes of interest for the purpose of synthesizing desired bioproducts. An expression system comprises one or more transcriptional units.

A “transcription factor” is a protein that controls the rate of transcription from a cognate promoter by binding to one or more specific DNA sequences in or around the promoter. In some embodiments, a “synthetic transcription factor” refers to a transcription factor that does not occur in nature.

As used herein, a “homolog” is a gene or protein which is similar in structure to a gene or protein in another species or has characteristics (e.g., sequence and/or function) that are similar to a gene or protein from a different species because the species come from a common ancestor. In some embodiments, homologous proteins may arise in organisms without a common ancestor, if the proteins have similar structures and/or functions, and may have resulted from convergent evolution.

The term “cell,” as used herein, may refer to a single cell or a population of cells, such as a population of cells belonging to the same cell line or strain. Use of the singular term “cell” or “host cell” should not be construed to refer explicitly to a single cell rather than a population of cells (e.g., a colony or a population of identical or nearly identical cells).

The term “genetically modified host cell” and like terms are used interchangeably and refer to host cells that have been genetically modified by, e.g., cloning and transformation methods, or by other methods known in the art (e.g., selective genetic editing methods), to include one or more heterologous sequences.

A “heterologous sequence” of a host cell refers to a sequence that is: situated non-naturally in the host cell; expressed recombinantly (e.g., with a promoter other than its natural promoter) in the host cell; modified or edited in the host cell; expressed in a non-natural copy number in the host cell; or expressed in a non-natural way in the host cell. In some embodiments, a heterologous sequence is a naturally-occurring sequence (e.g., a naturally-occurring gene or regulatory sequence) that originates from a different organism than the host cell, or it may be a nucleic acid that is endogenously expressed in the host cell. In some embodiments, a heterologous sequence is a synthetic sequence (e.g., a synthetic gene or synthetic regulatory sequence).

As used herein, a “reference host cell” or a “control host cell” may be a wild-type or naturally occurring host cell, such as a methylotrophic yeast host cell, or may be a genetically modified host cell. In some embodiments, a modified host cell and a control host cell are identical except for the modification(s) (e.g., the modified host cell and the control have the same genetic background), and they are grown under identical conditions when comparisons are made of the expression, abundance and/or activity of a heterologous protein produced by them.

A “methylotrophic yeast host cell” is one that naturally (i.e., prior to any manipulation by a human) has an ability to utilize reduced one-carbon compounds, such as methanol or methane, and multi-carbon compounds that contain no carbon-carbon bonds, such as dimethyl ether and dimethylamine, as a carbon source(s) for its growth. Methylotrophic yeasts are known in the art, and include, for example, those in the genera Pichia, Komagataella, Hansenula, and Candida. A yeast host cell that is naturally methylotrophic, such as one from among the genera Pichia, Komagataella, Hansenula, or Candida but has been rendered unable to utilize methanol or methane, e.g. by genetic engineering, is still considered to be a methylotrophic yeast host cell for purposes of this disclosure.

As used in this application, the terms “bioreactor” and “fermenter” are interchangeably used and refer to an enclosure, or partial enclosure, in which a biological, biochemical and/or chemical reaction takes place, involving a living organism, part of a living organism, or purified proteins. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

### EXAMPLES

In order that the invention described in this application may be more fully understood, the following examples are set forth. The examples described in this application are offered to illustrate the systems and methods provided in this application and are not to be construed as limiting their scope.

### Example 1: Secretion Signal Library

To identify secretion signals that would improve expression and/or secretion of heterologous proteins in a host cell, a library of over 500 candidate secretion signals was constructed (FIGS. 2A-2B). The candidate secretion signals comprise, in order from N- to C-terminus: any of 14 Pre sequences selected from several different species or sources (including those listed in TABLE 1), and/or any of 21 Pro sequences selected from several different species or sources (including those listed in TABLE 2), with or without a terminal cleavage sequence (e.g., KR and/or EAEA, SEQ ID NO: 104).

Additional information regarding the secretion signals, the library and the assays are provided elsewhere herein, including in the subsequent examples.

### Example 2: Screening of Secretion Signal Library in Pichia

### Strain Construction

Pichia pastoris strains were constructed to test whether the secretion signals of the secretion library of Example 1 are capable of promoting protein secretion. Bovine lactoferrin (bLF, UniProt P24627) was selected as an exemplary protein for screening the secretion signals (FIG. 1).

Pichia pastoris (or Komagataella phaffii) strain BG11 was used as the initial strain. Different linear DNA fragments, each containing a unique secretion signal (from the library of Example 1) as opposed to bLF's original secretion signal (the first 19 amino acids of native bLF sequence, i.e., MKLFVPALLSLGALGLCLA (SEQ ID NO: 13) linked to codon-optimized bLF sequence, were transformed individually into the base strain using the PEG/LiAC/ssDNA transformation method. The integration occurred via single cross-over recombination. The bLF expression cassette included the AOX1 promoter and the AOX1 terminator (both linked to the sequence encoding bLF). The presence of expression cassettes in the library strains was validated through whole genome sequencing (WGS).

### Signal Library Screening

To screen the secretion signal library, 5 uL of glycerol stock of transformation clones were inoculated in 250 mL YPD media in 96-deepwell plates and incubated at 30° C., 1000 rpm shaking speed, 80% humidity, for 24 hours. 5 uL of these cultures were then transferred into 250 uL of BMDY media in 96-deepwell plates and incubated at 30° C., 1000 rpm shaking speed, 80% humidity for 18-24 hours. Afterwards, 25 uL of carbon source solution (an aqueous solution containing 0.5%-v of glycerol and 0.5%-v of methanol) were added into the cultures as well as 24 and 48 hours after inoculation. At 72 hours after inoculation, the cultures were pelleted and supernatant was used to quantify bLF. bLF quantification was done using a commercial ELISA kit and performed according to manufacturer's instructions.

In a primary screening, each clone tested was cultured without replicate except for the control strain (i.e., bLF having the secretion signal from Saccharomyces cerevisiae alpha mating factor), for which two cultivations were performed in each deepwell plate. Of the over 500 candidate secretion signals screened, roughly 410 were found to be capable of promoting secretion of bLF (TABLE 15).

In addition, several pre-, pro-sequence combinations were screened, wherein the pre- and pro-sequences were both from the same protein (i.e., pre-, pro-sequence combinations found in naturally occurring secretion signals). Two (sectag228 and sectag994) were found to be particularly efficacious. Sectag228, also designated PreProl+EAEA, yielded bLF at a titer of 1.71× the control (Mating Factor alpha1 of Saccharomyces cerevisiae). Sectag228 comprises the sequence MKFTLATLLVLATAAIAAPVAAPEAEAGGRGNFGNSGPPIWKR (SEQ ID NO: 119) and is from an uncharacterized protein in Cutaneotrichosporon oleaginosum, A0A0J0XX36. Sectag994, also designated PrePro9+EAEA, yielded bLF at a titer of 1.65× the control. Sectag994 comprises the sequence MKLKYFLLIFVFTTVLAKPQHYKR (SEQ ID NO: 139), and is from Plectin-like isoform x3, Rhizophagus irregularis (strain DAOM 181602/DAOM 197198/MUCL 43194) (Arbuscular mycorrhizal fungus) (Glomus intraradices), A0A2H5S501.

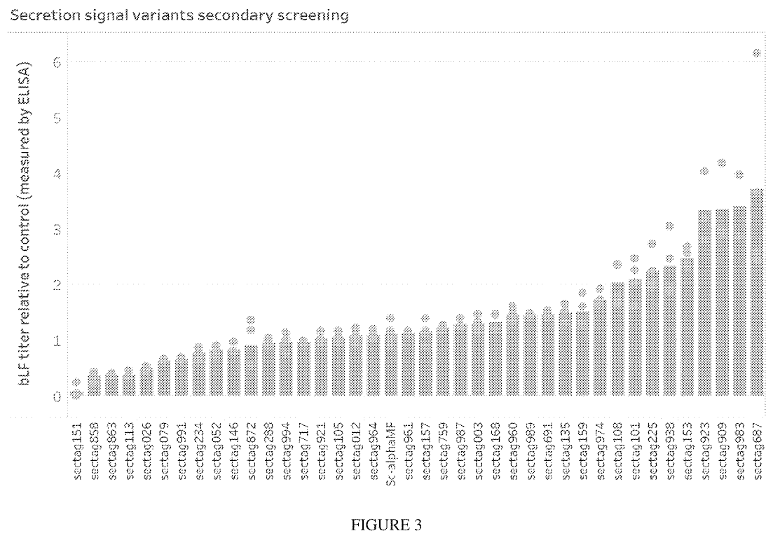

A secondary screen was performed to validate the results from the primary screen, using a subset (i.e., 40) of the secretion signals identified from the primary screen. In the secondary screening, each clone tested was cultured in at least three replicates. One ELISA measurement was performed for each of the replicates. Like the primary screen, the control strain for the secondary screen expressed bLF with a secretion signal from Saccharomyces cerevisiae alpha mating factor. The secondary screen confirmed that the secretion signals identified by the primary screen are capable of promoting secretion of bLF (TABLE 16; FIG. 3).

Upon reviewing the results from the primary and secondary screen, various secretion signals were found to be more effective in promoting bLF expression than the control. Descriptions of the top producers are provided in TABLE 17.

TABLE 15
Secretion Signals Found to Promote Protein Secretion in Primary Screen

| Pichia | | | | |
|---|---|---|---|---|
| Strain | SecTag | | | |
| No. | ID: | Pre-Seq ID: | Pro-Seq ID | Cleavage site |
| 1 | Sc-alphaMF | Sc-alphaMF | Sc-alphaMF | KREAEA (SEQ ID NO: 105) |
| 2 | sectag667 | 3075305 (Pre3) | 3075285 (Pro14) | KREAEA (SEQ ID NO: 105) |
| 3 | sectag668 | 3075305 (Pre3) | 3075287 (Pro15) | KREAEA (SEQ ID NO: 105) |
| 4 | sectag669 | 3075305 (Pre3) | 3075289 (Pro16) | KREAEA (SEQ ID NO: 105) |
| 5 | sectag673 | 3075305 (Pre3) | 3075297 (Pro20) | KREAEA (SEQ ID NO: 105) |
| 6 | sectag677 | 3075321 (Pre11) | 3075289 (Pro16) | KREAEA (SEQ ID NO: 105) |
| 7 | sectag677 | 3075321 (Pre11) | 3075289 (Pro16) | KREAEA (SEQ ID NO: 105) |
| 8 | sectag678 | 3075325 (Pre13) | 3075277 (Pro10) | KREAEA (SEQ ID NO: 105) |
| 9 | sectag679 | 3075321 (Pre11) | 3075291 (Pro17) | KREAEA (SEQ ID NO: 105) |
| 10 | sectag681 | 3075321 (Pre11) | 3075293 (Pro18) | KREAEA (SEQ ID NO: 105) |
| 11 | sectag682 | 3075327 (Pre14) | 3075267 (Pro5) | KREAEA (SEQ ID NO: 105) |
| 12 | sectag683 | 3075321 (Pre11) | 3075295 (Pro19) | KREAEA (SEQ ID NO: 105) |
| 13 | sectag685 | 3075321 (Pre11) | 3075297 (Pro20) | KREAEA (SEQ ID NO: 105) |
| 14 | sectag687 | 3075321 (Pre11) | 3075299 (Pro21) | KREAEA (SEQ ID NO: 105) |
| 15 | sectag691 | 3075307 (Pre4) | 3075259 (Pro1) | KREAEA (SEQ ID NO: 105) |
| 16 | sectag698 | 3075323 (Pre12) | 3075277 (Pro10) | KR |
| 17 | sectag707 | 3075301 (Pre1) | 3075259 (Pro1) | KR |
| 18 | sectag708 | 3075321 (Pre11) | 3075287 (Pro15) | KR |
| 19 | sectag710 | 3075321 (Pre11) | 3075289 (Pro16) | KR |
| 20 | sectag711 | 3075321 (Pre11) | 3075293 (Pro18) | KR |
| 21 | sectag713 | 3075321 (Pre11) | 3075295 (Pro19) | KR |
| 22 | sectag715 | 3075321 (Pre11) | 3075297 (Pro20) | KR |
| 23 | sectag716 | 3075309 (Pre5) | 3075297 (Pro20) | KR |
| 24 | sectag717 | 3075321 (Pre11) | 3075299 (Pro21) | KR |
| 25 | sectag718 | 3075309 (Pre5) | 3075299 (Pro21) | KR |
| 26 | sectag730 | 3075325 (Pre13) | 3075265 (Pro4) | KR |
| 27 | sectag732 | 3075325 (Pre13) | 3075267 (Pro5) | KR |
| 28 | sectag742 | 3075311 (Pre6) | 3075267 (Pro5) | KR |
| 29 | sectag743 | 3075323 (Pre12) | 3075267 (Pro5) | KR |
| 30 | sectag749 | 3075323 (Pre12) | 3075283 (Pro13) | KR |
| 31 | sectag757 | 3075309 (Pre5) | 3075281 (Pro12) | KREAEA (SEQ ID NO: 105) |
| 32 | sectag759 | 3075309 (Pre5) | 3075291 (Pro17) | KREAEA (SEQ ID NO: 105) |
| 33 | sectag760 | 3075325 (Pre13) | 3075283 (Pro13) | KR |
| 34 | sectag761 | 3075309 (Pre5) | 3075297 (Pro20) | KREAEA (SEQ ID NO: 105) |
| 35 | sectag762 | 3075325 (Pre13) | 3075285 (Pro14) | KR |
| 36 | sectag763 | 3075313 (Pre7) | 3075275 (Pro9) | KREAEA (SEQ ID NO: 105) |
| 37 | sectag764 | 3075325 (Pre13) | 3075287 (Pro15) | KR |
| 38 | sectag765 | 3075313 (Pre7) | 3075283 (Pro13) | KREAEA (SEQ ID NO: 105) |
| 39 | sectag766 | 3075325 (Pre13) | 3075289 (Pro16) | KR |
| 40 | sectag781 | 3075325 (Pre13) | 3075291 (Pro17) | KR |
| 41 | sectag783 | 3075313 (Pre7) | 3075291 (Pro17) | KREAEA (SEQ ID NO: 105) |
| 42 | sectag785 | 3075313 (Pre7) | 3075295 (Pro19) | KREAEA (SEQ ID NO: 105) |
| 43 | sectag786 | 3075327 (Pre14) | 3075263 (Pro3) | KR |
| 44 | sectag787 | 3075313 (Pre7) | 3075297 (Pro20) | KREAEA (SEQ ID NO: 105) |
| 45 | sectag788 | 3075327 (Pre14) | 3075265 (Pro4) | KR |
| 46 | sectag791 | 3075315 (Pre8) | 3075261 (Pro2) | KREAEA (SEQ ID NO: 105) |
| 47 | sectag793 | 3075315 (Pre8) | 3075265 (Pro4) | KREAEA (SEQ ID NO: 105) |
| 48 | sectag795 | 3075315 (Pre8) | 3075267 (Pro5) | KREAEA (SEQ ID NO: 105) |
| 49 | sectag796 | 3075327 (Pre14) | 3075279 (Pro11) | KR |
| 50 | sectag798 | 3075327 (Pre14) | 3075285 (Pro14) | KR |
| 51 | sectag801 | 3075325 (Pre13) | 3075295 (Pro19) | KR |
| 52 | sectag802 | 3075313 (Pre7) | 3075283 (Pro13) | KR |
| 53 | sectag803 | 3075325 (Pre13) | 3075297 (Pro20) | KR |
| 54 | sectag805 | 3075325 (Pre13) | 3075299 (Pro21) | KR |
| 55 | sectag809 | 3075327 (Pre14) | 3075267 (Pro5) | KR |
| 56 | sectag813 | 3075327 (Pre14) | 3075273 (Pro8) | KR |
| 57 | sectag815 | 3075315 (Pre8) | 3075273 (Pro8) | KREAEA (SEQ ID NO: 105) |
| 58 | sectag816 | 3075327 (Pre14) | 3075287 (Pro15) | KR |
| 59 | sectag818 | 3075327 (Pre14) | 3075289 (Pro16) | KR |
| 60 | sectag819 | 3075315 (Pre8) | 3075277 (Pro10) | KREAEA (SEQ ID NO: 105) |
| 61 | sectag820 | 3075327 (Pre14) | 3075293 (Pro18) | KR |
| 62 | sectag822 | 3075327 (Pre14) | 3075295 (Pro19) | KR |
| 63 | sectag823 | 3075315 (Pre8) | 3075281 (Pro12) | KREAEA (SEQ ID NO: 105) |
| 64 | sectag824 | 3075327 (Pre14) | 3075297 (Pro20) | KR |
| 65 | sectag825 | 3075315 (Pre8) | 3075283 (Pro13) | KREAEA (SEQ ID NO: 105) |
| 66 | sectag826 | 3075327 (Pre14) | 3075299 (Pro21) | KR |
| 67 | sectag827 | 3075315 (Pre8) | 3075285 (Pro14) | KREAEA (SEQ ID NO: 105) |
| 68 | sectag828 | 3075303 (Pre2) | 3075275 (Pro9) | KREAEA (SEQ ID NO: 105) |
| 69 | sectag829 | 3075315 (Pre8) | 3075287 (Pro15) | KREAEA (SEQ ID NO: 105) |
| 70 | sectag830 | 3075303 (Pre2) | 3075295 (Pro19) | KREAEA (SEQ ID NO: 105) |
| 71 | sectag835 | 3075327 (Pre14) | 3075291 (Pro17) | KR |
| 72 | sectag837 | 3075301 (Pre1) | 3075259 (Pro1) | KREAEA (SEQ ID NO: 105) |
| 73 | sectag839 | 3075301 (Pre1) | 3075261 (Pro2) | KREAEA (SEQ ID NO: 105) |
| 74 | sectag841 | 3075301 (Pre1) | 3075263 (Pro3) | KREAEA (SEQ ID NO: 105) |
| 75 | sectag842 | 3075315 (Pre8) | 3075291 (Pro17) | KR |
| 76 | sectag843 | 3075301 (Pre1) | 3075265 (Pro4) | KREAEA (SEQ ID NO: 105) |
| 77 | sectag845 | 3075301 (Pre1) | 3075267 (Pro5) | KREAEA (SEQ ID NO: 105) |
| 78 | sectag847 | 3075315 (Pre8) | 3075291 (Pro17) | KREAEA (SEQ ID NO: 105) |
| 79 | sectag848 | 3075309 (Pre5) | 3075269 (Pro6) | KREAEA (SEQ ID NO: 105) |
| 80 | sectag265 | 3075301 (Pre1) | | N/A |
| 81 | sectag851 | 3075315 (Pre8) | 3075297 (Pro20) | KREAEA (SEQ ID NO: 105) |
| 82 | sectag852 | 3075309 (Pre5) | 3075271 (Pro7) | KREAEA (SEQ ID NO: 105) |
| 83 | sectag854 | 3075309 (Pre5) | 3075273 (Pro8) | KREAEA (SEQ ID NO: 105) |
| 84 | sectag855 | 3075317 (Pre9) | 3075259 (Pro1) | KREAEA (SEQ ID NO: 105) |
| 85 | sectag856 | 3075303 (Pre2) | | N/A |
| 86 | sectag858 | 3075309 (Pre5) | 3075275 (Pro9) | KREAEA (SEQ ID NO: 105) |
| 87 | sectag859 | 3075317 (Pre9) | 3075265 (Pro4) | KREAEA (SEQ ID NO: 105) |
| 88 | sectag860 | 3075305 (Pre3) | | N/A |
| 89 | sectag862 | 3075307 (Pre4) | | N/A |
| 90 | sectag863 | 3075301 (Pre1) | 3075269 (Pro6) | KREAEA (SEQ ID NO: 105) |
| 91 | sectag865 | 3075301 (Pre1) | 3075271 (Pro7) | KREAEA (SEQ ID NO: 105) |
| 92 | sectag867 | 3075301 (Pre1) | 3075273 (Pro8) | KREAEA (SEQ ID NO: 105) |
| 93 | sectag868 | 3075317 (Pre9) | 3075259 (Pro1) | KR |
| 94 | sectag870 | 3075311 (Pre6) | 3075281 (Pro12) | KREAEA (SEQ ID NO: 105) |
| 95 | sectag871 | 3075301 (Pre1) | 3075277 (Pro10) | KREAEA (SEQ ID NO: 105) |
| 96 | sectag872 | 3075311 (Pre6) | 3075283 (Pro13) | KREAEA (SEQ ID NO: 105) |
| 97 | sectag874 | 3075311 (Pre6) | 3075285 (Pro14) | KREAEA (SEQ ID NO: 105) |
| 98 | sectag875 | 3075301 (Pre1) | 3075281 (Pro12) | KREAEA (SEQ ID NO: 105) |
| 99 | sectag876 | 3075311 (Pre6) | 3075287 (Pro15) | KREAEA (SEQ ID NO: 105) |
| 100 | sectag877 | 3075301 (Pre1) | 3075283 (Pro13) | KREAEA (SEQ ID NO: 105) |
| 101 | sectag878 | 3075311 (Pre6) | 3075289 (Pro16) | KREAEA (SEQ ID NO: 105) |
| 102 | sectag883 | 3075317 (Pre9) | 3075275 (Pro9) | KREAEA (SEQ ID NO: 105) |
| 103 | sectag885 | 3075317 (Pre9) | 3075277 (Pro10) | KREAEA (SEQ ID NO: 105) |
| 104 | sectag889 | 3075317 (Pre9) | 3075281 (Pro12) | KREAEA (SEQ ID NO: 105) |
| 105 | sectag890 | 3075311 (Pre6) | 3075263 (Pro3) | KREAEA (SEQ ID NO: 105) |
| 106 | sectag891 | 3075317 (Pre9) | 3075283 (Pro13) | KREAEA (SEQ ID NO: 105) |
| 107 | sectag893 | 3075317 (Pre9) | 3075285 (Pro14) | KREAEA (SEQ ID NO: 105) |
| 108 | sectag895 | 3075301 (Pre1) | 3075285 (Pro14) | KREAEA (SEQ ID NO: 105) |
| 109 | sectag896 | 3075311 (Pre6) | 3075291 (Pro17) | KREAEA (SEQ ID NO: 105) |
| 110 | sectag898 | 3075311 (Pre6) | 3075293 (Pro18) | KREAEA (SEQ ID NO: 105) |
| 111 | sectag899 | 3075301 (Pre1) | 3075289 (Pro16) | KREAEA (SEQ ID NO: 105) |
| 112 | sectag901 | 3075301 (Pre1) | 3075291 (Pro17) | KREAEA (SEQ ID NO: 105) |
| 113 | sectag902 | 3075311 (Pre6) | 3075297 (Pro20) | KREAEA (SEQ ID NO: 105) |
| 114 | sectag903 | 3075301 (Pre1) | 3075293 (Pro18) | KREAEA (SEQ ID NO: 105) |
| 115 | sectag905 | 3075301 (Pre1) | 3075295 (Pro19) | KREAEA (SEQ ID NO: 105) |
| 116 | sectag907 | 3075301 (Pre1) | 3075297 (Pro20) | KREAEA (SEQ ID NO: 105) |
| 117 | sectag909 | 3075301 (Pre1) | 3075299 (Pro21) | KREAEA (SEQ ID NO: 105) |
| 118 | sectag910 | 3075327 (Pre14) | 3075287 (Pro15) | KREAEA (SEQ ID NO: 105) |
| 119 | sectag911 | 3075317 (Pre9) | 3075287 (Pro15) | KREAEA (SEQ ID NO: 105) |
| 120 | sectag912 | 3075315 (Pre8) | 3075269 (Pro6) | KREAEA (SEQ ID NO: 105) |
| 121 | sectag913 | 3075317 (Pre9) | 3075289 (Pro16) | KREAEA (SEQ ID NO: 105) |
| 122 | sectag915 | 3075317 (Pre9) | 3075291 (Pro17) | KREAEA (SEQ ID NO: 105) |
| 123 | sectag917 | 3075317 (Pre9) | 3075293 (Pro18) | KREAEA (SEQ ID NO: 105) |
| 124 | sectag918 | 3075315 (Pre8) | 3075295 (Pro19) | KREAEA (SEQ ID NO: 105) |
| 125 | sectag919 | 3075317 (Pre9) | 3075295 (Pro19) | KREAEA (SEQ ID NO: 105) |
| 126 | sectag921 | 3075317 (Pre9) | 3075297 (Pro20) | KREAEA (SEQ ID NO: 105) |
| 127 | sectag923 | 3075317 (Pre9) | 3075299 (Pro21) | KREAEA (SEQ ID NO: 105) |
| 128 | sectag924 | 3075317 (Pre9) | 3075263 (Pro3) | KREAEA (SEQ ID NO: 105) |
| 129 | sectag927 | 3075303 (Pre2) | 3075259 (Pro1) | KREAEA (SEQ ID NO: 105) |
| 130 | sectag928 | 3075327 (Pre14) | 3075289 (Pro16) | KREAEA (SEQ ID NO: 105) |
| 131 | sectag929 | 3075303 (Pre2) | 3075261 (Pro2) | KREAEA (SEQ ID NO: 105) |
| 132 | sectag931 | 3075303 (Pre2) | 3075263 (Pro3) | KREAEA (SEQ ID NO: 105) |
| 133 | sectag933 | 3075303 (Pre2) | 3075265 (Pro4) | KREAEA (SEQ ID NO: 105) |
| 134 | sectag935 | 3075303 (Pre2) | 3075267 (Pro5) | KREAEA (SEQ ID NO: 105) |
| 135 | sectag937 | 3075303 (Pre2) | 3075269 (Pro6) | KREAEA (SEQ ID NO: 105) |
| 136 | sectag938 | 3075327 (Pre14) | 3075299 (Pro21) | KREAEA (SEQ ID NO: 105) |
| 137 | sectag939 | 3075303 (Pre2) | 3075271 (Pro7) | KREAEA (SEQ ID NO: 105) |
| 138 | sectag940 | 3075303 (Pre2) | 3075273 (Pro8) | KREAEA (SEQ ID NO: 105) |
| 139 | sectag941 | 3075319 (Pre10) | 3075261 (Pro2) | KREAEA (SEQ ID NO: 105) |
| 140 | sectag942 | 3075317 (Pre9) | 3075271 (Pro7) | KREAEA (SEQ ID NO: 105) |
| 141 | sectag945 | 3075319 (Pre10) | 3075265 (Pro4) | KREAEA (SEQ ID NO: 105) |
| 142 | sectag949 | 3075319 (Pre10) | 3075269 (Pro6) | KREAEA (SEQ ID NO: 105) |
| 143 | sectag951 | 3075319 (Pre10) | 3075273 (Pro8) | KREAEA (SEQ ID NO: 105) |
| 144 | sectag954 | 3075319 (Pre10) | 3075293 (Pro18) | KREAEA (SEQ ID NO: 105) |
| 145 | sectag955 | 3075319 (Pre10) | 3075277 (Pro10) | KREAEA (SEQ ID NO: 105) |
| 146 | sectag957 | 3075303 (Pre2) | 3075277 (Pro10) | KREAEA (SEQ ID NO: 105) |
| 147 | sectag960 | 3075303 (Pre2) | 3075283 (Pro13) | KREAEA (SEQ ID NO: 105) |
| 148 | sectag961 | 3075303 (Pre2) | 3075285 (Pro14) | KREAEA (SEQ ID NO: 105) |
| 149 | sectag962 | 3075303 (Pre2) | 3075287 (Pro15) | KREAEA (SEQ ID NO: 105) |
| 150 | sectag963 | 3075303 (Pre2) | 3075289 (Pro16) | KREAEA (SEQ ID NO: 105) |
| 151 | sectag964 | 3075303 (Pre2) | 3075291 (Pro17) | KREAEA (SEQ ID NO: 105) |
| 152 | sectag965 | 3075319 (Pre10) | 3075281 (Pro12) | KREAEA (SEQ ID NO: 105) |
| 153 | sectag966 | 3075319 (Pre10) | 3075295 (Pro19) | KREAEA (SEQ ID NO: 105) |
| 154 | sectag967 | 3075319 (Pre10) | 3075283 (Pro13) | KREAEA (SEQ ID NO: 105) |
| 155 | sectag969 | 3075319 (Pre10) | 3075285 (Pro14) | KREAEA (SEQ ID NO: 105) |
| 156 | sectag970 | 3075319 (Pre10) | 3075297 (Pro20) | KREAEA (SEQ ID NO: 105) |
| 157 | sectag971 | 3075319 (Pre10) | 3075287 (Pro15) | KREAEA (SEQ ID NO: 105) |
| 158 | sectag973 | 3075319 (Pre10) | 3075289 (Pro16) | KREAEA (SEQ ID NO: 105) |
| 159 | sectag974 | 3075319 (Pre10) | 3075299 (Pro21) | KREAEA (SEQ ID NO: 105) |
| 160 | sectag975 | 3075319 (Pre10) | 3075291 (Pro17) | KREAEA (SEQ ID NO: 105) |
| 161 | sectag976 | 3075321 (Pre11) | 3075259 (Pro1) | KREAEA (SEQ ID NO: 105) |
| 162 | sectag979 | 3075321 (Pre11) | 3075265 (Pro4) | KREAEA (SEQ ID NO: 105) |
| 163 | sectag980 | 3075321 (Pre11) | 3075263 (Pro3) | KREAEA (SEQ ID NO: 105) |
| 164 | sectag981 | 3075303 (Pre2) | 3075293 (Pro18) | KREAEA (SEQ ID NO: 105) |
| 165 | sectag982 | 3075303 (Pre2) | 3075297 (Pro20) | KREAEA (SEQ ID NO: 105) |
| 166 | sectag983 | 3075303 (Pre2) | 3075299 (Pro21) | KREAEA (SEQ ID NO: 105) |
| 167 | sectag984 | 3075305 (Pre3) | 3075259 (Pro1) | KREAEA (SEQ ID NO: 105) |
| 168 | sectag985 | 3075305 (Pre3) | 3075261 (Pro2) | KREAEA (SEQ ID NO: 105) |
| 169 | sectag986 | 3075305 (Pre3) | 3075263 (Pro3) | KREAEA (SEQ ID NO: 105) |
| 170 | sectag987 | 3075305 (Pre3) | 3075265 (Pro4) | KREAEA (SEQ ID NO: 105) |
| 171 | sectag988 | 3075305 (Pre3) | 3075267 (Pro5) | KREAEA (SEQ ID NO: 105) |
| 172 | sectag989 | 3075321 (Pre11) | 3075267 (Pro5) | KREAEA (SEQ ID NO: 105) |
| 173 | sectag990 | 3075321 (Pre11) | 3075269 (Pro6) | KREAEA (SEQ ID NO: 105) |
| 174 | sectag991 | 3075321 (Pre11) | 3075271 (Pro7) | KREAEA (SEQ ID NO: 105) |
| 175 | sectag993 | 3075321 (Pre11) | 3075273 (Pro8) | KREAEA (SEQ ID NO: 105) |
| 176 | sectag995 | 3075321 (Pre11) | 3075275 (Pro9) | KREAEA (SEQ ID NO: 105) |
| 177 | sectag997 | 3075321 (Pre11) | 3075277 (Pro10) | KREAEA (SEQ ID NO: 105) |
| 178 | sectag998 | 3075321 (Pre11) | 3075287 (Pro15) | KREAEA (SEQ ID NO: 105) |
| 179 | sectag001 | 3075321 (Pre11) | 3075281 (Pro12) | KREAEA (SEQ ID NO: 105) |
| 180 | sectag003 | 3075321 (Pre11) | 3075283 (Pro13) | KREAEA (SEQ ID NO: 105) |
| 181 | sectag005 | 3075305 (Pre3) | 3075269 (Pro6) | KREAEA (SEQ ID NO: 105) |
| 182 | sectag006 | 3075305 (Pre3) | 3075271 (Pro7) | KREAEA (SEQ ID NO: 105) |
| 183 | sectag007 | 3075305 (Pre3) | 3075273 (Pro8) | KREAEA (SEQ ID NO: 105) |
| 184 | sectag008 | 3075305 (Pre3) | 3075275 (Pro9) | KREAEA (SEQ ID NO: 105) |
| 185 | sectag009 | 3075305 (Pre3) | 3075277 (Pro10) | KREAEA (SEQ ID NO: 105) |
| 186 | sectag010 | 3075305 (Pre3) | 3075279 (Pro11) | KREAEA (SEQ ID NO: 105) |
| 187 | sectag011 | 3075305 (Pre3) | 3075281 (Pro12) | KREAEA (SEQ ID NO: 105) |
| 188 | sectag012 | 3075305 (Pre3) | 3075283 (Pro13) | KREAEA (SEQ ID NO: 105) |
| 189 | sectag013 | 3075317 (Pre9) | 3075289 (Pro16) | KR |
| 190 | sectag016 | 3075317 (Pre9) | 3075297 (Pro20) | KR |
| 191 | sectag017 | 3075317 (Pre9) | 3075299 (Pro21) | KR |
| 192 | sectag020 | 3075319 (Pre10) | 3075263 (Pro3) | KR |
| 193 | sectag021 | 3075319 (Pre10) | 3075299 (Pro21) | KR |
| 194 | sectag022 | 3075309 (Pre5) | 3075263 (Pro3) | KR |
| 195 | sectag022 | 3075309 (Pre5) | 3075263 (Pro3) | KR |
| 196 | sectag025 | 3075321 (Pre11) | 3075261 (Pro2) | KR |
| 197 | sectag026 | 3075309 (Pre5) | 3075267 (Pro5) | KR |
| 198 | sectag027 | 3075321 (Pre11) | 3075263 (Pro3) | KR |
| 199 | sectag028 | 3075309 (Pre5) | 3075269 (Pro6) | KR |
| 200 | sectag030 | 3075309 (Pre5) | 3075271 (Pro7) | KR |
| 201 | sectag031 | 3075321 (Pre11) | 3075269 (Pro6) | KR |
| 202 | sectag032 | 3075309 (Pre5) | 3075273 (Pro8) | KR |
| 203 | sectag033 | 3075321 (Pre11) | 3075271 (Pro7) | KR |
| 204 | sectag034 | 3075309 (Pre5) | 3075275 (Pro9) | KR |
| 205 | sectag036 | 3075309 (Pre5) | 3075277 (Pro10) | KR |
| 206 | sectag038 | 3075319 (Pre10) | 3075267 (Pro5) | KR |
| 207 | sectag039 | 3075319 (Pre10) | 3075269 (Pro6) | KR |
| 208 | sectag040 | 3075319 (Pre10) | 3075271 (Pro7) | KR |
| 209 | sectag041 | 3075321 (Pre11) | 3075291 (Pro17) | KR |
| 210 | sectag042 | 3075323 (Pre12) | 3075263 (Pro3) | KREAEA (SEQ ID NO: 105) |
| 211 | sectag043 | 3075323 (Pre12) | 3075273 (Pro8) | KR |
| 212 | sectag046 | 3075301 (Pre1) | 3075263 (Pro3) | KR |
| 213 | sectag047 | 3075321 (Pre11) | 3075275 (Pro9) | KR |
| 214 | sectag048 | 3075309 (Pre5) | 3075279 (Pro11) | KR |
| 215 | sectag049 | 3075321 (Pre11) | 3075277 (Pro10) | KR |
| 216 | sectag050 | 3075309 (Pre5) | 3075281 (Pro12) | KR |
| 217 | sectag052 | 3075309 (Pre5) | 3075283 (Pro13) | KR |
| 218 | sectag053 | 3075321 (Pre11) | 3075283 (Pro13) | KR |
| 219 | sectag054 | 3075309 (Pre5) | 3075285 (Pro14) | KR |
| 220 | sectag055 | 3075321 (Pre11) | 3075285 (Pro14) | KR |
| 221 | sectag056 | 3075309 (Pre5) | 3075287 (Pro15) | KR |
| 222 | sectag057 | 3075309 (Pre5) | 3075289 (Pro16) | KR |
| 223 | sectag058 | 3075307 (Pre4) | 3075263 (Pro3) | KREAEA (SEQ ID NO: 105) |
| 224 | sectag059 | 3075309 (Pre5) | 3075291 (Pro17) | KR |
| 225 | sectag060 | 3075307 (Pre4) | 3075265 (Pro4) | KREAEA (SEQ ID NO: 105) |
| 226 | sectag062 | 3075301 (Pre1) | 3075265 (Pro4) | KR |
| 227 | sectag063 | 3075323 (Pre12) | 3075271 (Pro7) | KREAEA (SEQ ID NO: 105) |
| 228 | sectag064 | 3075301 (Pre1) | 3075267 (Pro5) | KR |
| 229 | sectag065 | 3075323 (Pre12) | 3075273 (Pro8) | KREAEA (SEQ ID NO: 105) |
| 230 | sectag066 | 3075301 (Pre1) | 3075269 (Pro6) | KR |
| 231 | sectag067 | 3075323 (Pre12) | 3075277 (Pro10) | KREAEA (SEQ ID NO: 105) |
| 232 | sectag068 | 3075301 (Pre1) | 3075271 (Pro7) | KR |
| 233 | sectag069 | 3075323 (Pre12) | 3075279 (Pro11) | KREAEA (SEQ ID NO: 105) |
| 234 | sectag071 | 3075323 (Pre12) | 3075281 (Pro12) | KREAEA (SEQ ID NO: 105) |
| 235 | sectag073 | 3075323 (Pre12) | 3075283 (Pro13) | KREAEA (SEQ ID NO: 105) |
| 236 | sectag075 | 3075323 (Pre12) | 3075285 (Pro14) | KREAEA (SEQ ID NO: 105) |
| 237 | sectag076 | 3075301 (Pre1) | 3075279 (Pro11) | KR |
| 238 | sectag077 | 3075307 (Pre4) | 3075267 (Pro5) | KREAEA (SEQ ID NO: 105) |
| 239 | sectag078 | 3075307 (Pre4) | 3075269 (Pro6) | KREAEA (SEQ ID NO: 105) |
| 240 | sectag079 | 3075307 (Pre4) | 3075271 (Pro7) | KREAEA (SEQ ID NO: 105) |
| 241 | sectag083 | 3075307 (Pre4) | 3075279 (Pro11) | KREAEA (SEQ ID NO: 105) |
| 242 | sectag084 | 3075307 (Pre4) | 3075281 (Pro12) | KREAEA (SEQ ID NO: 105) |
| 243 | sectag085 | 3075323 (Pre12) | 3075287 (Pro15) | KREAEA (SEQ ID NO: 105) |
| 244 | sectag086 | 3075301 (Pre1) | 3075281 (Pro12) | KR |
| 245 | sectag088 | 3075301 (Pre1) | 3075283 (Pro13) | KR |
| 246 | sectag090 | 3075301 (Pre1) | 3075285 (Pro14) | KR |
| 247 | sectag092 | 3075301 (Pre1) | 3075287 (Pro15) | KR |
| 248 | sectag094 | 3075301 (Pre1) | 3075289 (Pro16) | KR |
| 249 | sectag095 | 3075323 (Pre12) | 3075297 (Pro20) | KREAEA (SEQ ID NO: 105) |
| 250 | sectag096 | 3075301 (Pre1) | 3075291 (Pro17) | KR |
| 251 | sectag097 | 3075323 (Pre12) | 3075299 (Pro21) | KREAEA (SEQ ID NO: 105) |
| 252 | sectag098 | 3075301 (Pre1) | 3075293 (Pro18) | KR |
| 253 | sectag099 | 3075325 (Pre13) | 3075259 (Pro1) | KREAEA (SEQ ID NO: 105) |
| 254 | sectag100 | 3075301 (Pre1) | 3075295 (Pro19) | KR |
| 255 | sectag101 | 3075307 (Pre4) | 3075283 (Pro13) | KREAEA (SEQ ID NO: 105) |
| 256 | sectag102 | 3075307 (Pre4) | 3075285 (Pro14) | KREAEA (SEQ ID NO: 105) |
| 257 | sectag103 | 3075307 (Pre4) | 3075287 (Pro15) | KREAEA (SEQ ID NO: 105) |
| 258 | sectag104 | 3075307 (Pre4) | 3075289 (Pro16) | KREAEA (SEQ ID NO: 105) |
| 259 | sectag105 | 3075307 (Pre4) | 3075291 (Pro17) | KREAEA (SEQ ID NO: 105) |
| 260 | sectag106 | 3075307 (Pre4) | 3075293 (Pro18) | KREAEA (SEQ ID NO: 105) |
| 261 | sectag107 | 3075307 (Pre4) | 3075295 (Pro19) | KREAEA (SEQ ID NO: 105) |
| 262 | sectag108 | 3075307 (Pre4) | 3075297 (Pro20) | KREAEA (SEQ ID NO: 105) |
| 263 | sectag109 | 3075325 (Pre13) | 3075261 (Pro2) | KREAEA (SEQ ID NO: 105) |
| 264 | sectag110 | 3075301 (Pre1) | 3075297 (Pro20) | KR |
| 265 | sectag111 | 3075325 (Pre13) | 3075263 (Pro3) | KREAEA (SEQ ID NO: 105) |
| 266 | sectag112 | 3075301 (Pre1) | 3075299 (Pro21) | KR |
| 267 | sectag113 | 3075325 (Pre13) | 3075267 (Pro5) | KREAEA (SEQ ID NO: 105) |
| 268 | sectag114 | 3075303 (Pre2) | 3075259 (Pro1) | KR |
| 269 | sectag115 | 3075325 (Pre13) | 3075269 (Pro6) | KREAEA (SEQ ID NO: 105) |
| 270 | sectag116 | 3075303 (Pre2) | 3075261 (Pro2) | KR |
| 271 | sectag117 | 3075325 (Pre13) | 3075271 (Pro7) | KREAEA (SEQ ID NO: 105) |
| 272 | sectag118 | 3075303 (Pre2) | 3075263 (Pro3) | KR |
| 273 | sectag119 | 3075325 (Pre13) | 3075273 (Pro8) | KREAEA (SEQ ID NO: 105) |
| 274 | sectag120 | 3075303 (Pre2) | 3075265 (Pro4) | KR |
| 275 | sectag121 | 3075325 (Pre13) | 3075275 (Pro9) | KREAEA (SEQ ID NO: 105) |
| 276 | sectag122 | 3075303 (Pre2) | 3075267 (Pro5) | KR |
| 277 | sectag124 | 3075303 (Pre2) | 3075269 (Pro6) | KR |
| 278 | sectag126 | 3075309 (Pre5) | 3075259 (Pro1) | KREAEA (SEQ ID NO: 105) |
| 279 | sectag127 | 3075309 (Pre5) | 3075261 (Pro2) | KREAEA (SEQ ID NO: 105) |
| 280 | sectag128 | 3075309 (Pre5) | 3075263 (Pro3) | KREAEA (SEQ ID NO: 105) |
| 281 | sectag129 | 3075309 (Pre5) | 3075265 (Pro4) | KREAEA (SEQ ID NO: 105) |
| 282 | sectag130 | 3075309 (Pre5) | 3075267 (Pro5) | KREAEA (SEQ ID NO: 105) |
| 283 | sectag131 | 3075309 (Pre5) | 3075277 (Pro10) | KREAEA (SEQ ID NO: 105) |
| 284 | sectag132 | 3075309 (Pre5) | 3075283 (Pro13) | KREAEA (SEQ ID NO: 105) |
| 285 | sectag133 | 3075325 (Pre13) | 3075281 (Pro12) | KREAEA (SEQ ID NO: 105) |
| 286 | sectag135 | 3075325 (Pre13) | 3075283 (Pro13) | KREAEA (SEQ ID NO: 105) |
| 287 | sectag137 | 3075325 (Pre13) | 3075285 (Pro14) | KREAEA (SEQ ID NO: 105) |
| 288 | sectag138 | 3075303 (Pre2) | 3075275 (Pro9) | KR |
| 289 | sectag139 | 3075325 (Pre13) | 3075287 (Pro15) | KREAEA (SEQ ID NO: 105) |
| 290 | sectag140 | 3075303 (Pre2) | 3075277 (Pro10) | KR |
| 291 | sectag141 | 3075325 (Pre13) | 3075289 (Pro16) | KREAEA (SEQ ID NO: 105) |
| 292 | sectag143 | 3075325 (Pre13) | 3075291 (Pro17) | KREAEA (SEQ ID NO: 105) |
| 293 | sectag144 | 3075303 (Pre2) | 3075281 (Pro12) | KR |
| 294 | sectag145 | 3075325 (Pre13) | 3075293 (Pro18) | KREAEA (SEQ ID NO: 105) |
| 295 | sectag146 | 3075303 (Pre2) | 3075283 (Pro13) | KR |
| 296 | sectag147 | 3075325 (Pre13) | 3075295 (Pro19) | KREAEA (SEQ ID NO: 105) |
| 297 | sectag148 | 3075303 (Pre2) | 3075287 (Pro15) | KR |
| 298 | sectag150 | 3075309 (Pre5) | 3075287 (Pro15) | KREAEA (SEQ ID NO: 105) |
| 299 | sectag151 | 3075309 (Pre5) | 3075293 (Pro18) | KREAEA (SEQ ID NO: 105) |
| 300 | sectag152 | 3075309 (Pre5) | 3075295 (Pro19) | KREAEA (SEQ ID NO: 105) |
| 301 | sectag153 | 3075309 (Pre5) | 3075299 (Pro21) | KREAEA (SEQ ID NO: 105) |
| 302 | sectag154 | 3075311 (Pre6) | 3075259 (Pro1) | KREAEA (SEQ ID NO: 105) |
| 303 | sectag155 | 3075311 (Pre6) | 3075265 (Pro4) | KREAEA (SEQ ID NO: 105) |
| 304 | sectag157 | 3075325 (Pre13) | 3075297 (Pro20) | KREAEA (SEQ ID NO: 105) |
| 305 | sectag158 | 3075303 (Pre2) | 3075289 (Pro16) | KR |
| 306 | sectag159 | 3075325 (Pre13) | 3075299 (Pro21) | KREAEA (SEQ ID NO: 105) |
| 307 | sectag160 | 3075303 (Pre2) | 3075291 (Pro17) | KR |
| 308 | sectag161 | 3075327 (Pre14) | 3075259 (Pro1) | KREAEA (SEQ ID NO: 105) |
| 309 | sectag162 | 3075303 (Pre2) | 3075293 (Pro18) | KR |
| 310 | sectag163 | 3075327 (Pre14) | 3075261 (Pro2) | KREAEA (SEQ ID NO: 105) |
| 311 | sectag164 | 3075303 (Pre2) | 3075295 (Pro19) | KR |
| 312 | sectag165 | 3075327 (Pre14) | 3075263 (Pro3) | KREAEA (SEQ ID NO: 105) |
| 313 | sectag166 | 3075303 (Pre2) | 3075297 (Pro20) | KR |
| 314 | sectag168 | 3075303 (Pre2) | 3075299 (Pro21) | KR |
| 315 | sectag169 | 3075327 (Pre14) | 3075269 (Pro6) | KREAEA (SEQ ID NO: 105) |
| 316 | sectag171 | 3075327 (Pre14) | 3075271 (Pro7) | KREAEA (SEQ ID NO: 105) |
| 317 | sectag172 | 3075305 (Pre3) | 3075261 (Pro2) | KR |
| 318 | sectag173 | 3075311 (Pre6) | 3075269 (Pro6) | KREAEA (SEQ ID NO: 105) |
| 319 | sectag174 | 3075311 (Pre6) | 3075271 (Pro7) | KREAEA (SEQ ID NO: 105) |
| 320 | sectag176 | 3075311 (Pre6) | 3075275 (Pro9) | KREAEA (SEQ ID NO: 105) |
| 321 | sectag180 | 3075313 (Pre7) | 3075263 (Pro3) | KREAEA (SEQ ID NO: 105) |
| 322 | sectag181 | 3075327 (Pre14) | 3075273 (Pro8) | KREAEA (SEQ ID NO: 105) |
| 323 | sectag182 | 3075305 (Pre3) | 3075263 (Pro3) | KR |
| 324 | sectag184 | 3075305 (Pre3) | 3075265 (Pro4) | KR |
| 325 | sectag185 | 3075327 (Pre14) | 3075277 (Pro10) | KREAEA (SEQ ID NO: 105) |
| 326 | sectag186 | 3075305 (Pre3) | 3075267 (Pro5) | KR |
| 327 | sectag187 | 3075327 (Pre14) | 3075281 (Pro12) | KREAEA (SEQ ID NO: 105) |
| 328 | sectag188 | 3075305 (Pre3) | 3075269 (Pro6) | KR |
| 329 | sectag191 | 3075327 (Pre14) | 3075285 (Pro14) | KREAEA (SEQ ID NO: 105) |
| 330 | sectag194 | 3075305 (Pre3) | 3075275 (Pro9) | KR |
| 331 | sectag195 | 3075311 (Pre6) | 3075283 (Pro13) | KR |
| 332 | sectag196 | 3075305 (Pre3) | 3075277 (Pro10) | KR |
| 333 | sectag197 | 3075313 (Pre7) | 3075265 (Pro4) | KREAEA (SEQ ID NO: 105) |
| 334 | sectag198 | 3075313 (Pre7) | 3075267 (Pro5) | KREAEA (SEQ ID NO: 105) |
| 335 | sectag200 | 3075313 (Pre7) | 3075271 (Pro7) | KREAEA (SEQ ID NO: 105) |
| 336 | sectag201 | 3075313 (Pre7) | 3075273 (Pro8) | KREAEA (SEQ ID NO: 105) |
| 337 | sectag202 | 3075313 (Pre7) | 3075277 (Pro10) | KREAEA (SEQ ID NO: 105) |
| 338 | sectag204 | 3075313 (Pre7) | 3075281 (Pro12) | KREAEA (SEQ ID NO: 105) |
| 339 | sectag205 | 3075311 (Pre6) | 3075285 (Pro14) | KR |
| 340 | sectag207 | 3075311 (Pre6) | 3075289 (Pro16) | KR |
| 341 | sectag208 | 3075305 (Pre3) | 3075281 (Pro12) | KR |
| 342 | sectag209 | 3075311 (Pre6) | 3075291 (Pro17) | KR |
| 343 | sectag210 | 3075305 (Pre3) | 3075283 (Pro13) | KR |
| 344 | sectag211 | 3075313 (Pre7) | 3075261 (Pro2) | KR |
| 345 | sectag212 | 3075305 (Pre3) | 3075285 (Pro14) | KR |
| 346 | sectag214 | 3075305 (Pre3) | 3075287 (Pro15) | KR |
| 347 | sectag216 | 3075305 (Pre3) | 3075289 (Pro16) | KR |
| 348 | sectag218 | 3075305 (Pre3) | 3075291 (Pro17) | KR |
| 349 | sectag220 | 3075305 (Pre3) | 3075293 (Pro18) | KR |
| 350 | sectag221 | 3075313 (Pre7) | 3075285 (Pro14) | KREAEA (SEQ ID NO: 105) |
| 351 | sectag222 | 3075313 (Pre7) | 3075287 (Pro15) | KREAEA (SEQ ID NO: 105) |
| 352 | sectag223 | 3075313 (Pre7) | 3075289 (Pro16) | KREAEA (SEQ ID NO: 105) |
| 353 | sectag224 | 3075313 (Pre7) | 3075293 (Pro18) | KREAEA (SEQ ID NO: 105) |
| 354 | sectag225 | 3075313 (Pre7) | 3075299 (Pro21) | KREAEA (SEQ ID NO: 105) |
| 355 | sectag226 | 3075315 (Pre8) | 3075263 (Pro3) | KREAEA (SEQ ID NO: 105) |
| 356 | sectag230 | 3075305 (Pre3) | 3075295 (Pro19) | KR |
| 357 | sectag231 | 3075313 (Pre7) | 3075293 (Pro18) | KR |
| 358 | sectag232 | 3075305 (Pre3) | 3075297 (Pro20) | KR |
| 359 | sectag233 | 3075313 (Pre7) | 3075297 (Pro20) | KR |
| 360 | sectag234 | 3075307 (Pre4) | 3075259 (Pro1) | KR |
| 361 | sectag235 | 3075313 (Pre7) | 3075299 (Pro21) | KR |
| 362 | sectag236 | 3075307 (Pre4) | 3075261 (Pro2) | KR |
| 363 | sectag238 | 3075307 (Pre4) | 3075263 (Pro3) | KR |
| 364 | sectag239 | 3075315 (Pre8) | 3075267 (Pro5) | KR |
| 365 | sectag240 | 3075307 (Pre4) | 3075265 (Pro4) | KR |
| 366 | sectag242 | 3075307 (Pre4) | 3075267 (Pro5) | KR |
| 367 | sectag243 | 3075317 (Pre9) | 3075261 (Pro2) | KR |
| 368 | sectag245 | 3075303 (Pre2) | 3075285 (Pro14) | KR |
| 369 | sectag246 | 3075305 (Pre3) | 3075299 (Pro21) | KR |
| 370 | sectag247 | 3075307 (Pre4) | 3075287 (Pro15) | KR |
| 371 | sectag248 | 3075311 (Pre6) | 3075281 (Pro12) | KR |
| 372 | sectag250 | 3075311 (Pre6) | 3075293 (Pro18) | KR |
| 373 | sectag251 | 3075311 (Pre6) | 3075295 (Pro19) | KR |
| 374 | sectag252 | 3075313 (Pre7) | 3075267 (Pro5) | KR |
| 375 | sectag253 | 3075317 (Pre9) | 3075263 (Pro3) | KR |
| 376 | sectag254 | 3075307 (Pre4) | 3075271 (Pro7) | KR |
| 377 | sectag255 | 3075317 (Pre9) | 3075265 (Pro4) | KR |
| 378 | sectag256 | 3075307 (Pre4) | 3075273 (Pro8) | KR |
| 379 | sectag257 | 3075317 (Pre9) | 3075271 (Pro7) | KR |
| 380 | sectag259 | 3075317 (Pre9) | 3075291 (Pro17) | KR |
| 381 | sectag260 | 3075307 (Pre4) | 3075277 (Pro10) | KR |
| 382 | sectag261 | 3075319 (Pre10) | 3075273 (Pro8) | KR |
| 383 | sectag262 | 3075307 (Pre4) | 3075279 (Pro11) | KR |
| 384 | sectag263 | 3075319 (Pre10) | 3075275 (Pro9) | KR |
| 385 | sectag264 | 3075307 (Pre4) | 3075281 (Pro12) | KR |
| 386 | sectag265 | 3075319 (Pre10) | 3075277 (Pro10) | KR |
| 387 | sectag266 | 3075307 (Pre4) | 3075283 (Pro13) | KR |
| 388 | sectag268 | 3075307 (Pre4) | 3075285 (Pro14) | KR |
| 389 | sectag269 | 3075313 (Pre7) | 3075285 (Pro14) | KR |
| 390 | sectag272 | 3075313 (Pre7) | 3075291 (Pro17) | KR |
| 391 | sectag273 | 3075313 (Pre7) | 3075295 (Pro19) | KR |
| 392 | sectag275 | 3075315 (Pre8) | 3075263 (Pro3) | KR |
| 393 | sectag276 | 3075317 (Pre9) | 3075267 (Pro5) | KR |
| 394 | sectag277 | 3075319 (Pre10) | 3075281 (Pro12) | KR |
| 395 | sectag278 | 3075307 (Pre4) | 3075289 (Pro16) | KR |
| 396 | sectag279 | 3075319 (Pre10) | 3075285 (Pro14) | KR |
| 397 | sectag280 | 3075307 (Pre4) | 3075291 (Pro17) | KR |
| 398 | sectag282 | 3075307 (Pre4) | 3075293 (Pro18) | KR |
| 399 | sectag284 | 3075307 (Pre4) | 3075295 (Pro19) | KR |
| 400 | sectag285 | 3075319 (Pre10) | 3075291 (Pro17) | KR |
| 401 | sectag286 | 3075307 (Pre4) | 3075297 (Pro20) | KR |
| 402 | sectag288 | 3075307 (Pre4) | 3075299 (Pro21) | KR |
| 403 | sectag289 | 3075319 (Pre10) | 3075295 (Pro19) | KR |
| 404 | sectag290 | 3075309 (Pre5) | 3075259 (Pro1) | KR |
| 405 | sectag291 | 3075319 (Pre10) | 3075297 (Pro20) | KR |
| 406 | sectag292 | 3075309 (Pre5) | 3075261 (Pro2) | KR |
| 407 | sectag293 | 3075317 (Pre9) | 3075273 (Pro8) | KR |
| 408 | sectag295 | 3075317 (Pre9) | 3075277 (Pro10) | KR |
| 409 | sectag298 | 3075317 (Pre9) | 3075283 (Pro13) | KR |
| 410 | sectag300 | 3075317 (Pre9) | 3075287 (Pro15) | KR |
| 411 | sectag994 | | | |

TABLE 16
Results from Secondary Screen Showing bLF Titer Levels. bLF titer
levels measured using ELISA were normalized to the titer of control
strain 581281 (Sc-alphaMF) (shown below as Strain No. 1).

| | Titer | | Titer |
|---|---|---|---|
| Pichia | relative to | Pichia | relative to |
| Strain No. | control | Strain No. | control |
| 1 | 1 | 411 | 0.907 |
| 14 | 3.466 | 180 | 1.217 |
| 15 | 1.369 | 188 | 1.017 |
| 24 | 0.908 | 197 | 0.462 |
| 32 | 1.144 | 217 | 0.774 |
| 86 | 0.34 | 240 | 0.597 |
| 90 | 0.349 | 255 | 1.95 |
| 96 | 0.847 | 259 | 0.97 |
| 117 | 2.35 | 262 | 1.903 |
| 126 | 0.966 | 267 | 0.354 |
| 127 | 2.337 | 286 | 1.084 |
| 136 | 2.183 | 295 | 0.781 |
| 147 | 1.355 | 299 | 0.063 |
| 148 | 1.063 | 301 | 2.314 |
| 151 | 1.018 | 304 | 1.067 |
| 159 | 1.619 | 306 | 1.414 |
| 166 | 1.596 | 314 | 1.246 |
| 170 | 1.198 | 354 | 2.092 |
| 172 | 1.02 | 360 | 0.717 |
| 174 | 0.617 | 402 | 0.892 |

TABLE 17
Top bLF Producers from the secondary screen

| Pichia | Pre- | | Pro- | |
|---|---|---|---|---|
| Strain No. | Seq ID | Pre-sequence Description | Seq ID | Pro-sequence Description |
| 14 | 3075321 | Beta-2-microglobulin \| | 3075299 | Mf(alpha)1p [Saccharomyces cerevisiae |
| | | P01888.2 \| Bos taurus \|d21- | | YJM1383] \| d1-19 d86-186 V22A G40D |
| | | 118 | | L42S V50A V52A F55L L64S F65S |
| | | | | I66T |
| 166 | 3075317 | Endo-1,3(4)-beta-glucanase | 3075299 | Mf(alpha)1p [Saccharomyces cerevisiae |
| | | 1 \| P53753.1 \| | | YJM1383] \| d1-19 d86-186 V22A G40D |
| | | Saccharomyces cerevisiae | | L42S V50A V52A F55L L64S F65S |
| | | S288C \| d17-1117 | | I66T |
| 117 | 3075301 | M1 killer toxin | 3075299 | Mf(alpha)1p [Saccharomyces cerevisiae |
| | | [Saccharomyces paradoxus] | | YJM1383] \| d1-19 d86-186 V22A G40D |
| | | \| AII19506.1 \|d27-316 | | L42S V50A V52A F55L L64S F65S |
| | | | | I66T |
| 127 | 3075317 | Endo-1,3(4)-beta-glucanase | 3075299 | Mf(alpha)1p [Saccharomyces cerevisiae |
| | | 1 \| P53753.1 \| | | YJM1383] \| d1-19 d86-186 V22A G40D |
| | | Saccharomyces cerevisiae | | L42S V50A V52A F55L L64S F65S |
| | | S288C \| d17-1117 | | I66T |
| 301 | 3075309 | Mf(alpha)1p | 3075299 | Mf(alpha)1p [Saccharomyces cerevisiae |
| | | [Saccharomyces cerevisiae | | YJM1383] \| d1-19 d86-186 V22A G40D |
| | | YJM1383] \| AJW01277.1 \| | | L42S V50A V52A F55L L64S F65S |
| | | d20-186 | | I66T |
| 136 | 3075327 | Bactenecin-1 \| P22226.2 \| | 3075299 | Mf(alpha)1p [Saccharomyces cerevisiae |
| | | Bos taurus \| d30-155 | | YJM1383] \| d1-19 d86-186 V22A G40D |
| | | | | L42S V50A V52A F55L L64S F65S |
| | | | | I66T |
| 354 | 3075313 | Protein disulfide-isomerase | 3075299 | Mf(alpha)1p [Saccharomyces cerevisiae |
| | | \|P17967.2 \| Saccharomyces | | YJM1383] \| d1-19 d86-186 V22A G40D |
| | | cerevisiae S288C \| d23-522 | | L42S V50A V52A F55L L64S F65S |
| | | | | I66T |
| 255 | 3075307 | Oligosaccharyl transferase | 3075283 | Pichia sorbitophila (strain ATCC MYA- |
| | | subunit \| P41543.1 \| | | 4447 / BCRC 22081 / CBS 7064 / |
| | | P41543.1 \| Saccharomyces | | NBRC 10061 / NRRL Y-12695) (Hybrid |
| | | cerevisiae S288C \|d23-477 | | yeast) \| Piso0_003304 protein \| d1-19 |
| | | | | d106-317 |
| 262 | 3075307 | Oligosaccharyl transferase | 3075297 | Mf(alpha)1p [Saccharomyces cerevisiae |
| | | subunit \| P41543.1 \| | | YJM1383] \| d1-19 d86-186 V22A L64S |
| | | P41543.1 \| Saccharomyces | | |
| | | cerevisiae S288C \|d23-478 | | |
| 159 | 3075319 | Carboxypeptidase Y- | 3075299 | Mf(alpha)1p [Saccharomyces cerevisiae |
| | | deficient protein 4 \| | | YJM1383] \| d1-19 d86-186 V22A G40D |
| | | P07267.1 \| Saccharomyces | | L42S V50A V52A F55L L64S F65S |
| | | cerevisiae S288C \| d23-405 | | I66T |

### Secretion Signal Sequence Analysis

The amino acid sequences of secretion signals that were found by screening to be capable of promoting secretion of bLF were analyzed for structural similarities. Various common structural motifs were identified (TABLE 18).

TABLE 18
Secretion Signal Common Structural Motifs

| SEQ | |
|---|---|
| ID | |
| NO: | Motif Description |
| 15 | LXXXXLL, wherein X is independently chosen from any amino acid |
| 16 | LX²X³X⁴X⁵LL, wherein: X² is phenylalanine (F), proline (P), threonine (T), |
| | serine (S), valine (V), or leucine (L); X³ is phenylalanine (F), tryptophan |
| | (W), proline (P), alanine (A), valine (V), or leucine (L); X⁴ is proline |
| | (P), serine (S), alanine (A), valine (V), isoleucine (I), or leucine (L); |
| | and X⁵ is phenylalanine (F), glycine (G), alanine (A), serine (S), |
| | threonine (T), or leucine (L) |
| 17 | LX²X³X⁴X⁵LLX⁸X⁹X¹⁰X¹¹X¹², wherein: X² is phenylalanine (F), proline (P), |
| | threonine (T), serine (S), valine (V), or leucine (L); X³ is phenylalanine |
| | (F), tryptophan (W), proline (P), alanine (A), valine (V), or leucine (L); |
| | X⁴ is proline (P), serine (S), alanine (A), valine (V), isoleucine (I), or |
| | leucine (L); X⁵ is phenylalanine (F), glycine (G), alanine (A), serine (S), |
| | threonine (T), or leucine (L); X⁸ is histidine (H), serine (S), threonine |
| | (T), valine (V), or leucine (L); X⁹ is phenylalanine (F), serine (S), |
| | threonine (T), alanine (A), valine (V), or leucine (L); X¹⁰ is serine |
| | (S), threonine (T), glycine (G), alanine (A), or valine (V); X¹¹ is glycine |
| | (G), alanine (A), valine (V), asparagine (N), glutamic acid (E), serine (S), |
| | or tyrosine (Y); and X¹² is alanine (A), valine (V), asparagine (N), |
| | glutamic acid (E), cytosine (C), or leucine (L) |
| 18 | WFSWIVG |
| 19 | MRFPSIFTAVLF |
| 20 | SSALA |
| 21 | IVGLF |
| 22 | MTKPTQVLV |
| 23 | MKLATAFTILTA |
| 24 | ETPRASLSLGRW |
| 25 | WHAVMVFVLCG |
| 222 | MRFPSIFT |
| 26 | MKX³X⁴X⁵X⁶AX⁸LSX¹¹X¹²X¹³LX¹⁴L, wherein: X³ is phenylalanine (F) or leucine |
| | (L); X⁴ is serine (S) or phenylalanine (F); X⁵ is alanine (A) or valine (V); |
| | X⁶ is glycine (G) or proline (P); X⁸ is valine (V) or leucine (L); X¹¹ is |
| | tryptophan (W) or leucine (L); X¹² is serine (S) or glycine (G); X¹³ is |
| | serine (S) or alanine (A); and X¹⁴ is leucine (L) or glycine (G) |
| 223 | MX²X³X⁴X⁵, wherein: X² is arginine (R) or glutamine (Q); X³ is histidine (H) |
| | or glutamine (Q); X⁴ is valine (V) or phenylalanine (F); and X⁵ is leucine |
| | (L) or tryptophan (W) |
| 173 | M-A′-Q-B′-L-C′-L-D′-LL-E′, where M is methionine, Q is glutamine, and L is |
| | leucine, and A′ is 0-5 amino acids in length, B′ is 0-3 amino acids in |
| | length, C′ is 0-7 amino acids in length, D′ is 0-5 amino acids in length, |
| | and E′ is 0-6 amino acids in length, wherein any amino acid of A′, B′, C′, |
| | D′ and E′ is any amino acid |
| 62 | NTTIXXXA, wherein X is independently chosen from any amino acid |
| 64 | RYVVGDDEQ |
| 65 | IVAKSGI |
| 66 | IPDEAIAN |
| 67 | QTSISDDEEPIVVEINGQKV |
| 68 | INTTLTEEALEKSGISIDDL |
| 69 | PVFAEIDNK |
| 70 | DDLKESYAN |
| 71 | PVENVDD |
| 72 | IDQEQLTNG |
| 73 | PVDSGAKGKYSR |
| 74 | NDGVGVGMSTIKEEDFGKHF |
| 224 | TTIASIA |
| 225 | YVVGDDEQ |
| 226 | PVFAEIDNKPVVYIVNTTKA |
| 227 | ESIVAKSGITLDDLKESYAN |
| 63 | NTTIX⁵X⁶X⁷A, wherein: X⁵ is alanine (A), leucine (L), or tyrosine (Y); X⁶ |
| | is alanine (A), serine (S), asparagine (N), or glutamic acid (E); and X⁷ |
| | is alanine (A), isoleucine (I), serine (S), glutamic acid (E), or glutamine |
| | (Q) |
| 221 | AAX³EEGX⁷SLDKR, wherein: X³ is lysine (K) or alanine (A); and X⁷ is valine |
| | (V) or serine (S) |
| 75 | X¹NTTIAX⁷X⁸AX¹⁰X¹¹EEGVX¹⁶, wherein: X¹ is valine (V) or isoleucine (I); X⁷ |
| | is aspartic acid (D), serine (S), or glutamic acid (E); X⁸ is isoleucine |
| | (I) or glutamine (Q); X¹⁰ is alanine (A) or leucine (L); X¹¹ is alanine |
| | (A) or lysine (K); and X¹⁶ is serine (S) or leucine (L) |
| 76 | X¹X²X³X⁴X⁵DDEX⁹, wherein: X¹ is arginine (R) or glutamine (Q); X² is |
| | tyrosine (Y) or threonine (T); X³ is valine (V) or serine (S); X⁴ is |
| | valine (V) or isoleucine (I); X⁵ is glycine (G) or serine (S); and |
| | X⁹ is glutamine (Q) or glutamic acid (E) |
| 77 | AX²LPFSNX⁸TNX¹¹GX¹³X¹⁴FX¹⁶NTTI, wherein: X² is valine (V) or leucine (L); |
| | X⁸ is serine (S) or glycine (G); X¹¹ is asparagine (N) or threonine (T); |
| | X¹³ is isoleucine (I) or leucine (L); X¹⁴ is serine (S), leucine (L), or |
| | methionine (M); and X¹⁶ is valine (V) or isoleucine (I) |
| 78 | X¹AQX⁴PAEAX⁹IGX¹²LDLX¹⁶X¹⁷X¹⁸X¹⁹D, wherein: X¹ is threonine (T) or serine |
| | (S); X⁴ is isoleucine (I) or valine (V); X⁹ is valine (V) or isoleucine |
| | (I); X¹² is tyrosine (Y) or phenylalanine (F); X¹⁶ is glutamic acid (E) |
| | or threonine (T); X¹⁷ is aspartic acid (D) or glycine (G); X¹⁸ is |
| | aspartic acid (D), serine (S), or alanine (A); and X¹⁹ is glutamic acid |
| | (E) or phenylalanine (F) |
| 79 | X¹GX³X⁴X⁵X⁶X⁷DX⁹IX¹¹P, wherein: X¹ is lysine (K) or serine (S); X³ is |
| | lysine (K) or arginine (R); X⁴ is tyrosine (Y) or phenylalanine (F); |
| | X⁵ is serine (S) or leucine (L); X⁶ is arginine (R) or glutamic acid |
| | (E); X⁷ is glutamine (Q) or threonine (T); X⁹ is leucine (L) or isoleucine |
| | (I); and X¹¹ is isoleucine (I) or phenylalanine (F) |
| 80 | X¹X²NX⁴TX⁶E, wherein: X¹ is asparagine (N) or proline (P); X² is glycine |
| | (G) or alanine (A); X⁴ is glycine (G) or threonine (T); and X⁶ is serine |
| | (S) or threonine (T) |
| 228 | PAEAVIX⁷Y, wherein X⁷ is aspartic acid (D) or glycine (G) |
| 229 | KEEX⁴X⁵X⁶X⁷X⁸KR, wherein: X⁴ is glycine (G) or glutamic acid (E); X⁵ is |
| | valine (V) or alanine (A); X⁶ is serine (S) or lysine (K); X⁷ is leucine |
| | (L) or asparagine (N); and X⁸ is aspartic acid (D) or glycine (G) |
| 239 | GDFDX⁵AX⁷LP, wherein: X⁵ is valine (V) or alanine (A); and X⁷ is valine |
| | (V) or alanine (A) |
| 231 | X¹SNST, wherein X¹ is leucine (L) or phenylalanine (F) |
| 232 | GLSX⁴TN, wherein X⁴ is serine (S) or phenylalanine (F) |
| 233 | PX²SNSTNNGLSX¹²TNTTIASI, wherein: X² is leucine (L) or phenylalanine (F); |
| | and X¹² is serine (S) or phenylalanine (F) |
| 234 | X¹X²X³IPX⁶EAX⁹X¹⁰X¹¹X¹²X¹³X¹⁴X¹⁵X¹⁶X¹⁷DX¹⁹X²⁰, wherein: X¹ is threonine |
| | (T) or aspartic acid (D); X² is alanine (A) or leucine (L); X³ is |
| | glutamine (Q) or isoleucine (I); X⁶ is alanine (A) or aspartic acid (D); |
| | X⁹ is valine (V) or isoleucine (I); X¹⁰ is isoleucine (I) or alanine (A); |
| | X¹¹ is aspartic acid (D), glycine (G), or asparagine (N); X¹² is |
| | tyrosine (Y) or arginine (R); X¹³ serine (S) or tyrosine (Y); X¹⁴ is |
| | aspartic acid (D) or valine (V); X¹⁵ is leucine (L) or valine (V); X¹⁶ is |
| | glutamic acid (E) or glycine (G); X¹⁷ is glycine (G) or aspartic acid |
| | (D); X¹⁹ is phenylalanine (F) or glutamic acid (E); and X²⁰ is aspartic |
| | acid (D) or glutamine (Q) |
| 146 | CX²X³X⁴X⁵X⁶X⁷X⁸APX¹¹NTTT, wherein: X² is leucine (L), phenylalanine (F), |
| | or glycine (G); X³ is leucine (L), phenylalanine (F),or valine (V); |
| | X⁴ is asparagine (N) or valine (V); X⁵ is valine (V) or leucine (L); |
| | X⁶ is serine (S), alanine (A), or valine (V); X⁷ is serine (S) or |
| | alanine (A); X⁸ is alanine (A) or glycine (G); and X¹¹ is valine (V) |
| | or alanine (A) |
| 147 | X¹AAPX⁵X⁶TTTEDE, wherein: X¹ is leucine (L), serine (S), or alanine (A); |
| | X⁵ is alanine (A) or valine (V); and X⁶ is asparagine (N) or serine (S) |
| 148 | AAPIX⁵X⁶X⁷X⁸S, wherein: X⁵ is asparagine (N) or lysine (K); X⁶ is |
| | isoleucine (I) or phenylalanine (F); X⁷ is threonine (T) or asparagine |
| | (N); and X⁸ is serine (S) or aspartic acid (D) |
| 149 | X¹X²X³X⁴X⁵X⁶X⁷X⁸X⁹, wherein: X¹ is glutamine (Q) or asparagine (N); X² |
| | is valine (V) or histidine (H); X³ is tryptophan (W) or phenylalanine |
| | (F); X⁴ is phenylalanine (F), leucine (L), or histidine (H); X⁵ is |
| | serine (S) or alanine (A); X⁶ is tryptophan (W), leucine (L), or valine |
| | (V); X⁷ is isoleucine (I), leucine (L), or methionine (M); X⁸ is valine |
| | (V) or leucine (L); and X⁹ is glycine (G), alanine (A), or phenylalanine |
| | (F) |
| 150 | X¹X²X³X⁴X⁵X⁶AX⁸X⁹, wherein: X¹ lysine (K) or asparagine (N); X² is |
| | glycine (G), asparagine (N), or aspartic acid (D); X³ is asparagine |
| | (N), glycine (G), or lysine (K); X⁴ is leucine (L), tyrosine (Y), or |
| | glycine (G); X⁵ is serine (S) or asparagine (N); X⁶ is serine (S), |
| | arginine (R), or glycine (G); X⁸ is asparagine (N), aspartic acid (D), |
| | or serine (S); and X⁹ is threonine (T), leucine (L), or glutamic acid (E) |
| 151 | X¹RX³X⁴X⁵X⁶X⁷X⁸X⁹X¹⁰X¹¹X¹²X¹³X¹⁴X¹⁵X¹⁶, wherein: X¹ is methionine (M), |
| | valine (V), or glutamine (Q); X³ is phenylalanine (F) or glutamine (Q); |
| | X⁴ is leucine (L) or valine (V); X⁵ is serine (S) or tryptophan (W); X⁶ |
| | is phenylalanine (F) or leucine (L); X⁷ is leucine (L) or serine (S); X⁸ |
| | is threonine (T), leucine (L), phenylalanine (F), or tryptophan (W); X⁹ |
| | is alanine (A), leucine (L), or isoleucine (I); X¹⁰ is valine (V) or |
| | leucine (L); X¹¹ is leucine (L), glycine (G), or serine (S); X¹² is |
| | leucine (L) or phenylalanine (F); X¹³ is valine (V), leucine (L), |
| | or phenylalanine (F); X¹⁴ is valine (V) or leucine (L); X¹⁵ is |
| | serine (S) or cytosine (C); and X¹⁶ is alanine (A) or phenylalanine |
| | (F) |
| 152 | X¹X²X³X⁴X⁵X⁶X⁷X⁸X⁹X¹⁰X¹¹X¹²X¹³X¹⁴X¹⁵X¹⁶X¹⁷IX¹⁹X²⁰, wherein: X¹ is |
| | aspartic acid (D), valine (V), or glutamic acid (E); X² is valine |
| | (V), tyrosine (Y), or proline (P); X³ is proline (P), isoleucine (I), |
| | or serine (S); X⁴ is glycine (G) or valine (V); X⁵ is threonine (T), |
| | asparagine (N), or arginine (R); X⁶ is serine (S), threonine (T), or |
| | phenylalanine (F); X⁷ is glutamine (Q), threonine (T), or leucine |
| | (L); X⁸ is glycine (G), lysine (K), or glutamic acid (E); X⁹ is valine |
| | (V), alanine (A), or glutamine (Q); X¹⁰ is glutamic acid (E) or aspartic |
| | acid (D); X¹¹ is phenylalanine (F), serine (S), or isoleucine (I); X¹² |
| | is isoleucine (I) or proline (P); X¹³ is phenylalanine (F) or valine |
| | (V); X¹⁴ is alanine (A) or proline (P); X¹⁵ is lysine (K) or glutamine |
| | (Q); X¹⁶ is glutamic acid (E), serine (S), or glutamine (Q); X¹⁷ is |
| | alanine (A) or glycine (G); X¹⁹ is isoleucine (I), threonine (T), or |
| | asparagine (N); and X²⁰ is glutamic acid (E), leucine (L), or |
| | alanine (A) |
| 235 | AAPX⁴X⁵X⁶X⁷X⁸X⁹X¹⁰X¹¹X¹², wherein: X⁴ is alanine (A) or valine (V); |
| | X⁵ is asparagine (N) or aspartic acid (D); X⁶ is serine (S) or |
| | threonine (T); X⁷ is threonine (T) or glycine (G); X⁸ is threonine |
| | (T) or alanine (A); X⁹ is glutamic acid (E) or lysine (K); X¹⁰ is |
| | glycine (G) or aspartic acid (D); X¹¹ is glutamic acid (E) or lysine |
| | (K); and X¹² is threonine (T) or tyrosine (Y) |
| 236 | AX²KEEX⁶X⁷X⁸X⁹X¹⁰KREAEA, wherein: X² is alanine (A) or threonine (T); |
| | X⁶ is glycine (G) or glutamic acid (E); X⁷ is valine (V) or alanine |
| | (A); X⁸ is serine (S) or lysine (K); X⁹ is leucine (L) or asparagine |
| | (N); and X¹⁰ is aspartic acid (D) or glycine (G) |
| 237 | SLLX⁴X⁵SX⁷X⁸LAAPX¹³NTTTEDE, wherein: X⁴ is alanine (A), phenylalanine |
| | (F), leucine (L), or serine (S); X⁵ is leucine (L) or alanine (A); X⁷ |
| | is leucine (L) or serine (S); X⁸ is leucine (L) or valine (V); and X¹³ |
| | is alanine (A) or valine (V) |
| 238 | MX²X³X⁴X⁵X⁶X⁷X⁸X⁹, wherein: X² is alanine (A), lysine (K), or arginine |
| | (R); X³ is leucine (L), glutamine (Q), or arginine (R); X⁴ is |
| | phenylalanine (F) or valine (V); X⁵ is valine (V) or tryptophan (W); X⁶ |
| | is alanine (A), phenylalanine (F), or proline (P); X⁷ is leucine (L), |
| | alanine (A), or serine (S); X⁸ is leucine (L), valine (V), or |
| | tryptophan (W); and X⁹ is leucine (L) or isoleucine (I) |

### Example 3: Testing of Effect of HAC1 Overexpression on Secretion Signal Functionality

### Strain Construction

The functionality of a subset of the top-producing secretion signals (i.e., sectag909, sectag923, and sectag983) were further validated by measuring bLF secretion, with and without HAC1 overexpression. This validation was performed with bLF expression driven by a GAP promoter and in a strain expressing S. cerevisiae HAC1 gene (UniProt P41546-1) (FIG. 4C).

Pichia pastoris BG11 strain was modified by integrating DNA fragments, each containing a bLF expression cassette with a unique secretion signal (i.e., sectag909, sectag923, sectag983, or secretion signal from Saccharomyces cerevisiae alpha mating factor) as described in Example 1. The bLF expression cassette in this example included a GAP promoter and the FDH1 terminator (both linked to the sequence encoding bLF) (FIG. 4A). On-target integration was verified by WGS. Into these strains, ScHAC1 expression cassette was introduced via ends-in integration of the linear DNA at GAP locus.

### Screening

The strains were cultivated in bioreactors and samples for bLF quantitation were taken at 168 hours after inoculation. The bioreactor operated continuously while maintaining constant pH, temperature and dissolved oxygen levels. Please refer to “Culturing Host Cells” above. Titer measurements were made using ELISA.

These experiments again verified that the secretion signals promote bLF section. Moreover, these experiments demonstrated that HAC1 overexpression can further stimulate protein secretion (TABLE 19), as has been reported previously by others (see e.g., Resina et al., New Biotechnology. 2009. 25(6): 396-403.).

TABLE 19
Analysis of bLF Production Over Time, With or Without
HAC1 Overexpression. Titers were measured using ELISA.

| | | | bLF titer, | |
|---|---|---|---|---|
| Pichia | | Secretion | average | Std |
| Strain No. | Reactor | signal | [mg/L] | deviation |
| 412 | R1 | Control | 6.19 | 0.50 |
| 412 | R2 | Control | 5.90 | 0.56 |
| 413 | R3 | sectag909 | 10.37 | 0.83 |
| 413 | R4 | sectag909 | 10.83 | 1.50 |
| 414 | R5 | sectag923 | 7.90 | 0.66 |
| 414 | R6 | sectag923 | 9.00 | 1.36 |
| 415 | R7 | sectag983 | 12.27 | 2.21 |
| 415 | R8 | sectag983 | 12.42 | 1.44 |
| 416 | R9 | sectag909 | 248.77 | 34.00 |
| | | without HAC1 | | |
| 416 | R10 | sectag909 | 246.74 | 40.78a |
| | | without HAC1 | | |
| 417 | R11 | Non-producer | 0.00 | 0.00 |
| 417 | R12 | Non-producer | 0.04 | 0.05 |

### Example 4: Testing of Chaperones in Pichia

### Strain Construction

Additional Pichia pastoris strains were constructed to test whether overexpression of chaperone proteins (i.e., Calreticulin proteins and PDIA3 proteins) promote protein secretion. Again, bovine lactoferrin (bLF, UniProt P24627) was selected as an exemplary protein for screening the chaperones.

Pichia pastoris strain YB-4290 was used as the base strain. A linear DNA containing expression cassettes for bLF and S. cerevisiae HAC1 (UniProt P41546-1) was introduced into this initial strain for genomic integration thereby generating a base strain for further genetic modification. The integration simultaneously removed the promoter and ORF of the AOX1 gene (NCBI AT249_GQ6705214). The bLF expression cassette included a synthetic (thiamine sensitive) promoter, bLF ORF with native secretion signal replaced with sectag983 sequence, and the TEF1 terminator (both linked to the sequence encoding bLF). The ScHAC1 expression cassette included a constitutive promoter and the AOX1 terminator.

The base strain was further modified by genetic integration of a PDI1 (NCBI GQ68_05219T0) expression cassette and a GPX1 (NCBI GQ68_00445T0) expression cassette (FIG. 4D). Integration of PDI1 and GPX1 simultaneously removed the promoter and ORF of GAS1 gene (NCBI GQ68_02762T0) via homologous recombination. These modifications were previously shown to increase the production of secreted protein (see e.g., Resina et al. New Biotechnology. 2009. 25(6): 396-403; Delic et al., Free Radical Biology and Medicine. 2000-2012. 52(9): 2000-2012). This strain was further modified by genetic integration of a Calreticulin protein (e.g., UniProt A0A397UMK4) expression cassette and a PDIA3 (a.k.a. ERp57) protein (e.g., UniProt A0A1Y1VSB5) expression cassette (FIG. 4B).

Four clones were picked from each transformation plate for screening, grown in YPD culture, mixed with 40% Glycerol solution at equal volume, and stored at −80° C. To screen a library of human Calreticulin and human PDIA3 homologs, at least four clones were picked for screening without WGS validation.

### Chaperone Screening

2 uL of glycerol stock of transformation clones were inoculated in a preculture media 1, which was a mineral media with glucose as carbon source and thiamine to repress bLF expression, contained in a deepwell plate. The cultivation plates were incubated at 30° C., 1000 rpm shaking speed for 24-48 h. bLF expression was induced as described in WO 2022/051696 A1. At least four clones from the library transformation were tested. Each clone was tested in at least two cultures.

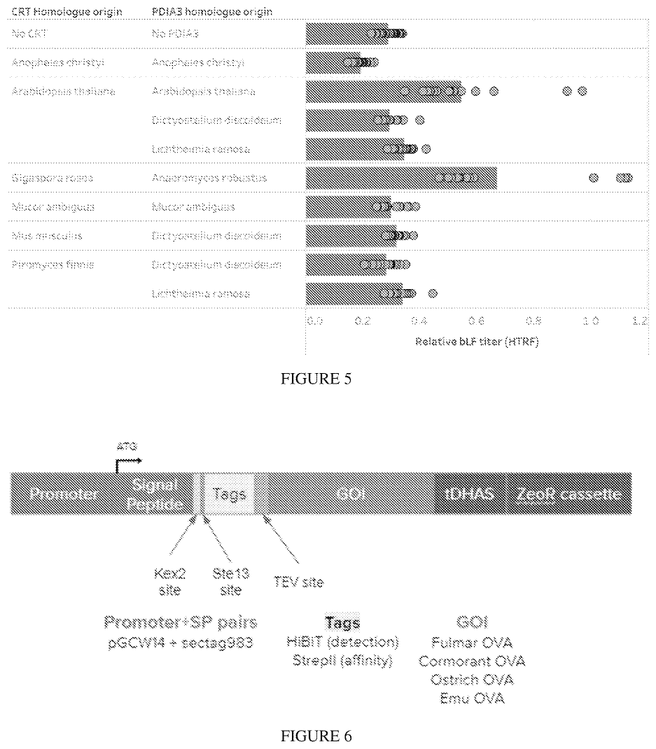

Various Calreticulin and PDIA3 combinations induced production of bLF to a greater extent than the control (i.e., no Calreticulin protein and no PDIA3 protein) (TABLE 20, FIG. 5).

TABLE 20
bLF Production with Calreticulin and PDIA3 Overexpression.
Titers were measured using homogenous time
resolved fluorescence (HTRF).

| | CRT | PDIA3 | bLF titer, | | |
|---|---|---|---|---|---|
| Pichia | Homologue | homologue | average | Std | Number of |
| Strain No. | origin | origin | [mg/L] | dev | Replicates |
| 418 | No CRT | No PDIA3 | 0.290 | 0.031 | 34 |
| 419 | Anopheles | Anopheles | 0.193 | 0.024 | 16 |
| | christyi | christyi | | | |
| 420 | Arabidopsis | Dictyostelium | 0.295 | 0.039 | 16 |
| | thaliana | discoideum | | | |
| 421 | Arabidopsis | Lichtheimia | 0.345 | 0.034 | 16 |
| | thaliana | ramose | | | |
| 422 | Arabidopsis | Arabidopsis | 0.545 | 0.174 | 16 |
| | thaliana | thaliana | | | |
| 423 | Gigaspora | Anaeromyces | 0.673 | 0.254 | 16 |
| | rosea | robustus | | | |
| 424 | Mucor | Mucor | 0.300 | 0.042 | 16 |
| | ambiguus | ambiguus | | | |
| 425 | Mus | Dictyostelium | 0.318 | 0.025 | 16 |
| | musculus | discoideum | | | |
| 426 | Piromyces | Dictyostelium | 0.284 | 0.041 | 16 |
| | finnis | discoideum | | | |
| 427 | Piromyces | Lichtheimia | 0.340 | 0.040 | 16 |
| | finnis | ramose | | | |

### Example 5: Expression of OVA in Pichia

### Strain Construction

Pichia pastoris strains were constructed to test the extent to which the secretion signals identified in Example 2 as promoting bLF section in Pichia also promote secretion of additional proteins. Here, the secretion of various ovalbumin proteins was tested (FIG. 6).

Pichia pastoris strain Y-7556 with AOX1 and AOX2 genes deleted was used as the initial strain. Into this strain, a linear DNA containing HiBiT tagged, codon-optimized nucleotide sequence of Emu Ovalbumin (OVA, UniProt E2RVI8), or Fulmar OVA (NCBI XP_009580141.1), or Cormorant OVA (NCBI XP_009507609.1), or Ostrich OVA (NCBI XP_009676351.1), along with zeocin-resistance expression cassettes, was introduced by ends-out recombination at a chromosomal genetic locus. In each case, the OVA gene was expressed under Pichia pastoris native GCW14 promoter and native Dihydroxyacetone synthase (DHAS) terminator. Sectag983 was used to replace the start methionine amino acid of the original ovalbumin sequences. Each secretion signal contained a signal peptidase recognition site of KREAEA (SEQ ID NO: 105) at its C-terminal end.

Linear DNA sequences, each containing a sectag983 linked to one of the OVA sequences discussed above, were transformed individually into the initial strain using PEG/LiAC/ssDNA transformation method and plated on YPD+Zeocin agar plates. Twenty-four clones were picked from each transformation plate for screening, grown in YPD culture until saturated, mixed with 40% Glycerol solution at equal volume, and stored at −80° C.

### Screening

Glycerol stocked strains were screened for activity by inoculating glycerol stock into preculture media in 384-well plates. Preculture media contained salt-modified FM-22, dextrose, PTM4 and water. Pre-culture plates were incubated at 30° C., 1000 rpm, 80% humidity for ˜24 hours. Pre-culture plates were then stamped into secondary pre-culture plates that contained the same pre-culture media as described above and were incubated at 30° C., 1000 rpm, 80% humidity for ˜24 hours. The secondary pre-culture plates were then stamped into the production plate, which contained the same pre-culture media as described above, and which was incubated at 30° C., 1000 rpm, 80% humidity for ˜48 hours. DWP strain performance was measured using luminescence readout of the supernatant using Promega's Nano-Glo® HiBiT Lytic Detection System and protocol, or a variant thereof. Some strains were advanced from the primary DWP screen to lab scale fermentation. These strains were cultivated in bioreactors. After 24 hours, samples for OVA quantitation were taken. The bioreactor operated continuously while maintaining constant pH, temperature and dissolved oxygen levels. Please refer to “Culturing Host Cells” above.

Approximately 2 ml of fermentation whole broth was collected, filtered, and then denatured by mixing the sample into a mixture of Tris-Glycine SDS Sample Buffer and a reducing agent containing dithiothreitol (DTT). The resulting mixture was boiled at 90° C. for 10 minutes. Samples were then loaded onto Tris-Glycine SDS-PAGE gels along with a protein ladder and a HiBiT control protein. This was performed in duplicate. One of the resulting gels was stained using the Coomassie stain method, while the other gel had its protein transferred to a nitrocellulose membrane and probed using the Promega Nano-Glo® HiBiT Blotting System and protocol. Resulting Coomassie stain gels and blotted membranes were then imaged, and ovalbumin titers were calculated. Exemplary ovalbumin titers for Emu, Fulmar, Cormorant, and Ostrich OVA were at least 10 mg/L as shown in SDS-PAGE gels having a limit of detection of 10 mg/L. These experiments demonstrated that sectag983 is capable of promoting secretion of each of these ovalbumin proteins in Pichia.

### Example 6: Expression of bLF in Aspergillus

### Strain Construction

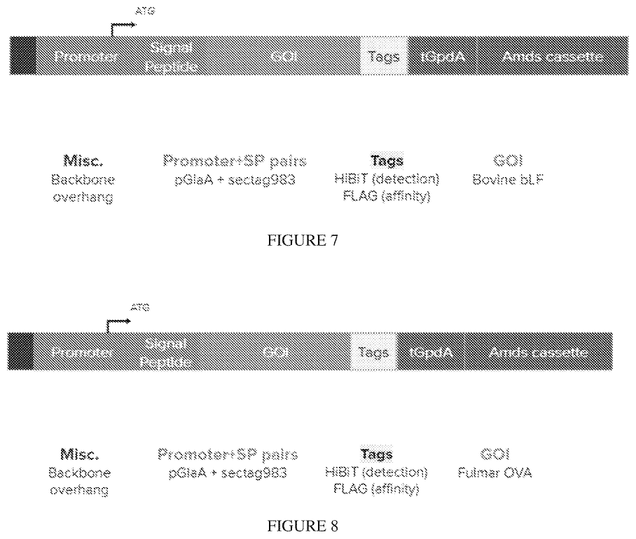

Aspergillus strains were constructed to test the extent to which the secretion signals identified in Example 2 as promoting bLF section in Pichia also promote protein secretion in other species. As in Examples 2, bovine lactoferrin (bLF, UniProt P24627) was selected as an exemplary protein for testing (FIG. 7).

An Aspergillus niger strain engineered for low viscosity was used as a host strain. A linear DNA containing an expression cassette including a HiBiT tagged, codon-optimized nucleotide sequence of lactoferrin (bLF, UniProt P24627), along with an amdS expression cassette, was introduced into the initial strain by random integration (as described below). The bLF expression cassette included the A. niger GlaA promoter and GpdA terminator (both linked to the sequence encoding bLF). Secretion signal 983 was used to replace bLF's original secretion tag (the first 19 amino acids of native bLF sequence, i.e. MKLFVPALLSLGALGLCLA (SEQ ID NO: 13). For expression in Aspergillus niger, the secretion signal contained a signal peptidase recognition site of KR at its C-terminal end.

To introduce the expression cassettes, a starter culture was generated by inoculating strain spores into a shake flask with liquid fermentation complete medium (CM) (see e.g., Barratt et al., 1965). The shake flasks were incubated at 35° C., 300 rpm at 80% humidity for approximately 72 hours. Mycelium was then harvested by filtering over miracloth. To generate protoplasts, semi-dried mycelium was transferred into a lysing enzyme and CaCl₂)/NaCl buffer and incubated for 1 hour at 35° C., 80 rpm. Approximately, 5 ug of linearized DNA was added to 100 ml of protoplast and incubated for 30 minutes, followed by the addition of PEG4000-Tris-CaCl₂) buffer and incubated for 30 minutes. The mixture was centrifuged to pellet the cells, then washed and resuspended with Sorbitol-Tris-CaCl₂ buffer followed by plating on solid minimal media (see e.g., Pontecorvo et al., 1953) with acetamide, as the sole Nitrogen source, and CsCl₂ added (see e.g., Carvallho et al., 2011). The transformation plates were incubated for 5 to 12 days at 35° C. until transformants became visible. The transformants were then picked and inoculated into pre-culture media containing corn steep liquor (CSL) and cultured at 35° C., 1000 rpm, 80% humidity for ˜48 hours. The preculture was stamped onto sporulation media (e.g, complete media with sorbitol) and incubated at 35° C. until strains sporulated. Spores were collected for screening.

### Screening

To perform a primary screen on the generated strains, the aforementioned CSL pre-culture after the ˜48 hours of incubation was stamped into fermentation complete medium (CM) in deepwell plates and incubated at 35° C., 1000 rpm, 80% humidity for 48 hours. DWP strain performance was measured using luminescence readout of the supernatant using Promega's Nano-Glo® HiBiT Lytic Detection System and protocol, or a variant thereof.

Spores from strains that were advanced to a purification step were struck out onto solid minimal media (see e.g., Pontecorvo et al., 1953) with acetamide as the sole nitrogen source, and CsCl₂ was added (see e.g., Carvallho et al., 2011). After 5 to 12 days, transformants became visible and were picked into pre-culture media containing corn steep liquor (CSL) and cultured at 35° C., 1000 rpm, 80% humidity for ˜48 hours. Cryostocking and a luminescence assay were repeated as described above. Fresh spores from strains that were advanced to lab scale fermentation were used to inoculate the shake flask pre-culture that were incubated at 30° C. and 220 rpm for 72 hours. These seed shake flasks were used to inoculate the lab scale fermenters. The bioreactor operated continuously while maintaining constant pH, temperature and dissolved oxygen levels. Please refer to “Culturing Host Cells” above. After 48 hours of fermentation, samples were collected. Sample analysis was performed according to steps described in Example 5. The chemiluminescence imaging of the HiBiT blot for these samples demonstrated an absence of visible signal in the negative control (supernatant from the host strain), presence of visible signal in the positive control (a diluted sample of Promega HiBiT Control Protein), and presence of visible signal for the experimental sample visualized at the expected secreted length of the heterologous bLF construct. Exemplary titers of more than 5 mg/L bLF were achieved. These experiments demonstrated that sectag983 is capable of promoting secretion of bLF in Aspergillus.

### Example 7: Expression of OVA in Aspergillus

### Strain Construction

Aspergillus strains were constructed to test the extent to which the functional secretion signals identified in Example 2 also promote secretion of ovalbumin in Aspergillus (FIG. 8).

An Aspergillus niger strain engineered for low viscosity was used as the host strain. Into this strain linear DNA containing HiBiT tagged, codon-optimized nucleotide sequence of fulmar ovalbumin (OVA, NCBI XP_009580141.1), along with an amdS expression cassette was introduced by random integration. The OVA gene was expressed under A. niger GlaA promoter and GpdA terminator. Sectag983 was used to replace the start methionine amino acid of the original ovalbumin sequences. Each secretion signal contains a signal peptidase recognition site of KR at its C-terminal. Expression cassettes were introduced as described above, in Example 6.

### DWP HiBiT Screening

Transformants were generated and underwent a primary screening as described above in Example 6, achieving titers of up to 1 mg/L in deepwell plates. These experiments demonstrated that sectag983 is capable of promoting secretion of ovalbumin in Aspergillus.

### Example 8: Expression of bLG in Pichia

### Strain Construction

Pichia pastoris strains are constructed to confirm that the secretion signals identified in Example 2 promote secretion of additional proteins, such as lactoglobulin.

Pichia pastoris strain Y-7556 is the initial strain. Into this strain, linear DNA containing HiBiT tagged, codon-optimized nucleotide sequence of Horse beta-lactoglobulin (bLG, UniProt P02758), or Reindeer bLG (UniProt Q00P86), or Subantarctic Fur Seal bLG (UniProt W5QN41), or Cow bLG (UniProt B5B0D4), or Goat bLG (UniProt P02756), or Sheep bLG (UniProt P67976), along with zeocin-resistance expression cassettes is introduced by ends-out recombination at a chromosomal genetic locus. The bLG gene is expressed under a synthetic (thiamine sensitive) promoter, or a synthetic (glucose sensitive) promoter and the Pichia pastoris native Dihydroxyacetone synthase (DHAS) terminator. Sectag909, sectag923, and sectag983 are used to replace the native secretion signal (the first 18 amino acids of native Horse bLG sequence, i.e., MKCLLLALGLALMCGIQA (SEQ ID NO: 215), the first 18 amino acids of native Reindeer bLG sequence, i.e., MKCLLITLGLALACGAQA (SEQ ID NO: 216), the first 16 amino acids of native Cow bLG sequence, i.e., MKCLLLALALTCGAQA (SEQ ID NO: 217), the first 18 amino acids of native Sheep bLG sequence, i.e., MKCLLLALGLALACGVQA (SEQ ID NO: 218), the first 18 amino acids of native Goat bLG sequence, i.e., MKCLLLALGLALACGIQA (SEQ (ID NO: 219), the first 18 amino acids of native Subantarctic Fur Seal bLG sequence, i.e., MRCLLLALGLALVCGIQA, SEQ ID NO: 220). Each secretion signal contains a signal peptidase recognition site of KREAEA (SEQ ID NO: 105) at its C-terminal end.

Different linear DNA sequences, each containing sectag909, sectag923, or sectag983 linked to bLG sequence, is transformed individually into the Initial Strain using PEG/LiAC/ssDNA transformation method and plated on YPD+Zeocin agar plates. Twenty-four clones are picked from each transformation plate for screening, grown in YPD culture until saturated, mixed with 40% Glycerol solution at equal volume, and stored at −80° C.

### Screening

Strains will be screened using the 384-well plate HiBiT screening as described above in Example 5.

### EQUIVALENTS

Those skilled in the art will recognize, or be able to ascertain using no more than routine experimentation, many equivalents to the specific embodiments of the invention described here. Such equivalents are intended to be encompassed by the following claims.

We claim:

1. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises, from N-terminus to C-terminus:
a) a pre-sequence comprising the structure of M-A′-Q-B′-L-C′-L-D′-LL-E′ (SEQ ID NO: 173), wherein M is methionine, Q is glutamine, and L is leucine, and A′ is 0-4 amino acids in length, B′ is 0-2 amino acids in length, C′ is 1-6 amino acids in length, D′ is 4 amino acids in length, and E′ is 5 amino acids in length, wherein any amino acid of A′, B′, C′, D′ and E′ is any amino acid; and
b) a pro-sequence comprising an amino acid sequence having at least 80% identity to the amino acid sequence:
(SEQ ID NO: 61)
APANTTTEDETAQIPAEAVIDYSDLEGDFDAAALPLSNSTNNGLSSTNTT
IASIAAKEEGVSLDKR.

2. The host cell of claim 1, wherein the polypeptide comprises the secretion signal, and wherein A′ is 0 or 4 amino acid amino acids in length.

3. The host cell of claim 1 or claim 2, wherein the polypeptide comprises the secretion signal, and wherein C′ is 1, 2 or 6 amino acids in length.

4. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises, from N-terminus to C-terminus:
a) a pre-sequence comprising the amino acid sequence LXXXXLL (SEQ ID NO: 15), wherein X is chosen from any amino acid; and
b) a pro-sequence comprising an amino acid sequence having at least 80% identity to the amino acid sequence:
(SEQ ID NO: 61)
APANTTTEDETAQIPAEAVIDYSDLEGDFDAAALPLSNSTNNGLSSTNTT
IASIAAKEEGVSLDKR.

5. The host cell of claim 4, wherein the polypeptide comprises the secretion signal, and wherein no more than 10 amino acids separate the amino acid sequence LXXXXLL (SEQ ID NO: 15) of the pre-sequence from the amino acid sequence having at least 80% identity to the amino acid sequence APANTTTEDETAQIPAEAVIDYSDLEGDFDAAALPLSNSTNNGLSSTNTTIASIAAKEE GVSLDKR (SEQ ID NO: 61) of the pro-sequence.

6. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises, from N-terminus to C-terminus:
a) a pre-sequence comprising the amino acid sequence LXXXXLL (SEQ ID NO: 15), wherein X is chosen from any amino acid; and
b) a pro-sequence comprising the amino acid sequence: APX³NX⁵TX⁷EX⁹EX¹¹X¹²QX¹⁴PAEAX¹⁹X²⁰X²¹YX²³X²⁴X²⁵EGDX²⁹DX³¹AX³³LPX³⁶X³⁷X³⁸STNX⁴²GX⁴⁴X⁴⁵X⁴⁶X⁴⁷NTTX⁵¹ASIAAKEEGVSLDKR (SEQ ID NO: 82), wherein: X³ is valine (V) or alanine (A); X⁵ is threonine (T) or alanine (A); X⁷ is threonine (T) or alanine (A); X⁹ is aspartic acid (D) or glycine (G); X¹¹ is threonine (T) or alanine (A); X¹² is threonine (T) or alanine (A); X¹⁴ is isoleucine (I) or threonine (T); X¹⁹ is valine (V) or alanine (A); X²⁰ is isoleucine (I) or alanine (A); X²¹ is glycine (G), aspartic acid (D), or threonine (T); X²³ is leucine (L), serine (S), or arginine I; X²⁴ is aspartic acid (D) or glycine (G); X²⁵ is leucine (L) or serine (S); X²⁹ is phenylalanine (F), serine (S), or valine (V); X³¹ is valine (V) or alanine (A); X³³ is valine (V) or alanine (A); X³⁶ is phenylalanine (F) or leucine (L); X³⁷ serine (S) or proline (P); X³⁸ is asparagine (N), serine (S), or aspartic acid (D); X⁴² is asparagine (N) or aspartic acid (D); X⁴⁴ is leucine (L) or serine (S); X⁴⁵ is leucine (L) or serine (S); X⁴⁶ is phenylalanine (F) or serine (S); X⁴⁷ is isoleucine (I) or threonine (T); and X⁵¹ is isoleucine (I) or threonine (T).

7. The host cell of claim 6, wherein the polypeptide comprises the secretion signal, and wherein no more than 10 amino acids separate the amino acid sequence LXXXXLL (SEQ ID NO: 15) of the pre-sequence from the amino acid sequence APX³NX⁵TX⁷EX⁹EX¹¹X¹²QX¹⁴PAEAX¹⁹X²⁰X²¹YX²³X²⁴X²⁵EGDX²⁹DX³¹AX³³LPX³⁶X³⁷X³⁸STNX⁴²GX⁴⁴X⁴⁵X⁴⁶X⁴⁷NTTX⁵¹ASIAAKEEGVSLDKR (SEQ ID NO: 82) of the pro-sequence.

8. The host cell of any one of claims 1-7, wherein the polypeptide comprises the secretion signal, and wherein the pre-sequence comprises a portion that is ten or more amino acids in length and that comprises the amino acid sequence LXXXXLL (SEQ ID NO: 15), and wherein at least five of the amino acids of the portion are selected from the group consisting of leucine (L) and isoleucine (I).

9. The host cell of any one of claims 1-8, wherein the polypeptide comprises the secretion signal, and wherein the pre-sequence comprises the amino acid sequence LX²X³X⁴X⁵LLX⁸X⁹X¹⁰X¹¹X¹² (SEQ ID NO: 17), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acI (E), or tyrosine (Y); and X¹² is alanine (A) or cytosine (C).

10. The host cell of any one of claims 1-9, wherein the polypeptide comprises the secretion signal, and wherein the pre-sequence comprises an amino acid sequence having at least 80% identity to one or more of: the amino acid sequence MTKPTQVLVRSVSILFFITLLHLVVA (SEQ ID NO: 1); the amino acid sequence MQLYLTLLFLLSFVEC (SEQ ID NO: 9); and the amino acid sequence
(SEQ ID NO: 2)
MQHFLSLLLAVSLLTTTYA.

11. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises, from N-terminus to C-terminus:
a) a pre-sequence comprising an amino acid sequence having at least 80% identity to one or more of: the amino acid sequence MTKPTQVLVRSVSILFFITLLHLVVA (SEQ ID NO: 1); the amino acid sequence MQLYLTLLFLLSFVEC (SEQ ID NO: 9); and the amino acid sequence MQHFLSLLLAVSLLTTTYA (SEQ ID NO: 2); and
b) a pro-sequence comprising an amino acid sequence having at least 80% identity to the amino acid sequence:
(SEQ ID NO: 61)
APANTTTEDETAQIPAEAVIDYSDLEGDFDAAALPLSNSTNNGLSSTNTT
IASIAAKEEGVSLDKR.

12. The host cell of claim 11, wherein the polypeptide comprises the secretion signal, and wherein no more than 10 amino acids separate the amino acid sequence having at least 80% identity to one or more of the amino acid sequence MTKPTQVLVRSVSILFFITLLHLVVA (SEQ ID NO: 1), the amino acid sequence MQLYLTLLFLLSFVEC (SEQ ID NO: 9), and the amino acid sequence MQHFLSLLLAVSLLTTTYA (SEQ ID NO: 2) of the pre-sequence from the amino acid sequence having at least 80% identity to the amino acid sequence APANTTTEDETAQIPAEAVIDYSDLEGDFDAAALPLSNSTNNGLSSTNTTIASIAAKEE GVSLDKR (SEQ ID NO: 61) of the pro-sequence.

13. The host cell of any one of claims 1-12, wherein the polypeptide comprises the secretion signal, and wherein the pre-sequence comprises the amino acid sequence of:
(SEQ ID NO: 1)
MTKPTQVLVRSVSILFFITLLHLVVA;
(SEQ ID NO: 9)
MQLYLTLLFLLSFVEC;
or
(SEQ ID NO: 2)
MQHFLSLLLAVSLLTTTYA.

14. The host cell of any one of claims 1-13, wherein the polypeptide comprises the secretion signal, and wherein the pro-sequence does not comprise the amino acid sequence of
(SEQ ID NO: 59)
APVNTTTEDETAQIPAEAVIGYLDLEGDFDVAVLPFSNSTNNGLLFINTT
IASIAAKEEGVSLDKR.

15. The host cell of any one of claims 1-14, wherein the polypeptide comprises the secretion signal, and wherein the pro-sequence comprises the amino acid sequence:
(SEQ ID NO: 61)
APANTTTEDETAQIPAEAVIDYSDLEGDFDAAALPLSNSTNNGLSSTNTT
IASIAAKEEGVSLDKR.

16. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises the amino acid sequence LX²X³X⁴X⁵LLX⁸X⁹X¹⁰X¹¹X¹²APX¹⁵NX¹⁷TX¹⁹EX²¹EX²³X²⁴QX²⁶PAEAX³¹X³²X³³YX³⁵X³⁶X³⁷EGDX⁴¹DX⁴³AX⁴⁵LPX⁴⁸X⁴⁹X⁵⁰STNX⁵⁴GX⁵⁶X⁵⁷X⁵⁸X⁵⁹NTTX⁶³ASIAAKEEGVSLDKR (SEQ ID NO: 153), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamiclid (E), or tyrosine (Y); X¹² is alanine (A) or cytosine (C); X¹⁵ is valine (V) or alanine (A); X¹⁷ is threonine (T) or alanine (A); X¹⁹ is threonine (T) or alanine (A); X²¹ is aspartic acid (D) or glycine (G); X²³ is threonine (T) or alanine (A); X²⁴ is threonine (T) or alanine (A); X²⁶ is isoleucine (I) or threonine (T); X³¹ is valine (V) or alanine (A); X³² is isoleucine (I) or alanine (A); X³³ is glycine (G), aspartic acid (D), or threonine (T); X³⁵ is leucine (L), serine (S), or Iinine (R); X³⁶ is aspartic acid (D) or glycine (G); X³⁷ is leucine (L) or serine (S); X⁴¹ is phenylalanine (F), serine (S), or valine (V); X⁴³ is valine (V) or alanine (A); X⁴⁵ is valine (V) or alanine (A); X⁴⁸ is phenylalanine (F) or leucine (L); X⁴⁹ serine (S) or proline (P); X⁵⁰ is asparagine (N), serine (S), or aspartic acid (D); X⁵⁴ is asparagine (N) or aspartic acid (D); X⁵⁶ is leucine (L) or serine (S); X⁵⁷ is leucine (L) or serine (S); X⁵⁸ is phenylalanine (F) or serine (S); X⁵⁹ is isoleucine (I) or threonine (T); and X⁶³ is isoleucine (I) or threonine (T).

17. The host cell of claim 16, wherein the polypeptide comprises the secretion signal, and wherein the secretion signal does not comprise the amino acid sequence of
(SEQ ID NO: 59)
APVNTTTEDETAQIPAEAVIGYLDLEGDFDVAVLPFSNSTNNGLLFINTT
IASIAAKEEGVSLDKR.

18. The host cell of claim 15 or claim 16, wherein the polypeptide comprises the secretion signal, and wherein the secretion signal comprises the amino acid sequence LX²X³X⁴X⁵LLX⁸X⁹X¹⁰X¹¹X¹²APANTTTEDETAQIPAEAVIDYSDLEGDFDAAALPLSNS TNNGLSSTNTTIASIAAKEEGVSLDKR (SEQ ID NO: 157), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glImic acid (E), or tyrosine (Y); and X¹² is alanine (A) or cytosine (C).

19. The host cell of any one of claims 16-18, wherein the polypeptide comprises the secretion signal, and wherein the secretion signal further comprises the amino acid sequence:
(SEQ ID NO: 154)
MTKPTQVLVRSVSI;
(SEQ ID NO: 155)
MQLY;
or
(SEQ ID NO: 156)
MQHFLSL.

20. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises an amino acid sequence having at least 80% identity to the one or more of:
(SEQ ID NO: 107)
MTKPTQVLVRSVSILFFITLLHLVVAAPANTTTEDETAQIPAEAVIDYSD
LEGDFDAAALPLSNSTNNGLSSTNTTIASIAAKEEGVSLDKR;
(SEQ ID NO: 108)
MQLYLTLLFLLSFVECAPANTTTEDETAQIPAEAVIDYSDLEGDFDAAAL
PLSNSTNNGLSSTNTTIASIAAKEEGVSLDKR;
and
(SEQ ID NO: 115)
MQHFLSLLLAVSLLTTTYAAPANTTTEDETAQIPAEAVIDYSDLEGDFDA
AALPLSNSTNNGLSSTNTTIASIAAKEEGVSLDKR.

21. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises the amino acid sequence of:
(SEQ ID NO: 107)
MTKPTQVLVRSVSILFFITLLHLVVAAPANTTTEDETAQIPAEAVIDYSD
LEGDFDAAALPLSNSTNNGLSSTNTTIASIAAKEEGVSLDKR;
(SEQ ID NO: 108)
MQLYLTLLFLLSFVECAPANTTTEDETAQIPAEAVIDYSDLEGDFDAAAL
PLSNSTNNGLSSTNTTIASIAAKEEGVSLDKR;
or
(SEQ ID NO: 115)
MQHFLSLLLAVSLLTTTYAAPANTTTEDETAQIPAEAVIDYSDLEGDFDA
AALPLSNSTNNGLSSTNTTIASIAAKEEGVSLDKR.

22. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises, from N-terminus to C-terminus: a pre-sequence from a first species (or derived from a pre-sequence from a first species) and a pro-sequence from a second species (or derived from a pro-sequence from a second species), wherein:
a) the pre-sequence comprises the amino acid sequence of: WFSWIVG (SEQ ID NO: 18); MRFPSIFTAVLF (SEQ ID NO: 19); SSALA (SEQ ID NO: 20); IVGLF (SEQ ID NO: 21); MTKPTQVLV (SEQ ID NO: 22); MKLATAFTILTA (SEQ ID NO: 23); ETPRASLSLGRW (SEQ ID NO: 24); WHAVMVFVLCG (SEQ ID NO: 25); MRFPSIFT (SEQ ID NO: 222); MKX³X⁴X⁵X⁶AX⁸LSX¹¹X¹²X¹³LX¹⁴L (SEQ ID NO: 26), wherein X³ is phenylalanine (F) or leucine (L), X⁴ is serine (S) or phenylalanine (F), X⁵ is alanine (A) or valine (V), X⁶ is glycine (G) or proline (P), X⁸ is valine (V) or leucine (L), X¹¹ is tryptophan (W) or leucine (L), X¹² is serine (S) or glycine (G), X¹³ is serine (S) or alanine (A), and X¹⁴ is leucine (L) or glycine (G); or MX²X³X⁴X⁵ (SEQ ID NO: 223), wherein I is arginine (R) or glutamine (Q), X³ is histidine (H) or glutamine (Q), X⁴ is valine (V) or phenylalanine (F), and X⁵ is leucine (L) or tryptophan (W);
b) the pro-sequence comprises the amino acid sequence of: RYVVGDDEQ (SEQ ID NO: 64); IVAKSGI (SEQ ID NO: 65); IPDEAIAN (SEQ ID NO: 66); QTSISDDEEPIVVEINGQKV (SEQ ID NO: 67); INTTLTEEALEKSGISIDDL (SEQ ID NO: 68); PVFAEIDNK (SEQ ID NO: 69); DDLKESYAN (SEQ ID NO: 70); PVENVDD (SEQ ID NO: 71); IDQEQLTNG (SEQ ID NO: 72); PVDSGAKGKYSR (SEQ ID NO: 73); NDGVGVGMSTIKEEDFGKHF (SEQ ID NO: 74); TTIASIA (SEQ ID NO: 224); YVVGDDEQ (SEQ ID NO: 225); PVFAEIDNKPVVYIVNTTKA (SEQ ID NO: 226); ESIVAKSGITLDDLKESYAN (SEQ ID NO: 227); NTTIX⁵X⁶X⁷A (SEQ ID NO: 63), wherein X⁵ is alanine (A), leucine (L), or tyrosine (Y), X⁶ is alanine (A), serine (S), asparagine (N), I glutamic acid (E), and X⁷ is alanine (A), isoleucine (I), serinIS), glutamic acid (E), or glutamine (Q); AAX³EEGX⁷SLDKR (SEQ ID NO: 221), wherein X³ is lysine (K) or alanine (A), and X⁷ is valine (V) or serine (S); X¹NTTIAX⁷X⁸AX¹⁰X¹¹EEGVX¹⁶ (SEQ ID NO: 75), wherein X¹ is valine (V) or isoleucine (I), X⁷ is aspartic acid (D), serinIS), or glutamic acid (E), X⁸ is isoleucine (I) or glutamine (Q), X¹⁰ is alanine (A) or leucine (L), X¹¹ is alanine (A) or lysine (K), and X¹⁶ is serine (S) or leucine (L); X¹X²X³X⁴X⁵DDEX⁹ (SEQ ID NO: 76), wherein X¹ is arginine (R) or glutamine (Q), X² is tyrosine (Y) or threonine (T), X³ is valine (V) or serine (S), X⁴ is valine (V) or isoleucine (I), X⁵ is glycine (G) or serine (S), and X⁹ is glutlne (Q) or glutamic acid (E); AX²LPFSNX⁸TNX¹¹GX¹³X¹⁴FX¹⁶NTTI (SEQ ID NO: 77), wherein X² is valine (V) or leucine (L), X⁸ is serine (S) or glycine (G), X¹¹ is asparagine (N) or threonine (T), X¹³ is isoleucine (I) or leucine (L), X¹⁴ is serine (S), leucine (L), or methionine (M), and X¹⁶ is valine (V) or isoleucine (I); X¹AQX⁴PAEAX⁹IGX¹²LDLX¹⁶X¹⁷X¹⁸X¹⁹D (SEQ ID NO: 78), wherein X¹ is threonine (T) or serine (S), X⁴ is isoleucine (I) or valine (V), X⁹ is valine (V) or isoleucine (I), X¹² is tyrosine (Y) or phenylalanI (F), X¹⁶ is glutamic acid (E) or threonine (T), X¹⁷ is aspartic acid (D) or glycine (G), X¹⁸ is aspartic acid (D), serine (S), or alaniI (A), and X¹⁹ is glutamic acid (E) or phenylalanine (F); X¹GX³X⁴X⁵X⁶X⁷DX⁹IX¹¹P (SEQ ID NO: 79), wherein X¹ is lysine (K) or serinIS), X³ is lysine (K) or arginine (R), X⁴ is tyrosine (Y) or phenylalanine (F), X⁵ is serI (S) or leucineI), X⁶ is arginine (R) or glutamic acid (E), X⁷ is glutamine (Q) or threonine (T), X⁹ is leucine (L) or isoleucine (I), and X¹¹ is isoleucine (I) or phenylalanine (F); X¹X²NX⁴TX⁶E (SEQ ID NO: 80), wherein X¹ is asparagine (N) or proline (P), X² is glycine (G) or alanine (A), X⁴ is glycine (G) or threonine (T), and X⁶ is serine (S) or threonine (T); PAEAVIX⁷Y (SEQ ID NO: 228), wherein X⁷ is aspartic acid (D) or glycine (G); KEEX⁴X⁵X⁶X⁷X⁸KR (SEQ ID NO: 229) wherein X⁴ is glycine (G) or glutamic acid (E), X⁵ is valine (V) or alanine (A), X⁶ is serine (S) or lysine (K), X⁷ is leucine (L) or asparagine (N), and X⁸ is aspartic acid (D) or glycine (G); GDFDX⁵AX⁷LP (SEQ ID NO: 230), wherein X⁵ is valine (V) or alanine (A), and X⁷ is valine (V) or alanine (A); X¹SNST (SEQ ID NO: 231), wherein X¹ is leucine (L) or phenylalanine (F); GLSX⁴TN (SEQ ID NO: 232), wherein X⁴ is serine (S) or phenylalanine (F); PX²SNSTNNGLSX¹²TNTTIASI (SEQ ID NO: 233), wherein X² is leucine (L) or phenylalanine (F), and X¹² is serine (S) or phenylalanine (F); or X¹X²X³IPX⁶EAX⁹X¹⁰X¹¹X¹²X¹³X¹⁴X¹⁵X¹⁶X¹⁷DX¹⁹X²⁰ (SEQ ID NO: 234), wherein X¹ is threonine (T) or aspartic acid (D), X² is alanine (A) or leucine (L), X³ is glutamine (Q) or isoleucine (I), X⁶ is alanine (A) or aspartic acid (D), X⁹ is valine (V) or isoleucine (I), X¹⁰ is isoleucine (I) or alanine (A), X¹¹ is aspartic acid (D), glycine (G), or Iaragine (N), X¹² is tyrosine (Y) or arginine (R), X¹³ serine (S) or tyrosine (Y), X¹⁴ is aspartic acid (D) or valine (V); X¹⁵ Ileucine (L) or valine (V), X¹⁶ is glutamic acid (E) or glycine (G), X¹⁷ is glycine (G) or aspartiIcid (D), X¹⁹ is phenylalanine (F) or glutamic acid (E), and X²⁰ is aspartic acid (D) or glutamine (Q); or
c) a combination thereof.

23. A host cell comprising an expression system for expressing a polypeptide, wherein the expression system comprises a polynucleotide encoding, from 5′ to 3′: a promoter; a nucleic acid sequence encoding the polypeptide; and a transcriptional terminator; which are operably linked to each other; wherein: (A) the host cell comprises one or more genetic modifications that result in the overexpression of a gene encoding a calreticulin (CRT) protein and/or a protein disulfide isomerase family A member 3 (PDIA3) protein; and/or (B) the polypeptide comprises a secretion signal, wherein the secretion signal comprises, from N-terminus to C-terminus: a pre-sequence from a first species (or derived from a pre-sequence from a first species) and a pro-sequence from a second species (or derived from a pro-sequence from a second species), wherein the secretion signal comprises the amino acid sequence: CX²X³X⁴X⁵X⁶X⁷X⁸APX¹¹NTTT (SEQ ID NO: 146), wherein X² is leucine (L), phenylalanine (F), or glycine (G), X³ is leucine (L), phenylalanine (F), or valine (V), X⁴ is asparagine (N) or valine (V), X⁵ is valine (V) or leucine (L), X⁶ is serine (S), alanine (A), or valine (V), X⁷ is serine (S) or alanine (A), X⁸ is alanine (A) or glycine (G), and X¹¹ is valine (V) or alanine (A); X¹AAPX⁵X⁶TTTEDE (SEQ ID NO: 147), wherein X¹ is leucine (L), serine (S), or alanine (A), X⁵ is alanine (A) or valine (V), and X⁶ is asparagine (N) or serine (S); AAPIX⁵X⁶X⁷X⁸S (SEQ ID NO: 148), wherein X⁵ is asparagine (N) or lysine (K), X⁶ is isoleucine (I) or phenylalanine (F), X⁷ is threonine (T) or asparagine (N), and X⁸ is serine (S) or aspartic acid (D); X¹X²X³X⁴X⁵X⁶X⁷X⁸X⁹ (SEQ ID NO: 149), wherein X¹ is glutamine (Q) or asparagine (N), X² is valine (V) or histidine (H), X³ is tryptophan (W) or phenylalanine (F), X⁴ is phenylalanine (F), leucine (L), or histidine (H), X⁵ is serine (S) or alanine (A), X⁶ is tryptophan (W), leucine (L), or valine (V), X⁷ is isoleucine (I), leucine (L), or methionine (M), X⁸ is valine (V) or leucine (L), and X⁹ is glycine (G), alanine (A), or phenylalanine (F); X¹X²X³X⁴X⁵X⁶AX⁸X⁹ (SEQ ID NO: 150), wherein X¹ lysine (K) or asparagine (N), X² is glycine (G), asparagine (N), or aspartic acid (D), X³ is asparagine (N), glycine (G), or lysine (K), X⁴ is leucine (L), tyrosine (Y), or glycine (G), X⁵ is line (S) or asparagine (N), X⁶ is serine (S), arginine (R), or glycine (G), X⁸ is asparagine (N), aspartic acid (D), or serine I, and X⁹ is threonine (T), leucine (L), or glutamic acid (E); X¹RX³X⁴X⁵X⁶X⁷X⁸X⁹X¹⁰X¹¹X¹²X¹³X¹⁴X¹⁵X¹⁶ (SEQ ID NO: 151), wherein X¹ is methionine (M), valine (V), or glutamine (Q), X³ is phenylalanine (F) or glutamine (Q), X⁴ is leucine (L) or valine (V), X⁵ is serine (S) or tryptophan (W), X⁶ is phenylalanine (F) or leucine (L), X⁷ is leucine (L) or serine (S), X⁸ is threonine (T), leucine (L), phenylalanine (F), or tryptophan (W), X⁹ is alanine (A), leucine (L), or isoleucine (I), X¹⁰ is valine (V) or leucine (L), X¹¹ is leucine (L), glycine (G), or serine (S), X¹² is leucine (L) or phenylalanine (F), X¹³ is valine (V), leucine (L), or phenylalanine (F), X¹⁴ is valine (V) or leucine (L), X¹⁵ is serine (S) or cytosine (C), and X¹⁶ is alanine (A) or phenylalanine (F); X¹X²X³X⁴X⁵X⁶X⁷X⁸X⁹X¹⁰X¹¹X¹²X¹³X¹⁴X¹⁵X¹⁶X¹⁷IX¹⁹X²⁰ (SEQ ID NO: 152), wherein X¹ is aspartic acid (D), valine (V), or glutamic acid (E), X² is valine (V), tyrosine (Y), or proline (P), X³ is proline (P), isoleucine (I), or serine (S), X⁴ is glycine (GIr valine (V), X⁵ is threonine (T), asparagine (N), or arginine (R), X⁶ is serine (S), threonine (T), or phenylalanine (F), X⁷ is glutamine (Q), threonine I, or leucine (L), X⁸ is glycine (G), lysine (K), or glutamic acid (E) I⁹ is valine (V), alanine (A), or glutamine (Q), X¹⁰ is glutamic acid (E) or aspartic acid (D), X¹¹ is phenylalanine (F), serine (S), or isoleucine (I), X¹² is isoleucine (I) or proline (P), X¹³ is phenylalanine (F) or valine (V), X¹⁴ is alanine (AIr proline (P), X¹⁵ is lysine (K) or glutamine (Q), X¹⁶ is glutamic acid (E), serine (S), or glutamine (Q), X¹⁷ is alanine (A) or glycine (G), X¹⁹ Iisoleucine (I), threonine (T), or asparagine (N), and X²⁰ is glutamic acid (E), leucine (L), or alanine (A); AAPX⁴X⁵X⁶X⁷X⁸X⁹X¹⁰X¹¹X¹² (SEQ ID NO: 235), wherein X⁴ is alanine (A) or valine (V), X⁵ is asparagine (N) or aspartic acid (D), X⁶ is serine (S) or threonine (T), X⁷ is threoIe (T) or glycine (G), X⁸ is threonine (T) or alanine (A), X⁹ is glutamic acI (E) or lysine (K), X¹⁰ is glycine (G) or aspartic acid (D), X¹¹ is glutamic acid (E) or lysine (K), and X¹² is threonine (T) or tyrosine (Y); AX²KEEX⁶X⁷X⁸X⁹X¹⁰KREAEA (SEQ ID NO: 236), wherein X² is alanine (A) or threonine (T), X⁶ is glycine (G) or glutamic acid (E), X⁷ is valine (V) or alanine (A); X⁸ is serine (S) or lysine (K), X⁹ is leucine (L) or asparagine (N), and X¹⁰ is aspartic acid (D) or glycine (G); SLLX⁴X⁵SX7X⁸LAAPX¹³NTTTEDE (SEQ ID NO: 237), wherein X⁴ is alanine (A), phenylalanine (F), leucine (L), or serine (S), X⁵ is leucine (L) or alanine (A), X⁷ is leucine (L) or serine (S), X⁸ is leucine (L) or valine (V), and X¹³ is alanine (A) or valine (V); or IX³X⁴X⁵X⁶X⁷X⁸X⁹ (SEQ ID NO: 238), wherein X² iIlanine (A), lysine (K), or arginine (R), X³ is leucine (L), glutamine (Q), or arginine (R), X⁴ is phenylalanine (F) or valine (V), X⁵ is valine (V) or tryptophan (W), X⁶ is alanine (A), phenylalanine (F), or proline (P), X⁷ is leucine (L), alanine (A), or serine (S), X⁸ is leucine (L), valine (V), or tryptophan (W), and X⁹ is leucine (L) or isoleucine (I).

24. The host cell of any one of claims 1-23, wherein the polypeptide comprises a secretion signal, and wherein the secretion signal further comprises a C-terminal cleavage sequence.

25. The host cell of claim 24, wherein the C-terminal cleavage sequence comprises the amino acid sequence of EAEA (SEQ ID NO: 104), KR, or a combination thereof.

26. The host cell of any one of claims 1-25, wherein the polypeptide comprises a secretion signal, and wherein the polypeptide further comprises the amino acid sequence of a protein of interest, wherein the amino acid sequence of the protein of interest is positioned C-terminal to the amino acid sequence of the secretion signal.

27. The host cell of claim 26, wherein the protein of interest is lactoferrin (LF), lactoglobulin (LG), or ovalbumin (Ova).

28. The host cell of claim 26, wherein the protein of interest is bovine lactoferrin (bLF), bovine lactoglobulin (bLG), or ovalbumin (Ova).

29. The host cell of claim 27, wherein the protein of interest is lactoferrin (LF), and wherein the nucleic acid sequence of polypeptide is operably linked to a constitutive promoter.

30. The host cell of claim 29, wherein the constitutive promoter comprises a GAP promoter.

31. A polypeptide comprising a secretion signal, wherein the secretion signal comprises, from N-terminus to C-terminus:
a) a pre-sequence comprising the structure of M-A′-Q-B′-L-C′-L-D′-LL-E′ (SEQ ID NO: 173), Where M is methionine, Q is glutamine, and L is leucine, and A′ is 0-4 amino acids in length, B′ is 0-2 amino acids in length, C′ is 1-6 amino acids in length, D′ is 4 amino acids in length, and E′ is 5 amino acids in length, wherein any amino acid of A′, B′, C′, D′ and E′ is any amino acid; and
b) a pro-sequence comprising an amino acid sequence having at least 80% identity to the amino acid sequence:
(SEQ ID NO: 61)
APANTTTEDETAQIPAEAVIDYSDLEGDFDAAALPLSNSTNNGLSSTNTT
IASIAAKEEGVSLDKR.

32. The polypeptide of claim 31, wherein A′ is 0 or 4 amino acid amino acids in length.

33. The polypeptide of claim 31 or claim 32, wherein C′ is 1, 2 or 6 amino acids in length.

34. A polypeptide comprising a secretion signal, wherein the secretion signal comprises, from N-terminus to C-terminus:
a) a pre-sequence comprising the amino acid sequence LXXXXLL (SEQ ID NO: 15), wherein X is chosen from any amino acid; and
b) a pro-sequence comprising an amino acid sequence having at least 80% identity to the amino acid sequence:
(SEQ ID NO: 61)
APANTTTEDETAQIPAEAVIDYSDLEGDFDAAALPLSNSTNNGLSSTNTT
IASIAAKEEGVSLDKR.

35. The polypeptide of claim 34, wherein no more than 10 amino acids separate the amino acid sequence LXXXXLL (SEQ ID NO: 15) of the pre-sequence from the amino acid sequence having at least 80% identity to the amino acid sequence APANTTTEDETAQIPAEAVIDYSDLEGDFDAAALPLSNSTNNGLSSTNTTIASIAAKEE GVSLDKR (SEQ ID NO: 61) of the pro-sequence.

36. A polypeptide comprising a secretion signal, wherein the secretion signal comprises, from N-terminus to C-terminus:
a) a pre-sequence comprising the amino acid sequence LXXXXLL (SEQ ID NO: 15), wherein X is chosen from any amino acid; and
b) a pro-sequence comprising the amino acid sequence: APX³NX⁵TX⁷EX⁹EX¹¹X¹²QX¹⁴PAEAX¹⁹X²⁰X²¹YX²³X²⁴X²⁵EGDX²⁹DX³¹AX³³LPX³⁶X³⁷X³⁸STNX⁴²GX⁴⁴X⁴⁵X⁴⁶X⁴⁷NTTX⁵¹ASIAAKEEGVSLDKR (SEQ ID NO: 82), wherein: X³ is valine (V) or alanine (A); X⁵ is threonine (T) or alanine (A); X⁷ is threonine (T) or alanine (A); X⁹ is aspartic acid (D) or glycine (G); X¹¹ is threonine (T) or alanine (A); X¹² is threonine (T) or alanine (A); X¹⁴ is isoleucine (I) or threonine (T); X¹⁹ is valine (V) or alanine (A); X²⁰ is isoleucine (I) or alanine (A); X²¹ is glycine (G), aspartic acid (D), or threonine (T); X²³ is leucine (L), serine (S), or arginine (R); X²⁴ is aspartic acid (D) or glycine (G); X²⁵ is leucine (L) or serine (S); X²⁹ is phenylalanine (F), serine (S), or valine (V); X³¹ is valine (V) or alanine (A); X³³ is valine (V) or alanine (A); X³⁶ is phenylalanine (F) or leucine (L); X³⁷ serine (S) or proline (P); X³⁸ is asparagine (N), serine (S), or aspartic acid (D); X⁴² is asparagine (N) or aspartic acid (D); X⁴ is leucine (L) or serine (S); X⁴⁵ is leucine (L) or serine (S); X⁴⁶ is phenylalanine (F) or serine (S); X⁴⁷ is isoleucine (I) or threonine (T); and X⁵¹ is isoleucine (I) or threonine (T).

37. The polypeptide of claim 36, wherein no more than 10 amino acids separate the amino acid sequence LXXXXLL (SEQ ID NO: 15) of the pre-sequence from the amino acid sequence APX³NX⁵TX⁷EX⁹EX¹¹X¹²QX¹⁴PAEAX¹⁹X²⁰X²¹YX²³X²⁴X²⁵EGDX²⁹DX³¹AX³³LPX³⁶X³⁷X³⁸STNX⁴²GX⁴⁴X⁴⁵X⁴⁶X⁴⁷NTTX⁵¹ASIAAKEEGVSLDKR (SEQ ID NO: 82) of the pro-sequence.

38. The polypeptide of any one of claims 31-37, wherein the pre-sequence comprises a portion that is ten or more amino acids in length and that comprises the amino acid sequence LXXXXLL (SEQ ID NO: 15), and wherein at least five of the amino acids of the portion are selected from the group consisting of leucine (L) and isoleucine (I).

39. The polypeptide of any one of claims 31-38, wherein the pre-sequence comprises the amino acid sequence LX²X³X⁴X⁵LLX⁸X⁹X¹⁰X¹¹X¹² (SEQ ID NO: 17), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); and X¹² is alanine (A) or cytosine (C).

40. The polypeptide of any one of claims 31-39, wherein the pre-sequence comprises an amino acid sequence having at least 80% identity to one or more of: the amino acid sequence MTKPTQVLVRSVSILFFITLLHLVVA (SEQ ID NO: 1); the amino acid sequence MQLYLTLLFLLSFVEC (SEQ ID NO: 9); and the amino acid sequence
(SEQ ID NO: 2)
MQHFLSLLLAVSLLTTTYA.

41. A polypeptide comprising a secretion signal, wherein the secretion signal comprises, from N-terminus to C-terminus:
a) a pre-sequence comprising an amino acid sequence having at least 80% identity to one or more of: the amino acid sequence MTKPTQVLVRSVSILFFITLLHLVVA (SEQ ID NO: 1); the amino acid sequence MQLYLTLLFLLSFVEC (SEQ ID NO: 9); and the amino acid sequence MQHFLSLLLAVSLLTTTYA (SEQ ID NO: 2); and
b) a pro-sequence comprising an amino acid sequence having at least 80% identity to the amino acid sequence:
(SEQ ID NO: 61)
APANTTTEDETAQIPAEAVIDYSDLEGDFDAAALPLSNSTNNGLSSTNTT
IASIAAKEEGVSLDKR.

42. The polypeptide of claim 41, wherein no more than 10 amino acids separate the amino acid sequence having at least 80% identity to one or more of the amino acid sequence MTKPTQVLVRSVSILFFITLLHLVVA (SEQ ID NO: 1), the amino acid sequence MQLYLTLLFLLSFVEC (SEQ ID NO: 9), and the amino acid sequence MQHFLSLLLAVSLLTTTYA (SEQ ID NO: 2) of the pre-sequence from the amino acid sequence having at least 80% identity to the amino acid sequence APANTTTEDETAQIPAEAVIDYSDLEGDFDAAALPLSNSTNNGLSSTNTTIASIAAKEE GVSLDKR (SEQ ID NO: 61) of the pro-sequence.

43. The polypeptide of any one of claims 31-42, wherein the pre-sequence comprises the amino acid sequence of: MTKPTQVLVRSVSILFFITLLHLVVA (SEQ ID NO: 1); MQLYLTLLFLLSFVEC (SEQ ID NO: 9); or MQHFLSLLLAVSLLTTTYA (SEQ ID NO: 2).

44. The polypeptide of any one of claims 31-43, wherein the pro-sequence does not comprise the amino acid sequence of
(SEQ ID NO: 59)
APVNTTTEDETAQIPAEAVIGYLDLEGDFDVAVLPFSNSTNNGLLFINTT
IASIAAKEEGVSLDKR.

45. The polypeptide of any one of claims 31-44, wherein the pro-sequence comprises the amino acid sequence:
(SEQ ID NO: 61)
APANTTTEDETAQIPAEAVIDYSDLEGDFDAAALPLSNSTNNGLSSTNTT
IASIAAKEEGVSLDKR.

46. A polypeptide comprising a secretion signal, wherein the secretion signal comprises the amino acid sequence LX²X³X⁴X⁵LLX⁸X⁹X¹⁰X¹¹X¹²APX¹⁵NX¹⁷TX¹⁹EX²¹EX²³X²⁴QX²⁶PAEAX³¹X³²X³³YX³⁵X³⁶X³⁷EGDX⁴¹DX⁴³AX⁴⁵LPX⁴⁸X⁴⁹X⁵⁰STNX⁵⁴GX⁵⁶X⁵⁷X⁵⁸X⁵⁹NTTX⁶³ASIAAKEEGVSLDKR (SEQ ID NO: 153), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); X¹² is alanine (A) or cytosine (C); X¹⁵ is valine (V) or alanine (A); X¹⁷ is threonine (T) or alanine (A); X¹⁹ is threonine (T) or alanine (A); X²¹ is aspartic acid (D) or glycine (G); X²³ is threonine (T) or alanine (A); X²⁴ is threonine (T) or alanine (A); X²⁶ is isoleucine (I) or threonine (T); X³¹ is valine (V) or alanine (A); X³² is isoleucine (I) or alanine (A); X³³ is glycine (G), aspartic acid (D), or threonine (T); X³⁵ is leucine (L), serine (S), or arginine (R); X³⁶ is aspartic acid (D) or glycine (G); X³⁷ is leucine (L) or serine (S); X⁴¹ is phenylalanine (F), serine (S), or valine (V); X⁴³ is valine (V) or alanine (A); X⁴⁵ is valine (V) or alanine (A); X⁴⁸ is phenylalanine (F) or leucine (L); X⁴⁹ serine (S) or proline (P); X⁵⁰ is asparagine (N), serine (S), or aspartic acid (D); X⁵⁴ is asparagine (N) or aspartic acid (D); X⁵⁶ is leucine (L) or serine (S); X⁵⁷ is leucine (L) or serine (S); X⁵⁸ is phenylalanine (F) or serine (S); X⁵⁹ is isoleucine (I) or threonine (T); and X⁶³ is isoleucine (I) or threonine (T).

47. The polypeptide of claim 46, wherein the secretion signal does not comprise the amino acid sequence of
(SEQ ID NO: 59)
APVNTTTEDETAQIPAEAVIGYLDLEGDFDVAVLPFSNSTNNGLLFINTT
IASIAAKEEGVSLDKR.

48. The polypeptide of claim 45 or claim 46, wherein the secretion signal comprises the amino acid sequence LX²X³X⁴X⁵LLX⁸X⁹X¹⁰X¹¹X¹²APANTTTEDETAQIPAEAVIDYSDLEGDFDAAALPLSNS TNNGLSSTNTTIASIAAKEEGVSLDKR (SEQ ID NO: 157), wherein: X² is phenylalanine (F), threonine (T), or leucine (L); X³ is phenylalanine (F), leucine (L), or alanine (A); X⁴ is isoleucine (I), leucine (L), or valine (V); X⁵ is threonine (T), phenylalanine (F), or serine (S); X⁸ is histidine (H), serine (S), or threonine (T); X⁹ is leucine (L), phenylalanine (F), or threonine (T); X¹⁰ is valine (V) or threonine (T); X¹¹ is valine (V), glutamic acid (E), or tyrosine (Y); and X¹² is alanine (A) or cytosine (C).

49. The polypeptide of any one of claims 46-48, wherein the secretion signal further comprises the amino acid sequence: MTKPTQVLVRSVSI (SEQ ID NO: 154); MQLY (SEQ ID NO: 155); or MQHFLSL (SEQ ID NO: 156).

50. A polypeptide comprising a secretion signal, wherein the secretion signal comprises an amino acid sequence having at least 80% identity to the one or more of:
(SEQ ID NO: 107)
MTKPTQVLVRSVSILFFITLLHLVVAAPANTTTEDETAQIPAEAVIDYSD
LEGDFDAAALPLSNSTNNGLSSTNTTIASIAAKEEGVSLDKR;
(SEQ ID NO: 108)
MQLYLTLLFLLSFVECAPANTTTEDETAQIPAEAVIDYSDLEGDFDAAAL
PLSNSTNNGLSSTNTTIASIAAKEEGVSLDKR;
and
(SEQ ID NO: 115)
MQHFLSLLLAVSLLTTTYAAPANTTTEDETAQIPAEAVIDYSDLEGDFDA
AALPLSNSTNNGLSSTNTTIASIAAKEEGVSLDKR.

51. The polypeptide of any one of claims 31-50, wherein the secretion signal comprises the amino acid sequence of:
(SEQ ID NO: 107)
MTKPTQVLVRSVSILFFITLLHLVVAAPANTTTEDETAQIPAEAVIDYSD
LEGDFDAAALPLSNSTNNGLSSTNTTIASIAAKEEGVSLDKR;
(SEQ ID NO: 108)
MQLYLTLLFLLSFVECAPANTTTEDETAQIPAEAVIDYSDLEGDFDAAAL
PLSNSTNNGLSSTNTTIASIAAKEEGVSLDKR;
or
(SEQ ID NO: 115)
MQHFLSLLLAVSLLTTTYAAPANTTTEDETAQIPAEAVIDYSDLEGDFDA
AALPLSNSTNNGLSSTNTTIASIAAKEEGVSLDKR.

52. A polypeptide comprising a secretion signal, wherein the secretion signal comprises, from N-terminus to C-terminus, a pre-sequence from a first species (or derived from a pre-sequence from a first species) and a pro-sequence from a second species (or derived from a pro-sequence from a second species), wherein:
a) the pre-sequence comprises the amino acid sequence of: WFSWIVG (SEQ ID NO: 18); MRFPSIFTAVLF (SEQ ID NO: 19); SSALA (SEQ ID NO: 20); IVGLF (SEQ ID NO: 21); MTKPTQVLV (SEQ ID NO: 22); MKLATAFTILTA (SEQ ID NO: 23); ETPRASLSLGRW (SEQ ID NO: 24); WHAVMVFVLCG (SEQ ID NO: 25); MRFPSIFT (SEQ ID NO: 222); MKX³X⁴X⁵X⁶AX⁸LSX¹¹X¹²X¹³LX¹⁴L (SEQ ID NO: 26), wherein X³ is phenylalanine (F) or leucine (L), X⁴ is serine (S) or phenylalanine (F), X⁵ is alanine (A) or valine (V), X⁶ is glycine (G) or proline (P), X⁸ is valine (V) or leucine (L), X¹¹ is tryptophan (W) or leucine (L), X¹² is serine (S) or glycine (G), X¹³ is serine (S) or alanine (A), and X¹⁴ is leucine (L) or glycine (G); or MX²X³X⁴X⁵ (SEQ ID NO: 223), wherein X² is arginine (R) or glutamine (Q), X³ is histidine (H) or glutamine (Q), X⁴ is valine (V) or phenylalanine (F), and X⁵ is leucine (L) or tryptophan (W);
b) the pro-sequence comprises the amino acid sequence of: RYVVGDDEQ (SEQ ID NO: 64); IVAKSGI (SEQ ID NO: 65); IPDEAIAN (SEQ ID NO: 66); QTSISDDEEPIVVEINGQKV (SEQ ID NO: 67); INTTLTEEALEKSGISIDDL (SEQ ID NO: 68); PVFAEIDNK (SEQ ID NO: 69); DDLKESYAN (SEQ ID NO: 70); PVENVDD (SEQ ID NO: 71); IDQEQLTNG (SEQ ID NO: 72); PVDSGAKGKYSR (SEQ ID NO: 73); NDGVGVGMSTIKEEDFGKHF (SEQ ID NO: 74); TTIASIA (SEQ ID NO: 224); YVVGDDEQ (SEQ ID NO: 225); PVFAEIDNKPVVYIVNTTKA (SEQ ID NO: 226); ESIVAKSGITLDDLKESYAN (SEQ ID NO: 227); NTTIX⁵X⁶X⁷A (SEQ ID NO: 63), wherein X⁵ is alanine (A), leucine (L), or tyrosine (Y), X⁶ is alanine (A), serine (S), asparagine (N), or glutamic acid (E), and X⁷ is alanine (A), isoleucine (I), serine (S), glutamic acid (E), or glutamine (Q); AAX³EEGX⁷SLDKR (SEQ ID NO: 221), wherein X³ is lysine (K) or alanine (A), and X⁷ is valine (V) or serine (S); X¹NTTIAX⁷X⁸AX¹⁰X¹¹EEGVX¹⁶ (SEQ ID NO: 75), wherein X¹ is valine (V) or isoleucine (I), X⁷ is aspartic acid (D), serine (S), or glutamic acid (E), X⁸ is isoleucine (I) or glutamine (Q), X¹⁰ is alanine (A) or leucine (L), X¹¹ is alanine (A) or lysine (K), and X¹⁶ is serine (S) or leucine (L); X¹X²X³X⁴X⁵DDEX⁹ (SEQ ID NO: 76), wherein X¹ is arginine (R) or glutamine (Q), X² is tyrosine (Y) or threonine (T), X³ is valine (V) or serine (S), X⁴ is valine (V) or isoleucine (I), X⁵ is glycine (G) or serine (S), and X⁹ is glutamine (Q) or glutamic acid (E); AX²LPFSNX⁸TNX¹¹GX¹³X¹⁴FX¹⁶NTTI (SEQ ID NO: 77), wherein X² is valine (V) or leucine (L), X⁸ is serine (S) or glycine (G), X¹¹ is asparagine (N) or threonine (T), X¹³ is isoleucine (I) or leucine (L), X¹⁴ is serine (S), leucine (L), or methionine (M), and X¹⁶ is valine (V) or isoleucine (I); X¹AQX⁴PAEAX⁹IGX¹²LDLX¹⁶X¹⁷X¹⁸X¹⁹D (SEQ ID NO: 78), wherein X¹ is threonine (T) or serine (S), X⁴ is isoleucine (I) or valine (V), X⁹ is valine (V) or isoleucine (I), X¹² is tyrosine (Y) or phenylalanine (F), X¹⁶ is glutamic acid (E) or threonine (T), X¹⁷ is aspartic acid (D) or glycine (G), X¹⁸ is aspartic acid (D), serine (S), or alanine (A), and X¹⁹ is glutamic acid (E) or phenylalanine (F); X¹GX³X⁴X⁵X⁶X⁷DX⁹IX¹¹P (SEQ ID NO: 79), wherein X¹ is lysine (K) or serine (S), X³ is lysine (K) or arginine (R), X⁴ is tyrosine (Y) or phenylalanine (F), X⁵ is serine (S) or leucine (L), X⁶ is arginine (R) or glutamic acid (E), X⁷ is glutamine (Q) or threonine (T), X⁹ is leucine (L) or isoleucine (I), and X¹¹ is isoleucine (I) or phenylalanine (F); X¹X²NX⁴TX⁶E (SEQ ID NO: 80), wherein X¹ is asparagine (N) or proline (P), X² is glycine (G) or alanine (A), X⁴ is glycine (G) or threonine (T), and X⁶ is serine (S) or threonine (T); PAEAVIX⁷Y (SEQ ID NO: 228), wherein X⁷ is aspartic acid (D) or glycine (G); KEEX⁴X⁵X⁶X⁷X⁸KR (SEQ ID NO: 229), wherein X⁴ is glycine (G) or glutamic acid (E), X⁵ is valine (V) or alanine (A), X⁶ is serine (S) or lysine (K), X⁷ is leucine (L) or asparagine (N), and X⁸ is aspartic acid (D) or glycine (G); GDFDX⁵AX⁷LP (SEQ ID NO: 230), wherein X⁵ is valine (V) or alanine (A), and X⁷ is valine (V) or alanine (A); X¹SNST (SEQ ID NO: 231), wherein X¹ is leucine (L) or phenylalanine (F); GLSX⁴TN (SEQ ID NO: 232), wherein X⁴ is serine (S) or phenylalanine (F); PX²SNSTNNGLSX¹²TNTTIASI (SEQ ID NO: 233), wherein X² is leucine (L) or phenylalanine (F), and X¹² is serine (S) or phenylalanine (F); or X¹X²X³IPX⁶EAX⁹X¹⁰X¹¹X¹²X¹³X¹⁴X¹⁵X¹⁶X¹⁷DX¹⁹X²⁰ (SEQ ID NO: 234), wherein X¹ is threonine (T) or aspartic acid (D), X² is alanine (A) or leucine (L), X³ is glutamine (Q) or isoleucine (I), X⁶ is alanine (A) or aspartic acid (D), X⁹ is valine (V) or isoleucine (I), X¹⁰ is isoleucine (I) or alanine (A), X¹¹ is aspartic acid (D), glycine (G), or asparagine (N), X¹² is tyrosine (Y) or arginine (R), X¹³ serine (S) or tyrosine (Y), X¹⁴ is aspartic acid (D) or valine (V); X¹⁵ is leucine (L) or valine (V), X¹⁶ is glutamic acid (E) or glycine (G), X¹⁷ is glycine (G) or aspartic acid (D), X¹⁹ is phenylalanine (F) or glutamic acid (E), and X²⁰ is aspartic acid (D) or glutamine (Q); or
c) a combination thereof.

53. A polypeptide comprising a secretion signal, wherein the secretion signal comprises, from N-terminus to C-terminus, a pre-sequence from a first species (or derived from a pre-sequence from a first species) and a pro-sequence from a second species (or derived from a pro-sequence from a second species), wherein the secretion signal comprises the amino acid sequence: CX²X³X⁴X⁵X⁶X⁷X⁸APX¹¹NTTT (SEQ ID NO: 146), wherein X² is leucine (L), phenylalanine (F), or glycine (G), X³ is leucine (L), phenylalanine (F), or valine (V), X⁴ is asparagine (N) or valine (V), X⁵ is valine (V) or leucine (L), X⁶ is serine (S), alanine (A), or valine (V), X⁷ is serine (S) or alanine (A), X⁸ is alanine (A) or glycine (G), and X¹¹ is valine (V) or alanine (A); X¹AAPX⁵X⁶TTTEDE (SEQ ID NO: 147), wherein X¹ is leucine (L), serine (S), or alanine (A), X⁵ is alanine (A) or valine (V), and X⁶ is asparagine (N) or serine (S); AAPIX⁵X⁶X⁷X⁸S (SEQ ID NO: 148), wherein X⁵ is asparagine (N) or lysine (K), X⁶ is isoleucine (I) or phenylalanine (F), X⁷ is threonine (T) or asparagine (N), and X⁸ is serine (S) or aspartic acid (D); X¹X²X³X⁴X⁵X⁶X⁷X⁸X⁹ (SEQ ID NO: 149), wherein X¹ is glutamine (Q) or asparagine (N), X² is valine (V) or histidine (H), X³ is tryptophan (W) or phenylalanine (F), X⁴ is phenylalanine (F), leucine (L), or histidine (H), X⁵ is serine (S) or alanine (A), X⁶ is tryptophan (W), leucine (L), or valine (V), X⁷ is isoleucine (I), leucine (L), or methionine (M), X⁸ is valine (V) or leucine (L), and X⁹ is glycine (G), alanine (A), or phenylalanine (F); X¹X²X³X⁴X⁵X⁶AX⁸X⁹ (SEQ ID NO: 150), wherein X¹ lysine (K) or asparagine (N), X² is glycine (G), asparagine (N), or aspartic acid (D), X³ is asparagine (N), glycine (G), or lysine (K), X⁴ is leucine (L), tyrosine (Y), or glycine (G), X⁵ is serine (S) or asparagine (N), X⁶ is serine (S), arginine (R), or glycine (G), X⁸ is asparagine (N), aspartic acid (D), or serine (S), and X⁹ is threonine (T), leucine (L), or glutamic acid (E); X¹RX³X⁴X⁵X⁶X⁷X⁸X⁹X¹⁰X¹¹X¹²X¹³X¹⁴X¹⁵X¹⁶ (SEQ ID NO: 151), wherein X¹ is methionine (M), valine (V), or glutamine (Q), X³ is phenylalanine (F) or glutamine (Q), X⁴ is leucine (L) or valine (V), X⁵ is serine (S) or tryptophan (W), X⁶ is phenylalanine (F) or leucine (L), X⁷ is leucine (L) or serine (S), X⁸ is threonine (T), leucine (L), phenylalanine (F), or tryptophan (W), X⁹ is alanine (A), leucine (L), or isoleucine (I), X¹⁰ is valine (V) or leucine (L), X¹¹ is leucine (L), glycine (G), or serine (S), X¹² is leucine (L) or phenylalanine (F), X¹³ is valine (V), leucine (L), or phenylalanine (F), X¹⁴ is valine (V) or leucine (L), X¹⁵ is serine (S) or cytosine (C), and X¹⁶ is alanine (A) or phenylalanine (F); X¹X²X³X⁴X⁵X⁶X⁷X⁸X⁹X¹⁰X¹¹X¹²X¹³X¹⁴X¹⁵X¹⁶X¹⁷IX¹⁹X²⁰ (SEQ ID NO: 152), wherein X¹ is aspartic acid (D), valine (V), or glutamic acid (E), X² is valine (V), tyrosine (Y), or proline (P), X³ is proline (P), isoleucine (I), or serine (S), X⁴ is glycine (G) or valine (V), X⁵ is threonine (T), asparagine (N), or arginine (R), X⁶ is serine (S), threonine (T), or phenylalanine (F), X⁷ is glutamine (Q), threonine (T), or leucine (L), X⁸ is glycine (G), lysine (K), or glutamic acid (E), X⁹ is valine (V), alanine (A), or glutamine (Q), X¹⁰ is glutamic acid (E) or aspartic acid (D), X¹¹ is phenylalanine (F), serine (S), or isoleucine (I), X¹² is isoleucine (I) or proline (P), X¹³ is phenylalanine (F) or valine (V), X¹⁴ is alanine (A) or proline (P), X¹⁵ is lysine (K) or glutamine (Q), X¹⁶ is glutamic acid (E), serine (S), or glutamine (Q), X¹⁷ is alanine (A) or glycine (G), X¹⁹ is isoleucine (I), threonine (T), or asparagine (N), and X²⁰ is glutamic acid (E), leucine (L), or alanine (A); AAPX⁴X⁵X⁶X⁷X⁸X⁹X¹⁰X¹¹X¹² (SEQ ID NO: 235), wherein X⁴ is alanine (A) or valine (V), X⁵ is asparagine (N) or aspartic acid (D), X⁶ is serine (S) or threonine (T), X⁷ is threonine (T) or glycine (G), X⁸ is threonine (T) or alanine (A), X⁹ is glutamic acid (E) or lysine (K), X¹⁰ is glycine (G) or aspartic acid (D), X¹¹ is glutamic acid (E) or lysine (K), and X¹² is threonine (T) or tyrosine (Y); AX²KEEX⁶X⁷X⁸X⁹X¹⁰KREAEA (SEQ ID NO: 236), wherein X² is alanine (A) or threonine (T), X⁶ is glycine (G) or glutamic acid (E), X⁷ is valine (V) or alanine (A); X⁸ is serine (S) or lysine (K), X⁹ is leucine (L) or asparagine (N), and X¹⁰ is aspartic acid (D) or glycine (G); SLLX⁴X⁵SX7X⁸LAAPX¹³NTTTEDE (SEQ ID NO: 237), wherein X⁴ is alanine (A), phenylalanine (F), leucine (L), or serine (S), X⁵ is leucine (L) or alanine (A), X⁷ is leucine (L) or serine (S), X⁸ is leucine (L) or valine (V), and X¹³ is alanine (A) or valine (V); or MX²X³X⁴X⁵X⁶X⁷X⁸X⁹ (SEQ ID NO: 238), wherein X² is alanine (A), lysine (K), or arginine (R), X³ is leucine (L), glutamine (Q), or arginine (R), X⁴ is phenylalanine (F) or valine (V), X⁵ is valine (V) or tryptophan (W), X⁶ is alanine (A), phenylalanine (F), or proline (P), X⁷ is leucine (L), alanine (A), or serine (S), X⁸ is leucine (L), valine (V), or tryptophan (W), and X⁹ is leucine (L) or isoleucine (I).

54. The polypeptide of any one of claims 31-53, wherein the secretion signal further comprises a C-terminal cleavage sequence.

55. The polypeptide of claim 54, wherein the C-terminal cleavage sequence comprises the amino acid sequence of EAEA (SEQ ID NO: 104), KR, or a combination thereof.

56. The polypeptide of any one of claims 31-55, further comprising the amino acid sequence of a protein of interest, wherein the amino acid sequence of the protein of interest is positioned C-terminal to the amino acid sequence of the secretion signal.

57. The polypeptide of claim 56, wherein the protein of interest is lactoferrin (LF), lactoglobulin (LG), or ovalbumin (Ova).

58. The polypeptide of claim 56, wherein the protein of interest is bovine lactoferrin (bLF), bovine lactoglobulin (bLG), or ovalbumin (Ova).

59. A nucleic acid encoding the polypeptide of any one of claims 31-58.

60. The nucleic acid of claim 59, further comprising a nucleic acid sequence of a promoter, wherein the nucleic acid sequence of the promoter is operably linked to the nucleic acid sequence encoding the polypeptide.

61. The nucleic acid sequence of claim 60, wherein the promoter is a constitutive promoter.

62. The nucleic acid of claim 61, wherein the promoter comprises GAP, and wherein the polypeptide comprises the amino acid sequence of lactoferrin (LF).

63. The nucleic acid sequence of claim 60, wherein the promoter is an inducible promoter.

64. The nucleic acid sequence of claim 63, wherein the inducible promoter is one that is activated during glucose or thiamine limitation.

65. The nucleic acid sequence of any one of claims 59-64, further comprising a nucleic acid sequence of a terminator, wherein the nucleic acid sequence of the terminator is operably linked to the nucleic acid sequence encoding the polypeptide.

66. The nucleic acid sequence of claim 65, wherein the terminator is a tFDH1 terminator, a tTEF1 terminator, or a tAOX1 terminator.

67. An expression vector comprising the nucleic acid sequence of any one of claims 59-66.

68. A host cell comprising the nucleic acid sequence of any one of claims 59-67.

69. A host cell comprising one or more genetic modifications that result in the overexpression of a gene encoding a calreticulin (CRT) protein (or a homolog thereof) and/or a protein disulfide isomerase family A member 3 (PDIA3) protein (or a homolog thereof).

70. The host cell of claim 69, further comprising one or more genetic modifications that result in the overexpression of a HAC1 protein (or a homolog thereof).

71. A host cell comprising:
a) one or more genetic modifications that result in the overexpression of a gene encoding a calreticulin (CRT) protein (or a homolog thereof) and/or a protein disulfide isomerase family A member 3 (PDIA3) protein (or a homolog thereof); and
b) a nucleic acid sequence encoding a polypeptide comprising a secretion signal and the amino acid sequence of a protein of interest.

72. The host cell of claim 71, further comprising one or more genetic modifications that result in the overexpression of a HAC1 protein (or a homolog thereof)

73. The host cell of claim 69 or claim 71, wherein the host cell comprises one or more genetic modifications that result in the overexpression of an endogenous CRT protein (or a homolog thereof).

74. The host cell of any one of claims 69-73, wherein the host cell comprises one or more genetic modifications that result in the overexpression of an exogenous CRT protein (or a homolog thereof).

75. The host cell of claim 73 or claim 74, wherein the CRT protein (or homolog thereof) is selected from the group consisting of Anopheles christyi CRT, Arabidopsis thaliana CRT1, Chlorocebus aethiops CALR, Gigaspora rosea Calreticulin family-domain-containing protein, Mucor ambiguus CRT, and Mus musculus CALR.

76. The host cell of any one of claims 69-75, wherein the host cell comprises one or more genetic modifications that result in the overexpression of an endogenous PDIA3 protein (or a homolog thereof).

77. The host cell of any one of claims 69-76, wherein the host cell comprises one or more genetic modifications that result in the overexpression of an exogenous PDIA3 protein (or a homolog thereof).

78. The host cell of claim 76 or claim 77, wherein the PDIA3 protein (or homolog thereof) is selected from the group consisting of Anopheles christyi Protein disulfide-isomerase, Arabidopsis thaliana Protein disulfide isomerase-like 1-1, Arabidopsis thaliana Protein disulfide isomerase-like 1-2, Arabidopsis thaliana Protein disulfide isomerase-like 1-6, Chlorocebus aethiops PDIA3, Dictyostelium discoideum Protein disulfide-isomerase 2, Mucor ambiguus Protein disulfide isomerase-like 2-1-like, and Mus musculus PDIA3.

79. The host cell of any one of claims 69-78, wherein at least one of the one or more genetic modifications is a genomic integration of an expression cassette encoding for a CRT protein (or a homolog thereof) or a PDIA3 protein (or a homolog thereof).

80. The host cell of any one of claims 69-79, wherein at least one of the one or more genetic modifications is a transformation of a vector comprising an expression cassette encoding for a CRT protein (or a homolog thereof) or a PDIA3 protein (or a homolog thereof).

81. A host cell comprising:
a) one or more genetic modifications that result in the overexpression of a gene encoding a calreticulin (CRT) protein (or a homolog thereof) and/or a protein disulfide isomerase family A member 3 (PDIA3) protein (or a homolog thereof); and
b) one or more genetic modifications that result in the overexpression of a HAC1 protein (or a homolog thereof).

82. The host cell of claim 81, wherein the host cell comprises one or more genetic modifications that result in the overexpression of an endogenous HAC1 protein (or a homolog thereof).

83. The host cell of claim 81 or claim 82, wherein the host cell comprises one or more genetic modifications that result in the overexpression of an exogenous HAC1 protein (or a homolog thereof).

84. The host cell of claim 82 or claim 83, wherein the HAC1 protein (or homolog thereof) is selected from the group consisting of Saccharomyces cerevisiae HAC1 and Homo sapiens XBP1.

85. The host cell of any one of claims 81-84, wherein the host cell comprises one or more genetic modifications that result in the overexpression of an endogenous CRT protein (or a homolog thereof).

86. The host cell of any one of claims 81-85, wherein the host cell comprises one or more genetic modifications that result in the overexpression of an exogenous CRT protein (or a homolog thereof).

87. The host cell of claim 85 or claim 86, wherein the CRT protein (or homolog thereof) is selected from the group consisting of Anopheles christyi CRT, Arabidopsis thaliana CRT, Aspergillus niger CRT, Chlorocebus aethiops CRT, Dictyostelium discoideum CRT, Lichtheimia ramosa CRT, Mucor ambiguus CRT, Mus musculus CRT, and Pichia pastoris CRT.

88. The host cell of any one of claims 85-87, wherein the host cell comprises one or more genetic modifications that result in the overexpression of an endogenous PDIA3 protein (or a homolog thereof).

89. The host cell of any one of claims 85-88, wherein the host cell comprises one or more genetic modifications that result in the overexpression of an exogenous PDIA3 protein (or a homolog thereof).

90. The host cell of claim 88 or claim 89, wherein the PDIA3 protein (or homolog thereof) is selected from the group consisting of Anopheles christyi PDIA3, Arabidopsis thaliana PDIA3, Aspergillus niger PDIA3, Chlorocebus aethiops PDIA3, Dictyostelium discoideum PDIA3, Lichtheimia ramosa PDIA3, Mucor ambiguus PDIA3, Mus musculus PDIA3, and Pichia pastoris PDIA3.

91. The host cell of any one of claims 85-90, wherein at least one of the one or more genetic modifications is a genomic integration of an expression cassette encoding for a CRT protein (or homolog thereof) or a PDIA3 protein (or a homolog thereof).

92. The host cell of any one of claims 85-91, wherein at least one of the one or more genetic modifications is a transformation of a vector comprising an expression cassette encoding for a CRT protein (or a homolog thereof) or a PDIA3 protein (or a homolog thereof).

93. The host cell of any one of claims 85-92, further comprising a nucleic acid sequence encoding a polypeptide, wherein the polypeptide comprises a secretion signal and the amino acid sequence of a protein of interest.

94. The host cell of any one of claims 68-93, wherein the host cell is a eukaryotic cell.

95. The host cell of claim 94, wherein the host cell is a yeast cell.

96. The host cell of claim 95, wherein the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica.

97. The host cell of claim 95, wherein the yeast cell is Pichia pastoris.

98. The host cell of claim 94, wherein the host cell is a mold cell.

99. The host cell of claim 98, wherein the mold cell is Aspergillus, Trichoderma, Humicola, Neurospora, Penicillium, Cephalosporium, Myceliophthora, Thermomyces, or Chrysosporium.

100. The host cell of claim 99, wherein the mold cell is Aspergillus niger, Aspergillus nidulans, Aspergillus awamori, Aspergillus sojae, Aspergillus oryzae, Trichoderma reesei, Trichoderma viride, Chrysosporium lucknowense, Fusarium gramineum, Fusarium venenatum, or Neurospora crassa.

101. The host cell of claim 99, wherein the mold cell is Aspergillus niger.

102. A method of manufacturing a polypeptide, the method comprising culturing the host cell of any one of claims 68, 71-79, and 93.

103. A method of manufacturing a polypeptide, the method comprising culturing the host cell of any one of claims 71-79 and 93, wherein the level of secretion of the polypeptide is increased relative to the respective level for a host cell that is the same except that it lacks the one or more genetic modifications.

104. The method of claim 102 or claim 103, further comprising obtaining the polypeptide from a culture, culture medium, cell-free spent culture medium, and/or cell-containing culture medium, and/or biomass used in, during or produced by culturing the host cell.

105. The method of any one of claims 102-104, wherein the host cell is a eukaryotic cell.

106. The method of claim 105, wherein the host cell is a yeast cell.

107. The method of claim 106, wherein the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica.

108. The method of claim 106, wherein the yeast cell is Pichia pastoris.

109. The method of claim 104, wherein the host cell is a mold cell.

110. The method of claim 109, wherein the mold cell is Aspergillus, Trichoderma, Humicola, Neurospora, Penicillium, Cephalosporium, Myceliophthora, Thermomyces, or Chrysosporium.

111. The method of claim 110, wherein the mold cell is Aspergillus niger, Aspergillus nidulans, Aspergillus awamori, Aspergillus sojae, Aspergillus oryzae, Trichoderma reesei, Trichoderma viride, Chrysosporium lucknowense, Fusarium gramineum, Fusarium venenatum, or Neurospora crassa.

112. The method of claim 110, wherein the mold cell is Aspergillus niger.


SEQUENCE LISTING (245 sequences)

SEQ ID NO: 1 - protein, 26 aa, synthetic construct
MTKPTQVLVRSVSILFFITLLHLVVA

SEQ ID NO: 2 - protein, 19 aa, synthetic construct
MQHFLSLLLAVSLLTTTYA

SEQ ID NO: 3 - protein, 20 aa, synthetic construct
MNVWHAVMVFVLCGVVVAAG

SEQ ID NO: 4 - protein, 22 aa, synthetic construct
MRQVWFSWIVGLFLCFFNVSSA

SEQ ID NO: 5 - protein, 19 aa, synthetic construct
MRFPSIFTAVLFAASSALA

SEQ ID NO: 6 - protein, 21 aa, synthetic construct
MKLATAFTILTAVLAAPLAAP

SEQ ID NO: 7 - protein, 22 aa, synthetic construct
MKFSAGAVLSWSSLLLASSVFA

SEQ ID NO: 8 - protein, 18 aa, synthetic construct
MKAFTSLLCGLGLSTTLA

SEQ ID NO: 9 - protein, 16 aa, synthetic construct
MQLYLTLLFLLSFVEC

SEQ ID NO: 10 - protein, 22 aa, synthetic construct
MFSLKALLPLALLLVSANQVAA

SEQ ID NO: 11 - protein, 20 aa, synthetic construct
MARFVALVLLGLLSLSGLDA

SEQ ID NO: 12 - protein, 19 aa, synthetic construct
MSLFTSLPFLLLTAVTASC

SEQ ID NO: 13 - protein, 19 aa, synthetic construct
MKLFVPALLSLGALGLCLA

SEQ ID NO: 14 - protein, 29 aa, synthetic construct
METPRASLSLGRWSLWLLLLGLALPSASA

SEQ ID NO: 15-17 - skipped (3 entries)

SEQ ID NO: 18 - protein, 7 aa, synthetic construct
WFSWIVG

SEQ ID NO: 19 - protein, 12 aa, synthetic construct
MRFPSIFTAVLF

SEQ ID NO: 20 - protein, 5 aa, synthetic construct
SSALA

SEQ ID NO: 21 - protein, 5 aa, synthetic construct
IVGLF

SEQ ID NO: 22 - protein, 9 aa, synthetic construct
MTKPTQVLV

SEQ ID NO: 23 - protein, 12 aa, synthetic construct
MKLATAFTILTA

SEQ ID NO: 24 - protein, 12 aa, synthetic construct
ETPRASLSLGRW

SEQ ID NO: 25 - protein, 11 aa, synthetic construct
WHAVMVFVLCG

SEQ ID NO: 26 - protein, 16 aa, synthetic construct
  VARIANT 3: note = X is phenylalanine (F) or leucine (L)
  VARIANT 4: note = X is serine (S) or phenylalanine (F)
  VARIANT 5: note = X is alanine (A) or valine (V)
  VARIANT 6: note = X is glycine (G) or proline (P)
  VARIANT 8: note = X is valine (V) or leucine (L)
  VARIANT 11: note = X is tryptophan (W) or leucine (L)
  VARIANT 12: note = X is serine (S) or glycine (G)
  VARIANT 13: note = X is serine (S) or alanine (A)
  VARIANT 14: note = X is leucine (L) or glycine (G)
MKXXXXAXLSXXXLXL

SEQ ID NO: 27 - DNA, 78 bp, unidentified
  source 1..78: mol_type = genomic DNA; note = Saccharomyces paradoxus
atgacgaagccgacacaagttcttgtgaggtcagtctctatattattctttatcactctgctccatctagttgtagct

SEQ ID NO: 28 - DNA, 57 bp, unidentified
  source 1..57: mol_type = genomic DNA; note = Trametes versicolor FP-101664 SS1
atgcagcatttcctttccctactgttagccgttagtttgctcactacaacgtacgct

SEQ ID NO: 29 - DNA, 60 bp, unidentified
  source 1..60: mol_type = genomic DNA; note = Gloeophyllum trabeum ATCC 11539
atgaacgtttggcacgcagtaatggtgtttgtcctatgtggggttgtagtggctgccggc

SEQ ID NO: 30 - DNA, 66 bp, Saccharomyces cerevisiae
  source 1..66: mol_type = genomic DNA
atgaggcaagtctggttttcatggatagttggccttttcttgtgttttttcaatgtaagcagtgca

SEQ ID NO: 31 - DNA, 57 bp, Saccharomyces cerevisiae
  source 1..57: mol_type = genomic DNA
atgcgattcccgtcaatctttactgccgtcttgtttgctgcgtctagcgcactagct

SEQ ID NO: 32 - DNA, 63 bp, unidentified
  source 1..63: mol_type = genomic DNA; note = Yarrowia lipolytica
atgaagctagccaccgcattcacaatacttacggctgtgctcgcggctcctttagctgcaccc

SEQ ID NO: 33 - DNA, 66 bp, Saccharomyces cerevisiae
  source 1..66: mol_type = genomic DNA
atgaaattttcagcaggggctgttttaagttggtcttccctcctgttggcctcgtctgtgttcgcg

SEQ ID NO: 34 - DNA, 54 bp, Saccharomyces cerevisiae
  source 1..54: mol_type = genomic DNA
atgaaggcgttcacatcgttgctttgcggattaggcctgagcaccactctagca

SEQ ID NO: 35 - DNA, 48 bp, Saccharomyces cerevisiae
  source 1..48: mol_type = genomic DNA
atgcaactatacttgacacttttattcctgctctcctttgtagagtgt

SEQ ID NO: 36 - DNA, 66 bp, Saccharomyces cerevisiae
  source 1..66: mol_type = genomic DNA
atgttcagtcttaaagcactattgccgttagccctgttgctcgtcagcgcgaaccaggtagctgct

SEQ ID NO: 37 - DNA, 60 bp, unidentified
  source 1..60: mol_type = genomic DNA; note = P. pastoris recode 1
atggcccgttttgtggcgttagtactactcggcctgctttcattgtccggtttggatgca

SEQ ID NO: 38 - DNA, 57 bp, Bos taurus
  source 1..57: mol_type = genomic DNA
atgtcactcttcacttccttgccctttctgctattaaccgccgtcacagcatcttgc

SEQ ID NO: 39 - DNA, 57 bp, Bos taurus
  source 1..57: mol_type = genomic DNA
atgaaactgttcgtgcccgcgcttttatcactaggcgccttggggttgtgtcttgca

SEQ ID NO: 40 - DNA, 87 bp, Bos taurus
  source 1..87: mol_type = genomic DNA
atggaaactcctcgagcctcattgagtcttggaagatggagcttatggctactgctgttggggctcgctttgccgtccgcatctgct

SEQ ID NO: 41 - protein, 26 aa, unidentified
  source 1..26: note = Cutaneotrichosporon oleaginosum
APVAAPEAEAGGRGNFGNSGPPIWKR

SEQ ID NO: 42 - protein, 63 aa, unidentified
  source 1..63: note = Cutaneotrichosporon oleaginosum
APINITSSDPAIPSESISGFLDLTDAEDLALLPVSNGTHSGVLIVNTTILAQAFGSDDVLTKR

SEQ ID NO: 43 - protein, 90 aa, unidentified
  source 1..90: note = Lachancea quebecensis
APIEAPTSDETPAPTEPSGRFLEQDIIFPQQAINAQTSISDDEEPIVVEINGQKVILLINTTLTEEALEKSGISIDDLNALAGNSTVSKR

SEQ ID NO: 44 - protein, 12 aa, unidentified
  source 1..12: note = Suhomyces tanzawaensis NRRL Y-17324
APVSISGQSLKR

SEQ ID NO: 45 - protein, 66 aa, unidentified
  source 1..66: note = Pachysolen tannophilus NRRL Y-2460
APVSGASNSTSMEVPGEAVKYFLDLSDSPDMALVPINNGNTTGIMFVNTTVIDQAYAETTSLSRKR

SEQ ID NO: 46 - protein, 68 aa, unidentified
  source 1..68: note = Hanseniaspora osmophila
AAIDQEQLTNGTYIDIPQESILSFLDLTDSPEVSVYPIKEGSKTGLIFVNSTIVDQAYSETTALTRKR

SEQ ID NO: 47 - protein, 63 aa, unidentified
  source 1..63: note = Hanseniaspora uvarum
APIKFNDSSPALPLESISGYLDLTGAEDLALLPVSNATHTGILVVNTTILASALASESNYNKR

SEQ ID NO: 48 - protein, 66 aa, unidentified
  source 1..66: note = Lachancea nothofagi CBS 11611 |
APVNTTTEDETAQIPAEAIIGYLDLEGDFDIAVLPFSNSTNNGLLFINTTIANIAAEEEGVTLNKR

SEQ ID NO: 49 - protein, 7 aa, unidentified
  source 1..7: note = Saccharomyces paradoxus
KPQHYKR

SEQ ID NO: 50 - protein, 73 aa, unidentified
  source 1..73: note = Rhizophagus irregularis
APVAKDATNTTDASSVQIPAEAVIGYLDLEQSNDVAMLQFSNSTNNGILFVNSTILKAAYAEANANSNSNTKR

SEQ ID NO: 51 - protein, 105 aa, unidentified
  source 1..105: note = Vanderwaltozyma polyspora
APVESIFANQPDSSLTDTNDGVGVGMSTIKEEDFGKHFVENQILDEAVIMSLKLRKGVNLFFLDDIGLATELIGNKIAQIEAIDLSERLAQSWTNIRKNRLFGKR

SEQ ID NO: 52 - protein, 67 aa, unidentified
  source 1..67: note = Komagataella phaffii
APVSVNDAKEIAATFPQEALLGFLDLTDAENIVILSLVDEEKSGIALVNKTIWATARSEQAAGISKR

SEQ ID NO: 53 - protein, 86 aa, unidentified
  source 1..86: note = Eremothecium cymbalariae
APVDSGAKGKYSRTDLIIPDEAIANRYVVGDDEQPVFAEIDNKPVVYIVNTTKAESIVAKSGITLDDLKESYANATKEEEAKNGKR

SEQ ID NO: 54 - protein, 74 aa, unidentified
  source 1..74: note = Pichia sorbitophila
APVENINIKDNGNGTSEADVPGTSQGVEFPFAKEAIIEAVSLGNDIAPIVLNDAVYFVNTTTVDKELESKLGKR

SEQ ID NO: 55 - protein, 71 aa, unidentified
  source 1..71: note = Wickerhamomyces ciferrii
APVNITESANGEAEADVPGTSQGVEFPFSKEAIIEAVSLGNDIAPIVLDDAVYFINTTIVDQELGSKLGKR

SEQ ID NO: 56 - protein, 66 aa, unidentified
  source 1..66: note = Wickerhamomyces ciferrii
APVSTETDIDDLPISVPEEALIGFIDLTGDEVSLLPVNNGTHTGILFLNTTIAEAAFADKDDLKKR

SEQ ID NO: 57 - protein, 65 aa, unidentified
  source 1..65: note = Kluyveromyces lactis
APVENVDDSAQVPEEAIIGYIDFEGASDVAILPFSNSTDSGLMFVNTTIYNEATTAVEGESVEKR

SEQ ID NO: 58 - protein, 10 aa, unidentified
  source 1..10: note = Kluyveromyces delphensis
APIDVSLAKR

SEQ ID NO: 59 - protein, 66 aa, unidentified
  source 1..66: note = Kuraishia capsulata
APVNTTTEDETAQIPAEAVIGYLDLEGDFDVAVLPFSNSTNNGLLFINTTIASIAAKEEGVSLDKR

SEQ ID NO: 60 - protein, 66 aa, Saccharomyces cerevisiae
APANTTTEDETAQIPAEAVIGYLDLEGDFDVAVLPFSNSTNNGLSFINTTIASIAAKEEGVSLDKR

SEQ ID NO: 61 - protein, 66 aa, Saccharomyces cerevisiae
APANTTTEDETAQIPAEAVIDYSDLEGDFDAAALPLSNSTNNGLSSTNTTIASIAAKEEGVSLDKR

SEQ ID NO: 62 - protein, 8 aa, synthetic construct
  VARIANT 5..7: note = X is independently chosen from any amino acid
NTTIXXXA

SEQ ID NO: 63 - protein, 8 aa, synthetic construct
  VARIANT 5: note = X is alanine (A), leucine (L), or tyrosine (Y)
  VARIANT 6: note = X is alanine (A), serine (S), asparagine (N), or glutamic acid (E)
  VARIANT 7: note = X is alanine (A), isoleucine (I), serine (S), glutamic acid (E), or glutamine (Q)
NTTIXXXA

SEQ ID NO: 64 - protein, 9 aa, synthetic construct
RYVVGDDEQ

SEQ ID NO: 65 - protein, 7 aa, synthetic construct
IVAKSGI

SEQ ID NO: 66 - protein, 8 aa, synthetic construct
IPDEAIAN

SEQ ID NO: 67 - protein, 20 aa, synthetic construct
QTSISDDEEPIVVEINGQKV

SEQ ID NO: 68 - protein, 20 aa, synthetic construct
INTTLTEEALEKSGISIDDL

SEQ ID NO: 69 - protein, 9 aa, synthetic construct
PVFAEIDNK

SEQ ID NO: 70 - protein, 9 aa, synthetic construct
DDLKESYAN

SEQ ID NO: 71 - protein, 7 aa, synthetic construct
PVENVDD

SEQ ID NO: 72 - protein, 9 aa, synthetic construct
IDQEQLTNG

SEQ ID NO: 73 - protein, 12 aa, synthetic construct
PVDSGAKGKYSR

SEQ ID NO: 74 - protein, 20 aa, synthetic construct
NDGVGVGMSTIKEEDFGKHF

SEQ ID NO: 75 - protein, 16 aa, synthetic construct
  VARIANT 1: note = X is valine (V) or isoleucine (I)
  VARIANT 7: note = X is aspartic acid (D), serine (S), or glutamic acid (E)
  VARIANT 8: note = X is isoleucine (I) or glutamine (Q)
  VARIANT 10: note = X is alanine (A) or leucine (L)
  VARIANT 11: note = X is alanine (A) or lysine (K)
  VARIANT 16: note = X is serine (S) or leucine (L)
XNTTIAXXAXXEEGVX

SEQ ID NO: 76 - skipped

SEQ ID NO: 77 - protein, 20 aa, synthetic construct
  VARIANT 2: note = X is valine (V) or leucine (L)
  VARIANT 8: note = X is serine (S) or glycine (G)
  VARIANT 11: note = X is asparagine (N) or threonine (T)
  VARIANT 13: note = X is isoleucine (I) or leucine (L)
  VARIANT 14: note = X is serine (S), leucine (L), or methionine (M)
  VARIANT 16: note = X is valine (V) or isoleucine (I)
AXLPFSNXTNXGXXFXNTTI

SEQ ID NO: 78 - protein, 20 aa, synthetic construct
  VARIANT 1: note = X is threonine (T) or serine (S)
  VARIANT 4: note = X is isoleucine (I) or valine (V)
  VARIANT 9: note = X is valine (V) or isoleucine (I)
  VARIANT 12: note = X is tyrosine (Y) or phenylalanine (F)
  VARIANT 16: note = X is glutamic acid (E) or threonine (T)
  VARIANT 17: note = X is aspartic acid (D) or glycine (G)
  VARIANT 18: note = X is aspartic acid (D), serine (S), or alanine (A)
  VARIANT 19: note = X is glutamic acid (E) or phenylalanine (F)
XAQXPAEAXIGXLDLXXXXD

SEQ ID NO: 79 - protein, 12 aa, synthetic construct
  VARIANT 1: note = X is lysine (K) or serine (S)
  VARIANT 3: note = X is lysine (K) or arginine (R)
  VARIANT 4: note = X is tyrosine (Y) or phenylalanine (F)
  VARIANT 5: note = X is serine (S) or leucine (L)
  VARIANT 6: note = X is arginine (R) or glutamic acid (E)
  VARIANT 7: note = X is glutamine (Q) or threonine (T)
  VARIANT 9: note = X is leucine (L) or isoleucine (I)
  VARIANT 11: note = X is isoleucine (I) or phenylalanine (F)
XGXXXXXDXIXP

SEQ ID NO: 80 - skipped

SEQ ID NO: 81 - protein, 66 aa, synthetic construct
  VARIANT 3: note = X is valine (V) or alanine (A)
  VARIANT 5: note = X is threonine (T) or alanine (A)
  VARIANT 7: note = X is threonine (T) or alanine (A)
  VARIANT 9: note = X is aspartic acid (D) or glycine (G)
  VARIANT 11: note = X is threonine (T) or alanine (A)
  VARIANT 12: note = X is threonine (T) or alanine (A)
  VARIANT 14: note = X is isoleucine (I) or threonine (T)
  VARIANT 19: note = X is valine (V), isoleucine (I), or alanine (A)
  VARIANT 20: note = X is isoleucine (I) or alanine (A)
  VARIANT 21: note = X is glycine (G), aspartic acid (D), or threonine (T)
  VARIANT 23: note = X is leucine (L), serine (S), or arginine (R)
  VARIANT 24: note = X is aspartic acid (D) or glycine (G)
  VARIANT 25: note = X is leucine (L) or serine (S)
  VARIANT 29: note = X is phenylalanine (F), serine (S), or valine (V)
  VARIANT 31: note = X is valine (V) or alanine (A)
  VARIANT 33: note = X is valine (V) or alanine (A)
  VARIANT 36: note = X is phenylalanine (F) or leucine (L)
  VARIANT 37: note = X serine (S) or proline (P)
  VARIANT 38: note = X is asparagine (N), serine (S), or aspartic acid (D)
  VARIANT 42: note = X is asparagine (N) or aspartic acid (D)
  VARIANT 44: note = X is leucine (L) or serine (S)
  VARIANT 45: note = X is leucine (L) or serine (S)
  VARIANT 46: note = X is phenylalanine (F) or serine (S)
  VARIANT 47: note = X is isoleucine (I) or threonine (T)
  VARIANT 51: note = X is isoleucine (I) or threonine (T)
  VARIANT 53: note = X is serine (S) or asparagine (N)
  VARIANT 62: note = X is serine (S) or threonine (T)
  VARIANT 64: note = X is asparagine (N) or aspartic acid (D)
APXNXTXEXEXXQXPAEAXXXYXXXEGDXDXAXLPXXXSTNXGXXXXNTTXAXIAAKEEGVXLXKR

SEQ ID NO: 82 - protein, 66 aa, synthetic construct
  VARIANT 3: note = X is valine (V) or alanine (A)
  VARIANT 5: note = X is threonine (T) or alanine (A)
  UNSURE 7: note = X is threonine (T) or alanine (A)
  VARIANT 9: note = X is aspartic acid (D) or glycine (G)
  VARIANT 11: note = X is threonine (T) or alanine (A)
  VARIANT 12: note = X is threonine (T) or alanine (A)
  VARIANT 14: note = X is isoleucine (I) or threonine (T)
  VARIANT 19: note = X is valine (V) or alanine (A)
  VARIANT 20: note = X is isoleucine (I) or alanine (A)
  VARIANT 21: note = X is glycine (G), aspartic acid (D), or threonine (T)
  VARIANT 23: note = X is leucine (L), serine (S), or arginine (R)
  VARIANT 24: note = X is aspartic acid (D) or glycine (G)
  VARIANT 25: note = X is leucine (L) or serine (S)
  VARIANT 29: note = X is phenylalanine (F), serine (S), or valine (V)
  VARIANT 31: note = X is valine (V) or alanine (A)
  VARIANT 33: note = X is valine (V) or alanine (A)
  VARIANT 36: note = X is phenylalanine (F) or leucine (L)
  VARIANT 37: note = X serine (S) or proline (P)
  VARIANT 38: note = X is asparagine (N), serine (S), or aspartic acid (D)
  VARIANT 42: note = X is asparagine (N) or aspartic acid (D)
  VARIANT 44: note = X is leucine (L) or serine (S)
  VARIANT 45: note = X is leucine (L) or serine (S)
  VARIANT 46: note = X is phenylalanine (F) or serine (S)
  VARIANT 47: note = X is isoleucine (I) or threonine (T)
  VARIANT 51: note = X is isoleucine (I) or threonine (T)
APXNXTXEXEXXQXPAEAXXXYXXXEGDXDXAXLPXXXSTNXGXXXXNTTXASIAAKEEGVSLDKR

SEQ ID NO: 83 - DNA, 78 bp, unidentified
  source 1..78: mol_type = genomic DNA; note = Cutaneotrichosporon oleaginosum
gcgcctgttgcagctcccgaggccgaagctgggggtagaggaaacttcggcaatagcggaccaccgatatggaagagg

SEQ ID NO: 84 - DNA, 189 bp, unidentified
  source 1..189: mol_type = genomic DNA; note = Cutaneotrichosporon oleaginosum
gcgccaataaacattacaagcagtgaccctgccatcccctccgagtcaatttctgggtttcttgatctgaccgatgcagaagatttagctttgctaccagtttctaatggaactcattctggcgtcttgatcgtgaatacgactattctcgctcaggcattcggatcggacgatgtacttacaaagagg

SEQ ID NO: 85 - DNA, 270 bp, unidentified
  source 1..270: mol_type = genomic DNA; note = Lachancea quebecensis
gcccctatcgaagcaccaacttcagatgagacacccgcgccaaccgaaccgagtggcaggtttctggagcaagacattatattccctcagcaagctattaatgctcagacgtccatctcggatgacgaagaacccattgtggtagagataaacggacaaaaagttattctcctaatcaacactacattgaccgaagaggcattggaaaagagcgggatatctattgatgacttaaatgctttggccggtaattctactgtctccaaacga

SEQ ID NO: 86 - DNA, 36 bp, unidentified
  source 1..36: mol_type = genomic DNA; note = Suhomyces tanzawaensis NRRL Y-17324
gcaccagtgtccattagcggccaaagtttgaagcga

SEQ ID NO: 87 - DNA, 198 bp, unidentified
  source 1..198: mol_type = genomic DNA; note = Pachysolen tannophilus NRRL Y-2460
gccccggtgtccggggcatctaactcaaccagtatggaagtaccaggcgaggctgtcaaatattttttagacctatcggatagccctgatatggctcttgttcccataaataatggaaacacaacgggtattatgttcgttaacactactgtcatcgaccaagcgtacgcagaaactacctctctgagtagaaagcgt

SEQ ID NO: 88 - DNA, 204 bp, unidentified
  source 1..204: mol_type = genomic DNA; note = Hanseniaspora osmophila
gcagccatagatcaggaacaattaaccaatggcacgtatatcgacattccacaagagtcaattctttcgttcctggatttgactgatagtccggaagtgtctgtttaccctatcaaagaggggtccaagacaggactcatttttgtaaacagcactatagttgaccaggcgtattctgaaacgaccgctctaacacgtaaaaga

SEQ ID NO: 89 - DNA, 189 bp, unidentified
  source 1..189: mol_type = genomic DNA; note = Hanseniaspora uvarum
gccccgataaagttcaacgattcgtccccagcactcccccttgagagcattagtgggtatttagacctgacaggtgcggaagatttggctctactgcctgtgtctaatgctacccacacgggaatcttggtcgtaaataccactattttagcctctgctttggcatcagaatcaaactacaacaaaaga

SEQ ID NO: 90 - DNA, 198 bp, unidentified
  source 1..198: mol_type = genomic DNA; note = Lachancea nothofagi CBS 11611 |
gcgcctgtaaatacgaccactgaggatgaaacagcacaaataccagccgaagctatcattggttacttggaccttgagggagattttgatattgccgtgttacccttcagtaactcaacaaataacggcctactctttatcaatactaccattgcaaacatagctgctgaagaagagggggttactttgaacaaacga

SEQ ID NO: 91 - DNA, 21 bp, unidentified
  source 1..21: mol_type = genomic DNA; note = Saccharomyces paradoxus
aagcctcagcattataaacga

SEQ ID NO: 92 - DNA, 219 bp, unidentified
  source 1..219: mol_type = genomic DNA; note = Rhizophagus irregularis
gccccagtcgcgaaagacgcaacgaacactacagatgctagctccgtgcaaatacctgctgaagctgtaattggttacctagatttagagcagtcaaatgacgttgccatgcttcaattttctaacagtactaataatgggatcttgttcgttaacagtaccatcctcaaggcagcttatgcagaagctaatgccaactctaattcaaacacaaagcga

SEQ ID NO: 93 - DNA, 315 bp, unidentified
  source 1..315: mol_type = genomic DNA; note = Vanderwaltozyma polyspora
gcccccgtagaatcaatatttgctaaccaacctgacagctccttgacggataccaatgatggggtgggagtcggcatgtcgactattaaggaggaagacttcggtaaacattttgttgagaatcagatcctggatgaagcagttattatgtctcttaaattacgaaagggtgttaacctattctttctcgacgatatcggattggctacagaacttataggtaataagattgcgcaaatagaggcaattgacttaagtgaacgtttggctcagtcttggactaacatccgcaaaaatcggctttttggaaagaga

SEQ ID NO: 94 - DNA, 201 bp, unidentified
  source 1..201: mol_type = genomic DNA; note = Komagataella phaffii
gctcctgtatcggtcaatgatgcgaaggaaattgccgcaactttcccgcaggaggctttactaggttttttggacctgaccgatgcagaaaacatagtgatcctctcacttgttgacgaggaaaaatccggcattgctttggttaataaaacgatatgggccacagccaggagcgagcaagctgctggaattagtaagcga

SEQ ID NO: 95 - DNA, 258 bp, unidentified
  source 1..258: mol_type = genomic DNA; note = Eremothecium cymbalariae
gctccagtagattcgggcgcaaaaggtaagtactctcgaaccgaccttataatccccgatgaggcgattgccaacaggtatgttgtgggagatgacgaacaacctgtctttgctgaaatcgataataaaccggttgtgtatattgttaatacgactaaggcagagtcaattgtcgctaagagcgggataacattggacgatctgaaagaaagttacgccaacgctactaaagaggaagaggcaaagaacggaaaacgg

SEQ ID NO: 96 - DNA, 222 bp, unidentified
  source 1..222: mol_type = genomic DNA; note = Pichia sorbitophila
gcccccgttgagaacataaatatcaaggacaatgggaacggtacctcagaagctgatgtccctggaacatcgcagggcgtggaatttccattcgcaaaagaggctattattgaagcggtatctctgggtaatgatattgcaccgatcgtactaaacgatgctgtctactttgttaatacgactactgttgacaaggagttggaaagtaaattaggaaaacgg

SEQ ID NO: 97 - DNA, 213 bp, unidentified
  source 1..213: mol_type = genomic DNA; note = Wickerhamomyces ciferrii
gcgccagttaatattacggagtccgccaacggggaagctgaagctgacgtcccgggcacatcacaaggagtggaatttcccttcagcaaggaggccataatcgaagcagtatctctaggtaacgatattgcacctatagttctggatgatgcggtatattttattaatactaccatcgtcgaccaggagcttggtagtaaactcggaaaacgt

SEQ ID NO: 98 - DNA, 198 bp, unidentified
  source 1..198: mol_type = genomic DNA; note = Wickerhamomyces ciferrii
gctccagtgtccacagaaacggacatcgatgatttgcccataagtgttccggaggaagcccttattggctttattgatctgaccggagatgaggtcagcttattgcctgtaaacaatggtactcatactgggatactattcctcaatacaactattgcggaagctgcatttgcagacaaggatgacttaaaaaagcgt

SEQ ID NO: 99 - DNA, 195 bp, unidentified
  source 1..195: mol_type = genomic DNA; note = Kluyveromyces lactis
gcaccagtagaaaacgtggatgactccgcccaggtccccgaggaagcgattatcggatacatagatttcgagggcgcttcggatgttgctattcttccttttagtaatagcacagactcagggttaatgtttgttaatacgaccatctataacgaagctactactgcggtcgagggtgaatctgttgaaaagcga

SEQ ID NO: 100 - DNA, 30 bp, unidentified
  source 1..30: mol_type = genomic DNA; note = Kluyveromyces delphensis
gctcctatcgatgtgtctcttgccaagcgt

SEQ ID NO: 101 - DNA, 198 bp, unidentified
  source 1..198: mol_type = genomic DNA; note = Kuraishia capsulata
gcaccggtcaatacaaccacggaagatgagactgcccaaatacccgctgaagcggtgatcggctacctggaccttgagggagatttcgacgttgctgttctaccatttagtaacagcactaataacgggttattgtttattaatacaacgattgcatctatcgccgccaaagaagagggtgtatcactggataagcga

SEQ ID NO: 102 - DNA, 198 bp, Saccharomyces cerevisiae
  source 1..198: mol_type = genomic DNA
gcaccggccaatacaaccacggaagatgagactgctcaaatacccgctgaagcggtgatcggctacctggaccttgagggagatttcgacgtcgcagttctaccatttagtaacagcactaataacgggttatcctttattaatacaacgattgcttctatcgccgcgaaagaagagggtgtatcactcgataagcga

SEQ ID NO: 103 - DNA, 198 bp, Saccharomyces cerevisiae
  source 1..198: mol_type = genomic DNA
gcaccggccaatacaaccacggaagatgagactgctcaaatacccgctgaagcggtgatcgactacagcgatcttgagggcgacttcgatgcagccgctctgccattatcgaacagtactaataacgggttgtcttcaaccaatacaacgattgcctctattgcagcaaaagaagagggagtatccctagataagcga

SEQ ID NO: 104 - protein, 4 aa, synthetic construct
EAEA

SEQ ID NO: 105 - protein, 6 aa, synthetic construct
KREAEA

SEQ ID NO: 106 - protein, 86 aa, synthetic construct
MARFVALVLLGLLSLSGLDAAPANTTTEDETAQIPAEAVIDYSDLEGDFDAAALPLSNSTNNGLSSTNTTIASIAAKEEGVSLDKR

SEQ ID NO: 107 - protein, 92 aa, synthetic construct
MTKPTQVLVRSVSILFFITLLHLVVAAPANTTTEDETAQIPAEAVIDYSDLEGDFDAAALPLSNSTNNGLSSTNTTIASIAAKEEGVSLDKR

SEQ ID NO: 108 - protein, 82 aa, synthetic construct
MQLYLTLLFLLSFVECAPANTTTEDETAQIPAEAVIDYSDLEGDFDAAALPLSNSTNNGLSSTNTTIASIAAKEEGVSLDKR

SEQ ID NO: 109 - protein, 85 aa, synthetic construct
MRFPSIFTAVLFAASSALAAPANTTTEDETAQIPAEAVIDYSDLEGDFDAAALPLSNSTNNGLSSTNTTIASIAAKEEGVSLDKR

SEQ ID NO: 110 - protein, 95 aa, synthetic construct
METPRASLSLGRWSLWLLLLGLALPSASAAPANTTTEDETAQIPAEAVIDYSDLEGDFDAAALPLSNSTNNGLSSTNTTIASIAAKEEGVSLDKR

SEQ ID NO: 111 - protein, 88 aa, synthetic construct
MKFSAGAVLSWSSLLLASSVFAAPANTTTEDETAQIPAEAVIDYSDLEGDFDAAALPLSNSTNNGLSSTNTTIASIAAKEEGVSLDKR

SEQ ID NO: 112 - protein, 108 aa, synthetic construct
MRQVWFSWIVGLFLCFFNVSSAAPVDSGAKGKYSRTDLIIPDEAIANRYVVGDDEQPVFAEIDNKPVVYIVNTTKAESIVAKSGITLDDLKESYANATKEEEAKNGKR

SEQ ID NO: 113 - protein, 88 aa, synthetic construct
MRQVWFSWIVGLFLCFFNVSSAAPANTTTEDETAQIPAEAVIGYLDLEGDFDVAVLPFSNSTNNGLSFINTTIASIAAKEEGVSLDKR

SEQ ID NO: 114 - protein, 88 aa, synthetic construct
MFSLKALLPLALLLVSANQVAAAPANTTTEDETAQIPAEAVIDYSDLEGDFDAAALPLSNSTNNGLSSTNTTIASIAAKEEGVSLDKR

SEQ ID NO: 115 - protein, 85 aa, synthetic construct
MQHFLSLLLAVSLLTTTYAAPANTTTEDETAQIPAEAVIDYSDLEGDFDAAALPLSNSTNNGLSSTNTTIASIAAKEEGVSLDKR

SEQ ID NO: 116 - protein, 85 aa, synthetic construct
MKLFVPALLSLGALGLCLAAPANTTTEDETAQIPAEAVIDYSDLEGDFDAAALPLSNSTNNGLSSTNTTIASIAAKEEGVSLDKR

SEQ ID NO: 117 - protein, 48 aa, synthetic construct
MRQVWFSWIVGLFLCFFNVSSAAPVAAPEAEAGGRGNFGNSGPPIWKR

SEQ ID NO: 118 - protein, 105 aa, synthetic construct
MQHFLSLLLAVSLLTTTYAAPVDSGAKGKYSRTDLIIPDEAIANRYVVGDDEQPVFAEIDNKPVVYIVNTTKAESIVAKSGITLDDLKESYANATKEEEAKNGKR

SEQ ID NO: 119 - protein, 43 aa, synthetic construct
MKFTLATLLVLATAAIAAPVAAPEAEAGGRGNFGNSGPPIWKR

SEQ ID NO: 120 - protein, 106 aa, synthetic construct
MARFVALVLLGLLSLSGLDAAPVDSGAKGKYSRTDLIIPDEAIANRYVVGDDEQPVFAEIDNKPVVYIVNTTKAESIVAKSGITLDDLKESYANATKEEEAKNGKR

SEQ ID NO: 121 - protein, 32 aa, synthetic construct
MNVWHAVMVFVLCGVVVAAGAPVSISGQSLKR

SEQ ID NO: 122 - protein, 84 aa, synthetic construct
MRFPSIFTAVLFAASSALAAPVENVDDSAQVPEEAIIGYIDFEGASDVAILPFSNSTDSGLMFVNTTIYNEATTAVEGESVEKR

SEQ ID NO: 123 - protein, 105 aa, synthetic construct
MKLFVPALLSLGALGLCLAAPVDSGAKGKYSRTDLIIPDEAIANRYVVGDDEQPVFAEIDNKPVVYIVNTTKAESIVAKSGITLDDLKESYANATKEEEAKNGKR

SEQ ID NO: 124 - protein, 85 aa, synthetic construct
MKLFVPALLSLGALGLCLAAPANTTTEDETAQIPAEAVIGYLDLEGDFDVAVLPFSNSTNNGLSFINTTIASIAAKEEGVSLDKR

SEQ ID NO: 125 - protein, 93 aa, synthetic construct
MQHFLSLLLAVSLLTTTYAAPVENINIKDNGNGTSEADVPGTSQGVEFPFAKEAIIEAVSLGNDIAPIVLNDAVYFVNTTTVDKELESKLGKR

SEQ ID NO: 126 - protein, 90 aa, synthetic construct
MARFVALVLLGLLSLSGLDAAPANTTTEDETAQIPAEAVIDYSDLEGDFDAAALPLSNSTNNGLSSTNTTIASIAAKEEGVSLDKREAEA

SEQ ID NO: 127 - protein, 96 aa, synthetic construct
MTKPTQVLVRSVSILFFITLLHLVVAAPANTTTEDETAQIPAEAVIDYSDLEGDFDAAALPLSNSTNNGLSSTNTTIASIAAKEEGVSLDKREAEA

SEQ ID NO: 128 - protein, 86 aa, synthetic construct
MQLYLTLLFLLSFVECAPANTTTEDETAQIPAEAVIDYSDLEGDFDAAALPLSNSTNNGLSSTNTTIASIAAKEEGVSLDKREAEA

SEQ ID NO: 129 - protein, 89 aa, synthetic construct
MRFPSIFTAVLFAASSALAAPANTTTEDETAQIPAEAVIDYSDLEGDFDAAALPLSNSTNNGLSSTNTTIASIAAKEEGVSLDKREAEA

SEQ ID NO: 130 - protein, 99 aa, synthetic construct
METPRASLSLGRWSLWLLLLGLALPSASAAPANTTTEDETAQIPAEAVIDYSDLEGDFDAAALPLSNSTNNGLSSTNTTIASIAAKEEGVSLDKREAEA

SEQ ID NO: 131 - protein, 92 aa, synthetic construct
MKFSAGAVLSWSSLLLASSVFAAPANTTTEDETAQIPAEAVIDYSDLEGDFDAAALPLSNSTNNGLSSTNTTIASIAAKEEGVSLDKREAEA

SEQ ID NO: 132 - protein, 112 aa, synthetic construct
MRQVWFSWIVGLFLCFFNVSSAAPVDSGAKGKYSRTDLIIPDEAIANRYVVGDDEQPVFAEIDNKPVVYIVNTTKAESIVAKSGITLDDLKESYANATKEEEAKNGKREAEA

SEQ ID NO: 133 - protein, 92 aa, synthetic construct
MRQVWFSWIVGLFLCFFNVSSAAPANTTTEDETAQIPAEAVIGYLDLEGDFDVAVLPFSNSTNNGLSFINTTIASIAAKEEGVSLDKREAEA

SEQ ID NO: 134 - protein, 92 aa, synthetic construct
MFSLKALLPLALLLVSANQVAAAPANTTTEDETAQIPAEAVIDYSDLEGDFDAAALPLSNSTNNGLSSTNTTIASIAAKEEGVSLDKREAEA

SEQ ID NO: 135 - protein, 89 aa, synthetic construct
MQHFLSLLLAVSLLTTTYAAPANTTTEDETAQIPAEAVIDYSDLEGDFDAAALPLSNSTNNGLSSTNTTIASIAAKEEGVSLDKREAEA

SEQ ID NO: 136 - protein, 89 aa, synthetic construct
MKLFVPALLSLGALGLCLAAPANTTTEDETAQIPAEAVIDYSDLEGDFDAAALPLSNSTNNGLSSTNTTIASIAAKEEGVSLDKREAEA

SEQ ID NO: 137 - protein, 52 aa, synthetic construct
MRQVWFSWIVGLFLCFFNVSSAAPVAAPEAEAGGRGNFGNSGPPIWKREAEA

SEQ ID NO: 138 - protein, 109 aa, synthetic construct
MQHFLSLLLAVSLLTTTYAAPVDSGAKGKYSRTDLIIPDEAIANRYVVGDDEQPVFAEIDNKPVVYIVNTTKAESIVAKSGITLDDLKESYANATKEEEAKNGKREAEA

SEQ ID NO: 139 - protein, 24 aa, synthetic construct
MKLKYFLLIFVFTTVLAKPQHYKR

SEQ ID NO: 140 - protein, 110 aa, synthetic construct
MARFVALVLLGLLSLSGLDAAPVDSGAKGKYSRTDLIIPDEAIANRYVVGDDEQPVFAEIDNKPVVYIVNTTKAESIVAKSGITLDDLKESYANATKEEEAKNGKREAEA

SEQ ID NO: 141 - protein, 36 aa, synthetic construct
MNVWHAVMVFVLCGVVVAAGAPVSISGQSLKREAEA

SEQ ID NO: 142 - protein, 109 aa, synthetic construct
MKLFVPALLSLGALGLCLAAPVDSGAKGKYSRTDLIIPDEAIANRYVVGDDEQPVFAEIDNKPVVYIVNTTKAESIVAKSGITLDDLKESYANATKEEEAKNGKREAEA

SEQ ID NO: 143 - protein, 89 aa, synthetic construct
MKLFVPALLSLGALGLCLAAPANTTTEDETAQIPAEAVIGYLDLEGDFDVAVLPFSNSTNNGLSFINTTIASIAAKEEGVSLDKREAEA

SEQ ID NO: 144 - protein, 97 aa, synthetic construct
MQHFLSLLLAVSLLTTTYAAPVENINIKDNGNGTSEADVPGTSQGVEFPFAKEAIIEAVSLGNDIAPIVLNDAVYFVNTTTVDKELESKLGKREAEA

SEQ ID NO: 145 - protein, 89 aa, synthetic construct
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 146 - protein, 15 aa, synthetic construct
  VARIANT 2: note = X is leucine (L), phenylalanine (F), or glycine (G)
  VARIANT 3: note = X is leucine (L), phenylalanine (F), or valine (V)
  VARIANT 4: note = X is asparagine (N) or valine (V)
  VARIANT 5: note = X is valine (V) or leucine (L)
  VARIANT 6: note = X is serine (S), alanine (A), or valine (V)
  VARIANT 7: note = X is serine (S) or alanine (A)
  VARIANT 8: note = X is alanine (A) or glycine (G)
  VARIANT 11: note = X is valine (V) or alanine (A)
CXXXXXXXAPXNTTT

SEQ ID NO: 147 - protein, 12 aa, synthetic construct
  VARIANT 1: note = X is leucine (L), serine (S), or alanine (A)
  VARIANT 5: note = X is alanine (A) or valine (V)
  VARIANT 6: note = X is asparagine (N) or serine (S)
XAAPXXTTTEDE

SEQ ID NO: 148 - protein, 9 aa, synthetic construct
  VARIANT 5: note = X is asparagine (N) or lysine (K)
  VARIANT 6: note = X is isoleucine (I) or phenylalanine (F)
  VARIANT 7: note = X is threonine (T) or asparagine (N)
  VARIANT 8: note = X is serine (S) or aspartic acid (D)
AAPIXXXXS

SEQ ID NO: 149-152 - skipped (4 entries)

SEQ ID NO: 153 - protein, 78 aa, synthetic construct
  VARIANT 2: note = X is phenylalanine (F), threonine (T), or leucine (L)
  VARIANT 3: note = X is phenylalanine (F), leucine (L), or alanine (A)
  VARIANT 4: note = X is isoleucine (I), leucine (L), or valine (V)
  VARIANT 5: note = X is threonine (T), phenylalanine (F), or serine (S)
  VARIANT 8: note = X is histidine (H), serine (S), or threonine (T)
  VARIANT 9: note = X is leucine (L), phenylalanine (F), or threonine (T)
  VARIANT 10: note = X is valine (V) or threonine (T)
  VARIANT 11: note = X is valine (V), glutamic acid (E), or tyrosine (Y)
  VARIANT 12: note = X is alanine (A) or cytosine (C)
  VARIANT 15: note = X is valine (V) or alanine (A)
  VARIANT 17: note = X is threonine (T) or alanine (A)
  VARIANT 19: note = X is threonine (T) or alanine (A)
  VARIANT 21: note = X is aspartic acid (D) or glycine (G)
  VARIANT 23: note = X is threonine (T) or alanine (A)
  VARIANT 24: note = X is threonine (T) or alanine (A)
  VARIANT 26: note = X is isoleucine (I) or threonine (T)
  VARIANT 31: note = X is valine (V) or alanine (A)
  VARIANT 32: note = X is isoleucine (I) or alanine (A)
  VARIANT 33: note = X is glycine (G), aspartic acid (D), or threonine (T)
  VARIANT 35: note = X is leucine (L), serine (S), or arginine (R)
  VARIANT 36: note = X is aspartic acid (D) or glycine (G)
  VARIANT 37: note = X is leucine (L) or serine (S)
  VARIANT 41: note = X is phenylalanine (F), serine (S), or valine (V)
  VARIANT 43: note = X is valine (V) or alanine (A)
  VARIANT 45: note = X is valine (V) or alanine (A)
  VARIANT 48: note = X is phenylalanine (F) or leucine (L)
  VARIANT 49: note = X serine (S) or proline (P)
  VARIANT 50: note = X is asparagine (N), serine (S), or aspartic acid (D)
  VARIANT 54: note = X is asparagine (N) or aspartic acid (D)
  VARIANT 56: note = X is leucine (L) or serine (S)
  VARIANT 57: note = X is leucine (L) or serine (S)
  VARIANT 58: note = X is phenylalanine (F) or serine (S)
  VARIANT 59: note = X is isoleucine (I) or threonine (T)
  VARIANT 63: note = X is isoleucine (I) or threonine (T)
LXXXXLLXXXXXAPXNXTXEXEXXQXPAEAXXXYXXXEGDXDXAXLPXXXSTNXGXXXXNTTXASIAAKEEGVSLDKR

SEQ ID NO: 154 - protein, 14 aa, synthetic construct
MTKPTQVLVRSVSI

SEQ ID NO: 155 - protein, 4 aa, synthetic construct
MQLY

SEQ ID NO: 156 - protein, 7 aa, synthetic construct
MQHFLSL

SEQ ID NO: 157 - protein, 78 aa, synthetic construct
  UNSURE 2: note = X is phenylalanine (F), threonine (T), or leucine (L)
  VARIANT 3: note = X is phenylalanine (F), leucine (L), or alanine (A)
  VARIANT 4: note = X is isoleucine (I), leucine (L), or valine (V)
  VARIANT 5: note = X is threonine (T), phenylalanine (F), or serine (S)
  VARIANT 8: note = X is histidine (H), serine (S), or threonine (T)
  VARIANT 9: note = X is leucine (L), phenylalanine (F), or threonine (T)
  VARIANT 10: note = X is valine (V) or threonine (T)
  VARIANT 11: note = X is valine (V), glutamic acid (E), or tyrosine (Y)
  VARIANT 12: note = X is alanine (A) or cytosine (C)
LXXXXLLXXXXXAPANTTTEDETAQIPAEAVIDYSDLEGDFDAAALPLSNSTNNGLSSTNTTIASIAAKEEGVSLDKR

SEQ ID NO: 158 - protein, 66 aa, synthetic construct
APVNTTTEDETAQIPAEAVIGYLDLEGDFDVAVLPFSNSTNNGLLFINTTIASIAAKEEGVQLDKR

SEQ ID NO: 159 - protein, 689 aa, synthetic construct
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 160 - protein, 162 aa, synthetic construct
TNIPQTMQDLDLQEVAGKWHSVAMAASDISLLDSESAPLRVYIEKLRPTPEDNLEIILREGENKGCAEKKIFAEKTESPAEFKINYLDEDTVFALDTDYKNYLFLCMKNAATPGQSLVCQYLARTQMVDEEIMEKFRRALQPLPGRVQIVPDLTRMAERCRI

SEQ ID NO: 161 - protein, 162 aa, synthetic construct
IIVTQTMKDLDVQKVAGTWYSLAMAASDISLLDAQSAPLRVYVEELKPTPGGDLEILLQKWENGKCAQKKIIAEKTEIPAVFKIDALNENKVLVLDTDYKKYLLFCMENSAEPEQSLACQCLVRTPEVDDEAMEKFDKALKALPMHIRLSFNPTQLEEQCRV

SEQ ID NO: 162 - protein, 162 aa, synthetic construct
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 163 - protein, 162 aa, synthetic construct
IIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 164 - protein, 162 aa, synthetic construct
IIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDKEALEKFDKALKALPMHIRLAFNPTQLEGQCHV

SEQ ID NO: 165 - protein, 162 aa, synthetic construct
IVVPQTVENLDLQKVAGTWHSLAIAASDISLLDAETAPLRVYVQELRPTPEGNLEIVLRKWEDGRCPEQKVVAEKTKVPAEFKINYLEENKIFVLDTDYKNYLFFCMENTDAPEQRLMCQYLARTLKVDNEVMGKFNRALETLPVHMQIIPDLTQGKEQCHV

SEQ ID NO: 166 - protein, 382 aa, synthetic construct
GSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 167 - protein, 385 aa, synthetic construct
GSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 168 - protein, 390 aa, synthetic construct
GSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 169 - protein, 385 aa, synthetic construct
GSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 170 - protein, 385 aa, synthetic construct
GSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 171 - DNA, 822 bp, synthetic construct
  source 1..822: mol_type = other DNA
caggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 172 - protein, 88 aa, synthetic construct
MRFPSIFTAVLFAASSALAAPVENVDDSAQVPEEAIIGYIDFEGASDVAILPFSNSTDSGLMFVNTTIYNEATTAVEGESVEKREAEA

SEQ ID NO: 173 - protein, 11 aa, synthetic construct
  SITE 2: note = X is 0 amino acids in length, 1 amino acid in length, 2 amino acids in length, 3 amino acids in length, 4 amino acids in length, or 5 amino acids in length. In some embodiments, X is 0-1 amino acids in length, 0-2 amino acids in length, 0-3 amino acids in length, 0-4 amino acids in length, 1-2 amino acids in length, 1-3 amino acids in length, 1-4 amino acids in length, 1-5 amino acids in length, 2-3 amino acids in length, 2-4 amino acids in length, 2-5 amino acids in length, 3-4 amino acids in length, 3-5 amino acids in length, or 4-5 amino acids in length.
  SITE 4: note = X is 0 amino acids in length, 1 amino acid in length, 2 amino acids in length, or 3 amino acids in length. In some embodiments, X is 0-1 amino acids in length, 0-2 amino acids in length, 1-2 amino acids in length, 1-3 amino acids in length, or 2-3 amino acids in length.
  VARIANT 2: note = X is independently selected from any naturally occurring amino acid.
  VARIANT 2: note = X is independently selected from the group consisting of threonine (T), lysine (K), and proline (P).
  VARIANT 4: note = X is independently selected from any naturally occurring amino acid.
  VARIANT 4: note = X is independently selected from the group consisting of valine (V), histidine (H), and phenylalanine (F).
  SITE 6: note = X is 0 amino acids in length, 1 amino acid in length, 2 amino acids in length, 3 amino acids in length, 4 amino acids in length, 5 amino acids in length, 6 amino acids in length, or 7 amino acids in length. In some embodiments, X is 0-1 amino acids in length, 0-2 amino acids in length, 0-3 amino acids in length, 0-4 amino acids in length, 0-5 amino acids in length, 0-6 amino acids in length, 1-2 amino acids in length, 1-3 amino acids in length, 1-4 amino acids in length, 1-5 amino acids in length, 1-6 amino acids in length, 1-7 amino acids in length, 2-3 amino acids in length, 2-4 amino acids in length, 2-5 amino acids in length, 2-6 amino acids in length, 2-7 amino acids in length, 3-4 amino acids in length, 3-5 amino acids in length, 3-6 amino acids in length, 3-7 amino acids in length, 4-5 amino acids in length, 4-6 amino acids in length, 4-7 amino acids in length, 5-6 amino acids in length, 5-7 amino acids in length, or 6-7 amino acids in length.
  VARIANT 6: note = X is independently selected from any naturally occurring amino acid.
  VARIANT 6: note = X is independently selected from the group consisting of valine (V), arginine (R), serine (S), isoleucine (I), and phenylalanine (F).
  SITE 8: note = X is 0 amino acids in length, 1 amino acid in length, 2 amino acids in length, 3 amino acids in length, 4 amino acids in length, or 5 amino acids in length. In some embodiments, X is 0-1 amino acids in length, 0-2 amino acids in length, 0-3 amino acids in length, 0-4 amino acids in length, 1-2 amino acids in length, 1-3 amino acids in length, 1-4 amino acids in length, 1-5 amino acids in length, 2-3 amino acids in length, 2-4 amino acids in length, 2-5 amino acids in length, 3-4 amino acids in length, 3-5 amino acids in length, or 4-5 amino acids in length.
  SITE 8: note = X is independently selected from any naturally occurring amino acid.
  VARIANT 8: note = X is independently selected from the group consisting of alanine (A), valine (V), threonine (T), serine (S), isoleucine (I), leucine (L), and phenylalanine (F).
  SITE 11: note = X is 0 amino acids in length, 1 amino acid in length, 2 amino acids in length, 3 amino acids in length, 4 amino acids in length, 5 amino acids in length, or 6 amino acids in length. In some embodiments, X is 0-1 amino acids in length, 0-2 amino acids in length, 0-3 amino acids in length, 0-4 amino acids in length, 0-5 amino acids in length, 1-2 amino acids in length, 1-3 amino acids in length, 1-4 amino acids in length, 1-5 amino acids in length, 1-6 amino acids in length, 2-3 amino acids in length, 2-4 amino acids in length, 2-5 amino acids in length, 2-6 amino acids in length, 3-4 amino acids in length, 3-5 amino acids in length, 3-6 amino acids in length, 4-5 amino acids in length, 4-6 amino acids in length, or 5-6 amino acids in length.
  VARIANT 11: note = X is independently selected from any naturally occurring amino acid.
  VARIANT 11: note = X is independently selected from the group consisting of alanine (A), valine (V), leucine (L), threonine (T), serine (S), tyrosine (Y), histidine (H), cytosine (C), glutamic acid (E), and phenylalanine (F).
MXQXLXLXLLX

SEQ ID NO: 174 - DNA, 691 bp, synthetic construct
  source 1..691: mol_type = other DNA
tgagttcatcctgcagaataccgcggcgttccacatctgatgccattggcggaggggtccggacggtcaggaacttagccttatgagatgaatgatggacgtgtctggcctcggaaaaggatatatggggatcataatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcc

SEQ ID NO: 175 - DNA, 558 bp, synthetic construct
  source 1..558: mol_type = other DNA
cgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacg

SEQ ID NO: 176 - DNA, 491 bp, synthetic construct
  source 1..491: mol_type = other DNA
gatccttttttgtagaaatgtcttggtgtcctcgaccaatcaggtagccatccctgaaatacctggctccgtggcaacaccgaacgacctgctggcaacgttaaattctccggggtaaaacttaaatgtggagtaatagaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaagtcccggccgtcgctggcaataactgcgggcggacgcatgtcttgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 177 - DNA, 492 bp, synthetic construct
  source 1..492: mol_type = other DNA
gatccttttttgtagaaatgtcttggtgtcctcgtccaatcaggtagccatctctgaaatatctggctccgttgcaactccgaacgacctgctggcaacgtaagatactccgaggtaaaacttaaatgtggagtaatggaaccagaaacgtctcttcctttctctctccttccaccgcccgttaccgtccctaggaagttttactctgctggagagcttcttctacggcccccttgcagcaatgctcttcccagcattacgttgcgggtaaaacggaggtcttgtacccgacctagcagcccagggatggaaaagtcccggccgttgctggcaataatagcgggcggacgcatgtcatgagattactggaaaccaccagaatcgaacataaaaggcgaacacctgtcctaaattaggtttctcctgacccaaagactttaaatttaatttatttgtccctatttcaatcaattgaacaactatcaaaacaca

SEQ ID NO: 178 - DNA, 350 bp, synthetic construct
  source 1..350: mol_type = other DNA
ccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatc

SEQ ID NO: 179 - DNA, 364 bp, synthetic construct
  source 1..364: mol_type = other DNA
gagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 180 - DNA, 336 bp, synthetic construct
  source 1..336: mol_type = other DNA
gagctccctaggtgattggatccaatcttgccccattcgctaagcccacattggatccaatagctagaccttacggattggtgcattggatccaatggtcgaacatctgctataagcgcattggatccaataaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 181 - DNA, 266 bp, synthetic construct
  source 1..266: mol_type = other DNA
gagctccctaggtgattggatccaatcttgccccattcgctaagcccacattggatccaataaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 182 - DNA, 238 bp, synthetic construct
  source 1..238: mol_type = other DNA
gagctccctaggtgtccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 183 - DNA, 532 bp, synthetic construct
  source 1..532: mol_type = other DNA
gagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagatcgtcgacctagctctgtcttagtccctatcagtgatagagataacatgcctctcactaacatggtccctatcagtgatagagactactggggccacgattcgtgtgtccctatcagtgatagagatctgcgtaatactactcgcgtgttccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 184 - DNA, 368 bp, synthetic construct
  source 1..368: mol_type = other DNA
gagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 185 - DNA, 650 bp, synthetic construct
  source 1..650: mol_type = other DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgcaagcttcaacgatgccaaaagggtataataagcgtcatttgcagcattgtgaagaaaactatgtggcaagccaagcctgcgaagaatgtattttaagtttgactttgatgtattcacttgattaagccataattctcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtt

SEQ ID NO: 186 - DNA, 204 bp, synthetic construct
  source 1..204: mol_type = other DNA
ttgaaatgtatttaatttgatattaagtaaatgaatgattatgactttatgaattcgcaatgttttctccttgattatttctgtattgtattggaatgattatagaatactcatatattgattatagtattagcacataaaacgtttgttgttaaactcacttccgtacgcaaccatttctatttctagctatcttgataaggt

SEQ ID NO: 187 - DNA, 199 bp, synthetic construct
  source 1..199: mol_type = other DNA
tactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactg

SEQ ID NO: 188 - DNA, 45 bp, synthetic construct
  source 1..45: mol_type = other DNA
tatatatatataactgtctagaaataaagagtatcatctttcaaa

SEQ ID NO: 189 - DNA, 472 bp, synthetic construct
  source 1..472: mol_type = other DNA
tagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgct

SEQ ID NO: 190 - DNA, 500 bp, synthetic construct
  source 1..500: mol_type = other DNA
acgggaagtctttacagttttagttaggagcccttatatatgacagtaatgctagtacgttttgttttgtttaattaataacttagtttatgttagcctagtatagactccatcaattttttttgttattacgtaagccgcgatgataatatctgatgaaaaattcctatcagaaaataatttatcaaaagtttcatgcgatatgagactaagtagaatagggactcccaaagtgtcagtcacaagggtcattcccgttcgtaatgtggtgatagcgaggagaaaacctgtcagagcaagtaacaccgacgcaaagacatggctaatgaaagaagagcagagaagaataagacagaaggagcaggagatgaaacaaaggctagaggaactagaaaggttcaaaacaaaagtacagaaatcatatataaggaaagaggataggcatttggcacaagagatagaaaaggatcttgacataatcactgatgattacaatttgg

SEQ ID NO: 191 - DNA, 634 bp, synthetic construct
  source 1..634: mol_type = other DNA
agatctaatcaggacggcaaactcaattcagaagtgtgctgtgagtgagactgattgccgagcgcagacgactctcgtggaacccggcttgtggagaagcttgagaaggtcttaactcctagcgtaaaagctcatgatgacgtacaatttaatgaaatgatacaatgttcatatttcccgttcaaatttccggccttggtcagtgcgtaagatgtccacgattgaatactaactcagtatgggtttggtagcattggcaatgtagttataagcatgcaccggttgaagacgtcggccccagatgcaatgctgcggtggtgactaagctctgcagtgaatggaatgcgtttctttgatcgacttcggcgtgccgcgggattttctcggcgcttctactggtgcagaaaggacgataccactggctttcggtccatgccacatcccagtctcccgggaaattcattgcatactttaagaaacaaactgatctccataatttccgtctttagagttcacttggtacttttgggtggatcgaggggtgtccgcggccatccaagtcacgtggagggcagctagaccacggattttagagctacattgatccaagactcctggaccggcctcatgggcc

SEQ ID NO: 192 - protein, 498 aa, unidentified
  source 1..498: note = Gigaspora rosea
MRRSTIFFVLGLLTLTSLTSADIYLKETFSDDDWEKRWVHSKHKEDLGKFKVTAGEFYAHEIESRGLQTTEDARFYAISTKFDKIIDNTDKDLVVQYSVKHEQNIDCGGGYVKLLPSEFDALSFKGESLYNIMFGPDICGMNRKVHFIVHHKGENKELKKSIKAPSDQVTHLYTLILKPDHTYKILIDNEEEASGTLEEDFDLLPPEEITDPTAKKPEDWEDLAEIPDPDDHKPEDWVDHPATIPDPDAKKPDDWDDEMDGDWEPPQISNPDYKGEWKPKKIPNPKYKGEWKAPMIPNPDYVPEPNLHAFKTEFIGFDLWQVRSGTIFDNILITDDVETAEKFANETFVKFRDAEKEAKKKLEELEKEQDEADDKDKKDGDKKDDDDDIIDLDVKLGDDGEVKVTKPEKDEKEKEKVKDEKEKEKVKDEKDKEKVKDEKEKESKDKVKTEEVKKEEKAKETKDEKDEMDKLLDDLEEELGLPPKPKKEDHKLPIKDEL

SEQ ID NO: 193 - protein, 417 aa, Chlorocebus aethiops
MLLSVPLLLGLLGLAAAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 194 - protein, 416 aa, Mus musculus
MLLSVPLLLGLLGLAAADPAIYFKEQFLDGDAWTNRWVESKHKSDFGKFVLSSGKFYGDLEKDKGLQTSQDARFYALSAKFEPFSNKGQTLVVQFTVKHEQNIDCGGGYVKLFPSGLDQKDMHGDSEYNIMFGPDICGPGTKKVHVIFNYKGKNVLINKDIRCKDDEFTHLYTLIVRPDNTYEVKIDNSQVESGSLEDDWDFLPPKKIKDPDAAKPEDWDERAKIDDPTDSKPEDWDKPEHIPDPDAKKPEDWDEEMDGEWEPPVIQNPEYKGEWKPRQIDNPDYKGTWIHPEIDNPEYSPDANIYAYDSFAVLGLDLWQVKSGTIFDNFLITNDEAYAEEFGNETWGVTKAAEKQMKDKQDEEQRLKEEEEDKKRKEEEEAEDKEDDDDRDEDEDEEDEKEEDEEESPGQAKDEL

SEQ ID NO: 195 - protein, 447 aa, unidentified
  source 1..447: note = Piromyces finnis
MRFGTTLAILSFCAAAFGKVYFHETFDDDSWEKHWVQSTYKDDYGKFKISNGKEFRADPVKSRGLQTSQNAKFYSISAPFDEAFNNKEKDLIVQFSVRHEQNIDCGGGYIKVLPPNIDPKEFNGETPYNIMFGSDICGANKKTHLILSYKGKNHLIKKEIPTEDDTYTHLYTLVIKPDQTYSVSIDNVEKASGSFEDWDFLEPKTIPDPEKTKPADWVDDEYIDDPNDKKPDDWDEDEPQYIDDPEAEKPEDWDDDMDGEWEAPKSENPKYKGKWTPKKIKNPEYKGKWIQPEIPNPDYFDDKEIYVYDSGFVGFDLWQVKAGSIFDDIVVTDDAEEAKTFATEVMDKIKAEKEDEEKVAAERKKIMDEKNKRAAKKAEEMNEKRRKLERENHPEAYADEEESNDDSEEEMEETKEEEKEEVKEETKEETKEEAKEEEKKENVKDEL

SEQ ID NO: 196 - protein, 508 aa, unidentified
  source 1..508: note = Anaeromyces robustus
MRFSKLFKIVASVALAAKVAAEGNVVSLTKDDYEVTLEEAPLALVKYFAPWCGHCKALAPEFVKAADALKEQNILLAEVDCTVESDICNEVGVRGYPTLKVYRNGKASDYKGQRTAESIISYMKKQSLPDLTAIKAEDFETFSTSDKVVVVGFVKEGSDEYKALEANAKELREQFVFGYIDDAELAKKAGAAVPGIVVYKQFDEGKAVLEGEITEESIKNFVNIESVPLMDELGPENYSKYMESGLPLVYLFTSDAEDKKTVGAWCEAIAKKVQGKLNFVYIDAVKFSGHGKFLGLKETWPAVIIQDVKHNTKYPFPQDKKIEKEELEKFIDDFQAGKLEPFLKSQDVPEKQEDGIYNVVAKTFDEVVLDKSKDVLLLFYAPWCGHCKKLAPTYKEIAEEINENNDKVLIARMDATENDIPSTSPWNNLEGFPTVILFKADDKNTAVVYEGDRSKESILEFIKKNAVNEFKVPEKKEEEKKEEEEKKEEKKEEEKKEEKDDEKVKDEL

SEQ ID NO: 197 - protein, 409 aa, unidentified
  source 1..409: note = Dictyostelium discoideum
MKLINICIFIFAIICIESTFGFYTDNSNVINLTKKNFQQQVLNSQQNWMVEFYAPWCGHCKSLKPEYEKVSNNLKGLVKIGAINCDEEKELCGQYQIQGFPTLKFFSTNPKTGKKGQPEDYQGARSASEIAKFSLAKLPSNHIQKVSQDNINKFLTGTSDAKALLFTDKPKTTDLYKALSVDFFKTLTLGEARNLNKETLEKFNIDKFPTLLVFTNDDGETFTKFDGKLTHSTIYKFLEPFSKKSNNDNNNNNNNNNNEESTKTTTTEKDPASEKFIEIKDEKSFEKSCSTGLCIVALFDQSSIDDKELNEKYLELLNTVSQNFIGRMKFVWVDVSVHDKIVPQFDLSGTPNIFVINNSKKRYTPFMGSFSDESLNSFFKSVLSGLKKAIPFTDSPKFNSQQKKQKDEL

SEQ ID NO: 198 - protein, 459 aa, unidentified
  source 1..459: note = Lichtheimia ramose
MAFFRNLFLLLLVVNAAFLGYDHFKGGDIAVSLVDNAKRLDVPTVQNHLQQTWTSIKSTNPEKLAGHVNAAFDQLKGFNSFSDVVDHVRAKVSGAVGGSADMGRIILEDNVFVLNDKNFDKVIDGSRPALVEFYAPWCGHCKKLAPTYAELGEAFSTVQDRVVIAKVNADEQRDLGARFGIQGFPTLKWFPKGVTTPDGIEDYRGGRDLDSLSKFVHEKSGVRPRVKSTKSDVVVLDSQNFNSIVKDPKTNVLVEFYAPWCGHCKNLAPTYEKVATAFANEPNCKVAKIDADSERAIGTEYEISGFPTIKFFAAGEDKEPVAYEGPRTEAGFIEFLNKQCGTHRLVGGSLDATAGRIADLDQLAIKFASTSDKVAREAIQKEATVVAGELGTRNAKFYGIVMKKVLEKGDGFIKTENARLDKIIKSNTVTASKVDDFTVRKNILAAFDKKAKPVTKDEL

SEQ ID NO: 199 - protein, 517 aa, Anopheles christyi
MQFNWNIKTVASILSALTLAQASDQEAIAPEDSHVVKLTEATFESFITSNPHVLAEFFAPWCGHCKKLGPELVSAAEILKDNEQVKIAQIDCTEEKELCQGYEIKGYPTLKVFHGEVEVPSDYQGQRQSQSIVSYMLKQSLPPVSEINATKDLDDTIAEAKEPVIVQVLPEDASNLESNTTFYGVAGTLREKFTFVSTKSTDYAKKYTSDSTPAYLLVRPGEEPSVYSGEELDETHLVHWIDIESKPLFGDIDGSTFKSYAEANIPLAYYFYENEEQRAAAADIIKPFAKEQRGKINFVGLDAVKFGKHAKNLNMDEEKLPLFVIHDLVSNKKFGVPQDQELTNKDVTELIEKFIAGEAEPIVKSEPIPEIQEEKVFKLVGKAHDEVVFDESKDVLVKYYAPWCGHCKRMAPAYEELATLYANDEDASSKVVIAKLDHTLNDVDNVDIQGYPTLILYPAGDKSNPQLYDGSRDLESLAEFVKERGTHKVDALALRPVEEEKEAEEEAESEADAHDEL

SEQ ID NO: 200 - skipped

SEQ ID NO: 201 - protein, 161 aa, unidentified
  source 1..161: note = Pichia pastoris
MSSFYDLAPLDKKGEPFPFEQLKGKVVLIVNVASKCGFTPQYTELEKLYKDHKDEGLTIVGFPCNQFGHQEPGNDEEIGQFCQLNFGVTFPILKKIDVNGSEADPVYEFLKSKKSGLLGFKGIKWNFEKFLIDKQGNVIERYSSLTKPSSIESKIEELLKK

SEQ ID NO: 202 - protein, 163 aa, Saccharomyces cerevisiae
MSEFYKLAPVDKKGQPFPFDQLKGKVVLIVNVASKCGFTPQYKELEALYKRYKDEGFTIIGFPCNQFGHQEPGSDEEIAQFCQLNYGVTFPIMKKIDVNGGNEDPVYKFLKSQKSGMLGLRGIKWNFEKFLVDKKGKVYERYSSLTKPSSLSETIEELLKEVE

SEQ ID NO: 203 - protein, 230 aa, Saccharomyces cerevisiae
MEMTDFELTSNSQSNLAIPTNFKSTLPPRKRAKTKEEKEQRRIERILRNRRAAHQSREKKRLHLQYLERKCSLLENLLNSVNLEKLADHEDALTCSHDAFVASLDEYRDFQSTRGASLDTRASSHSSSDTFTPSPLNCTMEPATLSPKSMRDSASDQETSWELQMFKTENVPESTTLPAVDNNNLFDAVASPLADPLCDDIAGNSLPFDNSIDLDNWRNPAVITMTRKLQ

SEQ ID NO: 204 - protein, 624 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSEFPPKKKRKVGSTSGSGKPGSGEGSTKGDALDDFDLDMLGSDALDDFDLDMLGSDALDDFDLDMLGSDALDDFDLDMLINSRSSGSPKKKRKVGSGGGSGGSGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 205 - protein, 392 aa, synthetic construct
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADEFPPKKKRKVGSTSGSGKPGSGEGSTKGEFPGITLRIQETDMLYKGDTLYLDWLEDGIAELVFDAPGSVNKLDTAVASLGEAIGVLEQQSDLIWETLTVKDAKVNFDSGLEKFEEAIPSADDFDPVAERRSSGEFRAERHSGGTDLCF

SEQ ID NO: 206 - protein, 536 aa, unidentified
  source 1..536: note = Pichia pastoris
MSDFPKVEQASPSEGMPQVQTNNAPEIEEIEDIIRDTPNVMDVLGNGSQSWDGTNSNGAAMGSLDTVPANKLVAYNKYDNNIYPDPLQPLSDTALPNSMGYYSMIKAGDKGLKPQSFRKTKKKPTPSGPKTRPAFVMKLWNMVHDPSNQAFIRWLPDGKSFQVTNREDFLKHVLPKYFKHNNFASFVRQLNMYGWHKVQDVGNGSLTANEELWQFENPNFIRDREDLLDQIVRNKSKPGEDDENIDFGLVLNELETIKMNQMAISEDLRRIRQDNETLWQEHYLARERHKTQAETLEKMMRFLASVYGNNSKLLSEPTNDEFQKSSGAPQRHDTSNISKPTNAASKKLLMLTDHAHKPSTNGSSSTSGAATGDVTPTVLPSHNSSVASQHPFIQEIVNRSNQNLAPINSMPSPGTFFPELNEQLNESASQKVKNHSSMMQNVEDNINQQGESIKQIHEWINKLAPTSSTTNSKKTDSDAIADDDFDVNDFFLPHTPVDEPGATSIPIIEELTPTDSLKRENGAGEGDNSASKRAKK

SEQ ID NO: 207 - protein, 536 aa, unidentified
  source 1..536: note = Pichia pastoris
MSDFPKVEQASPSEGMPQVQTNNAPEIEEIEDIIRDTPNVMDVLGNGSQSWDGTNSNGAAMGSLDTVPANKLVAYNKYDNNIYPDPLQPLSDTALPNSMGYYSMIKAGDKGLKPQSFRKTKKKPTPSGPKTRPAFVMKLWNMVHDPSNQAFIRWLPDGKSFQVTNSEDFLKHVLPKYFKHNNFASFVRQLNMYGWHKVQDVGNGSLTANEELWQFENPNFIRDREDLLDQIVRNKSKPGEDDENIDFGLVLNELETIKMNQMAISEDLRRIRQDNETLWQEHYLARERHKTQAETLEKMMRFLASVYGNNSKLLSEPTNDEFQKSSGAPQRHDTSNISKPTNAASKKLLMLTDHAHKPSTNGSSSTSGAATGDVTPTVLPSHNSSVASQHPFIQEIVNRSNQNLAPINSMPSPGTFFPELNEQLNESASQKVKNHSSMMQNVEDNINQQGESIKQIHEWINKLAPTSSTTNSKKTDSDAIADDDFDVNDFFLPHTPVDEPGATSIPIIEELTPTDSLKRENGAGEGDNSASKRAKK

SEQ ID NO: 208 - protein, 756 aa, unidentified
  source 1..756: note = Pichia pastoris
MDQDDLFPNRGSSSSSTLPDSTSLRSVDTELVKEVLEATDPVDLNHVDKLHFTNLFGMHLKQDQREFASDFEKFNKETLMEERLRSKVSKVLSSQQKSNHRLRVVGSSDTWDQKSDSNSFKTIPLNETEWNENVPIDLEKNFETIQDDVSSVEKGFIPVSRRNFVSEWYHKPKRYDIQRKLKTRNLLNIALGGTIGVGILLSSGKGFSIAGPLGCLIGFMITGMVVLATMLSFCEMVTLLPLCGGVSGVASRFVDDAFGFALGIGYWFSYTIGLPTEIIAATIMLSYYEHLHVPGPSTSAWVVFFIVVIVSINLCDVRVYGEVEYFSTIIKVLALLVLIIFMVVLNAGGVAPSHEYIGFRYWDSSKTNRTEFISNGPFRPTFDLADKGLGSFNGIGGNLGRFCSVLVACVLAAYSYVGTEIVLIAGGESQNPRKAIPAATKVIYWRIIFFYMLAIFVIGLNINSGDPRLLRFYTDGGAPADSQEQQDIQSVMDRNNGNNCHFTLLKWGGFSNGNQSPWIIALQSAGLCSFAAVLNAFLIYFALTAGSSQLYASSRTLYYLSIQGKVPKVFGICSKRGVPYISVLFTGSFSTLAFFAVEQNTVVVFSRYLSICASAGLIVWTGMCLSFIRFYYGLQLRPDIITRNDDNYPYRSPFQPYLAYFGFCMGSILVLSSGFVVFLNGHWSTTFFFTSYGSLILLFVCYFGYKILRRTSIQRLDQLDLDSGRREIDRIIWEEEKDYTVTVKGWIRFIIKKFVY

SEQ ID NO: 209 - protein, 1214 aa, unidentified
  source 1..1214: note = Pichia pastoris
MTVDSSFALRAAQSASRNQHDYQDQPIDKVVSSVMSANKRLSQASTNTNSSKRKSKNHVGPWKLGRTLGRGSTGRVRLAKHSTTGQLAAVKIVPKSSFLEQKAKDKGIAATSHRIDSNGLPYGIEREIIIMKLINHPNIMGLYDVWENKGELYLVLEYIEGGELFDYLIKNGRQPESEAVRYFKQIIDGVSYCHQFSICHRDLKPENLLLDKNSNIKIADFGMAALETKERLLETSCGSPHYASPEIIAGKDYHGSPSDVWSCGIILFALLTGRLPFDDPNIRNLLIKVQSGVFTMPEYLSKEAKDLITRMLHVDPTRRIKILDVYNHPLIQKYTDTLNFDHSYNQSTVNVVNSESPIDTVDEDILQNLQTLWKGVDRRDIISKLKNSNISSEKVFYRLLLKYRDDHSEYVLPSRRNSKKRLSNSLPRSTSIVTTTIKDDNGNTLESKTEIIRAAPTSLSSKSLAKDPIKFQRNNIKASTSRKHVSLKSSSSRKSLMKKNVSMNSVKSSAAPPRLPFANINENKSELKDFSFLCDHIFNSNRGFDEEPLLDPSSDFLFCEDETIVSNDVPLYNTPKKVNSNVLKDSTNVVTSAKRKEAKNLPKLPKDESYLSLAVTGDRTSIPDNSRNFSLDPRAASANKRNVSDTASSVLTKLGVRLSTIDIYKEYNGSSSSSTKSLNSNLNSNSSTTLTSASKPQAVTKPVGSKNAMSYQRESFLPLPTGLTFSGKLHYSSSSSTRDLASLLRPEAPKLTLKEYNSKVKKLDSVKEIAVSRKNSYRQSKAIVDEPAKKYEETILNNDTPACIDEFSFDYHDLTDSSVHVAEPVTFAKTKDVTYYSPIEEKFADVESSNDAQNTDHAISEKSIKHHRKVASDGTHDSNLNIIVQPFNDIRNSLFVDNTVNEIIEEDSRERVSEKSSNPRLSRFSQYSLSMGEPSFNQKRFTKVSIYGDPDETSNLNLEKLVNSTKDRGSLPTQYSTIFDVVDDEGNPIESKQQYESQPEKVQRGDQVKRASQFSNTLSDNDIDLGRAQSTRRRIMNQFHSIVPKRDAPRPPSHARVSPMDPIFKNSTEASTKETLQVNEKSLSKTGNEDSLKKPTWFSKLFNSLTKPKTTTKESKPNHTEVIQSSISSQRLWEIFKTTINNKQKEKTVSKVTYDHNERLISGVIPARLTGRALHFNVQIKDGEQSLVVITHHKGSKKAFRNLIIFVDQTIQVN

SEQ ID NO: 210 - protein, 78 aa, unidentified
  source 1..78: note = Pichia pastoris
MAGIKVGSGTSRNGKYQKQSKIQCIESEDIVLYQPFQTTLYAISKNGWDEILHRHDHWMQDHEVNFVRGNWFRFWEKV

SEQ ID NO: 211 - protein, 564 aa, unidentified
  source 1..564: note = Pichia pastoris
MPESRTSKGSIKSVPKKSAFVHKLYTMLSNEELNDLIWWTGPEESGTFALLPGAEFSKVLSTYFKHANVSSFVRQLHMYGFHKVSEQPLVQGDTIPKVTWEFRHSNGKFRKGNEDSLPLIKRRSTSSSSKSITTDYVKFRVHDQYSYFPQDLQYPQNPQQSNQEGETEHTQQPVLYHPQPTVYYHPNVGVPPAPPAPALAPPPFSHLGQPMQPMSHQQPQQQIQQQHHHQPQSQHGQPQQPQQQYRQYTSHPPPALLPFTPMYNMRPMTASPTNMEPMVLPTYHSMRLIETELEVKQSHLQSKSDALIKQVHDYNKHIPSLVQLIPPHFEPKVDRNIQHSRLSGIEASVRNRISKLSQPNPQPIHSAHSSFVSSKRNSSLVDPLQDLPLTAPVPNTGSGFLSAHRGFYVPKTNSVSSSSSLPVTHDLPARRTPTPFKDRNPGDSSVESSHSQLRPSIFRVHTKEEPVKAEKSSIFSNKDDSIFSNVHASSIFSQKTSIASQRSSLSMILNKPTVDSRDSITGSPLRKVSVHTIDEESTSSSNKRSHDDSNGFEDKRGKLMKLDS

SEQ ID NO: 212 - protein, 328 aa, unidentified
  source 1..328: note = Pichia pastoris
MVVAIEGGTGLGLMNLTWKPTPTPIDDAIETIRYAVEEAGVRYLNGGEFYNFPLDSNLNLQYIQEFAKRYPELYKKVSLSVKGAVSLVDVSPDSSPENLEKSISNITKHLPNNFLPIFEPARIDKRYSIEETIKNLSKFVEDGRIGGISLSEVGADTIRRAAKVAPIACVEVEFSLLTRDILHNGVLAACEDLNIPIIAYSPLGRGFLTGTINSKADIPEGDIRLSLERFNDDEVIEHNLKLVHGLKKIADKKGVTLAQLSLAWLRKFGDKHVKVLPIPSCSSPRRVAENTKEISLTDSEFQEITDFAESVPIKGGRYNKASEAVLNG

SEQ ID NO: 213 - protein, 991 aa, unidentified
  source 1..991: note = Pichia pastoris
MHHSSTEVSAIWPFFNNSYSNVAINKILQEIENEEHDNDQDAGLTAVQLLQTIRENDDIQKANDTQEPTTPSRSKESKSNKSSKTTSSGHLNKRLLNNLLIQPNLINELNAGKNSKLIAYITQEKVLSTLIDYCLESLDLKTDYVEDIDDDNEDDFNEDGSPVGHDEEEITFQYDEDNNTGKDNEKAAPADQSDSSSKRLLKRATIAMHILSSDQTPVVYNQFLSSHQYGLISKIWEGVFNRDIAEYFIDKRHNIVMMNGFIAIIENLAEMNVNGLMNFIRFQQTKDSDSLSKHFVNFIPYFPQFSDLLLKLISMDKPYNPIGLIELLVDQDLIGQILEKLRVYYDDCIIQDNLLIFLNGLVNISSNVGYWDDQQNNMENEMNDGNNGSNEANISAVNGNNTANIGPNDLTRDMVSTSKVNTMINIILNYGDYGLVTCISLFIEIIRKNNSDYDEFDWIMAANDLTSTPNSRDPIYLGVMLKLFIINLPAIVNKYLTDEYYERKQELNTFSSEGQRIDFDGKIRRKMIVSSIGKEIEPLGYERFKIMELIAELLHCSNMMLLNQSSKLDYLLFKRDELRRANQTEKLVHDALTDSILKPVEDAIEDLKIADDTSLHSFETLKKIDSKYIECTYDLSVGNSFKFSLLVSQALPRIILKMDKFPWNNFMHNVIFDLVQQIFNGKLIDDTEGTNDDENQDGDSEAEKSTEYEQNTHGFEDPLCFNKLLIVSLFGEYDAFDQDLPPDGRFARPKEIPGSFNLPAYILYACEKSKLSEEQANVKLGYMGHLVLVAQEVVKFQSIIENFGIHKEQLKTRKLEGEEIADETDDDEGETEDTEEEEQNTENGNTYDTVITNSEQFSSIKGDLPDIYKISSTKIYTRLYKQLCTSFGEGRFQKWTDFINNELSVVREQYNQVLGGVNEGEVEIDEVPRNPNAIVLDNGDSEEFRKPFDEQESETETEDEESEEEENNDNDRIREDEEAISDDNSSYDSDE

SEQ ID NO: 214 - protein, 569 aa, unidentified
  source 1..569: note = Pichia pastoris
MKIRVPFLILSCLSTAICIVNSSLAIYLCKKVSFEPTLVALAVGELIPLAGIVVVGTSQGGVLFMGCLLVSCVASLASGITWLARIVKNVERMAKYLESYYNLTICLISLFILSQVFKIVLTHFIPHSTPKIEFPKDEESFALETILNYKASAQTLVQETNIPTHLPNEGQATWCNDNNDSVKIHEPAQYSQDIKSSMQLGSERSSQQCFDPEEYKVHNHKESLKSKRSLEFEKNIIRNISNSLLPPVLQQGKSDISYLRNEMMGTPEKSHQDNENHDSEGKLFIDDLSDIPESYPQKDLWNSNRTGNISHVSLRNWNENYTDWNQRQERKGVTTELYQLNPEFRNTEQLFHNNDASSVEHMEQETSFGAPSLYSFSNNKLRQAESQDSSTVEANVGLTLMTNVGSKQTLIQEEEGLDIQSKGKQRRSSIATFKNTSPIKKLRELKNEIRSKNSVHHKSNSSLTSSIHVGMTFASAPTSPVKRKTHSLSKSMSCFHVSSQSIHRGDRSLRTVHGSPEKPNLSTLRHYPPLSDNPSSRESSQGSSCPSMFVGQYDREKWTKLKSKEEVIV

SEQ ID NO: 215 - protein, 18 aa, Equus caballus
MKCLLLALGLALMCGIQA

SEQ ID NO: 216 - protein, 18 aa, Rangifer tarandus
MKCLLITLGLALACGAQA

SEQ ID NO: 217 - protein, 16 aa, Bos taurus
MKCLLLALALTCGAQA

SEQ ID NO: 218 - protein, 18 aa, Ovis aries
MKCLLLALGLALACGVQA

SEQ ID NO: 219 - protein, 18 aa, Capra hircus
MKCLLLALGLALACGIQA

SEQ ID NO: 220 - protein, 18 aa, Arctocephalus tropicalis
MRCLLLALGLALVCGIQA

SEQ ID NO: 221 - protein, 12 aa, synthetic construct
  VARIANT 3: note = X is lysine (K) or alanine (A)
  VARIANT 7: note = X is valine (V) or serine (S)
AAXEEGXSLDKR

SEQ ID NO: 222 - protein, 8 aa, synthetic construct
MRFPSIFT

SEQ ID NO: 223 - skipped

SEQ ID NO: 224 - protein, 7 aa, synthetic construct
TTIASIA

SEQ ID NO: 225 - protein, 8 aa, synthetic construct
YVVGDDEQ

SEQ ID NO: 226 - protein, 20 aa, synthetic construct
PVFAEIDNKPVVYIVNTTKA

SEQ ID NO: 227 - protein, 20 aa, synthetic construct
ESIVAKSGITLDDLKESYAN

SEQ ID NO: 228 - protein, 8 aa, synthetic construct
  VARIANT 7: note = X is aspartic acid (D) or glycine (G)
PAEAVIXY

SEQ ID NO: 229 - protein, 10 aa, synthetic construct
  VARIANT 4: note = X is glycine (G) or glutamic acid (E)
  VARIANT 5: note = X5 is valine (V) or alanine (A)
  VARIANT 6: note = X is serine (S) or lysine (K)
  VARIANT 7: note = X is leucine (L) or asparagine (N)
  VARIANT 8: note = X is aspartic acid (D) or glycine (G)
KEEXXXXXKR

SEQ ID NO: 230 - protein, 9 aa, synthetic construct
  VARIANT 5: note = X is valine (V) or alanine (A)
  VARIANT 7: note = X is valine (V) or alanine (A)
GDFDXAXLP

SEQ ID NO: 231 - protein, 5 aa, synthetic construct
  VARIANT 1: note = X is leucine (L) or phenylalanine (F)
XSNST

SEQ ID NO: 232 - protein, 6 aa, synthetic construct
  VARIANT 4: note = X is serine (S) or phenylalanine (F)
GLSXTN

SEQ ID NO: 233 - protein, 20 aa, synthetic construct
  VARIANT 2: note = X is leucine (L) or phenylalanine (F)
  VARIANT 12: note = X is serine (S) or phenylalanine (F)
PXSNSTNNGLSXTNTTIASI

SEQ ID NO: 234 - protein, 20 aa, synthetic construct
  VARIANT 1: note = X is threonine (T) or aspartic acid (D)
  VARIANT 2: note = X is alanine (A) or leucine (L)
  VARIANT 3: note = X is glutamine (Q) or isoleucine (I)
  VARIANT 6: note = X is alanine (A) or aspartic acid (D)
  VARIANT 9: note = X is valine (V) or isoleucine (I)
  VARIANT 10: note = X is isoleucine (I) or alanine (A)
  VARIANT 11: note = X is aspartic acid (D), glycine (G), or asparagine (N)
  VARIANT 12: note = X is tyrosine (Y) or arginine (R)
  VARIANT 13: note = X serine (S) or tyrosine (Y)
  VARIANT 14: note = X is aspartic acid (D) or valine (V)
  VARIANT 15: note = X is leucine (L) or valine (V)
  VARIANT 16: note = X is glutamic acid (E) or glycine (G)
  VARIANT 17: note = X is glycine (G) or aspartic acid (D)
  VARIANT 19: note = X is phenylalanine (F) or glutamic acid (E)
  VARIANT 20: note = X is aspartic acid (D) or glutamine (Q)
XXXIPXEAXXXXXXXXXDXX

SEQ ID NO: 235 - skipped

SEQ ID NO: 236 - protein, 16 aa, synthetic construct
  VARIANT 2: note = X is alanine (A) or threonine (T)
  VARIANT 6: note = X is glycine (G) or glutamic acid (E)
  VARIANT 7: note = X is valine (V) or alanine (A)
  VARIANT 8: note = X is serine (S) or lysine (K)
  VARIANT 9: note = X is leucine (L) or asparagine (N)
  VARIANT 10: note = X is aspartic acid (D) or glycine (G)
AXKEEXXXXXKREAEA

SEQ ID NO: 237 - protein, 20 aa, synthetic construct
  VARIANT 4: note = X is alanine (A), phenylalanine (F), leucine (L), or serine (S
  VARIANT 5: note = X is leucine (L) or alanine (A)
  VARIANT 7: note = X is leucine (L) or serine (S)
  VARIANT 8: note = X is leucine (L) or valine (V)
  VARIANT 13: note = X is alanine (A) or valine (V)
SLLXXSXXLAAPXNTTTEDE

SEQ ID NO: 238 - skipped

SEQ ID NO: 239 - protein, 9 aa, synthetic construct
  VARIANT 5: note = X is valine (V) or alanine (A)
  VARIANT 7: note = X is valine (V) or alanine (A)
GDFDXAXLP

SEQ ID NO: 240 - protein, 403 aa, Anopheles christyi
MRTLAVLFAAFLAVNAKVYFEEGFKDDSWQKTWVQSEHKGVEYGKFVHTAGKFYNDAETDKGLQTSQDARFYALSNKFTPFSNKDDTLVIQFSVKHEQNIDCGGGYLKVFDCSKDLHGETPYLVMFGPDICGPGTKKVHVIFSYKGKNHLINKDIRCKDDVFTHFYTLVVRADNTYEVLIDNEKVESGSLEDDWDFLPPKKVKDPEAKKPEDWDDRATIPDPDDTKPEDWDKPEHIPDPDATKPDDWDDEMDGEWEPPMIDNPEYKGEWKPKQIDNPAYKGVWVHPEIDNPEYVEDNSLYLREEVCAVGIDVWQVKSGTIFDNFMVTNDLEEAKKVAASVKETQEGEKKVKDAQEAEERKKAEEEAAAEEAAKDDEDEDDDDDADNALPGEATELEDEGHDEL

SEQ ID NO: 241 - protein, 425 aa, Arabidopsis thaliana
MAKLNPKFISLILFALVVIVSAEVIFEEKFEDGWEKRWVKSDWKKDDNTAGEWKHTAGNWSGDANDKGIQTSEDYRFYAISAEFPEFSNKDKTLVFQFSVKHEQKLDCGGGYMKLLSDDVDQTKFGGDTPYSIMFGPDICGYSTKKVHAILTYNGTNHLIKKEVPCETDQLTHVYTFVLRPDATYSILIDNVEKQTGSLYSDWDLLPAKKIKDPSAKKPEDWDDKEYIPDPEDTKPAGYDDIPKEIPDTDAKKPEDWDDEEDGEWTAPTIPNPEYNGEWKPKKIKNPAYKGKWKAPMIDNPEFKDDPELYVFPKLKYVGVELWQVKSGSLFDNVLVSDDPEYAKKLAEETWGKHKDAEKAAFDEAEKKREEEESKDAPAESDAEEEAEDDDNEGDDSDNESKSEETKEAEETKEAEETDAAHDEL

SEQ ID NO: 242 - protein, 440 aa, unidentified
  source 1..440: note = Mucor ambiguus
MKIPTIAAVLGLAAVVSAEVFLHETFSDGEGWKDRWTASEHREDLGKLEVSPGKWFADEAYNAGLRTTEDYRFYATSTKIPKPFNNKGKDLVIQFDVKNEQDIDCGGSYLKIFGDLDPKTFNGDSEYNIMFGPDICGPKAMVHAIFNYNGTNHDLKKSISAPKDTLTHTYTLVVKPDQTYEILIDGKSKADGSLLEDWDFLPPKTIKDPNASKPEDWVEEAMIVDETDVKPANYDDIPEFIPDPEAKKPEDWDDDMDGEWEAPSIANPEYQGEWSPKKIPNPLYKGEWVHPEIDNPEYKVDNEIYAYDFANVGIDVWQVKSGTVFDNILITDDIEEAKKVLDETKALHSSEEAAQAAFNEKIQAEAKAKAEAEGAATPDEGAEKIDLEQFEPPVKFDEVPPAAAEALEKAKEEEILEAIQEEAEKKVEEEDAKKPVKDEL

SEQ ID NO: 243 - protein, 488 aa, Anopheles christyi
MSCRLVLVCLCALVAVAFAGEADVLDLTDSDFSTRVAETETTLVMFYAPWCGHCKKLKPEYAKAAELLRGEDPPIALAKVDCTEGGKDTCNKFSVSGYPTLKIFKNGEVSQEYNGPREATGIAKYMKSIVGPASKDLLTLEAFEAFLKVQETSVVGFFQKESDLKGVFLKYADSQRERLRFGHSSAPAVLEKQGDTDAVYLFRARQLANKFEPDFVKFEGTSKQELADFVKANFHGLAGVRSRDTTSDFKNPLVVVYYAVDYVKNPKGTNYWRNRVLKVAKEFVGRVNFAVSAKDDFQHELNEYGYDYTGDKPLVLARDAKNQKFIMKDEFSVENLQAFATELEEGSLEPYVKSEPVPESNDGPVKVAVAKNFDDVVVNNGVDTLVEFYAPWCGHCKKLTPTLEELGTKLKDEAVSIVKMDATANDVPSHFEVRGFPTLYWLPKDAKSSPTRYEGGREVDDFVKYIAKHATSELKGFDRSGSAKKTEL

SEQ ID NO: 244 - protein, 447 aa, unidentified
  source 1..447: note = Mucor ambiguus
MSFFKKLFFLLVAVNAAFLGYDYYQGGNIAFNLVENAKQLNGDKLHGYLAEIKSTTPEKLAGHVNNAFAQLKNINSASDILTIIREKTAPVTGAGAGSIEWDGNVVVLTDANFKNVIDGSKPALVEFYAPWCGHCKNLAPVYAQLGDAFSSSKDKVLVAKIDADQHRDTGALFGVQGFPTLKWFPKGVHSPEGVEDYKGGRDLNSLAAFIKEKSGVAPRIKSQKSDVVTLTTKNFHEVALNPKKNVLVEFYASWCGHCKNLAPIWEKIGSTFANEENCVIAKIDADEERDIGSEFDISGFPTIKFFPAGESEPVAYEGGRTEAAFVEFLNKHCGTQRKVGGGLEAAAGRIAKLDELAIRFIKNAGEREKIHAEAVEAAKEIGTRYGTYYAKIMEKMLANGEKFLETERARLAKIAGSDDVSSAKLDDFGIRQNILGAFDKKASPVKN

SEQ ID NO: 245 - protein, 508 aa, Arabidopsis thaliana
MAFKGFACFSILLLLSLFVSSIRSEETKEFVLTLDHSNFTETISKHDFIVVEFYAPWCGHCQKLAPEYEKAASELSSHNPPLALAKIDASEEANKEFANEYKIQGFPTLKILRNGGKSVQDYNGPREAEGIVTYLKKQSGPASVEIKSADSATEVVGEKNVVAVGVFPKLSGDEFDSFMALAEKLRADYDFAHTLDAKFLPRGESVEGPAVRLFKPFDELFVDSKDFNGEALEKFVKESSIPLVTVFDSDPNNHPYVAKFFESPATKAMMFVNFTGATAEALKSKYREVATSNKDQSLAFLVGDAESSQGAFQYFGLEESQVPLIIIQTPDNKKYLKVNVEVDQIESWFKDFQDGKVAVHKKSQPIPAENNEPVKVVVAESLDDIVFKSGKNVLIEFYAPWCGHCQKLAPILDEVALSFQNDPSVIIAKLDATANDIPSDTFDVKGFPTIYFRSASGNVVVYEGDRTKEDFINFVEKNSEKKPTSHGEESTKSEEPKKTEETAAKDEL